(19) United States
(12) Patent Application Publication
(10) Pub. No.: US 2025/0345339 A1
(43) Pub. Date: Nov. 13, 2025

(54) DOSES AND REGIMENS OF HER2 INHIBITORS

(71) Applicant: IAMBIC THERAPEUTICS, INC., San Diego, CA (US)

(72) Inventors: Neil Cary JOSEPHSON, Seattle, WA (US); Thomas Francis MILLER, III, La Jolla, CA (US); Frederick Roy MANBY, Bristol (GB); Chao ZHANG, Oakland, CA (US); Jacqueline Mary WALLING, Hillsborough, CA (US); Iriny BOTROUS, San Diego, CA (US); Joseph Michael DENNIS, San Diego, CA (US); Svitlana KULYK, San Diego, CA (US); Laurent GOMEZ, San Diego, CA (US); Shawn Eric WRIGHT, San Diego, CA (US); Wallace Deshaughn DERRICOTTE, Atlanta, GA (US); Zhongdong HUANG, Rancho Santa Fe, CA (US); Rampurna Prasad GULLAPALLI, San Diego, CA (US)

(21) Appl. No.: 19/057,843

(22) Filed: Feb. 19, 2025

Related U.S. Application Data
(63) Continuation of application No. PCT/US2025/016504, filed on Feb. 19, 2025.
(60) Provisional application No. 63/555,860, filed on Feb. 20, 2024.
(60) Provisional application No. 63/574,166, filed on Apr. 3, 2024.
(60) Provisional application No. 63/683,618, filed on Aug. 15, 2024.
(60) Provisional application No. 63/743,932, filed on Jan. 10, 2025.

(51) Int. Cl. A61K 31/53; A61P 35/00

(52) U.S. Cl. CPC A61K 31/53; A61P 35/00

(57) ABSTRACT

Provided herein are pharmaceutical compositions comprising a compound of having Formula I as described in this disclosure, or a pharmaceutically acceptable salt thereof, and optionally one or more pharmaceutical excipients, wherein the composition is in a unit dosage form; and methods for treating cancer modulated by HER2 in a subject, the method comprising administering to the subject an effective amount of the pharmaceutical composition or a compound having Formula II as described in this disclosure, or a pharmaceutically acceptable salt thereof, and optionally one or more additional therapeutic agents.

### CROSS-REFERENCE

This application is a continuation of International Application No.: PCT/US2025/016504, filed Feb. 19, 2025, which claims benefit to U.S. Provisional Application No. 63/555,860, filed Feb. 20, 2024, U.S. Provisional Application No. 63/574,166, filed Apr. 3, 2024, U.S. Provisional Application No. 63/683,618, filed Aug. 15, 2024, and U.S. Provisional Application No. 63/743,932, filed Jan. 10, 2025, each of which is hereby incorporated by reference in its entirety herein.

### BACKGROUND

HER2 (also referred to as Her2) belongs to the epidermal growth factor receptor (EGFR) family. This family is composed of four HER receptors: human epidermal growth factor receptor 1 (HER1) (also termed EGFR), HER2, human epidermal growth factor receptor 3 (HER3), and human epidermal growth factor receptor 4 (Her4). The HER2 receptor is a 185 kDa transmembrane protein that is encoded by the HER2 (also known as erb-b2 receptor tyrosine kinase 2 [ERBB2]) gene. HER2 is normally expressed on cell membranes of epithelial cells of several organs like the lungs, breast and the skin, as well as gastrointestinal, reproductive, and urinary tract. HER2 in normal cells is expressed at low levels, whereas in HER2-positive cancer cells, there is an increase in the number of HER2 gene copies (gene amplification) and HER2 receptors with up to 40-to-100-fold increase in protein overexpression. The increased amount of cell surface HER2 receptors associated with HER2 overexpression leads to increased receptor-receptor interactions, provoking a sustained tyrosine phosphorylation of the kinase domain and therefore constant activation of the signaling pathways.

Tumors driven by HER2 mutations or HER2 wild type over expression may benefit from tyrosine kinase inhibitors that target HER2.

Current irreversible HER2 tyrosine kinase inhibitors in clinical development include Poziotinib and Pyrotinib that both lack selectivity for HER2 mutated tumors vs. EGFR and have adverse event profiles consistent with EGFR-related toxicities. Specifically, subjects receiving poziotinib experienced Grade 3 skin rash, among other Grade 3 adverse events, that was difficult to tolerate, leading to significant dose reductions. In addition, subjects receiving pyrotinib also experienced various Grade 3 adverse events including an increase of 7 or more stools a day which usually requires hospitalization.

In many cancer types, tumor cells make extra copies of the gene that produces the HER2 protein, known as gene amplification. The resulting flood of HER2 protein causes cancer cells to grow uncontrollably, and cancer cells may also become dependent on the extra HER2 such that stopping the production of the HER2 protein can cause the cancer cells to stop growing or die. In breast cancer, 15-20% of tumors overexpress HER2 and HER2-targeted treatments are commonly used.

HER2 overexpression has been described in not only breast and gastric/gastroesophageal junction carcinomas, but somatic HER2 mutations have also been described at low frequencies in a variety of human cancers including non-small cell lung cancer, colorectal cancer, and bladder cancer. Breast cancer is a heterogeneous disease comprising various molecular subtypes, with approximately 15-20% of cases characterized by HER2-positive overexpression. Targeted therapies, such as trastuzumab, have demonstrated substantial clinical benefits for these subjects, although challenges persist, including the development of treatment resistance. Given the central role of HER2 expression in driving the disease, combining HER2-directed agents with trastuzumab has gained attention as a strategy to address HER2-related aspects of the disease from multiple angles, offering potential for improved treatment outcomes. Despite recent advances in the treatment of metastatic NSCLC, the absolute number of long-term survivors remains low.

In metastatic CRC, 3% to 5% of subjects present with HER2 alterations, and the prognosis for subjects with metastatic colorectal cancer remains poor with 5-year survival rates of 5% or less. The 5-year relative survival rate for subjects with metastatic bladder cancer is only 8%. Bladder cancer ranks third among all cancers in terms of HER2 overexpression, carrying as much as 6% to 17% of gene mutations and/or amplification in tumor tissue samples. HER2 overexpression is associated with pathological malignancy and poor prognosis indicators including carcinoma in situ, multifocal tumor, large tumor size, high tumor stage and grade, lymph node metastasis, progression, recurrence, and papillary tumor.

In recent years, increasing attention has been paid to dual anti-HER2 therapies with the aim of resolving the occurrence of toxic reactions and the development of resistance. Trastuzumab (marketed as Herceptin) is a monoclonal antibody that binds to the extracellular domain of the HER2 receptor. Tucatinib is a specific and reversible inhibitor of the protein tyrosine kinase activity of HER2, and the binding of tucatinib to the intracellular HER2 tyrosine kinase domain occurs intracellularly. Thus, tucatinib and trastuzumab block the activity of HER2 proteins but in different ways. Food and Drug Administration (FDA) granted accelerated approval to the combination of two HER2 targeted drugs, tucatinib (Tukysa) and a trastuzumab (Herceptin) for people with advanced colorectal cancer that produces an excess amount of a protein called HER2. Trastuzumab also been used for the treatment for HER2-positive breast cancer, and tucatinib has also been used in combination with trastuzumab in breast cancer.

However, most existing tyrosine kinase inhibitors, such as neratinib and poziotinib, are dual HER2-EGFR inhibitors and display significant toxicity from inhibition of EGFR. Tucatinib is the only approved HER2-selective tyrosine kinase inhibitor (approved in combination with trastuzumab for the treatment of HER2-positive colorectal cancer), but it lacks potency against exon 20 insertion mutations. There is therefore an urgent unmet need for novel anti-HER2 monotherapies and dual therapies designed to treat subjects with HER2-driven cancers, which exhibit robust activity against both HER2 and HER2 mutants (such as YVMA HER2 exon 20 insertion mutations), while preserving wild-type (WT) EGFR.

### SUMMARY

In one embodiment, this disclosure relates to a pharmaceutical composition comprising a compound and optionally one or more pharmaceutical excipients, wherein:
the composition is in a unit dosage form; and
the compound has a structure of Formula (I):

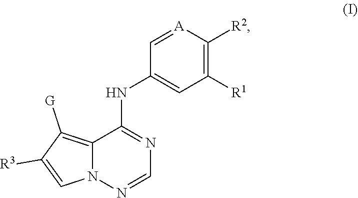

or a pharmaceutically acceptable salt thereof, wherein:
   A is N or CH;
   R¹ is C₁-C₄ alkyl, C₁-C₄ haloalkyl or halogen;
   R² is —O-(5-10 membered) aryl, —O-(5-10 membered) heteroaryl, —O-(4-7 membered) cycloalkyl, —O-(4-7 membered) heterocycloalkyl, —O-(5-10 membered) heteroaryl-C1-C4alkylene-phenyl, —NH-(5-10 membered) aryl, or —NH-(5-10 membered) heteroaryl, wherein each of the aryl, heteroaryl, cycloalkyl and heterocycloalkyl moieties are optionally substituted with 1-3 J¹ groups;
   R³ is H or F;
   G is -L¹-R³, L^{1a}-R^{3a}, or —W—X—Y;
   L¹ is a bond, —C(O)—, —S(O)₂—, —N(R^{c})—, alkylene, aryl, heteroaryl, heterocycloalkyl, or cycloalkyl, wherein the alkylene, aryl, heteroaryl, heterocycloalkyl, or cycloalkyl are each optionally substituted with 1-4 J² groups, provided that when L¹ is CH₂, L¹ is not attached to carbon or nitrogen of a saturated ring;
   L^{1a} is —C₀-C₆alkylene-C(O)N(H)—, —C₀-C₆alkylene-S(O)₂N(H)—;
   R³ is a 4-9 membered heterocyclic ring containing at least one nitrogen ring atom, wherein R³ is optionally substituted with 1-4 J³ groups, and wherein one nitrogen atom of R³ is substituted with -L²-R; or R³ is a 7-11 membered spirocyclic group containing at least one nitrogen ring atom, wherein the 7-11 membered spirocyclic group containing at least one nitrogen ring atom is optionally substituted with 1-4 J³ groups, and wherein one nitrogen atom of the 7-11 membered spirocyclic group is substituted with -L²-R;
   R^{3a} is C₁-C₆alkylene-NR^{a}R^{b} optionally substituted with 1-4 J² groups;
   W is a bond, —C(O)— or —S(O)₂—;
   X is aryl, heteroaryl, heterocycloalkyl, or cycloalkyl, each of which is optionally substituted with 1-4 J² groups;
   Y is —C₀-C₄alkylene-N(R^{d})-L²-R, —C(O)-4-7 membered heterocycloalkyl containing at least one nitrogen atom and substituted with 1-2 oxo groups, -4-7 membered heterocycloalkyl-L²R, —C₀-C₄alkylene-1-yl-1H-pyrrole-2,5-dione, —C₀-C₄alkylene-C(H)═C(O)—NH₂, —C₀-C₄alkylene-C(H)═C(H)—C(O)—O-alkyl, —C₀-C₄alkylene-ethynylene-C(O)—O-alkyl, —C₀-C₄alkylene-C(H)═C(H)—CN, —C₀-C₄alkylene-N═C═S, —C₀-C₄-etheyny, —C₀-C₄alkylene-ethynyl, —C₀-C₄alkylene-CN, —C₀-C₄alkylene-C(H)═N—N(H)Boc, —C₀-C₄alkylene-C(O)—CH₂—Br, —C₀-C₄alkylene-CH₂—Cl, —C₀-C₄alkylene-oxiranyl, —C₀-C₄alkylene-SH, —C₀-C₄alkylene-F, and —C₀-C₄alkylene-C(H)=O, wherein the C₀-C₄alkylene moiety is optionally substituted with 1-4 groups independently selected from halogen, cycloalkyl, alkoxy alkoxyalkyl, or hydroxy;
   L² is —SO₂— or —C(O)—;
   R is ethenyl optionally substituted with 1-3 Q groups, ethynyl optionally substituted with Q, C₁-C₄ alkylene-NR^{a}R^{b}, —CH₂—CN, or haloalkyl wherein one halogen of haloalkyl is on the carbon atom adjacent to L²;
   each Q is independently selected from the group consisting of halogen, haloalkyl, alkyl, alkene, alkyne, —NR^{a}R^{b}, —C₁-C₆alkylene-NR^{a}R^{b}, —C₁-C₆alkylene-OR^{c}, cyano, hydroxyalkyl, —C₀-C₆alkylene-C(O)OH, —C₁-C₆alkylene-C(O)O-alkyl, alkoxyalkyl, —C₀-C₄alkylene-cycloalkyl optionally substituted with 1-3 J⁴ groups, —C₀-C₄alkylene-cycloalkenyl optionally substituted with 1-3 J⁴ groups, —C₀-C₄alkylene-7-11 membered spirocyclic cycloalkyl, optionally substituted with 1-3 J⁴ groups, —C₀-C₄alkylene-7-11 membered spirocyclic heterocycloalkyl optionally substituted with 1-3 J⁴ groups, —C₀-C₄alkylene-heterocycloalkyl optionally substituted with 1-3 J⁴ groups, and —C₀-C₄alkylene-heterocycloalkenyl optionally substituted with 1-3 J⁴ groups;
   or -L²-R is —C═N—OH;
   each J¹ is independently selected from the group consisting of halogen, C₁-C₆alkyl, C₁-C₆haloalkyl, hydroxy, C₁-C₆hydroxyalkyl, —C₀-C₄alkylene-N(H)R^{c}, C₁-C₆alkoxy, and —C₁-C₆alkyl-C₁-C₆alkoxy;
   each J² is independently selected from the group consisting of halogen, alkyl, haloalkyl, hydroxy, hydroxyalkyl, alkoxy, and alkoxyalkyl;
   each J³ is attached to a carbon atom and is independently selected from the group consisting of halogen, haloalkyl, CN, alkyl, hydroxy, hydroxyalkyl, alkoxy, and alkoxyalkyl, or two of the optional 1-4 J³ groups form an oxo group or a 3-6 membered spiro group, or two of the optional 1-4 J³ groups are on different ring carbon and join to form a 1-3 carbon bridge;
   each J⁴ is independently selected from the group consisting of halogen, alkyl, haloalkyl, hydroxy, hydroxyalkyl, alkoxy, alkoxyalkyl, oxo, and —C₀-C₄alkylene-NR^{a}R^{b}, provided that J⁴ groups can only include up to two oxo groups and up to one —C₀-C₄alkylene-NR^{a}R^{b} group;
   R^{a} and R^{b} each are independently selected from the group consisting of H, alkyl, alkenyl, alkynyl, haloalkyl, hydroxyalkyl, alkoxyalkyl, and —C₀-C₃alkylene-alkynyl optionally substituted with alkyl, alkenyl, alkynyl, haloalkyl, hydroxyalkyl, or alkoxyalkyl; and
   R^{c} is selected from the group consisting of H, alkyl, haloalkyl, hydroxyalkyl, alkoxyalkyl, cycloalkyl, heterocycloalkyl, aryl, and heteroaryl, wherein the cycloalkyl, heterocycloalkyl, aryl, and heteroaryl groups are each optionally substituted with 1-3 groups selected from the group consisting of halogen, alkyl, alkoxy and alkoxyalkyl; and
   R^{d} is selected from the group consisting of H, alkyl, and haloalkyl.

Another embodiment of this disclosure relates to a method for treating cancer modulated by HER2 in a subject, the method comprising administering to the subject an effective amount of the pharmaceutical composition comprising a compound of Formula (I) as described in this disclosure and optionally one or more pharmaceutical excipients, wherein the composition is in a unit dosage form.

Another embodiment of this disclosure relates to a method of treating cancer modulated by HER2 in a subject, comprising administering to the subject:
(a) a compound having Formula (II):

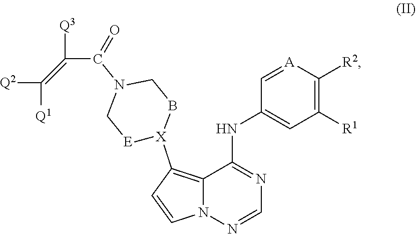

or a pharmaceutically acceptable salt thereof, wherein:
   Ais CH or N;
   B is CH₂;
   E is CH₂;
   X is CH, CF, C(OH) or N, or X is CH and B and E are both absent;
   Q¹ is selected from the group consisting of H, —C₁-C₆alkyl, F, and Cl;
   Q² is selected from the group consisting of H, F, —C₁-C₆alkylene-NR^{a}R^{b};
   Q³ is H or F;
   R^{a} and R^{b} each are independently selected from the group consisting of H, C₁-C₆alkyl, C₁-C₆haloalkyl, C₁-C₆hydroxyalkyl, and —C₁-C₆alkyl-C₁-C₆alkoxy, provided that at least one of R^{a} or R^{b} is not H;
   R¹ is alkyl, haloalkyl or halogen; and
   R² is

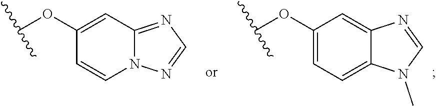 [SMILES: *Oc1ccn2ncnc2c1 ; *Oc1ccc2c(c1)ncn2C]

and (b) optionally one or more additional therapeutic agents.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1 illustrates tucatinib having weak or no activity for treating HER2+ and KRAS G12C mutation cancer.

FIG. 2 illustrates resistance to KRAS inhibitor AMG510.

FIG. 3 illustrates Compound 5 inhibiting HER2+ KRAS mutant KYSE410 tumor.

FIG. 4 illustrates Combination of Compound 5 and KRAS G12C inhibitor Adagrasib (MRTX849) synergistically inhibiting HER2+ KRAS mutant KYSE410 cell growth.

FIG. 5A illustrates Compound 5 tumor exposure (top line) and plasma concentration (bottom line) after a single PO dose of Compound 5 at 300 mg/kg in a Ba/F3 HER2YVMA xenograft model.

FIG. 5B illustrates tumor inhibition and regression in a Ba/F3-YVMA tumor model with Compound 5 dosing at 100 mg/kg (middle line) and at 300 mg/kg (bottom line) respectively compared against a vehicle (top line).

FIG. 6 illustrates the co-crystal structure of Compound 5 covalently binding to the inactive DFG-out conformation of HER2 WT (3.4 Å) overlaid with PDB structure 7PCD.

FIG. 7A illustrates tumor regression with Compound 5 at 250 mg/kg (QD, PO) and Compound 5 at 100 mg/kg (QD, PO) in comparison to tucatinib (100 mg/kg QD, PO) and T-DXd (10 mg/kg, Q3W) in N-terminally truncated HER2 (p95HER2) tumor model resistant to HER2 ADC T-DXd.

FIG. 7B illustrated weight change with various dosage levels of Compound 5 at 250 mg/kg (QD, PO) and 100 mg/kg (QD, PO) in comparison to tucatinib (100 mg/kg QD, PO) and T-DXd (10 mg/kg, Q3W).

FIG. 7C illustrates cell proliferation IC₅₀s with Compound 5 and tucatinib in Ba/F3 HER2 (p95HER2).

FIG. 8A illustrates tumor volume regression in PIK3CA H1047R mutant SKOV3 TGI model after dosing with Compound 5 at 250 mg/kg PO QD.

FIG. 8B illustrates a comparison of Compound 5 at 250 mg/kg (QD, PO) and at 100 mg/kg (QD, PO) to tucatinib (100 mg/kg QD, PO) and T-DXd (10 mg/kg, Q3W) in PIK3CA H1047R mutant HCC1954 TGI model.

FIG. 9 illustrates a brain PK profile in Ba/F3 HER2 YVMA tumor bearing mice 2 to 48 hours after dosing with Compound 5 at 300 mg/kg.

FIG. 10 illustrates that a higher selectivity threshold is needed for durable inactivation of HER2. Upregulation of HER3 and other feedback mechanisms cause hyperactivation of HER2 in HER2 driven cancers. This can raise threshold and effective dose level by 100-fold. Baseline selectivity against EGFR helps to reduce dose-limited toxicity associated with EGFR inhibition.

FIG. 11 shows that Compound 5 inhibits HER2 WT and mutants while sparing EGFR. The figure shows the potency of Compound 5 in a panel of Ba/F3 cells whose growth is driven by HER2 or EGFR expression. Compound 5 displayed low nanomolar potency across 23 representative HER2 mutations, while showing minimum effect on the growth of Ba/F3 EGFR cells. For comparison, the potency of Tucatinib in these cell lines were indicated in *.

FIG. 12A shows the comparison of full tumor and plasma PK curves in a mouse xenograft model (BaF3-HER2-YVMA). Dose of Compound is 100 mg/kg.

FIG. 12B shows the comparison of tumor PK in a mouse xenograft model (BaF3-HER2-YVMA) between 100 and 300 mg/kg doses of Compound 5.

FIG. 12C shows the peak comparison of pHER2 (Y1248) between 100 and 300 mg/kg doses of Compound 5 at various time points.

FIG. 13 illustrates that Compound 5 dosed at 100 and 250 mg/kg demonstrates tumors without a loss in body weight in PDX tumor model LU11717 (HER2-YVMA).

FIG. 14 illustrates that Compound 5 treatment lead to a dose-dependent decrease in pHER2, pHER3, pAkt and pErk1/2, which is indicative of the targeted blockage of HER2 on oncogenic signaling in cell culture. The inhibition was sustained at 24 h even when total HER3 was increased.

FIG. 15 illustrates that Compound 5 treatment lead to a robust reduction in pHER2 in xenograft tumor models. Panel (A) shows sustained inhibition by Compound 5 in Ba/F3 HER2 Y VMA xenograft tumors and (B) shows the correlation of pHER2 recovery with Compound 5 concentration in corresponding tumors.

FIG. 16A illustrates that Compound 5 (100 mg/kg) has superior efficacy when compared to tucatinib (100 mg/kg). In the MDAMB453 breast cancer model (HER2+, PIK3CA-H1047R), dosing of Compound 5 at 100 mg/kg PO daily for 14 days and caused TGI (tumor growth inhibition) of 174% in the animals (N=4 per group), whereas dosing of tucatinib was dosed in a group of mice at 100 mg/kg PO daily for 14 days and caused TGI of 90%.

FIG. 16B illustrates that Compound 5 dosed at 100 mg/kg provides for regression following tucatinib resistance. In MDAMB453 breast tumor model (HER2-amplified), the animals (N=4 per group) were dosed with tucatinib at 100 mg/kg PO daily for 14 days and caused TGI of 90%. In post tucatinib dosing, the same mice were dosed with Compound 5 at 100 mg/kg PO daily for an extended 14 days and achieved TGI of 157% in the MDAMB453 breast cancer model (HER2+, PIK3CA-H1047R).

FIG. 17 illustrates that Compound 5 is efficacious in a brain metastasis tumor model. Nude mice were intracranially implanted with NCI-N87 luciferase cells. In N87 gastric intracranial tumor model (HER2-amplified), the animals (N=10 per group) were dosed with compound 5 at 250 and 100 mg/kg PO daily for 26 days and caused TGI (tumor growth inhibition) of 169% and 120%, respectively. Enhertu was dosed in a group of mice at 5 mg/kg IV Q3W for 26 days and caused TGI of 96%. Tucatinib was dosed in a group of mice at 100 mg/kg PO daily for 26 days and caused TGI of 30%.

FIG. 18 illustrates that Compound 5 dosed at 250 and 100 mg/kg caused TGI of 182% and 169%, respectively in a NCI-H1693 NSCLC model (HER2-amplified). Dosing of enhertu at 10 mg/kg caused TGI of 119%.

FIG. 19 illustrated post enhertu dosing. In NCI-H1693 NSCLC tumor model (HER2-amplified), the animals (N=4 per group) were dosed with enhertu at 10 mg/kg IV once for 21 days and caused TGI of 119%. Post enhertu dosing, the same group of mice were dosed of compound 5 at 250 mg/kg PO daily for an extended 21 days and achieved TGI of 182%.

FIG. 20 illustrates mouse tumor growth post administration of Compound 5. In NCI-H1693 NSCLC tumor model (HER2-amplified), the animals (N=4 per group) were dosed with compound 5 at 250 and 100 mg/kg PO daily for 21 days and caused TGI (tumor growth inhibition) of 182% and 169%, respectively. After dosing stopped, the mouse tumor growth was followed up for 21 days (recovery period). In the recovery period, the tumors in the mice dosed with compound 5 at 250 mg/kg retained a TGI of 166%, where the tumors in the mice dosed with compound 5 at 100 mg/kg retained a TGI of 130%.

FIG. 21 illustrates that Compound 5 and enhertu have synergistic effect on inhibiting HER2-low NSCLC cancer. In NCI-H2030 tumor model (NSCLC cancer with Her2-low and Kras mutation), the animals (N=8 per group) were dosed with compound 5 at 250 mg/kg PO daily for 30 days and caused TGI (tumor growth inhibition) of 43%. In another group of mice, dosing of enhertu at 10 mg/kg IV Q3W for 30 days caused TGI of 84%. Combined dosing of compound 5 at 250 mg/kg PO daily and enhertu at 10 mg/kg IV Q3W for 30 days caused TGI of 99.5%. A synergy of was observed for the combination of Compound 5 and enhertu.

FIG. 22 illustrates that Compound 5 in combination with and a Kras inhibitor has synergistic effect on inhibiting NSCLC cancer. In NCI-H2122 tumor model (NSCLC cancer with Her2-low and Kras mutation), the animals (N=8 per group) were dosed with compound 5 at 100 mg/kg PO daily for 14 days and caused TGI (tumor growth inhibition) of 36%. In another group of mice, dosing of MRTX-849 at 100/75 mg/kg PO daily for 6 (at 100 mg/kg) then 8 days (at 7.5 mg/kg) caused TGI of 95%. Combined dosing of compound 5 at 100 mg/kg PO daily and MRTX-849 at 100/75 mg/kg PO daily for 6 (at 100 mg/kg) then 8 days (at 75 mg/kg) caused TGI of 99.5%. A strong synergy with the combination of Compound 5 and MRTX-849 was observed.

FIG. 23 illustrates that Compound 5 and a Kras inhibitor have synergistic effect on inhibiting pancreatic cancer. In ASPC-1 tumor model (Pancreatic cancer with Her2-low and Kras mutation), the animals (N=4 per group) were dosed with compound 5 at mostly 100 mg/kg PO daily for 21 days and caused TGI (tumor growth inhibition) of 32%. In another group of mice, dosing of RMC-6236 at 10/7.5 mg/kg PO daily for 15 (at 10 mg/kg) then 6 days (at 7.5 mg/kg) caused TGI of 89%. Combined dosing of compound 5 at mostly 100 mg/kg PO daily and RMC-6236 at 10/7.5 mg/kg PO daily for 21 days caused TGI of 171%. A strong synergy with the combination of Compound 5 and RMC-6236 was observed.

FIG. 24 illustrates that Compound 5 shows significant in vivo activity in BaF3 p95Her2 cancer model resistant to enhertu. P95Her2 is a truncated form of Her2 without extracellular domain (ECD), which is needed for enhertu to bind to. It was considered as a mechanism of resistance to Her2 antibody therapies, including enhertu. In BaF3 P95Her2 tumor model (engineered to overexpress P95Her2), the animals (N=6 per group) were dosed with compound 5 at 250 and 100 mg/kg PO daily for 20 days and caused TGI (tumor growth inhibition) of 193% and 190%, respectively. Enhertu was dosed in a group of mice at 10 mg/kg IV once for 20 days and caused TGI of 19%, showing that this model is resistant to enhertu treatment.

FIG. 25 illustrates that Compound 5 shows superior in vivo activity in HER2+ breast cancer resistant to tucatinib. In HCC1954 breast tumor model (Her2-amplified), the animals (N=8 per group) were dosed with compound 5 at 250 and 100 mg/kg PO daily for 21 days and caused TGI (tumor growth inhibition) of 184% and 176%, respectively. Enhertu was dosed in a group of mice at 10 mg/kg IV once for 21 days and caused TGI of 179%. Tucatinib was dosed in a group of mice at 100 mg/kg PO daily for 21 days and caused TGI of 30%. These results show that the model is sensitive to Compound 5 treatment.

FIG. 26 illustrates that Compound 5 and a PIK3CA inhibitor have a synergistic effect on inhibiting breast cancer. In JIMT-1 breast tumor model (Her2-amplified), the animals (N=6 per group) were dosed with compound 5 at 250 mg/kg PO daily for 21 days and caused TGI (tumor growth inhibition) of 57%. In another group of mice, dosing of alpilisib at 20/15 mg/kg PO daily for 9 (at 20 mg/kg) then 12 days (at 15 mg/kg) caused TGI of 64%. Combined dosing of compound 5 at mostly 250 mg/kg PO daily and alpilisib at 20/1.5 mg/kg PO daily for 21 days caused TGI of 164%. Overall, a strong synergy of combo with Compound 5+alpelisib was observed.

FIG. 27 illustrates the response of Compound 5 across various tumor models. Compound 5 was seen to be selective over EGFR signaling, PI3K signaling, and KRAS in most models. Compound 5 shows an inhibitory effect to MET signaling in many models.

FIG. 28 illustrates a comparison of Compound 5 (100 and 250 mg/kg), tucatinib (100 mg/kg), and enhertu (10 mg/mg) in HER2 extracellular truncation mouse xenograft models (p95 HER2 mutation Ba/F3 cell line). In BaF3 P95Her2 tumor model (engineered to overexpress P95Her2), the animals (N=6 per group) were dosed with compound 5 at 250 and 100 mg/kg PO daily for 20 days and caused TGI (tumor growth inhibition) of 193% and 190%, respectively. Enhertu was dosed in a group of mice at 10 mg/kg IV once for 20 days and caused TGI of 19%. Tucatinib was dosed in a group of mice at 100 mg/kg PO daily for 20 days and caused TGI of 99%. Application of Compound 5 achieved complete responsiveness (tumor volume=0) in half of treated mice.

FIG. 29 illustrates a comparison of Compound 5 (100 and 250 mg/kg), tucatinib (100 mg/kg), and enhertu (10 mg/mg) in HER2 amplified/PIK3CA mutation cell line mouse xenograft models (HCC1954 epithelial breast cancer cell line). Tucatinib did not achieve tumor stasis while Compound 5 achieved strong tumor regression.

FIG. 30 shows that Enhertu activity is reduced by active MET/EGFR/FGFR2/PIK3CA. Compound 5 showed activity on tumors with active MET/EGFR/PIK3CA/Kras.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

Compounds of this disclosure, which include Formula I, II, IIa, and IIb, are highly potent and irreversible tyrosine kinase inhibitor (TKI) that selectively targets human epidermal growth factor 2 (HER2) and HER2 mutants, including the exon 20 insertion mutations. The compounds of this disclosure are highly potent and selective irreversible inhibitors of both HER2 wild-type and HER2 oncogenic mutants. In another embodiment, the compounds of this disclosure selectively inhibit wild-type HER2 and/or mutant HER2 over wild-type EGFR and therefore have less EGFR-related toxicity liabilities. In another embodiment, the compounds of this disclosure selectively inhibit YVMA HER2 exon 20 insertion mutations over wild type EGFR and therefore have less EGFR-related toxicity liabilities.

Profiling data of structurally validated type II inhibitors supports the conclusion that validated type II inhibitors are generally more selective than type I inhibitors. Surprisingly, it has been found that the compounds of this disclosure bind to HER2 in a type II DFG-out conformation which was verified by a co-crystal structure. This type II DFG-out conformation is a unique binding mechanism for HER2 inhibitors that has not been previously observed with other HER2 inhibitors. Significantly, this type II DFG-out conformation locks the enzyme in the inactive conformation, and this binding mechanism underlies the strong interactions and high cellular potency against HER2, including N-terminally truncated HER2 (p95HER2) and HER2 kinase domain mutants, while sparing wild type EGFR. In certain embodiments compounds of Formula II IIa or IIb bind to HER2 in a type II DFG-out conformation.

Selectivity can be measured by the kinact/Ki is a rate constant describing the efficiency of covalent bond formation resulting from the potency (Ki) of the first reversible binding event and the maximum potential rate (kinact) of inactivation. It has been observed that compounds in this disclosure are significantly more potent as measured by Kinact/Ki) than other HER2 inhibitors.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

Unless a point of attachment indicates otherwise, the chemical moieties listed in the definitions of the variables of Formula (I) of this disclosure, and all the embodiments thereof, are to be read from left to right, wherein the right-hand side is directly attached to the parent structure as defined. However, if a point of attachment (e.g., a dash “-”) is shown on the left-hand side of the chemical moiety (e.g., —C₁-C₆alkyl-N(R⁶)₂), then the left-hand side of this chemical moiety is attached directly to the parent moiety as defined.

It is assumed that when considering generic descriptions of compounds described herein for the purpose of constructing a compound, such construction results in the creation of a stable structure. That is, one of ordinary skill in the art would recognize that, theoretically, some constructs would not normally be considered as stable compounds (that is, sterically practical and/or synthetically feasible).

“Alkyl,” by itself, or as part of another substituent, means, unless otherwise stated, a straight or branched chain hydrocarbon, having the number of carbon atoms designated (e.g. C₁-C₆ means one to six carbons). Representative alkyl groups include straight and branched chain alkyl groups having 1, 2, 3, 4, 5, 6, 7, 8, 9, 10, 11 or 12 carbon atoms. Further representative alkyl groups include straight and branched chain alkyl groups having 1, 2, 3, 4, 5, 6, 7 or 8 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like. For each of the definitions herein (e.g., alkyl, alkoxy, heterocycloalkylalkyl, heteroarylalkyl, etc.), when a prefix is not included to indicate the number of carbon atoms in an alkyl portion, the alkyl moiety or portion thereof will have 12 or fewer main chain carbon atoms or 8 or fewer main chain carbon atoms or 6 or fewer main chain carbon atoms. For example, C₁-C₆alkyl refers to a straight or branched hydrocarbon having 1, 2, 3, 4, 5 or 6 carbon atoms and includes, but is not limited to, —CH₃, C₂alkyl, C₃alkyl, C₄alkyl, C₅alkyl, C₆alkyl, C₁-C₂alkyl, C₂alkyl, C₃alkyl, C₁-C₃alkyl, C₁. C₄alkyl, C₁-C₅alkyl, C₁-C₆alkyl, C₂-C₃alkyl, C₂-C₄alkyl, C₂-C₅alkyl, C₂-C₆alkyl, C₃-C₄alkyl, C₃-C₅alkyl, C₃-C₆alkyl, C₄-C₅alkyl, C₄-C₆alkyl, C₅-C₆ alkyl and C₆alkyl. It is understood that substitutions are attached at any available atom to produce a stable compound.

“Alkylene” by itself or as part of another substituent means a linear or branched saturated divalent hydrocarbon moiety derived from an alkane having the number of carbon atoms indicated in the prefix. For example, (e.g., C₁-C₆ means one to six carbons; C₁-C₆alkylene is meant to include methylene, ethylene, propylene, 2-methylpropylene, pentylene, hexylene and the like). C₁-C₄ alkylene includes methylene —CH₂—, ethylene —CH₂CH₂—, propylene —CH₂CH₂CH₂—, and isopropylene —CH(CH₃)CH₂—, —CH₂CH(CH₃)—, —CH₂—(CH₂)₂CH₂—, —CH₂—CH(CH₃)CH₂—, —CH₂—C(CH₃)₂—CH₂—CH₂CH(CH₃)—. Typically, an alkyl (or alkylene) group will have from 1 to 24 carbon atoms, with those groups having 10 or fewer, 8 or fewer, or 6 or fewer carbon atoms. When a prefix is not included to indicate the number of carbon atoms in an alkylene portion, the alkylene moiety or portion thereof will have 12 or fewer main chain carbon atoms or 8 or fewer main chain carbon atoms, 6 or fewer main chain carbon atoms, or 4 or fewer main chain carbon atoms, or 3 or fewer main chain carbon atoms, or 2 or fewer main chain carbon atoms, or 1 carbon atom.

“Alkoxy” or “alkoxyl” refers to a —O-alkyl group, where alkyl is as defined herein. By way of example, “C₁-C₆alkoxy” refers to a —O—C₁-C₆alkyl group, where alkyl is as defined herein. While it is understood that substitutions on alkoxy are attached at any available atom to produce a stable compound, substitution of alkoxy is such that O, S, or N (except where N is a heteroaryl ring atom), are not bound to the alkyl carbon bound to the alkoxy O. Further, where alkoxy is described as a substituent of another moiety, the alkoxy oxygen is not bound to a carbon atom that is bound to an O, S, or N of the other moiety (except where N is a heteroaryl ring atom), or to an alkene or alkyne carbon of the other moiety.

“Amino” or “amine” denotes the group NH₂.

“Aryl” by itself, or as part of another substituent, unless otherwise stated, refers to a monocyclic, bicyclic or polycyclic polyunsaturated aromatic hydrocarbon radical containing 6 to 14 ring carbon atoms, which can be a single ring or multiple rings (up to three rings) which are fused together or linked covalently. Aryl, however, does not encompass or overlap in any way with heteroaryl defined below. If one or more aryl rings are fused with a heteroaryl ring, the resulting ring system is heteroaryl. Non-limiting examples of unsubstituted aryl groups include phenyl, 1-naphthyl and 2-naphthyl. The term “arylene” refers to a divalent aryl, wherein the aryl is as defined herein.

“Cycloalkyl” or “Carbocycle” or “Carbocyclic” by itself, or as part of another substituent, unless otherwise stated, refers to saturated or partially unsaturated, nonaromatic monocyclic ring, bridged rings, spiro rings, fused rings (e.g., bicyclic or tricyclic carbon ring systems), or cubane, having the number of carbon atoms indicated in the prefix or if unspecified having 3-6, also 4-6, and also 5-6 ring members per ring, such as cyclopropyl, cyclopentyl, cyclohexyl, where one or two ring carbon atoms may optionally be replaced by a carbonyl. Further, the term cycloalkyl is intended to encompass ring systems fused to an aromatic ring (e.g., of an aryl or heteroaryl), regardless of the point of attachment to the remainder of the molecule. Cycloalkyl refers to hydrocarbon rings having the indicated number of ring atoms (e.g., C₃-C₆ cycloalkyl and 3-6 membered cycloalkyl both mean three to six ring carbon atoms). The term “cycloalkenyl” refers to a cycloalkyl having at least one unit of unsaturation. A substituent of a cycloalkyl or cycloalkenyl may be at the point of attachment of the cycloalkyl or cycloalkenyl group, forming a quaternary center.

“Halogen” or “halo” refers to all halogens, that is, chloro (Cl), fluoro (F), bromo (Br), or iodo (I).

“Heteroatom” is meant to include oxygen (O), nitrogen (N), and sulfur (S).

“Heteroaryl” refers to a monocyclic or bicyclic aromatic ring radical containing 5-9 ring atoms (also referred to in this disclosure as a 5-9 membered heteroaryl, including monocyclic aromatic ring radicals containing 5 or 6 ring atoms (also referred to in this disclosure as a 5-6 membered heteroaryl), containing one or more, 14, 13, or 12, heteroatoms independently selected from the group consisting of O, S, and N. Any aromatic ring or ring system containing at least one heteroatom is a heteroaryl regardless of the point of attachment (e.g., through any one of the fused rings). Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is produced. Examples of heteroaryl groups include, but are not limited to, pyridyl, pyridazinyl, pyrazinyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, indolyl, triazinyl, quinoxalinyl, cinnolinyl, phthalazinyl, benzotriazinyl, benzimidazolyl, benzopyrazolyl, benzotriazolyl, benzisoxazolyl, isobenzofuryl, isoindolyl, indolizinyl, benzotriazinyl, thienopyridyl, thienopyrimidinyl, pyrazolopyrimidinyl, imidazopyridines, benzothiaxolyl, benzothienyl, quinolyl, isoquinolyl, indazolyl, pteridinyl and thiadiazolyl. “Nitrogen containing heteroaryl” refers to heteroaryl wherein at least one of the ring heteroatoms is N.

The term “heteroarylalkyl” refers to an alkyl group substituted with a heteroaryl group, where both terms are as defined herein.

The terms “heterocycle” or “heterocyclic ring” are interchangeable and comprise heterocycloalkyl rings and heteroaryl rings as they are defined herein. A heterocycle may be a saturated, unsaturated, or aromatic ring comprising one or more heteroatoms. Exemplary heteroatoms include N, O, P, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits.

The term “heterocycloalkyl” refers to a saturated or unsaturated non-aromatic cycloalkyl group that contains from one to five heteroatoms selected from N, O, S (including S(O) and S(O)₂), or P (including phosphine oxide) wherein the nitrogen, sulfur, and phosphorous atoms are optionally oxidized, and the nitrogen atom(s) are optionally quarternized, the remaining ring atoms being C, where one or two C atoms may optionally be present as a carbonyl. A heterocycloalkyl group can have one or more carbon-carbon double bonds or carbon-heteroatom double bonds in the ring as long as the ring is not rendered aromatic by their presence. Further, the term heterocycloalkyl is intended to encompass any ring or ring system containing at least one heteroatom that is not a heteroaryl, regardless of the point of attachment to the remainder of the molecule. Heterocycloalkyl groups include those having a ring with a formally charge-separated aromatic resonance structure, for example, N-methylpyridonyl. The heterocycloalkyl may be substituted with one or two oxo groups, and can include sulfone and sulfoxide derivatives. The heterocycloalkyl may be a monocyclic, a bridged ring system, a fused bicyclic or a fused polycyclic ring system of 3 to 12, 4 to 10, 5 to 10, or 5 to 6 ring atoms in which one to five ring atoms are heteroatoms selected from —N=, —N—, —O—, —S—, —S(O)—, or —S(O)₂— and further wherein one or two ring atoms are optionally replaced by a —C(O)— group. As an example, a 4-9 membered heterocycloalkyl is a heterocycloalkyl with 4-9 ring members having at least one heteroatom. The heterocycloalkyl can also be a heterocyclic alkyl ring fused with a cycloalkyl. Non limiting examples of heterocycloalkyl groups include pyrrolidine, piperidine, morpholine, pyridone, pyrrolidine, azepane, 1,4-diazepane, azetidine, 8-azabicylo[3.2.1]octane, 8-azabicylo[3.2.1]octene, and 3,9-diazabicyclo[4.2.1]nonane and the like. A heterocycloalkyl group can be attached to the remainder of the molecule through a ring carbon or a heteroatom. “Heterocycloalkenyl” refers to a heterocycloalkyl having at least one unit of unsaturation. A substituent of a heterocycloalkyl or heterocycloalkenyl may be at the point of attachment of the heterocycloalkyl or heterocycloalkenyl group, forming a quaternary center.

The term “heterocycloalkylalkyl” refers to an alkyl group substituted with a heterocycloalkyl group. Examples include, but are not limited to, azetidinylmethyl, morpholinomethyl, and the like.

The term “C₁-C₆haloalkyl” refers to C₁-C₆ alkyl as defined herein that is substituted with one or more halogen atoms.

The term “—C₁-C₄alkylene-NR^{a}R^{b}” refers to a “—C₁-C₄alkylene- that is attached to the parent moiety, and which substituted with NR^{a}R^{b}.

The term “C₁-C₆hydroxyalkyl” refers to C₁-C₆ alkyl as defined herein that is substituted with one or more hydroxy groups as defined herein.

The term “—C₀-C₄alkylene-C₃-C₇cycloalkyl” refers to —C₀-C₄alkylene- that is attached to the parent moiety, and which is substituted with a C₃-C₇cycloalkyl group as defined herein.

The term “oxo” refers to C(═O) or (O). In some embodiments, two possible points of attachment on a carbon form an oxo group

“Hydroxyl” or “hydroxy” refers to the group OH. The term “hydroxyalkyl” or “hydroxyalkylene” refers to an alkyl group or alkylene group, respectively as defined herein, substituted with 1-5 hydroxy groups.

The term “substituent” is an atom or group of atoms substituted in place of hydrogen atom(s) of the parent molecule. Non-limiting examples of substituents in this disclosure include J⁴ which can include monovalent or divalent substituents. Monovalent substituents are bonded to the parent moiety by replacing one hydrogen atom of the parent moiety through a single bond. The hydrogen atom that the monovalent substituent replaces may be an available hydrogen atom from a carbon or nitrogen atom of the parent moiety. Divalent substituents are bonded to the parent moiety by replacing two available hydrogen atoms of the parent moiety through a double bond. It is understood that substituents described in this disclosure cannot be attached to a parent moiety in a way that would result in an unstable molecule.

“Optional substituents” or “optionally substituted” as used throughout the disclosure means that the substitution on a compound may or may not occur, and that the description includes instances where the substitution occurs and instances in which the substitution does not. For example, the phrase “optionally substituted with 1-3 J¹ groups” means that the J¹ group may but need not be present. It is assumed in this disclosure that optional substitution on a compound occurs in a way that would result in a stable compound.

Unit dosage form” refers to a composition intended for a single administration to treat a subject suffering from a disease or medical condition. Each unit dosage form typically comprises each of the active ingredients of this disclosure plus pharmaceutically acceptable excipients. Examples of unit dosage forms are individual tablets, individual capsules, bulk powders, liquid solutions, ointments, creams, eye drops, suppositories, emulsions or suspensions. Treatment of the disease or condition may require periodic administration of unit dosage forms, for example: one unit dosage form two or more times a day, one with each meal, one every four hours or other interval, or only one per day. The expression “oral unit dosage form” indicates a unit dosage form designed to be taken orally.

In some embodiments, the unit dosage comprises the compound in an amount from about 120 mg to about 2,500 mg. In some embodiments, the unit dosage comprises the compound in an amount from about 120 mg to about 2,160 mg. In some embodiments, the unit dosage comprises the compound in an amount from about 120 mg to about 1,920 mg. In some embodiments, the unit dosage comprises the compound in an amount from about 120 mg to about 240 mg, about 120 mg to about 360 mg, about 120 mg to about 480 mg, about 120 mg to about 600 mg, about 120 mg to about 720 mg, about 120 mg to about 840 mg, about 120 mg to about 960 mg, about 120 mg to about 1,080 mg, about 120 mg to about 1,200 mg, about 120 mg to about 1,560 mg, about 120 mg to about 1,920 mg, about 240 mg to about 360 mg, about 240 mg to about 480 mg, about 240 mg to about 600 mg, about 240 mg to about 720 mg, about 240 mg to about 840 mg, about 240 mg to about 960 mg, about 240 mg to about 1,080 mg, about 240 mg to about 1,200 mg, about 240 mg to about 1,560 mg, about 240 mg to about 1,920 mg, about 360 mg to about 480 mg, about 360 mg to about 600 mg, about 360 mg to about 720 mg, about 360 mg to about 840 mg, about 360 mg to about 960 mg, about 360 mg to about 1,080 mg, about 360 mg to about 1,200 mg, about 360 mg to about 1,560 mg, about 360 mg to about 1,920 mg, about 480 mg to about 600 mg, about 480 mg to about 720 mg, about 480 mg to about 840 mg, about 480 mg to about 960 mg, about 480 mg to about 1,080 mg, about 480 mg to about 1,200 mg, about 480 mg to about 1,560 mg, about 480 mg to about 1,920 mg, about 600 mg to about 720 mg, about 600 mg to about 840 mg, about 600 mg to about 960 mg, about 600 mg to about 1,080 mg, about 600 mg to about 1,200 mg, about 600 mg to about 1,560 mg, about 600 mg to about 1,920 mg, about 720 mg to about 840 mg, about 720 mg to about 960 mg, about 720 mg to about 1,080 mg, about 720 mg to about 1,200 mg, about 720 mg to about 1,560 mg, about 720 mg to about 1,920 mg, about 840 mg to about 960 mg, about 840 mg to about 1,080 mg, about 840 mg to about 1,200 mg, about 840 mg to about 1,560 mg, about 840 mg to about 1,920 mg, about 960 mg to about 1,080 mg, about 960 mg to about 1,200 mg, about 960 mg to about 1,560 mg, about 960 mg to about 1,920 mg, about 1,080 mg to about 1,200 mg, about 1,080 mg to about 1,560 mg, about 1,080 mg to about 1,920 mg, about 1,200 mg to about 1,560 mg, about 1,200 mg to about 1,920 mg, or about 1,560 mg to about 1,920 mg. In some embodiments, the unit dosage comprises the compound in an amount from about 120 mg, about 240 mg, about 360 mg, about 480 mg, about 600 mg, about 720 mg, about 840 mg, about 960 mg, about 1,080 mg, about 1,200 mg, about 1,560 mg, or about 1,920 mg. In some embodiments, the unit dosage comprises the compound in an amount from at least about 120 mg, about 240 mg, about 360 mg, about 480 mg, about 600 mg, about 720 mg, about 840 mg, about 960 mg, about 1,080 mg, about 1,200 mg, or about 1,560 mg. In some embodiments, the unit dosage comprises the compound in an amount from at most about 240 mg, about 360 mg, about 480 mg, about 600 mg, about 720 mg, about 780 mg, about 840 mg, about 960 mg, about 1,080 mg, about 1,200 mg, about 1,560 mg, about 1,920 mg, about 2,160 mg, or about 2,500 mg.

As used herein in connection with compounds of the disclosure, the term “synthesizing” and like terms means chemical synthesis from one or more precursor materials.

As used herein, the term “composition” refers to a formulation suitable for administration to an intended animal subject for therapeutic purposes that contains at least one pharmaceutically active compound and at least one pharmaceutically acceptable carrier or excipient.

The term “pharmaceutically acceptable” indicates that the indicated material does not have properties that would cause a reasonably prudent medical practitioner to avoid administration of the material to a subject, taking into consideration the disease or conditions to be treated and the respective route of administration. For example, it is commonly required that such a material be essentially sterile, e.g., for injectables.

“Pharmaceutically acceptable salt” refers to a salt which is acceptable for administration to a subject, such as a mammal (e.g., salts having acceptable mammalian safety for a given dosage regime). Contemplated pharmaceutically acceptable salt forms include, without limitation, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug. Such salts can be derived from pharmaceutically acceptable inorganic or organic bases and from pharmaceutically-acceptable inorganic or organic acids, depending on the particular substituents found on the compounds described herein.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

When compounds of the present disclosure contain relatively acidic functionalities, base addition salts can be obtained by contacting the neutral form of such compounds with a sufficient amount of the desired base (e.g. a primary, secondary, tertiary, quaternary, or cyclic amine; an alkali metal hydroxide; alkaline earth metal hydroxide; or the like), either neat or in a suitable inert solvent. The desired acid can be, for example, a pyranosidyl acid (such as glucuronic acid or galacturonic acid), an alpha-hydroxy acid (such as citric acid or tartaric acid), an amino acid (such as aspartic acid or glutamic acid), an aromatic acid (such as benzoic acid or cinnamic acid), a sulfonic acid (such as p-toluenesulfonic acid or ethanesulfonic acid), or the like. In some embodiments, salts can be derived from pharmaceutically acceptable acids such as acetic, trifluoroacetic, propionic, ascorbic, benzenesulfonic, benzoic, camphorsulfonic, citric, ethanesulfonic, fumaric, glycolic, gluconic, glucoronic, glutamic, hippuric, hydrobromic, hydrochloric, isethionic, lactic, lactobionic, maleic, malic, malonic, mandelic, oxalic, methanesulfonic, mucic, naphthalenesulfonic, nicotinic, nitric, pamoic, pantothenic, phosphoric, succinic, sulfuric, sulfamic, hydroiodic, carbonic, tartaric, p-toluenesulfonic, pyruvic, aspartic, benzoic, cinnamic, anthranilic, mesylic, salicylic, p-hydroxybenzoic, phenylacetic, embonic (pamoic), ethanesulfonic, benzenesulfonic, 2-hydroxyethanesulfonic, sulfanilic, stearic, cyclohexylsulfamic, cyclohexylaminosulfonic, quinic, algenic, hydroxybutyric, galactaric and galacturonic acid and the like.

Also included are salts of amino acids such as arginate and the like, and salts of organic acids like glucuronic or galactunoric acids and the like (see, for example, Berge, S. M. et al., “Pharmaceutical Salts,” J. Pharmaceutical Science, 1977, 66:1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

The term “deuterated” as used herein alone or as part of a group, means substituted deuterium atoms. The term “deuterated analog” as used herein alone or as part of a group, means substituted deuterium atoms in place of hydrogen. The deuterated analog of the disclosure may be a fully or partially deuterium substituted derivative. In some embodiments, the deuterium substituted derivative of the disclosure holds a fully or partially deuterium substituted alkyl, aryl or heteroaryl group.

The disclosure also embraces isotopically-labeled compounds of the present disclosure which are identical to those recited herein, but for the fact that one or more atoms are replaced by an atom having an atomic mass or mass number different from the atomic mass or mass number usually found in nature. All isotopic variations of the compounds of the present disclosure, whether radioactive or not, are intended to be encompassed within the scope of the present disclosure. Examples of isotopes that can be incorporated into compounds of the disclosure include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Unless otherwise stated, when a position is designated specifically as “H” or “hydrogen,” the position is understood to have hydrogen at its natural abundance isotopic composition or its isotopes, such as deuterium (D) or tritium (3H). Certain isotopically-labeled compounds of the present disclosure (e.g., those labeled with ³H and ¹⁴C) are useful in compound and/or substrate tissue distribution assays. Tritiated (e.g., ³H) and carbon-14 (e.g., ¹⁴C) and fluorine-18 (¹⁸F) isotopes are useful for their ease of preparation and detectability. Further, substitution with heavier isotopes such as deuterium (e.g., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those described in the Schemes and in the Examples herein below, by substituting an isotopically labeled reagent for a non-isotopically labeled reagent.

“Prodrugs” means any compound which releases an active parent drug according to Formula (I) in vivo when such prodrug is administered to a subject. Prodrugs of a compound of Formula (I) are prepared by modifying functional groups present in the compound of Formula (I) in such a way, either in routine manipulation or in vivo, that the modifications may be cleaved in vivo to release the parent compound. Prodrugs may proceed from prodrug form to active form in a single step or may have one or more intermediate forms which may themselves have activity or may be inactive. Some prodrugs are activated enzymatically to yield the active compound, or a compound which, upon further chemical reaction, yields the active compound. Prodrugs include compounds of Formula (I) wherein a hydroxy, amino, carboxyl or sulfhydryl group in a compound of Formula (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds of Formula (I), and the like. Other examples of prodrugs include, without limitation, carbonates, ureides, solvates, or hydrates of the active compound. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

As described in The Practice of Medicinal Chemistry, Ch. 31-32 (Ed. Wermuth, Academic Press, San Diego, CA, 2001), prodrugs can be conceptually divided into two non-exclusive categories, bioprecursor prodrugs and carrier prodrugs. Generally, bioprecursor prodrugs are compounds that are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the follow types: (1) oxidative reactions: oxidative reactions are exemplified without limitation to reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions; (2) reductive reactions: reductive reactions are exemplified without limitation to reactions such as reduction of carbonyl functionalities, reduction of alcohol functionalities and carbon-carbon double bonds, reduction of nitrogen-containing functional groups, and other reduction reactions; or (3) reactions without change in the oxidation state: reactions without change in the state of oxidation are exemplified without limitation to reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. (See, e.g., Cheng et al., U.S. Patent Publ. No. 2004/0077595, incorporated herein by reference.) Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g. stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols.

The term “carrier” is also meant to include microspheres, liposomes, micelles, nanoparticles (naturally-equipped nanocarriers, for example, exosomes), and the like. It is known that exosomes can be highly effective drug carriers, and there are various ways in which drugs can be loaded into exosomes, including those techniques described in J Control Release. 2015 December 10; 219: 396-405, the contents of which are incorporated by reference in its entirety.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic process in the body of a subject. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques known in the art. See, e.g., Bertolini et al., 1997, J. Med. Chem., 40:2011-2016; Shan et al., 1997, J Pharm Sci 86(7):756-757; Bagshawe, 1995, Drug Dev. Res., 34:220-230.

“Tautomer” means compounds produced by the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The tautomers also refer to one of two or more structural isomers that exist in equilibrium and are readily converted from one isomeric form to another. Examples of include keto-enol tautomers, such as acetone/propen-2-ol, imine-enamine tautomers and the like, ring-chain tautomers, such as glucose/2,3,4,5,6-pentahydroxy-hexanal and the like, the tautomeric forms of heteroaryl groups containing a —N═C(H)—NH— ring atom arrangement, such as pyrazoles, imidazoles, benzimidazoles, triazoles, and tetrazoles. Where the compound contains, for example, a keto or oxime group or an aromatic moiety, tautomeric isomerism (‘tautomerism’) can occur. The compounds described herein may have one or more tautomers and therefore include various isomers. A person of ordinary skill in the art would recognize that other tautomeric ring atom arrangements are possible. All such isomeric forms of these compounds are expressly included in the present disclosure.

“Isomers” mean compounds that have identical molecular Formulae but differ in the nature or sequence of bonding of their atoms or in the arrangement of their atoms in space. Isomers that differ in the arrangement of their atoms in space are termed “stereoisomers.” “Stereoisomer” and “stereoisomers” refer to compounds that exist in different stereoisomeric forms, for example, if they possess one or more asymmetric centers or a double bond with asymmetric substitution and, therefore, can be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers.” When a compound has an asymmetric center, for example, an atom such as carbon bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (e.g., as (+) or (−)-isomers respectively). A chiral compound can exist as either individual enantiomer or as a mixture thereof. A mixture containing equal proportions of the enantiomers is called a “racemic mixture.” As another example, stereoisomers include geometric isomers, such as cis- or trans-orientation of substituents on adjacent carbons of a double bond. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 6th edition J. March, John Wiley and Sons, New York, 2007) differ in the chirality of one or more stereocenters.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

By “assaying” is meant the creation of experimental conditions and the gathering of data regarding a particular result of the exposure to specific experimental conditions. For example, enzymes can be assayed based on their ability to act upon a detectable substrate. A compound can be assayed based on its ability to bind to a particular target molecule or molecules.

As used herein, the terms “ligand” and “modulator” are used equivalently to refer to a compound that changes (e.g., increases or decreases) the activity of a target biomolecule, e.g., an enzyme such as those described herein. Generally a ligand or modulator will be a small molecule, where “small molecule refers to a compound with a molecular weight of 1500 Daltons or less, 1000 Daltons or less, 800 Daltons or less, or 600 Daltons or less. Thus, an “improved ligand” is one that possesses better pharmacological and/or pharmacokinetic properties than a reference compound, where “better” can be defined by one skilled in the relevant art for a particular biological system or therapeutic use. By way of example, the HER2 modulators of this disclosure are meant to include compounds that can treat any cancer characterized by HER2 aberrations such as amplification, overexpression, mutation, fusion, or truncation.

The term “binds” in connection with the interaction between a target and a potential binding compound indicates that the potential binding compound associates with the target to a statistically significant degree as compared to association with proteins generally (e.g., non-specific binding). Thus, the term “binding compound” refers to a compound that has a statistically significant association with a target molecule. In some embodiments, a binding compound interacts with a specified target with a dissociation constant (K_{D}) of 10 mM or less, 1,000 μM or less, 5000 nM or less, 3000 nM or less, 1500 nM or less, 1,000 nM or less, 500 nM or less, 200 nM or less, 100 nM or less, 50 nM or less, or 25 nM or less.

The term “selective” indicates that the compound binds more tightly than a reference compound, or than the same compound in a reference condition, e.g., with a lower dissociation constant. Certain compounds of this disclosure selectively inhibit wild-type HER2 and/or mutant Her to over wild-type EGFR thereby reducing EGFR dose-limiting toxicities.

In some embodiments, the greater affinity of one or more compounds in Table 1 is at least 1.5, 2, 3, 4, 5, 8, 10, 50, 100, 200, 400, 500, 1000, or 10,000-fold greater affinity. Certain compounds of this disclosure selectively inhibit wild-type HER2 and/or mutant Her to over wild-type EGFR thereby reducing EGFR dose-limiting toxicities.

The terms “modulate,” “modulation,” and the like refer to the ability of a compound to increase or decrease the function and/or expression of a target, such as the interaction between HER2 (including mutated forms thereof), where such function may include transcription regulatory activity and/or binding. Modulation may occur in vitro or in vivo. Modulation, as described herein, includes the inhibition, antagonism, partial antagonism, activation, agonism or partial agonism of a function or characteristic associated with HER2, either directly or indirectly, and/or the upregulation or downregulation of the expression HER2, either directly or indirectly. In another embodiment, the modulation is direct. Inhibitors or antagonists are compounds that, e.g., bind to, partially or totally block stimulation, decrease, prevent, inhibit, delay activation, inactivate, desensitize, or downregulate signal transduction. Activators or agonists are compounds that, e.g., bind to, stimulate, increase, open, activate, facilitate, enhance activation, activate, sensitize or upregulate signal transduction. In another example, compounds that modulate HER2 can do so by inhibiting HER2 by way of irreversible or covalent binding to the HER2 tyrosine kinase. In another example, compounds that modulate HER2 can do so by inhibiting HER2 by way of reversible or non-covalent binding to the HER2 tyrosine kinase.

As used herein, the terms “treat,” “treating,” “therapy,” “therapies,” and like terms refer to the administration of material, e.g., any one or more compound(s) as described herein in an amount effective to inhibit HER2, including wild-type HER2 and mutant HER2 such as HER2 with YVMA insertion mutations. In other embodiments of this disclosure, these terms apply to the administration of the compounds of this disclosure to subjects that have disease states associated with HER2 overexpression and/or HER2 amplification. In other embodiments, the terms “treat,” “treating,” “therapy,” “therapies,” and like terms refer to the administration of material, e.g., any one or more compound(s) as described herein is an amount effective to prevent, alleviate, or ameliorate one or more symptoms of a disease or condition, e.g., indication, and/or to prolong the survival of the subject being treated. In other embodiments of this disclosure, these terms apply to the administration of the compounds of this disclosure to subjects that have disease states associated with HER2 overexpression and/or HER2 amplification.

The terms “prevent,” “preventing,” “prevention” and grammatical variations thereof as used herein, refers to a method of partially or completely delaying or precluding the onset or recurrence of a disease, disorder or condition and/or one or more of its attendant symptoms or barring a subject from acquiring or reacquiring a disorder or condition or reducing a subject's risk of acquiring or requiring a disorder or condition or one or more of its attendant symptoms.

As used herein, the term “subject,” “animal subject,” and the like refers to a living organism including, but not limited to, human and non-human vertebrates, e.g. any mammal, such as a human, other primates, sports animals and animals of commercial interest such as cattle, horses, ovines, or porcines, rodents, or pets such as dogs and cats.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

In the present context, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. In general, satisfactory results in subjects are indicated to be obtained at a daily dosage of from about 0.1 to about 10 g/kg subject body weight. In some embodiments, a daily dose ranges from about 0.10 to 10.0 mg/kg of body weight, from about 1.0 to 3.0 mg/kg of body weight, from about 3 to 10 mg/kg of body weight, from about 3 to 150 mg/kg of body weight, from about 3 to 100 mg/kg of body weight, from about 10 to 100 mg/kg of body weight, from about 10 to 150 mg/kg of body weight, or from about 150 to 1000 mg/kg of body weight. The dosage can be conveniently administered, e.g., in divided doses up to four times a day or in sustained-release form.

In some embodiments, the compound is administered in a unit dosage amount from about 120 mg to about 2,500 mg. In some embodiments, the compound is administered in a unit dosage amount from about 120 mg to about 2,160 mg. In some embodiments, the compound is administered in a unit dosage amount from about 120 mg to about 1,920 mg. In some embodiments, the compound is administered in a unit dosage amount from about 120 mg to about 240 mg, about 120 mg to about 360 mg, about 120 mg to about 480 mg, about 120 mg to about 600 mg, about 120 mg to about 720 mg, about 120 mg to about 840 mg, about 120 mg to about 960 mg, about 120 mg to about 1,080 mg, about 120 mg to about 1,200 mg, about 120 mg to about 1,560 mg, about 120 mg to about 1,920 mg, about 240 mg to about 360 mg, about 240 mg to about 480 mg, about 240 mg to about 600 mg, about 240 mg to about 720 mg, about 240 mg to about 840 mg, about 240 mg to about 960 mg, about 240 mg to about 1,080 mg, about 240 mg to about 1,200 mg, about 240 mg to about 1,560 mg, about 240 mg to about 1,920 mg, about 360 mg to about 480 mg, about 360 mg to about 600 mg, about 360 mg to about 720 mg, about 360 mg to about 840 mg, about 360 mg to about 960 mg, about 360 mg to about 1,080 mg, about 360 mg to about 1,200 mg, about 360 mg to about 1,560 mg, about 360 mg to about 1,920 mg, about 480 mg to about 600 mg, about 480 mg to about 720 mg, about 480 mg to about 840 mg, about 480 mg to about 960 mg, about 480 mg to about 1,080 mg, about 480 mg to about 1,200 mg, about 480 mg to about 1,560 mg, about 480 mg to about 1,920 mg, about 600 mg to about 720 mg, about 600 mg to about 840 mg, about 600 mg to about 960 mg, about 600 mg to about 1,080 mg, about 600 mg to about 1,200 mg, about 600 mg to about 1,560 mg, about 600 mg to about 1,920 mg, about 720 mg to about 840 mg, about 720 mg to about 960 mg, about 720 mg to about 1,080 mg, about 720 mg to about 1,200 mg, about 720 mg to about 1,560 mg, about 720 mg to about 1,920 mg, about 840 mg to about 960 mg, about 840 mg to about 1,080 mg, about 840 mg to about 1,200 mg, about 840 mg to about 1,560 mg, about 840 mg to about 1,920 mg, about 960 mg to about 1,080 mg, about 960 mg to about 1,200 mg, about 960 mg to about 1,560 mg, about 960 mg to about 1,920 mg, about 1,080 mg to about 1,200 mg, about 1,080 mg to about 1,560 mg, about 1,080 mg to about 1,920 mg, about 1,200 mg to about 1,560 mg, about 1,200 mg to about 1,920 mg, or about 1,560 mg to about 1,920 mg. In some embodiments, the compound is administered in a unit dosage amount from about 120 mg, about 240 mg, about 360 mg, about 480 mg, about 600 mg, about 720 mg, about 840 mg, about 960 mg, about 1,080 mg, about 1,200 mg, about 1,560 mg, or about 1,920 mg. In some embodiments, the compound is administered in a unit dosage amount from at least about 120 mg, about 240 mg, about 360 mg, about 480 mg, about 600 mg, about 720 mg, about 840 mg, about 960 mg, about 1,080 mg, about 1,200 mg, or about 1,560 mg. In some embodiments, the compound is administered in a unit dosage amount from at most about 240 mg, about 360 mg, about 480 mg, about 600 mg, about 720 mg, about 780 mg, about 840 mg, about 960 mg, about 1,080 mg, about 1,200 mg, about 1,560 mg, about 1,920 mg, about 2,160 mg, or 2,500 mg.

In some embodiments, the compound is administered in an amount from about 120 mg to about 2,500 mg. In some embodiments, the compound is administered in an amount from about 120 mg to about 2,160 mg. In some embodiments, the compound is administered in an amount from about 120 mg to about 1,920 mg. In some embodiments, the compound is administered in an amount from about 120 mg to about 240 mg, about 120 mg to about 360 mg, about 120 mg to about 480 mg, about 120 mg to about 600 mg, about 120 mg to about 720 mg, about 120 mg to about 840 mg, about 120 mg to about 960 mg, about 120 mg to about 1,080 mg, about 120 mg to about 1,200 mg, about 120 mg to about 1,560 mg, about 120 mg to about 1,920 mg, about 240 mg to about 360 mg, about 240 mg to about 480 mg, about 240 mg to about 600 mg, about 240 mg to about 720 mg, about 240 mg to about 840 mg, about 240 mg to about 960 mg, about 240 mg to about 1,080 mg, about 240 mg to about 1,200 mg, about 240 mg to about 1,560 mg, about 240 mg to about 1,920 mg, about 360 mg to about 480 mg, about 360 mg to about 600 mg, about 360 mg to about 720 mg, about 360 mg to about 840 mg, about 360 mg to about 960 mg, about 360 mg to about 1,080 mg, about 360 mg to about 1,200 mg, about 360 mg to about 1,560 mg, about 360 mg to about 1,920 mg, about 480 mg to about 600 mg, about 480 mg to about 720 mg, about 480 mg to about 840 mg, about 480 mg to about 960 mg, about 480 mg to about 1,080 mg, about 480 mg to about 1,200 mg, about 480 mg to about 1,560 mg, about 480 mg to about 1,920 mg, about 600 mg to about 720 mg, about 600 mg to about 840 mg, about 600 mg to about 960 mg, about 600 mg to about 1,080 mg, about 600 mg to about 1,200 mg, about 600 mg to about 1,560 mg, about 600 mg to about 1,920 mg, about 720 mg to about 840 mg, about 720 mg to about 960 mg, about 720 mg to about 1,080 mg, about 720 mg to about 1,200 mg, about 720 mg to about 1,560 mg, about 720 mg to about 1,920 mg, about 840 mg to about 960 mg, about 840 mg to about 1,080 mg, about 840 mg to about 1,200 mg, about 840 mg to about 1,560 mg, about 840 mg to about 1,920 mg, about 960 mg to about 1,080 mg, about 960 mg to about 1,200 mg, about 960 mg to about 1,560 mg, about 960 mg to about 1,920 mg, about 1,080 mg to about 1,200 mg, about 1,080 mg to about 1,560 mg, about 1,080 mg to about 1,920 mg, about 1,200 mg to about 1,560 mg, about 1,200 mg to about 1,920 mg, or about 1,560 mg to about 1,920 mg. In some embodiments, the compound is administered in an amount from about 120 mg, about 240 mg, about 360 mg, about 480 mg, about 600 mg, about 720 mg, about 840 mg, about 960 mg, about 1,080 mg, about 1,200 mg, about 1,560 mg, or about 1,920 mg. In some embodiments, the compound is administered in an amount from at least about 120 mg, about 240 mg, about 360 mg, about 480 mg, about 600 mg, about 720 mg, about 780 mg, about 840 mg, about 960 mg, about 1,080 mg, about 1,200 mg, or about 1,560 mg. In some embodiments, the compound is administered in an amount from at most about 240 mg, about 360 mg, about 480 mg, about 600 mg, about 720 mg, about 840 mg, about 960 mg, about 1,080 mg, about 1,200 mg, about 1,560 mg, about 1,920 mg, about 2,160 mg, or about 2,500 mg.

As used herein, the term “HER2 mediated disease or condition” (which is also meant to mean “HER2 mediated disease or condition” as well as “wild-type HER2 and/or mutant HER2 mediated disease or condition”) refers to a disease or condition in which the biological function of HER2 affect the development and/or course of the disease or condition, and/or in which modulation of the interaction of HER2 alters the development, course, and/or symptoms. A of HER2 mediated disease or condition includes a disease or condition for which the disruption HER2 interactions (for example, by inhibiting HER2 with YVMA insertion mutations) provides a therapeutic benefit, e.g. wherein treatment with HER2 inhibitors, including compounds described herein, provides a therapeutic benefit to the subject suffering from or at risk of the disease or condition. A HER2 mediated disease or condition is intended to include a cancer or tumor that harbors loss of function mutations in HER2, or a cancer where there is activation of HER2. In another embodiments of this disclosure, HER2 mediated diseases or conditions are associated with HER2 overexpression and/or HER2 amplification. A HER2 mediated disease or condition is also intended to include various human carcinomas, including those of the lung, breast, stomach, ovary, colon, bladder, pancreatic cancer, biliary cancer, endometrial cancer, lung, uterine cervix, head and neck, gastric and esophageal cancer as well as uterine serous endometrial carcinoma, as well any associated comorbidities such as pulmonary disorder, hypertension, hypercholesterolemia, cardiovascular disease, renal function disorder, thyroid disorder, obesity, depression anxiety, osteoporosis, liver disorder, autoimmune disease, dementia, Alzheimer's disease.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. Typically, the specificity is with reference to a limited set of other biomolecules, e.g., in the case of HER2 or HER2+ mutations. In particular embodiments, the greater specificity is at least 2, 3, 4, 5, 8, 10, 20, 50, 100, 200, 400, 500, or 1000-fold greater specificity.

As used herein in connection with binding compounds or ligands, the terms “specific for HER2,” (which is intended to include either wild-type HER2, mutant HER2, or both wild-type HER2 and mutant HER2) and terms of like import mean that a particular compound binds to HER2 to a statistically greater extent than to other targets that may be present in a particular sample such as wild-type EGFR. Also, where biological activity other than binding is indicated, the terms “specific for HER2” indicates that a particular compound has greater biological effect associated with binding HER2 than to other enzymes, e.g., enzyme activity inhibition. In addition, abbreviations as used herein have respective meanings as illustrated in Table A.

TABLE A
Non-limiting examples of abbreviations

| | |
|---|---|
| ACN, MeCN | Acetonitrile |
| ° C. | Degree Celsius |
| BOC | tert-butoxycarbonyl |
| DBU | 1,8-Diazabicyclo[5.4.0]undec-7-ene |
| DCE | Dichloroethane |
| DCM | Dichloromethane |
| DIEA or DIPEA | Diisopropylethylamine |
| DMF | Dimethylformamide |
| DMSO | Dimethylsulfoxide |
| EA, ETOAc, ETAC | Ethyl acetate |
| EDC, EDAC or | 1-Ethyl-3-(3- |
| EDCI | dimethylaminopropyl)carbodiimide |
| ESI | Electrospray ionization |
| HATU | Hexafluorophosphate Azabenzotriazole |
| | Tetramethyl Uronium |
| HOBt | Hydroxybenzotriazole |
| HPLC | High Performance Liquid Chromatography |
| IC₅₀ | Half minimal (50%) inhibitory |
| | concentration |
| LCMS | Liquid Chromatography Mass |
| | Spectrometry |
| [M + H+]+ or | Mass peak plus hydrogen |
| (MH)+ | |
| [M − H−]− or (MH)− | Mass peak minus hydrogen |
| Me | Methyl |
| MeOH | Methanol |
| MS | Mass spectrometry |
| N | Normal |
| PMB | (4-methoxyphenyl)methanamine or para- |
| | methoxy benzyl |
| PyBroP | bromotri(pyrrolidino)phosphonium |
| | hexafluorophosphate |
| RP | Reverse phase |
| RT or rt | Room temperature |
| TLC | Thin-layer chromatography |
| THF | Tetrahydrofuran |
| TFA | Trifluoroacetic acid |
| ADC | antibody-drug conjugates |
| CDX | cell line derived xenograft |
| CR | complete response |
| EGFR | epidermal growth factor receptor |
| hERG | human ether-a-go-go related gene |
| HER2 | human epidermal growth factor receptor 2 |
| Kp | tumor/plasma concentration ratio |
| MTD | maximum tolerated dose |
| PD | pharmacodynamics |
| PDX | patient-derived xenograft |
| pEGFR | phosphorylated EGFR |
| pHER2 | phosphorylated HER2 |
| PK | pharmacokinetic |
| Q3W | once every 3 weeks |
| SC | subcutaneous |
| T-DXd | fam-trastuzumab deruxtecan-nxki |
| TGI | tumor growth inhibition |
| TKI | tyrosine kinase inhibitor |
| TR | tumor regression |
| WT | wild-type |

### Treatment

The treatment described in this disclosure are exemplary in nature and are not meant to limit the scope this disclosure including embodiments that are not described in these treatment protocols. In certain embodiments, a compound of this disclosure is administered as a monotherapy to subjects that have HER2 bladder cancer, colorectal cancer (CRC), or non-small cell lung cancer (NSCLC); all with or without brain metastases. In another embodiment of this monotherapy, the compound is a compound of Formula II. In another embodiment of this monotherapy, the compound is Compound 5. Compound 5 is (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-(dimethylamino)but-2-en-1-one and depicted below

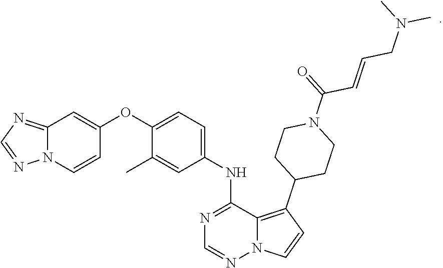 [SMILES: Cc1cc(Nc2ncnn3ccc(C4CCN(C(=O)/C=C/CN(C)C)CC4)c23)ccc1Oc1ccn2ncnc2c1]

In another embodiment, a compound of this disclosure is administered in combination with an HER2 monoclonal antibody or a HER monoclonal antibody drug conjugate to subjects that have breast cancer with brain metastases. In another embodiment of this combination therapy, the compound is a compound of Formula II. In another embodiment of this combination therapy, the compound is Compound 5.

In another embodiment, total duration of intervention for each subject can comprise a period of prescreening and screening: up to 28 days prior to the first dose of a compound described herein. In some embodiments, total duration of intervention for each subject can comprise a period of treatment: daily administration of a compound described herein in 21-day treatment cycles until disease progression, unacceptable toxicity, or withdrawal. For monotherapy dosing, subject with confirmed, relapsed/refractory malignancy with documented diagnosis of HER2 alterations can be treated sequentially into the dose cohorts starting with Cohort 1 (120 mg). Once a subject completes a 21-day treatment cycle, in the absence of a grade 2 or greater adverse event or dose limiting toxicity, escalation may proceed to the next dosing (Cohort 2) which is 240 milligrams. This dose escalation may continue to proceed in this manner to Cohort 3 (360 mg), Cohort 4 (480 mg), Cohort 5 (960 mg), and then to Cohort 6 (1560 mg). In some embodiments, the dose escalation may continue to proceed in this manner to Cohort 3 (480 mg), Cohort 4 (960 mg), Cohort 5 (1560 mg), and then to Cohort 6 (1080 mg dosed twice a day). An initial maximum tolerated dose (MTD) can be determined, and then to optimize dose selection, additional cohorts may be opened to further evaluate the safety and preliminary efficacy of the compounds of this disclosure. In another embodiment of this monotherapy dosing, the compound is a compound of Formula II. In another embodiment of this monotherapy dosing, the compound is Compound 5.

Once an initial MTD is determined, dosing may be further optimized by opening additional cohorts based on emerging data to further evaluate the safety and preliminary efficacy of the compounds of this disclosure in the following settings: monotherapy dose optimization or dose level. In some embodiments, the monotherapy dose optimization includes treating colorectal cancer subjects with or without brain metastases or non-small lung cancer subjects with brain metastases. In some embodiments, the dose levels can include administering a compound described herein to subjects in at least 2 potential dose levels with 1:1 randomization of a certain number of subjects at each dose level. In another embodiment of these monotherapy dose levels, the compound is a compound of Formula II. In another embodiment of these monotherapy dose levels, the compound is Compound 5.

In some embodiments, a compound of this disclosure can be administered to subjects at 1 dose level below the monotherapy MTD in combination with a HER2 monoclonal antibody or a HER2 monoclonal antibody drug conjugate. In another embodiment, a compound of this disclosure is dosed at the monotherapy MTD in combination with a HER2 monoclonal antibody or a HER2 monoclonal antibody drug conjugate. In another embodiment, a compound of this disclosure is dosed in combination with a HER2 monoclonal antibody or a HER2 monoclonal antibody drug conjugate in at least 2 potential dose levels with 1:1 randomization of a certain number of subjects at each dose level. In another embodiment of these combination therapy dose levels, the compound is a compound of Formula II. In another embodiment of these combination therapy dose levels, the compound is Compound 5. In another embodiment of these combination therapy dose levels, the HER2 monoclonal antibody is trastuzumab.

In some embodiments, compounds of this disclosure can be evaluated as monotherapy, and in combination with a HER2 monoclonal antibody (such as trastuzumab) or a HER2 monoclonal antibody drug conjugate by utilizing a Simon 2-stage minimax design in each of the following HER2 positive subject groups as by way of example: for subjects with bladder cancer, colorectal cancer, or non-small cell lung cancer (all with or without brain metastases, a compound of this disclosure (e.g., a compound of Formula II or Compound 5) can be administered monotherapy at the confirmed MTD or for subjects with breast cancer with brain metastases. In some embodiments, Compound 5 can be administered at the confirmed MTD in combination with trastuzumab. Subjects may continue to receive a compound of this disclosure (e.g., a compound of Formula II or Compound 5) if trastuzumab is discontinued, but a subject can be discontinued from all study treatment if the compound of this disclosure is discontinued. In another embodiment, a compound of this disclosure is dosed at the monotherapy MTD in combination with a HER2 monoclonal antibody or a HER2 monoclonal antibody drug conjugate. In another embodiment, a compound of this disclosure is dosed in combination with a HER2 monoclonal antibody or a HER2 monoclonal antibody drug conjugate in at least 2 potential dose levels with 1:1 randomization of a certain number of subjects at each dose level. In another embodiment of these combination therapy dose levels, the compound is a compound of Formula II. In another embodiment of these combination therapy dose levels, the compound is Compound 5. In another embodiment of these combination therapy dose levels, the HER2 monoclonal antibody is trastuzumab. In another embodiment of the Simon 2-stage evaluation, the treatment period can be daily dosing in 21-day treatment cycles until disease progression, unacceptable toxicity, or withdrawal.

In another embodiment, the subject population includes adult subjects with relapsed/refractory malignancy with documented HER2 alterations. The selection of the subject population for the compounds of this disclosure is guided by a tangible and pressing medical need, e.g., effectively targeting HER2 oncogenic drivers in a range of solid tumors. This focus is driven by the role played by HER2 overexpression and mutation in various cancer types, including breast cancer, CRC, NSCLC, and bladder cancer.

In other embodiments of the treatment protocols of this disclosure, the compound of this disclosure is administered to subjects with water under fasting conditions. In other embodiments of the treatment protocols of this disclosure, subjects fast for at least 2 hours before and 1 hour after taking the compound of this disclosure.

In other embodiments of the treatment protocols of this disclosure, the compound is administered to subjects as an oral, immediate release, two-piece hard gelatin capsule containing about 30 mg or about 120 mg of the compound. In another embodiment, the compounds that is administered in the treatment protocols of this disclosure is a compound of Formula II. In another embodiment, the compounds that is administered in the treatment protocols of this disclosure, the compound that is administered is Compound 5.

In other embodiments of the treatment protocols of this disclosure, the compound of Formula II is administered to subjects in need thereof in combination with a recombinant humanized monoclonal antibody directed against HER2 such as, for example, trastuzumab. In other embodiments of the treatment protocols of this disclosure, Compound 5 is administered to subjects in need thereof with a recombinant humanized monoclonal antibody directed against HER2 such as, for example, trastuzumab. Trastuzumab is available as 150 mg lyophilized powder in a single-dose vial for reconstitution to be given by intravenous (IV) injection. Additional information about trastuzumab is available in the trastuzumab prescribing information.

In another embodiment in the treatment protocol of this disclosure, subjects with breast cancer are administered, in combination with the compound of this disclosure (such as Formula II or compound 5), trastuzumab as standard of care and as described in the prescribing information (4 mg/kg 90 minute IV followed by subsequent weekly doses of 2 mg/kg IV 30 minutes infusion weekly).

One variable that can be measured in the treatment protocols of this disclosure is overall response rate (ORR), defined as achieving a best response of complete response (CR) or partial response (PR). The analysis may be performed on the response evaluable set, including all subjects who have received at least one dose of the compound of this disclosure with or without trastuzumab, have at least one measurable lesion at baseline, and have at least one post-baseline response assessment. The summary statistic can be the ORR rate which can be supported by a 95% exact Clopper-Pearson confidence interval.

Another variable that can be measured in the treatment protocols of this disclosure is progression free survival (PFS) which is defined as the number of months from the date of first study treatment administration to the earliest of documented progressive disease or death without prior progression. A death will be considered a PFS event.

Another variable that can be measured in the treatment protocols of this disclosure is overall survival (OS) which is defined as the number of months from the date of first study treatment administration to the date of death, irrespective of cause.

Another variable that can be measured in the treatment protocols of this disclosure is best overall response (BoR) which is generally the best response across all assessments for a

Subject that can be measured from the first day of treatment up until disease progression.

Another variable that can be measured in the treatment protocols of this disclosure is duration of response defined as the time between first disease response and date of disease

progression or death due to any cause.

Another variable that can be measured in the treatment protocols of this disclosure is disease control rate (DCR) which is defined as the percentage of subjects who achieved disease response or stable disease (SD) consecutively for 3 months.

Another variable that can be measured in the treatment protocols of this disclosure is clinical benefit rate (CB) which is defined as the percentage of subjects who achieve complete response, partial response, or stable disease.

Another variable that can be measured in the treatment protocols for breast cancer and NSCLC subjects are efficacy endpoints that can be determined intracranially.

Described herein, in some aspects, is a method for treating cancer modulated by HER2 in a subject. In some embodiments, the method comprising administering to the subject an effective amount of a compound described herein or a pharmaceutical composition comprising the compound. In some embodiments, the administration comprises administering an additional therapeutic agent. In some embodiments, the additional therapeutic agent comprises a HER2 monoclonal antibody or a HER2 antibody drug conjugate. In some embodiments, the additional therapeutic agent comprises adagrasib, pertuzumab, margetuximab, or trastuzumab. The use of adagrasib, pertuzumab, margetuximab, or trastuzumab (drugs for treating cancer associated with KRAS mutation) in combination with HER2 treatment is unexpected as patient selection guideline for HER2 cancer treatment typically excludes the patients harboring KRAS mutation.

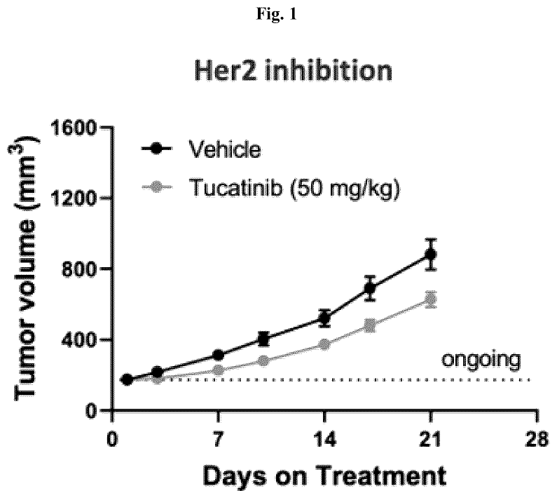

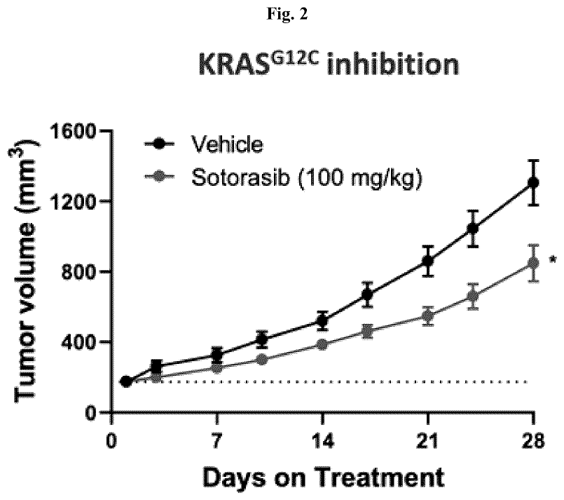

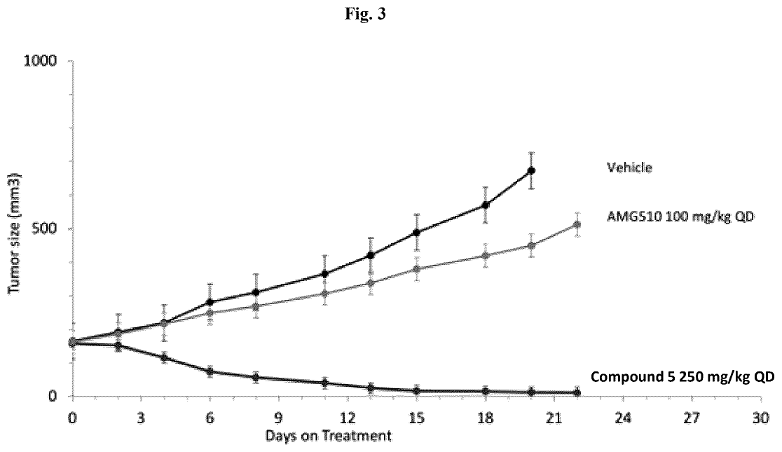

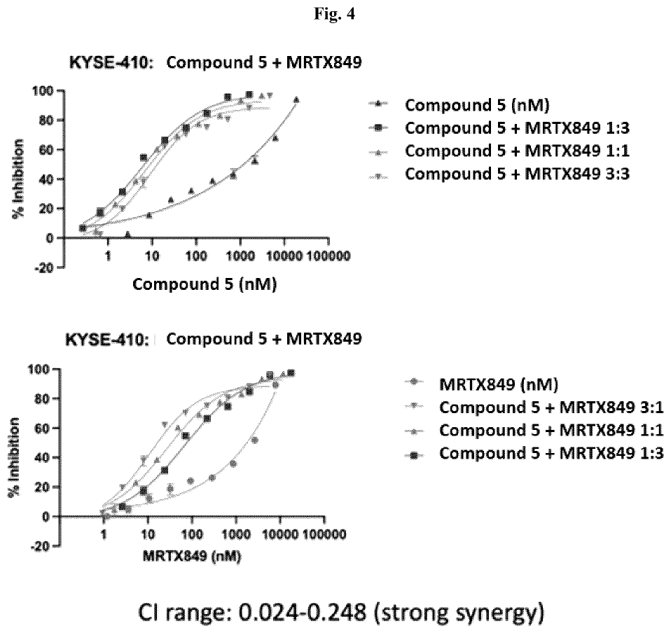

As shown in FIG. 1, tucatinib, a HER2 inhibitor, was ineffective in treating cancer harboring both HER2 overexpression and KRAS G12C mutation. As shown in FIG. 2, AMG510, a KRAS inhibitor, was similarly ineffective in treating cancer harboring both HER2 overexpression and KRAS G12C mutation. However, as shown in FIG. 3, Compound 5 at 250 mg/kg dosage caused near complete tumor regression. AMG510 showed marginal activity. These data suggest therapeutic efficacy of Compound 5 (and its derivatives with similar activity profile) for treatment for HER2+ KRAS mutant tumors and metastasis in subjects. The therapeutic effect of Compound 5 can be synergistically increased by utilizing Compound 5 in combination with a KRAS inhibitor. As shown in FIG. 4, the combinatorial effect of Compound 5 and KRAS G12C inhibitor MRTX849 was tested on inhibiting HER2+ KRAS mutant KYSE410 cell growth. The achieved combinatorial index (CI) of the Compound 5 and MRTX849 combination was in the range of 0.024-0.248, indicating that Compound 5 and MRTX849 synergistically inhibited HER2+ KRAS mutant KYSE410 cell growth. Accordingly, the compound of the present disclosure presents an improvement for treating cancer both modulated by HER2 and associated with KRAS mutation.

In some embodiments, the patient population will have failed at least one anti-cancer therapy. In some embodiments, the at least one anti-cancer therapy is zongertinib, ELVN-002, BI-4142, ponziotinib, or tucatinib. In some embodiments, the at least one anti-cancer therapy is tucatinib.

In some embodiments, the cancer to be treated by a compound described herein is a cancer associated with HER2 modulation or KRAS mutation. In some embodiments, the cancer is associated with HER2 modulation and KRAS mutation. In some embodiments, the HER2 modulation comprises HER2 overexpression, HER2 amplification, HER2 mutation, or a combination thereof. In some embodiments, the cancer is associated with HER2 overexpression and KRAS mutation. In some embodiments, the cancer is associated with HER2 amplification and KRAS mutation. In some embodiments, the cancer is associated with HER2 mutation and KRAS mutation. In some embodiments, the KRAS mutation comprises at least one mutation in exon 2 of KRAS. In some embodiments, the KRAS mutation comprises at least one mutation in exon 3 of KRAS. In some embodiments, the KRAS mutation comprises at least one mutation in exon 4 of KRAS. In some embodiments, the KRAS mutation comprises: G12A mutation (exon 2, codon 12); G12C mutation (exon 2, codon 12); G12D mutation (exon 2, codon 12); G12E mutation (exon 2, codon 12); G12F mutation (exon 2, codon 12); G12H mutation (exon 2, codon 12); G12I mutation (exon 2, codon 12); G12L mutation (exon 2, codon 12); G12R mutation (exon 2, codon 12); G12S mutation (exon 2, codon 12); G12V mutation (exon 2, codon 12); G12Y mutation (exon 2, codon 12); G13A mutation (exon 2, codon 13); G13C mutation (exon 2, codon 13); G13D mutation (exon 2, codon 13); G13E mutation (exon 2, codon 13); G13F mutation (exon 2, codon 13); G13P mutation (exon 2, codon 13); G13R mutation (exon 2, codon 13); G13V mutation (exon 2, codon 13); G13S mutation (exon 2, codon 13); G13Y mutation (exon 2, codon 13); A59E mutation (exon 3, codon 59); A59G mutation (exon 3, codon 59), A59T mutation (exon 3, codon 59); Q61A mutation (exon 3, codon 61); Q61E mutation (exon 3, codon 61); Q61H mutation (exon 3, codon 61); Q61K mutation (exon 3, codon 61); Q61L mutation (exon 3, codon 61); Q61P mutation (exon 3, codon 61); Q61R mutation (exon 3, codon 61); K117N mutation (exon 4, codon 117); A146P mutation (exon 4, codon 146); A146T mutation (exon 4, codon 146); A146V mutation (exon 4, codon 146), or a combination thereof.

In some embodiments, the method does not inhibit EGFR signaling. In some embodiments, the HER2 inhibition is more than 2 fold, 5 fold, or 10 fold higher than inhibition of EGFR signaling.

In some embodiments, the method comprises inhibiting one or more HER2 mutants while sparing EGFR signaling. In some embodiments, the one or more HER2 mutants is selected from P95HER2, A775-G776-ins-YVMA, A775-G776-ins-YVMA-R678Q, A775-G776-ins-YVMS, A775-G776-ins-C, A775-G776-ins-MMAY, A775-G776-ins-SVMA, A775-G776-ins-VVMA, G776VC, G776-del-ins-AVGC, G776-del-ins-IC, G776-del-ins-LC, G776-del-ins-VV, G776-V777-del-ins-CVC, P780-Y781-ins-GSP, S310F, S310Y, L755S, D769N, V777L, L786V, T798M, V842I, and L869R.

In some embodiments, the method additionally inhibits MET signaling. In some embodiments, the HER2 inhibition is at least 0.5 fold, 1 fold, 2 fold, 5 fold, or 10 fold lower than inhibition of PI3K signaling.

In some embodiments, the method does not inhibit PI3K signaling. In some embodiments, the HER2 inhibition is more than 2 fold, 5 fold, or 10 fold higher than inhibition of PI3K signaling.

### EMBODIMENTS

Embodiment 1 of this disclosure relates to a pharmaceutical composition comprising a compound and optionally one or more pharmaceutical excipients, wherein:
the composition is in a unit dosage form; and
the compound has a structure of Formula (I):

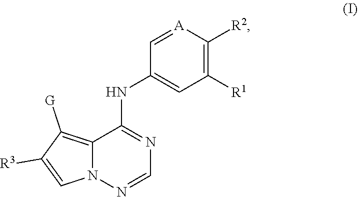

or a pharmaceutically acceptable salt thereof, wherein:
   A is N or CH;
   R¹ is C₁-C₄ alkyl, C₁-C₄ haloalkyl or halogen;
   R² is —O-(5-10 membered) aryl, —O-(5-10 membered) heteroaryl, —O-(4-7 membered) cycloalkyl, —O-(4-7 membered) heterocycloalkyl, —O-(5-10 membered) heteroaryl-C1-C4alkylene-phenyl, —NH-(5-10 membered) aryl, or —NH—-(5-10 membered) heteroaryl, wherein each of the aryl, heteroaryl, cycloalkyl and heterocycloalkyl moieties are optionally substituted with 1-3 J¹ groups;
   R³ is H or F;
   G is -L¹-R³, L^{1a}-R^{3a}, or —W—X—Y;
   L¹ is a bond, —C(O)—, —S(O)₂—, —N(R^{c})—, alkylene, aryl, heteroaryl, heterocycloalkyl, or cycloalkyl, wherein the alkylene, aryl, heteroaryl, heterocycloalkyl, or cycloalkyl are each optionally substituted with 1-4 J² groups, provided that when L¹ is CH₂, L¹ is not attached to carbon or nitrogen of a saturated ring;
   L^{1a} is —C₀-C₆alkylene-C(O)N(H)—, —C₀-C₆alkylene-S(O)₂N(H)—;
   R³ is a 4-9 membered heterocyclic ring containing at least one nitrogen ring atom, wherein R³ is optionally substituted with 1-4 J³ groups, and wherein one nitrogen atom of
   R³ is substituted with -L²-R; or R³ is a 7-11 membered spirocyclic group containing at least one nitrogen ring atom, wherein the 7-11 membered spirocyclic group containing at least one nitrogen ring atom is optionally substituted with 1-4 J³ groups, and wherein one nitrogen atom of the 7-11 membered spirocyclic group is substituted with -L²-R;
   R^{3a} is C₁-C₆alkylene-NR^{a}R^{b} optionally substituted with 1-4 J² groups;
   W is a bond, —C(O)— or —S(O)₂—;
   X is aryl, heteroaryl, heterocycloalkyl, or cycloalkyl, each of which is optionally substituted with 1-4 J² groups;
   Y is —C₀-C₄alkylene-N(R^{d})-L²-R, —C(O)-4-7 membered heterocycloalkyl containing at least one nitrogen atom and substituted with 1-2 oxo groups, -4-7 membered heterocycloalkyl-L²R, —C₀-C₄alkylene-1-yl-1H-pyrrole-2,5-dione, —C₀-C₄alkylene-C(H)═C(O)—NH₂, —C₀-C₄alkylene-C(H)═C(H)—C(O)—O-alkyl, —C₀-C₄alkylene-ethynylene-C(O)—O-alkyl, —C₀-C₄alkylene-C(H)═C(H)—CN, —C₀-C₄alkylene-N═C═S, —C₀-C₄-etheyny, —C₀-C₄alkylene-ethynyl, —C₀-C₄alkylene-CN, —C₀-C₄alkylene-C(H)=N—N(H)Boc, —C₀-C₄alkylene-C(O)—CH₂—Br, —C₀-C₄alkylene-CH₂—Cl, —C₀-C₄alkylene-oxiranyl, —C₀-C₄alkylene-SH, —C₀-C₄alkylene-F, and —C₀-C₄alkylene-C(H)=O, wherein the C₀-C₄alkylene moiety is optionally substituted with 1-4 groups independently selected from halogen, cycloalkyl, alkoxy alkoxyalkyl, or hydroxy;
   L² is —SO₂— or —C(O)—;
   R is ethenyl optionally substituted with 1-3 Q groups, ethynyl optionally substituted with Q, C₁-C₄ alkylene-NR^{a}R^{b}, —CH₂—CN, or haloalkyl wherein one halogen of haloalkyl is on the carbon atom adjacent to L²;
   each Q is independently selected from the group consisting of halogen, haloalkyl, alkyl, alkene, alkyne, —NR^{a}R^{b}, —C₁-C₆alkylene-NR^{a}R^{b}, —C₁-C₆alkylene-OR^{c}, cyano, hydroxyalkyl, —C₀-C₆alkylene-C(O)OH, —C₁-C₆alkylene-C(O)O-alkyl, alkoxyalkyl, —C₀-C₄alkylene-cycloalkyl optionally substituted with 1-3 J⁴ groups, —C₀-C₄alkylene-cycloalkenyl optionally substituted with 1-3 J⁴ groups, —C₀-C₄alkylene-7-11 membered spirocyclic cycloalkyl, optionally substituted with 1-3 J⁴ groups, —C₀-C₄alkylene-7-11 membered spirocyclic heterocycloalkyl optionally substituted with 1-3 J⁴ groups, —C₀-C₄alkylene-heterocycloalkyl optionally substituted with 1-3 J⁴ groups, and —C₀-C₄alkylene-heterocycloalkenyl optionally substituted with 1-3 J⁴ groups;
   or -L²-R is —C═N—OH;
   each J¹ is independently selected from the group consisting of halogen, C₁-C₆alkyl, C₁-C₆haloalkyl, hydroxy, C₁-C₆hydroxyalkyl, —C₀-C₄alkylene-N(H)R^{c}, C₁-C₆alkoxy, and —C₁-C₆alkyl-C₁-C₆alkoxy;
   each J² is independently selected from the group consisting of halogen, alkyl, haloalkyl, hydroxy, hydroxyalkyl, alkoxy, and alkoxyalkyl;
   each J³ is attached to a carbon atom and is independently selected from the group consisting of halogen, haloalkyl, CN, alkyl, hydroxy, hydroxyalkyl, alkoxy, and alkoxyalkyl, or two of the optional 1-4 J³ groups form an oxo group or a 3-6 membered spiro group, or two of the optional 1-4 J³ groups are on different ring carbon and join to form a 1-3 carbon bridge;
   each J⁴ is independently selected from the group consisting of halogen, alkyl, haloalkyl, hydroxy, hydroxyalkyl, alkoxy, alkoxyalkyl, oxo, and —C₀-C₄alkylene-NR^{a}R^{b}, provided that J⁴ groups can only include up to two oxo groups and up to one —C₀-C₄alkylene-NR^{a}R^{b} group;
   R^{a} and R^{b} each are independently selected from the group consisting of H, alkyl, alkenyl, alkynyl, haloalkyl, hydroxyalkyl, alkoxyalkyl, and —C₀-C₃alkylene-alkynyl optionally substituted with alkyl, alkenyl, alkynyl, haloalkyl, hydroxyalkyl, or alkoxyalkyl; and
   R^{c} is selected from the group consisting of H, alkyl, haloalkyl, hydroxyalkyl, alkoxyalkyl, cycloalkyl, heterocycloalkyl, aryl, and heteroaryl, wherein the cycloalkyl, heterocycloalkyl, aryl, and heteroaryl groups are each optionally substituted with 1-3 groups selected from the group consisting of halogen, alkyl, alkoxy and alkoxyalkyl; and
   R^{d} is selected from the group consisting of H, alkyl, and haloalkyl.

Embodiment 2 of this disclosure relates to the pharmaceutical composition of Embodiment 1, wherein:
G is -L¹-R³;
L¹ is a bond;
R³ is a 4-9 membered heterocyclic ring containing at least one nitrogen ring atom, wherein R³ is optionally substituted with 1-4 J³ groups, and wherein one nitrogen atom of R³ is substituted with -L²-R;
L² is —C(O)—;
R is ethenyl substituted with 1 Q group;
Q is —C₁-C₆alkylene-NR^{a}R^{b}; and
R^{a} and R^{b} are alkyl.

Embodiment 3 of this disclosure relates to the pharmaceutical composition of Embodiments 1 or 2, wherein A is CH.

Embodiment 4 of this disclosure relates the pharmaceutical composition of any one of Embodiments 1 to Error! Reference source not found., wherein R¹ is C₁-C₄alkyl.

Embodiment 5 of this disclosure relates to the pharmaceutical composition of any one of Embodiments 1 to Error! Reference source not found., wherein R² is —O-(5-10 membered) heteroaryl.

Embodiment 6 of this disclosure relates to the pharmaceutical composition of any one of Embodiments 1 to Error! Reference source not found., wherein:
A is CH;
R¹ is C₁-C₃ alkyl;
R² is —O-(5-10 membered) heteroaryl;
G is -L¹-R³;
L¹ is a bond;
R³ is a 5-6 membered heterocyclic ring containing at least one nitrogen ring atom, wherein one nitrogen atom of R³ is substituted with -L²-R;
L² is —C(O)—;
R is ethenyl substituted with 1 Q group;
Q is —C₁-C₃alkylene-NR^{a}R^{b}; and
R^{a} and R^{b} are methyl.

Embodiment 7 of this disclosure relates to the pharmaceutical composition of any one of Embodiments 1 to 3, wherein the compound is:

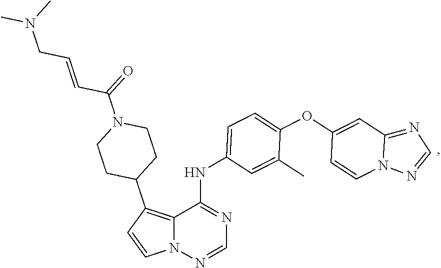 [SMILES: Cc1cc(Nc2ncnn3ccc(C4CCN(C(=O)/C=C/CN(C)C)CC4)c23)ccc1Oc1ccn2ncnc2c1]

or a pharmaceutically acceptable salt thereof.

Embodiment 8 of this disclosure relates to the pharmaceutical composition of any one of Embodiments 1 to 4, wherein the compound ranges from about 50 mg to about 600 mg per unit dose, 50 mg to about 720 mg per unit dose, 50 mg to about 840 mg per unit dose, 50 mg to about 960 mg per unit dose, 50 mg to about 1080 mg per unit dose, 50 mg to about 1200 mg per unit dose, 50 mg to about 1320 mg per unit dose, 50 mg to about 1440 mg per unit dose, 50 mg to about 1560 mg per unit dose, 50 mg to about 1680 mg per unit dose, 50 mg to about 1800 mg per unit dose, or 50 mg to about 1920 mg per unit dose.

Embodiment 9 of this disclosure relates to the pharmaceutical composition of any one of Embodiment 1 to Error! Reference source not found., wherein the compound ranges from about 120 mg to about 600 mg per unit dose, 120 mg to about 720 mg per unit dose, 120 mg to about 840 mg per unit dose, 120 mg to about 960 mg per unit dose, 120 mg to about 1080 mg per unit dose, 120 mg to about 1200 mg per unit dose, 120 mg to about 1320 mg per unit dose, 120 mg to about 1440 mg per unit dose, 120 mg to about 1560 mg per unit dose, 120 mg to about 1680 mg per unit dose, 120 mg to about 1800 mg per unit dose, 120 mg to about 1920 mg per unit dose, or 120 mg to about 2150 mg per unit dose.

Embodiment 10 of this disclosure relates to the pharmaceutical composition of Embodiment Error! Reference source not found., wherein the compound ranges from about 120 mg to about 2150 mg per unit dose.

Embodiment 11 of this disclosure relates to the pharmaceutical composition of Embodiment Error! Reference source not found., wherein the compound ranges from about 60 mg to about 1920 mg per unit dose.

Embodiment 12 of this disclosure relates to the pharmaceutical composition of any one of Embodiments 1 to 5, wherein the compound ranges from about 120 mg to about 1560 mg per unit dose.

Embodiment 13 of this disclosure relates to the pharmaceutical composition of any one of Embodiments 1 to 5, wherein the compound ranges from about 240 mg to about 1560 mg per unit dose.

Embodiment 14 of this disclosure relates to the pharmaceutical composition of any one of Embodiments 1 to 5, wherein the compound ranges from about 480 mg to about 1560 mg per unit dose.

Embodiment 15 of this disclosure relates to the pharmaceutical composition of any one of Embodiments 1 to 5, wherein the compound ranges from about 960 mg to about 1560 mg per unit dose.

Embodiment 16 of this disclosure relates to the pharmaceutical composition of Embodiment Error! Reference source not found., wherein the compound ranges from about 60 mg to about 480 mg per unit dose.

Embodiment 17 of this disclosure relates to the pharmaceutical composition of Embodiment 5, wherein the compound ranges from about 120 mg to about 360 mg per unit dose.

Embodiment 18 of this disclosure relates to the pharmaceutical composition of any one of Embodiment 1 to 9, wherein the unit dosage of the compound is about 60 mg, about 120 mg, about 180 mg, about 240 mg, about 360 mg, about 480 mg, about 600 mg, about 720 mg, about 780 mg, about 840 mg, about 960 mg, about 1080 mg, about 1200 mg, about 1320 mg, about 1440 mg, about 1560 mg, about 1680 mg, about 1800 mg, about 1920 mg, or about 2160 mg.

Embodiment 19 of this disclosure relates to the pharmaceutical composition of Embodiment 18, wherein the unit dosage of the compound is about 60 mg.

Embodiment 20 of this disclosure relates to a pharmaceutical composition of Embodiment 18, wherein the unit dosage of the compound is about 120 mg.

Embodiment 21 of this disclosure relates to the pharmaceutical composition of Embodiment 18, wherein the unit dosage of the compound is about 240 mg.

Embodiment 22 of this disclosure relates to the pharmaceutical composition of Embodiment 18, wherein the unit dosage of the compound is about 360 mg.

Embodiment 23 of this disclosure relates to the pharmaceutical composition of Embodiment 18, wherein the unit dosage of the compound is about 480 mg.

Embodiment 24 of this disclosure relates to the pharmaceutical composition of Embodiment 18, wherein the unit dosage of the compound is about 600 mg.

Embodiment 25 of this disclosure relates to the pharmaceutical composition of Embodiment 18, wherein the unit dosage of the compound is about 720 mg.

Embodiment 26 of this disclosure relates to the pharmaceutical composition of Embodiment 18, wherein the unit dosage of the compound is about 780 mg.

Embodiment 27 of this disclosure relates to the pharmaceutical composition of Embodiment 18, wherein the unit dosage of the compound is about 840 mg.

Embodiment 28 of this disclosure relates to the pharmaceutical composition of Embodiment 18, wherein the unit dosage of the compound is about 960 mg.

Embodiment 29 of this disclosure relates to the pharmaceutical composition of Embodiment 18, wherein the unit dosage of the compound is about 1080 mg.

Embodiment 30 of this disclosure relates to the pharmaceutical composition of Embodiment 18, wherein the unit dosage of the compound is about 1200 mg.

Embodiment 31 of this disclosure relates to the pharmaceutical composition of Embodiment 18, wherein the unit dosage of the compound is about 1320 mg.

Embodiment 32 of this disclosure relates to the pharmaceutical composition of Embodiment 18, wherein the unit dosage of the compound is about 1440 mg.

Embodiment 33 of this disclosure relates to the pharmaceutical composition of Embodiment 18, wherein the unit dosage of the compound is about 1560 mg.

Embodiment 34 of this disclosure relates to the pharmaceutical composition of Embodiment 18, wherein the unit dosage of the compound is about 1680 mg.

Embodiment 35 of this disclosure relates to the pharmaceutical composition of Embodiment 18, wherein the unit dosage of the compound is about 1800 mg.

Embodiment 36 of this disclosure relates to the pharmaceutical composition of Embodiment 18, wherein the unit dosage of the compound is about 1920 mg.

Embodiment 37 of this disclosure relates to the pharmaceutical composition of Embodiment 18, wherein the unit dosage of the compound is about 2160 mg.

Embodiment 38 of this disclosure relates to the pharmaceutical composition of any one of Embodiments 1 to 37, wherein the unit dosage is in capsule or tablet form.

Embodiment 39 of this disclosure relates to the pharmaceutical composition of Embodiment 38, wherein the unit dosage is a two-piece hard gelatin capsule.

Embodiment 40 of this disclosure relates to a method for treating cancer modulated by HER2 in a subject, the method comprising administering to the subject an effective amount of the pharmaceutical composition of any one of Embodiments 1 to 39.

Embodiment 40(a) of this disclosure relates to the method of Embodiment 37, wherein the compound in the pharmaceutical composition binds to HER2 in a type II DFG-out conformation.

Embodiment 41 of this disclosure relates to the method of any one of Embodiments 40 or 40a, wherein the subject has failed at least one cancer therapeutic.

Embodiment 42 of this disclosure relates to the method of Embodiment 41, wherein the at least one cancer therapeutic is zongertinib, ELVN-002, BI-4142, poziotinib, or tucatinib.

Embodiment 43 of this disclosure relates to the method of Embodiment 40 or 40(a), wherein the pharmaceutical composition is administered orally once daily.

Embodiment 44 of this disclosure relates to the method of Embodiment 40 or 40(a), wherein the pharmaceutical composition is administered orally twice daily.

Embodiment 45 of this disclosure relates to the method of any one of Embodiments 40 to 44, wherein the pharmaceutical composition is administered daily for about 21 days to about six months.

Embodiment 46 of this disclosure relates to the method of Embodiment 45, wherein the pharmaceutical composition is administered daily for about 21 days.

Embodiment 47 of this disclosure relates to the method of Embodiment 45, wherein the pharmaceutical composition is administered daily for about 3 months.

Embodiment 48 of this disclosure relates to the method of any one of Embodiments 40 to 47, wherein the method further comprises administering an additional therapeutic agent.

Embodiment 49 of this disclosure relates to the method of Embodiment 48, wherein the additional therapeutic agent is a HER2 monoclonal antibody or a HER2 antibody drug conjugate.

Embodiment 50 of this disclosure relates to the method of Embodiment 49, wherein the additional therapeutic agent is adagrasib, pertuzumab, margetuximab, or trastuzumab.

Embodiment 51 of this disclosure relates to the method of Embodiment 50, wherein the additional therapeutic agent is trastuzumab.

Embodiment 52 of this disclosure relates to the method of Embodiment 51, wherein the additional therapeutic agent is ado-trastuzumab emtansine or trastuzumab deruxtecan.

Embodiment 53 of this disclosure relates to the method of Embodiment 48, wherein the additional therapeutic agent is a KRAS inhibitor or a PIK3CA inhibitor.

Embodiment 54 of this disclosure relates to the method of Embodiment 48, wherein the additional therapeutic agent is alpilisib, or RMC-6236, MRTX-849.

Embodiment 55 of this disclosure relates to the method of Embodiment 40 to 53, wherein the cancer is colorectal cancer, breast cancer, bladder cancer, pancreatic cancer, biliary cancer, endometrial cancer, gastric cancer, esophageal squamous cancer, ovarian cancer, or non-small cell lung cancer.

Embodiment 56 of this disclosure relates to the method of Embodiment 55, wherein the cancer is breast cancer with brain metastasis, colorectal cancer with or without brain metastasis, bladder cancer with or without brain metastasis or non-small cell lung cancer with or without brain metastasis.

Embodiment 57 of this disclosure relates to the method of Embodiment 40 to 56, wherein the subject is in a fed state.

Embodiment 58 of this disclosure relates to the method of Embodiment 40 to 56, wherein the subject is in a fasted state.

Embodiment 59 of this disclosure relates to the method of Embodiment 58, wherein the subject has fasted for at least 2 hours.

Embodiment 60 of this disclosure relates to the method of Embodiment 58, wherein the subject has a low fat content diet.

Embodiment 61 of this disclosure relates to the method of Embodiment 40 to 60, wherein the cancer is associated with HER2 modulation or KRAS mutation.

Embodiment 62 of this disclosure relates to the method of Embodiment 40 to 61, wherein the cancer is associated with HER2 modulation and KRAS mutation.

Embodiment 63 of this disclosure relates to the method of Embodiment 53 or Embodiment 62, wherein the HER2 modulation comprises HER2 overexpression, HER2 amplification, HER2 mutation, or a combination thereof.

Embodiment 64 of this disclosure relates to the method of Embodiment 61 to 63, wherein the KRAS mutation comprises at least one mutation in exon 2 of KRAS.

Embodiment 65 of this disclosure relates to the method of Embodiment 61 to 63, wherein the KRAS mutation comprises at least one mutation in exon 3 of KRAS.

Embodiment 66 of this disclosure relates to the method of Embodiment 61 to 63, wherein the KRAS mutation comprises at least one mutation in exon 4 of KRAS.

Embodiment 67 of this disclosure relates to the method of Embodiment 61 to 66, wherein the KRAS mutation comprises: G12A mutation (exon 2, codon 12); G12C mutation (exon 2, codon 12); G12D mutation (exon 2, codon 12); G12E mutation (exon 2, codon 12); G12F mutation (exon 2, codon 12); G12H mutation (exon 2, codon 12); G12I mutation (exon 2, codon 12); G12L mutation (exon 2, codon 12); G12R mutation (exon 2, codon 12); G12S mutation (exon 2, codon 12); G12V mutation (exon 2, codon 12); G12Y mutation (exon 2, codon 12); G13A mutation (exon 2, codon 13); G13C mutation (exon 2, codon 13); G13D mutation (exon 2, codon 13); G13E mutation (exon 2, codon 13); G13F mutation (exon 2, codon 13); G13P mutation (exon 2, codon 13); G13R mutation (exon 2, codon 13); G13V mutation (exon 2, codon 13); G13S mutation (exon 2, codon 13); G13Y mutation (exon 2, codon 13); A59E mutation (exon 3, codon 59); A59G mutation (exon 3, codon 59), A59T mutation (exon 3, codon 59); Q61A mutation (exon 3, codon 61); Q61E mutation (exon 3, codon 61); Q61H mutation (exon 3, codon 61); Q61K mutation (exon 3, codon 61); Q61L mutation (exon 3, codon 61); Q61P mutation (exon 3, codon 61); Q61R mutation (exon 3, codon 61); K117N mutation (exon 4, codon 117); A146P mutation (exon 4, codon 146); A146T mutation (exon 4, codon 146); A146V mutation (exon 4, codon 146), or a combination thereof.

Embodiment 68 of this disclosure relates to the method of Embodiment 67, wherein the KRAS mutation comprises the G12C mutation.

Embodiment 69 of this disclosure relates to a method of treating cancer modulated by HER2 in a subject, comprising administering to the subject a compound having Formula (II):

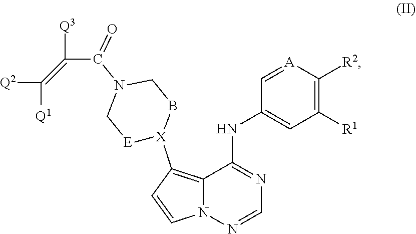

or a pharmaceutically acceptable salt thereof, wherein:
A is CH or N;
B is CH₂;
E is CH₂;
X is CH, CF, C(OH) or N, or X is CH and B and E are both absent;
Q¹ is selected from the group consisting of H, —C₁-C₆alkyl, F, and Cl;
Q² is selected from the group consisting of H, F, —C₁-C₆alkylene-NR^{a}R^{b};
Q³ is H or F;
R^{a} and R^{b} each are independently selected from the group consisting of H, C₁-C₆alkyl, C₁-C₆haloalkyl, C₁-C₆hydroxyalkyl, and —C₁-C₆alkyl-C₁-C₆alkoxy, provided that at least one of R^{a} or R^{b} is not H;
R¹ is alkyl, haloalkyl or halogen; and
R² is

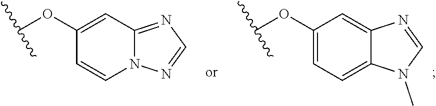 [SMILES: *Oc1ccn2ncnc2c1 ; *Oc1ccc2c(c1)ncn2C]

and (b) optionally one or more additional therapeutic agents.

Embodiment 69(a) of this disclosure relates to the method of Embodiment 69, wherein the compound of Formula (II) binds to HER2 in a type II DFG-out conformation.

Embodiment 70 relates to the method of Embodiment 69 or 69(a), wherein the compound is Formula (IIa)

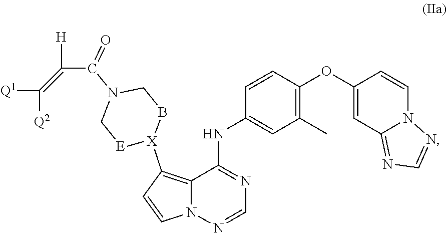

or a pharmaceutically acceptable salt thereof, wherein:
X is CH or N;
B is CH₂, E is CH₂, or A is CH and B and E are both absent;
Q¹ is selected from the group consisting of H, —C₁-C₃alkyl, F, and Cl;
Q² is selected from the group consisting of H, F, —C₁-C₃alkylene-NR^{a}R^{b}; and
R^{a} and R^{b} each are independently selected from the group consisting of H, C₁-C₃alkyl, C₁-C₃haloalkyl, C₃-C₃hydroxyalkyl, and —C₁-C₃alkyl-C₁-C₃alkoxy, provided that at least one of R^{a} or R^{b} is not H; and (b) optionally one or more additional therapeutic agents.

Embodiment 71 relates to the method of Embodiment 70, wherein X is CH.

Embodiment 72 relates to the method of Embodiment 70, wherein A is CH, and B and E are both absent to form a compound of Formula (IIb):

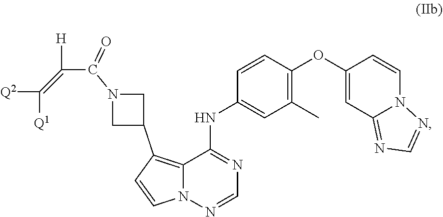

Embodiment 73 relates to the method of any one of Embodiments 69, 69(a), and 70 to 72, wherein Q¹ and Q² are H.

Embodiment 74 relates to the method of any one of Embodiments 69, 69(a), and 70 to 72, wherein Q¹ is H and Q² is —C₁-C₃alkylene-N(C₁-C₃alkyl)₂.

Embodiment 75 relates to the method of Embodiment 70, wherein the compound has one of the following structures:

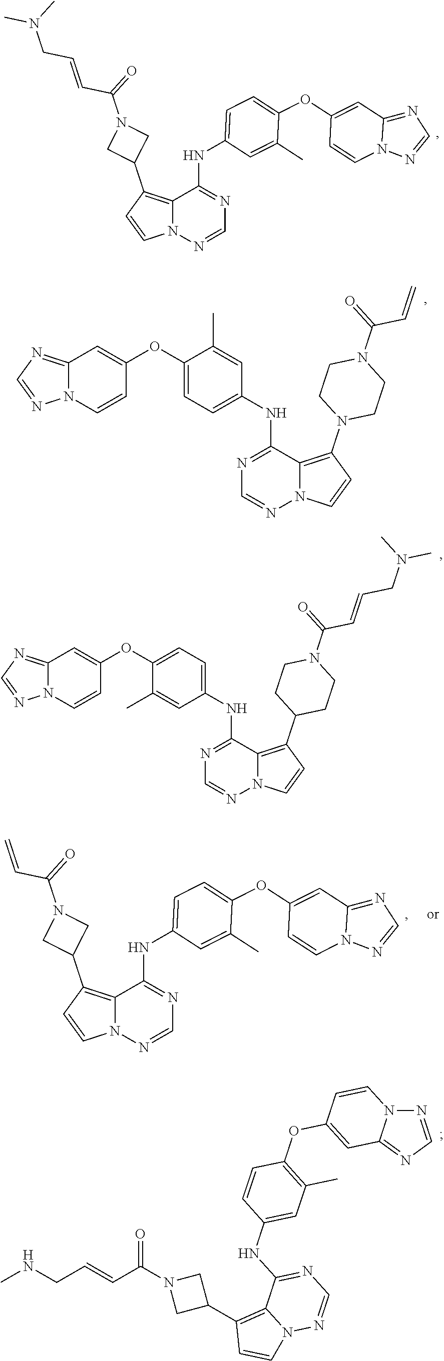

or a pharmaceutically acceptable salt thereof.

Embodiment 76 relates to the method of the method of Embodiment 75, wherein the compound has the following structure:

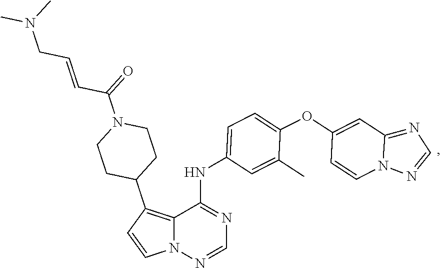 [SMILES: Cc1cc(Nc2ncnn3ccc(C4CCN(C(=O)/C=C/CN(C)C)CC4)c23)ccc1Oc1ccn2ncnc2c1]

or a pharmaceutically acceptable salt thereof.

Embodiment 77 of this disclosure relates to the method of Embodiment 69 to 76 (the range including 69(a) here and throughout), wherein the compound is administered in a unit dosage amount ranging from about 50 mg to about 600 mg, 50 mg to about 720 mg per unit dose, 50 mg to about 840 mg per unit dose, 50 mg to about 960 mg per unit dose, 50 mg to about 1080 mg per unit dose, 50 mg to about 1200 mg per unit dose, 50 mg to about 1320 mg per unit dose, 50 mg to about 1440 mg per unit dose, 50 mg to about 1560 mg per unit dose, 50 mg to about 1680 mg per unit dose, 50 mg to about 1800 mg per unit dose, 50 mg to about 1920 mg per unit dose, or 50 mg to about 2160 mg per unit dose.

Embodiment 78 of this disclosure relates to the method of Embodiment 69 to 77, wherein the compound ranges from about 120 mg to about 600 mg per unit dose, 120 mg to about 720 mg per unit dose, 120 mg to about 840 mg per unit dose, 120 mg to about 960 mg per unit dose, 120 mg to about 1080 mg per unit dose, 120 mg to about 1200 mg per unit dose, 120 mg to about 1320 mg per unit dose, 120 mg to about 1440 mg per unit dose, 120 mg to about 1560 mg per unit dose, 120 mg to about 1680 mg per unit dose, 120 mg to about 1800 mg per unit dose, 120 mg to about 1920 mg per unit dose, or 120 mg to about 2160 mg per unit dose.

Embodiment 79 of this disclosure relates to the method of Embodiment 78, wherein the compound ranges from about 60 mg to about 1920 mg per unit dose.

Embodiment 80 of this disclosure relates to the method of Embodiment 78, wherein the compound is administered in a unit dosage amount ranging from about 120 mg to about 1560 mg per unit dose.

Embodiment 81 of this disclosure relates to the method of Embodiment 78, wherein the compound is administered in a unit dosage amount ranging from about 240 mg to about 1560 mg per unit dose.

Embodiment 82 of this disclosure relates to the method of Embodiment 78, wherein the compound is administered in a unit dosage amount ranging from about 480 mg to about 1560 mg per unit dose.

Embodiment 83 of this disclosure relates to the method of Embodiment 78, wherein the compound is administered in a unit dosage amount ranging from about 960 mg to about 1560 mg per unit dose.

Embodiment 84 of this disclosure relates to the method of Embodiment 78, wherein the compound is administered in a unit dosage amount ranging from about 60 mg to about 480 mg per unit dose.

Embodiment 85 of this disclosure relates to the method of Embodiment 78, wherein the compound is administered in a unit dosage amount ranging from about 120 mg to about 360 mg per unit dose.

Embodiment 86 of this disclosure relates to the method of Embodiment 69 to 78, wherein the compound is administered in a unit dosage amount of about 60 mg, about 120 mg, about 180 mg, about 240 mg, about 360 mg, about 480 mg, about 600 mg, about 720 mg, about 780 mg, about 840 mg, about 960 mg, about 1080 mg, about 1200 mg, about 1320 mg, about 1440 mg, about 1560 mg, about 1680 mg, about 1800 mg, about 1920 mg, or about 2160 mg.

Embodiment 87 of this disclosure relates to the method of Embodiment 86, wherein the compound is administered in a unit dosage amount of 60 mg.

Embodiment 88 of this disclosure relates to the method of Embodiment 86, wherein the compound is administered in a unit dosage amount of about 120 mg.

Embodiment 89 of this disclosure relates to the method of Embodiment 86, wherein the compound is administered in a unit dosage amount of about 240 mg.

Embodiment 90 of this disclosure relates to the method of Embodiment 86, wherein the compound is administered in a unit dosage amount of about 360 mg.

Embodiment 91 of this disclosure relates to the method of Embodiment 86, wherein the compound is administered in a unit dosage amount of about 480 mg.

Embodiment 92 of this disclosure relates to the method of Embodiment 86, wherein the compound is administered in a unit dosage amount of about 600 mg.

Embodiment 93 of this disclosure relates to the method of Embodiment 86, wherein the compound is administered in a unit dosage amount of about 720 mg.

Embodiment 94 of this disclosure relates to the method of Embodiment 86, wherein the compound is administered in a unit dosage amount of about 780 mg.

Embodiment 95 of this disclosure relates to the method of Embodiment 86, wherein the compound is administered in a unit dosage amount of about 840 mg.

Embodiment 96 of this disclosure relates to the method of Embodiment 86, wherein the compound is administered in a unit dosage amount of about 960 mg.

Embodiment 97 of this disclosure relates to the method of Embodiment 86, wherein the compound is administered in a unit dosage amount of about 1080 mg.

Embodiment 98 of this disclosure relates to the method of Embodiment 86, wherein the compound is administered in a unit dosage amount of about 1200 mg.

Embodiment 99 of this disclosure relates to the method of Embodiment 86, wherein the compound is administered in a unit dosage amount of about 1320 mg.

Embodiment 100 of this disclosure relates to the method of Embodiment 86, wherein the compound is administered in a unit dosage amount of about 1440 mg.

Embodiment 101 of this disclosure relates to the method of Embodiment 86, wherein the compound is administered in a unit dosage amount of about 1560 mg.

Embodiment 102 of this disclosure relates to the method of Embodiment 86, wherein the compound is administered in a unit dosage amount of about 1680 mg.

Embodiment 103 of this disclosure relates to the method of Embodiment 86, wherein the compound is administered in a unit dosage amount of about 1800 mg.

Embodiment 104 of this disclosure relates to the method of Embodiment 86, wherein the compound is administered in a unit dosage amount of about 1920 mg.

Embodiment 105 of this disclosure relates to the method of Embodiment 86, wherein the compound is administered in a unit dosage amount of about 2160 mg.

Embodiment 106 of this disclosure relates to the method of Embodiment 69 to 105, wherein the unit dosage is in capsule or tablet form.

Embodiment 107 of this disclosure relates to the method of Embodiment 69 to 106, wherein the compound is administered orally once daily.

Embodiment 108 of this disclosure relates to the method of Embodiment 69 to 106, wherein the compound is administered orally twice daily.

Embodiment 109 of this disclosure relates to the method of Embodiment 69 to 108, wherein the compound is administered in an amount from about 50 mg to about 600 mg, 50 mg to about 720 mg, 50 mg to about 840 mg, 50 mg to about 960 mg, 50 mg to about 1080 mg, 50 mg to about 1200 mg, 50 mg to about 1320 mg, 50 mg to about 1440 mg, 50 mg to about 1560 mg, 50 mg to about 1680 mg, 50 mg to about 1800 mg, 50 mg to about 1920 mg, or 50 mg to about 2160 mg.

Embodiment 110 of this disclosure relates to the method of Embodiment 69 to 106, wherein the compound is administered in an amount from about 120 mg to about 2160 mg.

Embodiment 111 of this disclosure relates to the method of Embodiment 69 to 108, wherein the compound is administered in an amount from about 480 mg to about 1560 mg.

Embodiment 112 of this disclosure relates to the method of Embodiment 69 to 108, wherein the compound is administered in an amount from about 960 mg to about 1560 mg.

Embodiment 113 of this disclosure relates to the method of Embodiment 69 to 108, wherein the compound is administered in an amount from about 960 mg to about 1200 mg.

Embodiment 114 of this disclosure relates to the method of Embodiment 69 to 108, wherein the compound is administered in an amount from about 1200 mg to about 1560 mg.

Embodiment 115 of this disclosure relates to the method of Embodiment 67 to 104, wherein the compound is administered in an amount of about 60 mg, about 120 mg, about 180 mg, about 240 mg, about 360 mg, about 480 mg, about 600 mg, about 720 mg, about 780 mg, about 840 mg, about 960 mg, about 1080 mg, about 1200 mg, about 1320 mg, about 1440 mg, about 1560 mg, about 1680 mg, about 1800 mg, about 1920 mg, or about 2160 mg.

Embodiment 116 of this disclosure relates to the method of Embodiment 115, wherein the compound is administered in an amount of about 480 mg once per day.

Embodiment 117 of this disclosure relates to the method of Embodiment 115, wherein the compound is administered in an amount of about 600 mg once per day.

Embodiment 118 of this disclosure relates to the method of Embodiment 115, wherein the compound is administered in an amount of about 720 mg once per day.

Embodiment 119 of this disclosure relates to the method of Embodiment 115, wherein the compound is administered in an amount of about 780 mg once per day.

Embodiment 120 of this disclosure relates to the method of Embodiment 115, wherein the compound is administered in an amount of about 840 mg once per day.

Embodiment 121 of this disclosure relates to the method of Embodiment 115, wherein the compound is administered in an amount of about 960 mg once per day.

Embodiment 122 of this disclosure relates to the method of Embodiment 115, wherein the compound is administered in an amount of about 1080 mg once per day.

Embodiment 123 of this disclosure relates to the method of Embodiment 115, wherein the compound is administered in an amount of about 1200 mg once per day.

Embodiment 124 of this disclosure relates to the method of Embodiment 115, wherein the compound is administered in an amount of about 1320 mg once per day.

Embodiment 125 of this disclosure relates to the method of Embodiment 115, wherein the compound is administered in an amount of about 1440 mg once per day.

Embodiment 126 of this disclosure relates to the method of Embodiment 115, wherein the compound is administered in an amount of about 1560 mg once per day.

Embodiment 127 of this disclosure relates to the method of Embodiment 115, wherein the compound is administered in an amount of about 1680 mg once per day.

Embodiment 128 of this disclosure relates to the method of Embodiment 115, wherein the compound is administered in an amount of about 1800 mg once per day.

Embodiment 129 of this disclosure relates to the method of Embodiment 115, wherein the compound is administered in an amount of about 1920 mg once per day.

Embodiment 130 of this disclosure relates to the method of Embodiment 115, wherein the compound is administered in an amount of about 2160 mg once per day.

Embodiment 131 of this disclosure relates to the method of Embodiment 115, wherein the compound is administered in an amount of about 480 mg twice per day.

Embodiment 132 of this disclosure relates to the method of Embodiment 115, wherein the compound is administered in an amount of about 600 mg twice per day.

Embodiment 133 of this disclosure relates to the method of Embodiment 115, wherein the compound is administered in an amount of about 720 mg twice per day.

Embodiment 134 of this disclosure relates to the method of Embodiment 115, wherein the compound is administered in an amount of about 780 mg twice per day.

Embodiment 135 of this disclosure relates to the method of Embodiment 115, wherein the compound is administered in an amount of about 840 mg twice per day.

Embodiment 136 of this disclosure relates to the method of Embodiment 115, wherein the compound is administered in an amount of about 960 mg twice per day.

Embodiment 137 of this disclosure relates to the method of Embodiment 115, wherein the compound is administered in an amount of about 1080 mg twice per day.

Embodiment 138 of this disclosure relates to the method of Embodiment 115, wherein the compound is administered in an amount of about 1200 mg twice per day.

Embodiment 139 of this disclosure relates to the method of Embodiment 115, wherein the compound is administered in an amount of about 240 mg three times per day.

Embodiment 140 of this disclosure relates to the method of Embodiment 115, wherein the compound is administered in an amount of about 480 mg three times per day.

Embodiment 141 of this disclosure relates to the method of Embodiment 115, wherein the compound is administered in an amount of about 600 mg three times per day.

Embodiment 142 of this disclosure relates to the method of Embodiment 69 to 141, wherein the compound is administered daily for about 21 days to about six months.

Embodiment 143 of this disclosure relates to the method of Embodiment 142, wherein the compound is administered daily for about 21 days.

Embodiment 144 of this disclosure relates to the method of Embodiment 69 to 142, wherein the compound is administered daily for about 3 months.

Embodiment 145 of this disclosure relates to the method of Embodiment 69 to 144, wherein the subject has failed at least one anti cancer therapy.

Embodiment 146 of this disclosure relates to the method of Embodiment 145, wherein the at least one cancer therapy is zongertinib, ELVN-002, BI-4142, poziotinib, or tucatinib.

Embodiment 147 of this disclosure relates to the method of Embodiment 69 to 146, wherein the method further comprises administering an additional therapeutic agent.

Embodiment 148 of this disclosure relates to the method of Embodiment 147, wherein the additional therapeutic agent is a HER2 monoclonal antibody or a HER2 antibody drug conjugate.

Embodiment 149 of this disclosure relates to the method of Embodiment 148, wherein the additional therapeutic agent is adagrasib, pertuzumab, margetuximab, or trastuzumab.

Embodiment 150 of this disclosure relates to the method of Embodiment 149, wherein the additional therapeutic agent is trastuzumab.

Embodiment 151 of this disclosure relates to the method of Embodiment 150, wherein the additional therapeutic agent is ado-trastuzumab emtansine or trastuzumab-deruxtecan.

Embodiment 153 of this disclosure relates to the method of Embodiment 147, wherein the additional therapeutic agent is alpilisib, MRTX-849, or RMC-6336.

Embodiment 154 of this disclosure relates to the method of Embodiment 69 to 153, wherein the cancer is colorectal cancer, breast cancer, bladder cancer, pancreatic cancer, biliary cancer, endometrial cancer, gastric cancer, esophageal squamous cancer, ovarian cancer, or non-small cell lung cancer.

Embodiment 155 of this disclosure relates to the method of Embodiment 154, wherein the cancer is breast cancer with brain metastasis, colorectal cancer with or without brain metastasis, bladder cancer with or without brain metastasis or non-small cell lung cancer with or without brain metastasis.

Embodiment 155(a) relates to the method of Embodiment 155, wherein the cancer is breast cancer with brain metastasis.

Embodiment 155(b) relates to the method of Embodiment 155, wherein the cancer is colorectal cancer with brain metastasis.

Embodiment 155(c) relates to the method of Embodiment 155, wherein the cancer is colorectal cancer without brain metastasis.

Embodiment 155(d) relates to the method of Embodiment 155, wherein the cancer is bladder cancer with brain metastasis.

Embodiment 155(e) relates to the method of Embodiment 155, wherein the cancer is bladder cancer without brain metastasis.

Embodiment 155(f) relates to the method of Embodiment 155, wherein the cancer is non-small cell lung cancer with brain metastasis.

Embodiment 155(g) relates to the method of Embodiment 155, wherein the cancer is non-small cell lung cancer without brain metastasis.

Embodiment 156 of this disclosure relates to the method of Embodiment 69 to 155, wherein the subject is in fed state.

Embodiment 157 of this disclosure relates to the method of Embodiment 69 to 155, wherein the subject is in a fasted state.

Embodiment 158 of this disclosure relates to the method of Embodiment 157, wherein the subject has fasted for at least 2 hours.

Embodiment 159 of this disclosure relates to the method of Embodiment 156, wherein the subject has a low-fat content diet.

Embodiment 160 of this disclosure relates to the method of Embodiment 69 to 159, wherein the cancer is associated with HER2 modulation or KRAS mutation.

Embodiment 161 of this disclosure relates to the method of Embodiment 69 to 159, wherein the cancer is associated with HER2 modulation and KRAS mutation.

Embodiment 162 of this disclosure relates to the method of Embodiment 160 or 161, wherein the HER2 modulation comprises HER2 overexpression, HER2 amplification, HER2 mutation, or a combination thereof.

Embodiment 163 of this disclosure relates to the method of Embodiment 160 to 162, wherein the KRAS mutation comprises at least one mutation in exon 2 of KRAS.

Embodiment 164 of this disclosure relates to the method of Embodiment 160 to 162, wherein the KRAS mutation comprises at least one mutation in exon 3 of KRAS.

Embodiment 165 of this disclosure relates to the method of Embodiment 160 to 162, wherein the KRAS mutation comprises at least one mutation in exon 4 of KRAS.

Embodiment 166 of this disclosure relates to the method of Embodiment 160 to 165, wherein the KRAS mutation comprises: G12A mutation (exon 2, codon 12); G12C mutation (exon 2, codon 12); G12D mutation (exon 2, codon 12); G12E mutation (exon 2, codon 12); G12F mutation (exon 2, codon 12); G12H mutation (exon 2, codon 12); G12I mutation (exon 2, codon 12); G12L mutation (exon 2, codon 12); G12R mutation (exon 2, codon 12); G12S mutation (exon 2, codon 12); G12V mutation (exon 2, codon 12); G12Y mutation (exon 2, codon 12); G13A mutation (exon 2, codon 13); G13C mutation (exon 2, codon 13); G13D mutation (exon 2, codon 13); G13E mutation (exon 2, codon 13); G13F mutation (exon 2, codon 13); G13P mutation (exon 2, codon 13); G13R mutation (exon 2, codon 13); G13V mutation (exon 2, codon 13); G13S mutation (exon 2, codon 13); G13Y mutation (exon 2, codon 13); A59E mutation (exon 3, codon 59); A59G mutation (exon 3, codon 59), A59T mutation (exon 3, codon 59); Q61A mutation (exon 3, codon 61); Q61E mutation (exon 3, codon 61); Q61H mutation (exon 3, codon 61); Q61K mutation (exon 3, codon 61); Q61L mutation (exon 3, codon 61); Q61P mutation (exon 3, codon 61); Q61R mutation (exon 3, codon 61); K117N mutation (exon 4, codon 117); A146P mutation (exon 4, codon 146); A146T mutation (exon 4, codon 146); A146V mutation (exon 4, codon 146), or a combination thereof.

Embodiment 167 of this disclosure relates to the method of Embodiment 166, wherein the KRAS mutation comprises the G12C mutation.

Embodiment 168 of this disclosure relates to the method of Embodiment 69 to 167, wherein the method does not inhibit EGFR signaling.

Embodiment 169 of this disclosure relates to the method of Embodiment 69 to 168, wherein the method does not inhibit MET signaling.

Embodiment 170 of this disclosure relates to the method of Embodiment 69 to 169, wherein the method does not inhibit PI3K signaling.

Embodiment 171 of this disclosure relates to the method of Embodiment 69 to 170, wherein the method comprises inhibiting one or more HER2 mutants selected from P95HER2, A775-G776-ins-YVMA, A775-G776-ins-YVMA-R678Q, A775-G776-ins-YVMS, A775-G776-ins-C, A775-G776-ins-MMAY, A775-G776-ins-SVMA, A775-G776-ins-VVMA, G776VC, G776-del-ins-AVGC, G776-del-ins-IC, G776-del-ins-LC, G776-del-ins-VV, G776-V777-del-ins-CVC, P780-Y781-ins-GSP, S310F, S310Y, L755S, D769N, V777L, L786V, T798M, V842I, and L869R.

Embodiment 172 of this disclosure relates to the method of Embodiment 69 to 171, wherein the compound crosses the blood brain barrier (bbb).

Embodiment 173 of this disclosure relates to the method of Embodiment 172, wherein a brain plasma PK of compound 5 is at least 50, 100, 500, or 1000 ng/mL.

Embodiment 174 of this disclosure relates to the method of Embodiment 173, wherein the brain plasma PK of compound 5 is maintained for at least 12, 24, or 36 hours after dosing.

Embodiment 175 of this disclosure relates to the method of Embodiment 69 to 174, wherein an intra-tumor PK of compound 5 is at least about 50, 100, 500, or 1000 ng/mL and wherein the PK of compound 5 is maintained for at least 12, 24, or 36 hours after dosing.

In another embodiment, Compounds of Formula I bind to HER2 in a type II DFG-out conformation.

In another embodiment, Compounds of Formula II bind to HER2 in a type II DFG-out conformation.

In another embodiment, Compounds of Formula IIa bind to HER2 in a type II DFG-out conformation.

In another embodiment, Compounds of Formula IIb bind to HER2 in a type II DFG-out conformation.

In another embodiment, Compound 5 binds to HER2 in a type II DFG-out conformation.

In another embodiment, the compounds of Formula I, II, IIa or IIb have over 10, 50, 100, 200, 400, 500, or 1000-fold selectivity for HER2 wild type and/or HER2 mutants against EGFR.

In another embodiment, the compounds of Formula I, II, IIa or IIb have over 10-fold selectivity for HER2 wild type and/or HER2 mutants against EGFR.

In another embodiment, the compounds of Formula I, II, IIa or IIb have over 50-fold selectivity for HER2 wild type and/or HER2 mutants against EGFR.

In another embodiment, the compounds of Formula I, II, IIa or IIb have over 100-fold selectivity for HER2 wild type and/or HER2 mutants against EGFR.

In another embodiment, the compounds of Formula I, II, IIa or IIb have over 200-fold selectivity for HER2 wild type and/or HER2 mutants against EGFR.

In another embodiment, the compounds of Formula I, II, IIa or IIb have over 400-fold selectivity for HER2 wild type and/or HER2 mutants against EGFR.

In another embodiment, the compounds of Formula I, II, IIa or IIb have over 500-fold selectivity for HER2 wild type and/or HER2 mutants against EGFR.

In another embodiment, the compounds of Formula I, II, IIa or IIb have over 1000-fold selectivity for HER2 wild type and/or HER2 mutants against EGFR.

In another embodiment, the compounds of Formula I, II, IIa or IIb have over 10, 50, 100, 200, 400, 500, or 1000-fold selectivity for HER2 wild type and/or HER2 mutants against PI3K.

The section headings used herein are for organizational purposes only and are not to be construed as limiting the subject matter described.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Compound Examples

Compounds of Formula I, II, and IIa of the present disclosure may be synthesized in accordance with the schemes and examples described below. The examples may be altered by substitution of the starting materials with other materials having similar structures to result in corresponding products. The structure of the desired product will generally make apparent to a person of skill in the art the required starting materials.

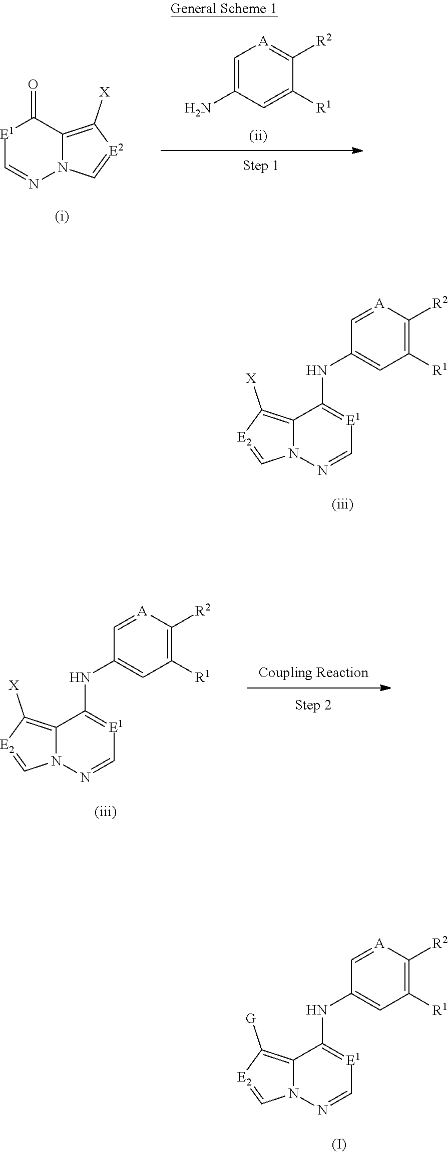

Step 1. Compound (i) can be converted to Compound (ii) through the application of a peptide coupling reagent such as PyBroP (by way of example) in appropriate reaction conditions which may be in the presence of a tertiary amine such as triethylamine. The reaction can take place in an appropriate solvent which may be aprotic solvent such as THE but may vary depending on the starting materials or intermediate compounds. Variables E1, E2, A, G, R1, and R2 in General Scheme 1 are as defined in this disclosure. Variable X in General Scheme 1 is an appropriate leaving group such as Br or Cl.

Step 2. Compound (ii) can be converted to Compound I by cross-coupling reactions by way of example such as palladium catalyzed Suzuki coupling with an organoborate such as

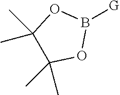

by way of example to arrive at Compound I. Variable G can be further modified one or more times by techniques described in this disclosure or by techniques known in the art.

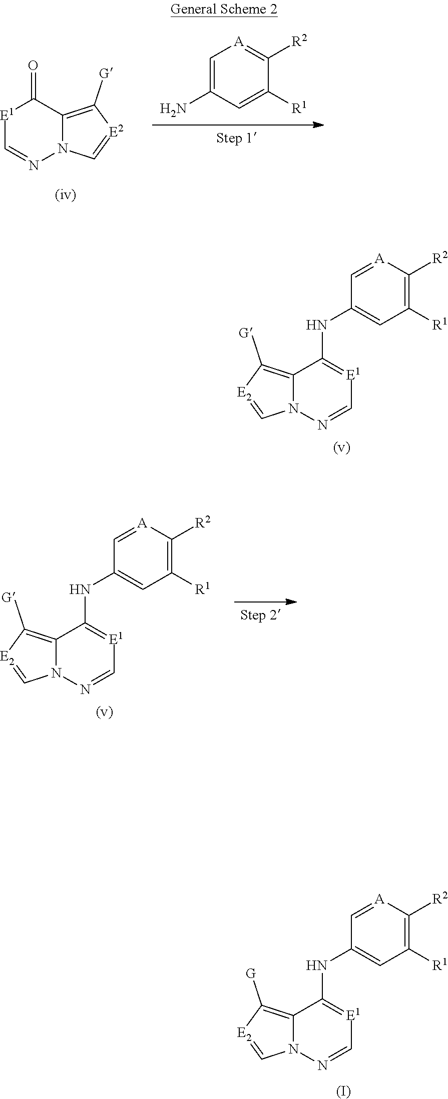

Step 1′. Compound (iv) can be converted to Compound (v) through the application of a peptide coupling reagent such as PyBroP by way of example in the presence of a tertiary amine such as triethylamine. The reaction can take place in an aprotic solvent such as THF. Variables E1, E2, A, R1, and R2 in General Scheme 1 are as defined in this disclosure. Variable G′ in General Scheme 1 can be a BOC-protected G group or another precursor that can be modified one or more times by techniques described in this disclosure or by techniques known in the art. G′ can also the same as variable G as described in this disclosure in which there would be no Step 2′ to modify variable G′.

Step 2′. Compound (v) can be converted to Compound I by one or more techniques described in this disclosure or known in the art. Such one or more techniques may include by way of example BOC deprotection, peptide coupling reactions with HATU, amide formation with HOBt, or nucleophilic substitution.

### Synthesis of Intermediate A

### Intermediate A

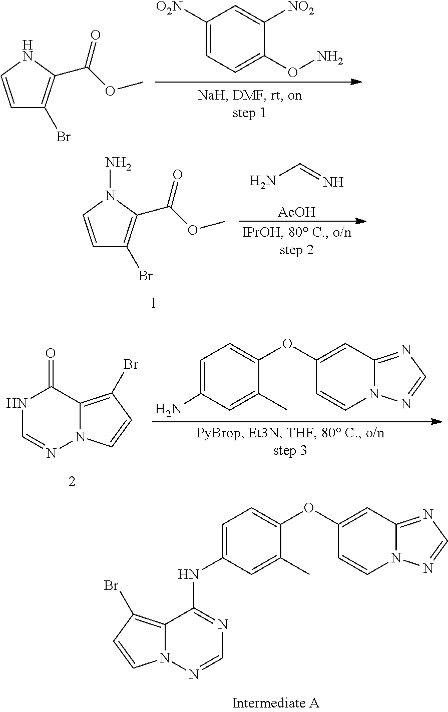

### Step 1. Methyl 1-amino-3-bromo-1H-pyrrole-2-carboxylate

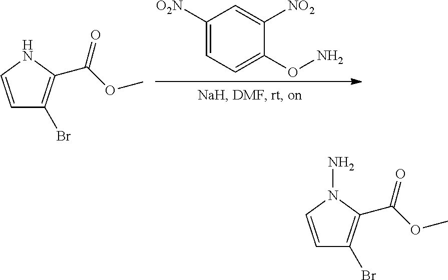

A solution of methyl 3-bromo-1H-pyrrole-2-carboxylate (25 g, 122.53 mmol) in DMF (200 mL) and THF (1000 mL) was treated with NaH (60% in mineral oil, 6.37 g, 159.25 mmol) for 1 hour at 0° C. followed by the addition of O-(2,4-dinitrophenyl)hydroxylamine (29.28 g, 147.04 mmol) at 0° C. and the mixture was stirred for 16 hours at room temperature. The reaction was quenched by the addition of saturated ammonium chloride aqueous solution (500 ml) at 0° C. The resulting mixture was diluted with water (1 L) and extracted with ethyl acetate (1.5 L×2). The combined organic layers were washed with brine (1.5 L×2), dried over anhydrous sodium sulfate, concentrated under reduced pressure. The residue was purified by silica gel column chromatography (0-20% ethyl acetate in hexanes) to provide methyl 1-amino-3-bromo-1H-pyrrole-2-carboxylate (22 g, 81.97%). LCMS (ESI-MS) m/z=219.0 [M+H]⁺.

### Step 2. 5-bromopyrrolo[2,1-f][1,2,4]triazin-4(3H)-one

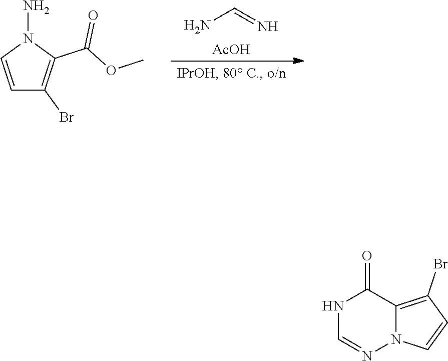

To a stirred solution of methyl 1-amino-3-bromo-1H-pyrrole-2-carboxylate (22 g, 100.43 mmol) in iPrOH (150 mL) was added formimidamide acetate (20.91 g, 200.87 mmol). The mixture was stirred at 80° C. overnight. The resulting mixture was diluted with water (300 ml). The precipitated solids were collected by filtration and washed with water (100 ml×3) and petroleum ether (200 ml) to afford 5-bromopyrrolo[2,1-f][1,2,4]triazin-4(3H)-one (13.2 g crude). LCMS (ESI-MS) m/z=214.0 [M+H]⁺.

### Step 3. N-(4-([1,2,4]triazolo[1,5-a]pyridin-7-yloxy)-3-methylphenyl)-5-bromopyrrolo[2,1-f][1,2,4]triazin-4-amine

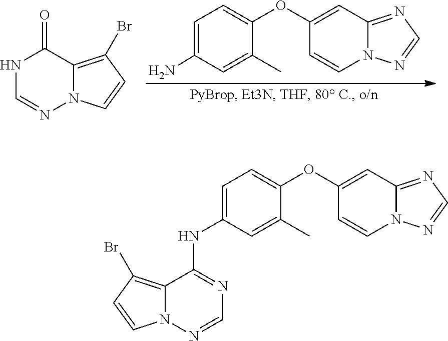

A solution of 5-bromopyrrolo[2,1-f][1,2,4]triazin-4(3H)-one (13.2 g, 61.97 mmol), 4-([1,2,4]triazolo[1,5-a]pyridin-7-yloxy)-3-methylaniline (14.87 g, 61.97 mmol), PyBrop (43.31 g, 92.95 mmol) and Et₃N (18.81 g, 185.91 mmol) in THF (300 mL) was stirred overnight at 80° C. The resulting mixture was purified by silica gel column chromatography (0-80% ethyl acetate in hexanes) to afford the title compound N-(4-([1,2,4]triazolo[1,5-a]pyridin-7-yloxy)-3-methylphenyl)-5-bromopyrrolo[2,1-f][1,2,4]triazin-4-amine, intermediate A (12 g, 27.33%). LCMS (ESI-MS) m/z=436.0 [M+H]⁺.

### Example 1. Compound 1

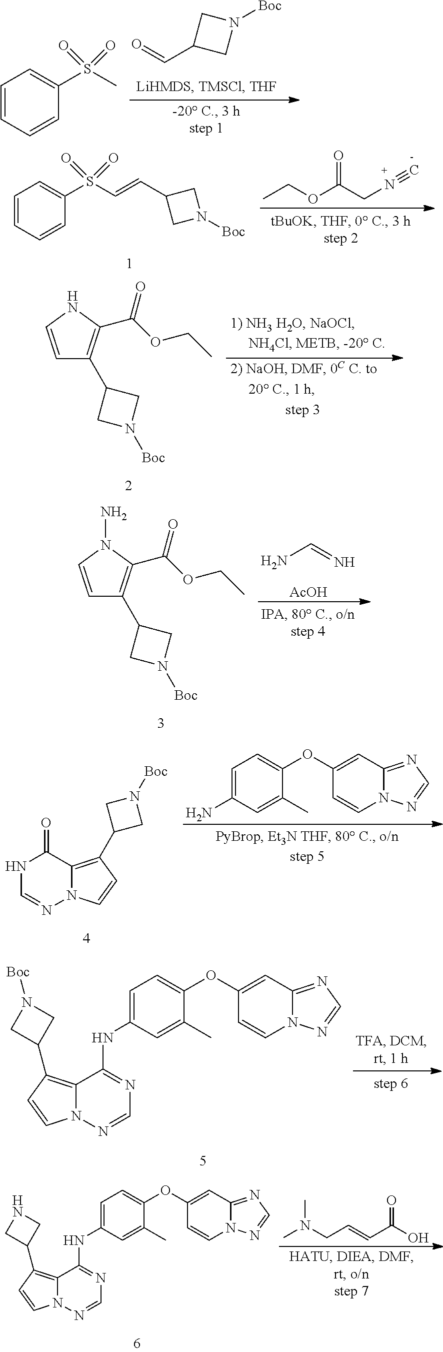

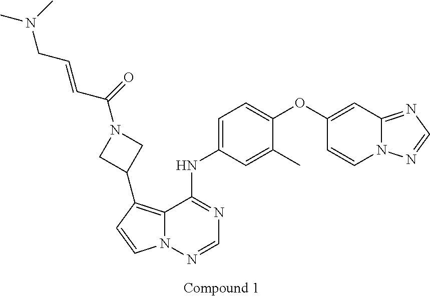 [SMILES: Cc1cc(Nc2ncnn3ccc(C4CN(C(=O)/C=C/CN(C)C)C4)c23)ccc1Oc1ccn2ncnc2c1]

### Step 1. Tert-butyl (E)-3-(2-(phenylsulfonyl)vinyl)azetidine-1-carboxylate

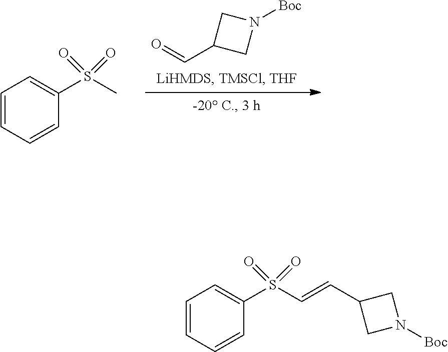

To a solution of methanesulfonylbenzene (16.7 g, 107 mmol) in anhydrous tetrahydrofuran (160 mL) at −20° C. under nitrogen atmosphere was added to lithium bis(trimethylsilyl)amide (1M solution in THF, 189 mL, 189 mmol) dropwise and the reaction was allowed to stir for 30 minutes at −20° C. To the reaction mixture was added chlorotimethylsilane (12.6 ml, 99.2 mmol) and slowed to stirred for a further 15 minutes. To the reaction mixture was added a solution of tert-butyl 3-formylazetidine-1-carboxylate (19.8 g, 106.9 mmol) in anhydrous tetrahydrofuran (200 mL) dropwise and allowed to stir at −20° C. for further 3 hours. The operation was repeated twice. The reaction mixture was quenched with saturated aqueous ammonium chloride (1 L) and extracted with ethyl acetate (2×1 L). The combined organics were dried over sodium sulfate filtered and concentrated under vacuum to afford the crude product. The crude product was purified by column chromatography (silica, petroleum ether/ethyl acetate, 15%) to give tert-butyl(E)-3-(2-(phenylsulfonyl)vinyl)azetidine-1-carboxylate (52 g, 47.6%). LCMS (ESI-MS) m/z=324.1 [M+H]⁺.

### Step 2. Ethyl 3-(1-(tert-butoxycarbonyl)azetidin-3-yl)-1H-pyrrole-2-carboxylate

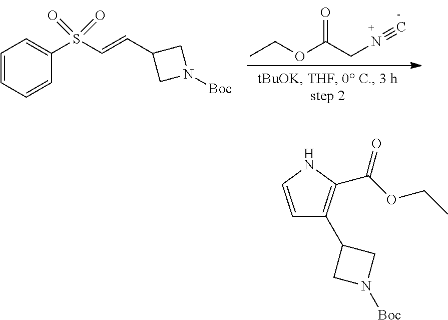

Potassium 2-methylpropan-2-olate (11.0 g, 98.2 mmol) was added to a mixture of ethyl 2-isocyanoacetate (8.4 g, 37.1 mmol) in tetrahydrofuran (100 mL) under nitrogen atmosphere at 0° C. and stirred for 10 minutes. Then tert-butyl (E)-3-(2-(phenylsulfonyl)vinyl)azetidine-1-carboxylate (20 g, 61.8 mmol) in THF (100 mL) was added to mixture and stirred at 25° C. over the time of one hour. The operation was repeated twice. The reaction was quenched by the addition of saturated aqueous ammonium chloride (500 mL) at 0° C. The resulting mixture was extracted with ethyl acetate (3×500 mL). The combined organic layers were washed with brine (3×500 mL), dried over anhydrous sodium sulfate. The filtrate was concentrated under reduced pressure. The residue was purified by silica gel column chromatography, eluted with petroleum ether/ethyl acetate (10%) to afford ethyl 3-(1-(tert-butoxycarbonyl)azetidin-3-yl)-1H-pyrrole-2-carboxylate (27 g, 42.2%). LCMS (ESI-MS) m/z=295.2 [M+H]⁺.

### Step 3. Ethyl 1-amino-3-(1-(tert-butoxycarbonyl)azetidin-3-yl)-1H-pyrrole-2-carboxylate

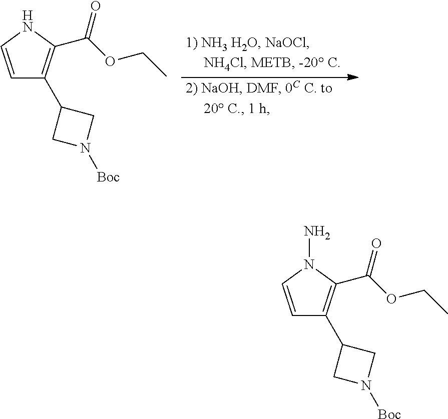 [SMILES: CCOC(=O)c1[nH]ccc1C1CN(C(=O)OC(C)(C)C)C1 ; CCOC(=O)c1c(C2CN(C(=O)OC(C)(C)C)C2)ccn1N]

To a flask was added MTBE (1 L) and ammonium chloride (30 g, 0.565 mol). The reaction was cooled to −20° C. Then concentrated aq. ammonium hydroxide (80 mL) was added to the reaction followed by slow addition of commercial-grade sodium hypochlorite solution (750 mL). After addition, the reaction was stirred at −20° C. for additional 30 minutes. The MTBE layer was separated and washed with brine and dried over anhydrous sodium sulfate. In a separate flask under nitrogen was added ethyl 3-(1-(tert-butoxycarbonyl)azetidin-3-yl)-1H-pyrrole-2-carboxylate (27 g, 91.8 mmol) and dry DMF (300 mL). The reaction was cooled to 0° C. and sodium hydroxide (7.3 g, 183.6 mmol) was added portion wise to the reaction. The reaction was stirred at 0° C. for additional 1 hour before it was cooled to −20° C. At this time, the previously prepared MTBE solution of chloramine was added slowly to the reaction and the mixture was stirred at −20° C. for 1 hour. The reaction was quenched with saturated sodium thiosulfate solution. The organic layer of the reaction was separated and washed with water and brine, dried over sodium sulfate, filtered and concentrated to afford ethyl 1-amino-3-(1-(tert-butoxycarbonyl)azetidin-3-yl)-1H-pyrrole-2-carboxylate (21 g, 60.3%). LCMS (ESI-MS) m/z=310.2 [M+H]⁺.

### Step 4. Tert-butyl 3-(4-oxo-3,4-dihydropyrrolo[2,1-f][1,2,4]triazin-5-yl)azetidine-1-carboxylate

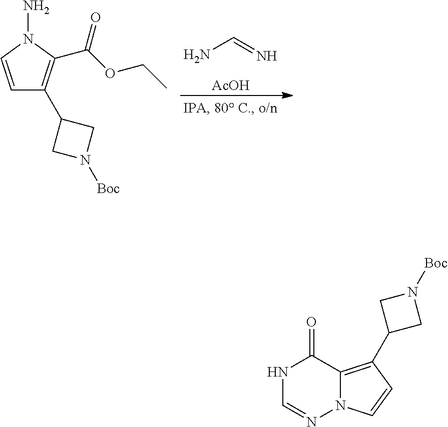

Acetic acid methanimidamide (7.06 g, 67.8 mmol) was added to a mixture of ethyl 1-amino-3-[1-(tert-butoxycarbonyl)azetidin-3-yl]pyrrole-2-carboxylate (4 g, 13.5 mmol) in iPrOH (15 mL). Then the reaction mixture was stirred at 80° C. for 12 hours. The reaction mixture was concentrated under vacuum and purified by silica gel column chromatography, eluted with PE/EA (37%) and concentrated to afford tert-butyl 3-(4-oxo-3,4-dihydropyrrolo[2,1-f][1,2,4]triazin-5-yl)azetidine-1-carboxylate (15 g, 76.1%). LCMS (ESI-MS) m/z=291.1 [M+H]⁺.

### Step 5. Tert-butyl 3-(4-((4-([1,2,4]triazolo[1,5-a]pyridin-7-yloxy)-3-methylphenyl)amino)pyrrolo[2,1-f][1,2,4]triazin-5-yl)azetidine-1-carboxylate

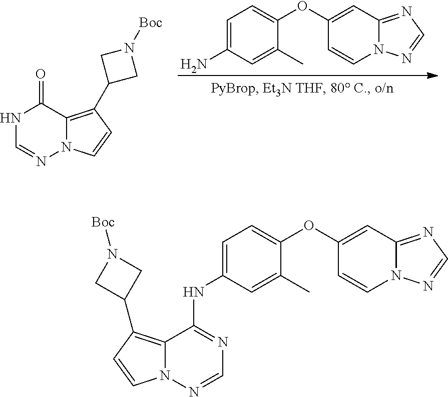 [SMILES: CC(C)(C)OC(=O)N1CC(c2ccn3nc[nH]c(=O)c23)C1 ; Cc1cc(N)ccc1Oc1ccn2ncnc2c1 ; Cc1cc(Nc2ncnn3ccc(C4CN(C(=O)OC(C)(C)C)C4)c23)ccc1Oc1ccn2ncnc2c1]

Bromotris(pyrrolidin-1-yl)phosphanium; hexafluoro-lambda5-phosphanuide (2.41 g, 5.16 mmol) was added to a mixture of tert-butyl 3-(4-oxo-3,4-dihydropyrrolo[2,1-f][1,2,4]triazin-5-yl)azetidine-1-carboxylate (1 g, 3.44 mmol), 4-([1,2,4]triazolo[1,5-a]pyridin-7-yloxy)-3-methylaniline (830 mg, 3.44 mmol) and triethylamine (1.1 g, 10.33 mmol) in THF (30 mL). Then the reaction mixture was stirred at 80° C. for overnight. The resulting mixture was cooled down to room temperature, filtered, the filter cake was washed with dichloromethane (3×50 mL). The filtrate was concentrated under reduced pressure to afford the crude and then purified by silica gel column chromatography, eluted with petroleum ether/ethyl acetate (1:6) to afford the crude 1.2 g (contains 40% of SM). Then the crude material was re-purified by reversed-phase flash chromatography with the following conditions: column, C18 silica gel; mobile phase, ACN in Water (10 mmol/L NH₄HCO₃), 10% to 50% gradient in 10 min; detector, UV 254 nm and concentrated to give tert-butyl 3-(4-((4-([1,2,4]triazolo[1,5-a]pyridin-7-yloxy)-3-methylphenyl)amino)pyrrolo[2,1-f][1,2,4]triazin-5-yl)azetidine-1-carboxylate (553 mg, 31.25%). LCMS (ESI-MS) m/z=513.2 [M+H]⁺.

### Step 6. N-(4-([1,2,4]triazolo[1,5-a]pyridin-7-yloxy)-3-methylphenyl)-5-(azetidin-3-yl)pyrrolo[2,1-f][1,2,4]triazin-4-amine

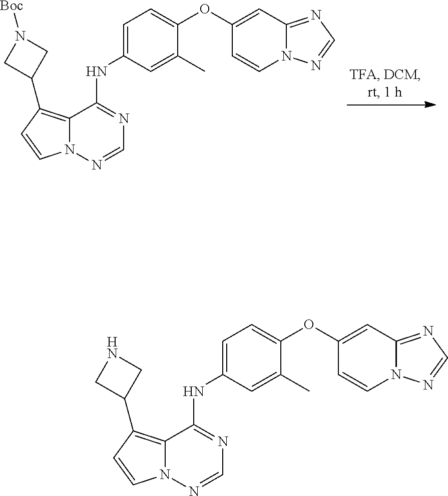 [SMILES: Cc1cc(Nc2ncnn3ccc(C4CN(C(=O)OC(C)(C)C)C4)c23)ccc1Oc1ccn2ncnc2c1 ; Cc1cc(Nc2ncnn3ccc(C4CNC4)c23)ccc1Oc1ccn2ncnc2c1]

A solution of TFA (1 mL) and tert-butyl 3-(4-((4-([1,2,4]triazolo[1,5-a]pyridin-7-yloxy)-3-methylphenyl)amino)pyrrolo[2,1-f][1,2,4]triazin-5-yl)azetidine-1-carboxylate (300 mg, 0.58 mmol) in DCM (2 mL) was stirred for 1 hour at 25° C. The resulting mixture was concentrated under vacuum to afford the crude N-(4-([1,2,4]triazolo[1,5-a]pyridin-7-yloxy)-3-methylphenyl)-5-(azetidin-3-yl)pyrrolo [2,1-f][1,2,4]triazin-4-amine (250 mg). LCMS (ESI-MS) m/z=413.2 [M+H]⁺.

### Step 7. (E)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-4-(dimethylamino)but-2-en-1-one

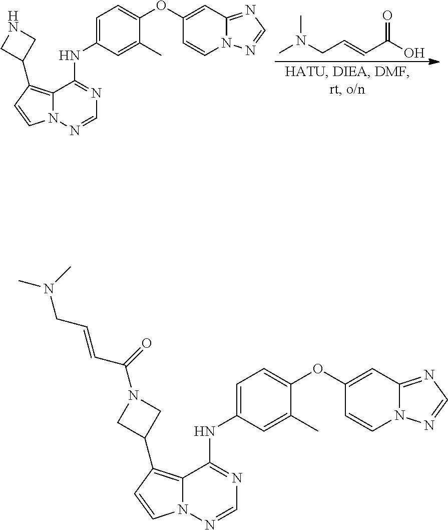

A solution of N-(4-([1,2,4]triazolo[1,5-a]pyridin-7-yloxy)-3-methylphenyl)-5-(azetidin-3-yl)pyrrolo[2,1-f][1,2,4]triazin-4-amine (250 mg, 0.61 mmol), (2E)-4-(dimethylamino)but-2-enoic acid (94 mg, 0.72 mmol), N,N,N,N-Tetramethyl-O-(7-azabenzotriazol-1-yl)uronium hexafluorophosphate (277 mg, 0.72 mmol) and N,N-Diisopropylethylamine (237 mg, 1.81 mmol) in DMF (5 mL) was stirred overnight at room temperature. The resulting mixture was purified by reverse phase flash with the following conditions (5 mmol/L NH₄HCO₃, Flow rate: 50 mL/min, 30%) to afford to afford (E)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-4-(dimethylamino)but-2-en-1-one, Compound 1 (138 mg, 42.9%). LCMS (ESI-MS) m/z=524.1 [M+H]⁺.

### Example 2. Compound 2

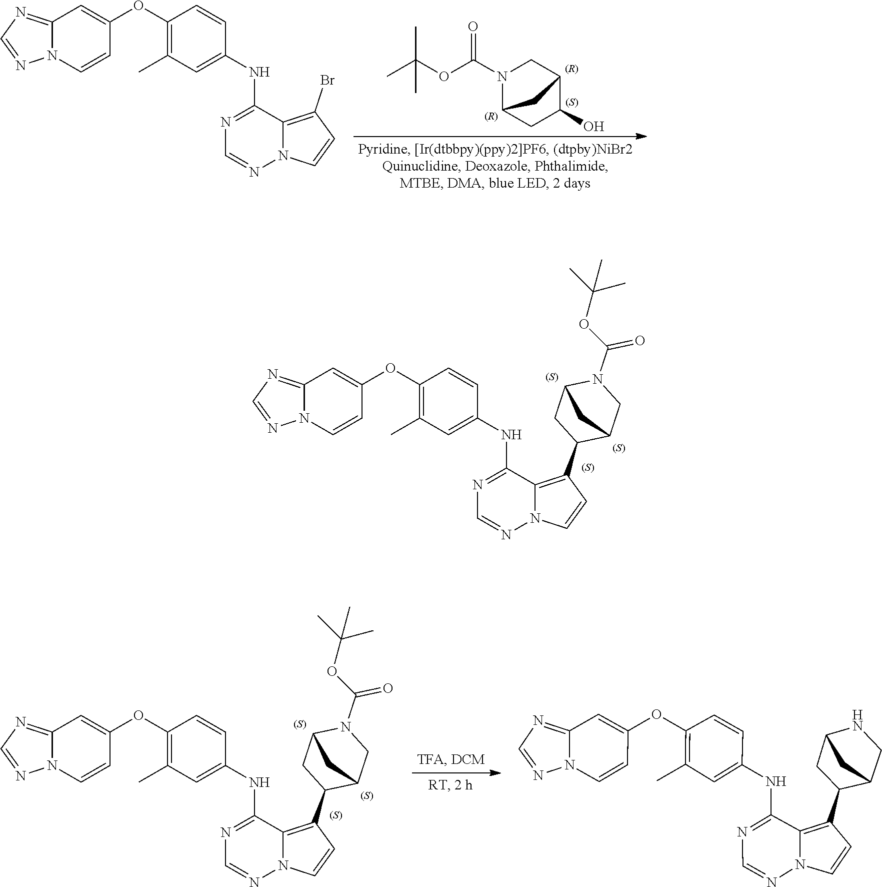

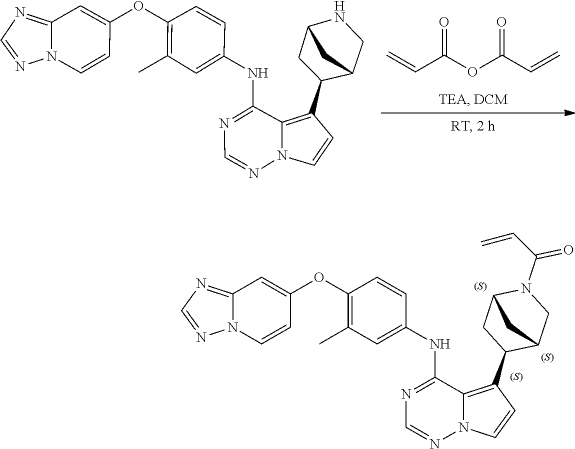

### Step 1: tert-butyl (1R,4R,5S)-5-{4-[(3-methyl-4-{[1,2,4]triazolo[1,5-a]pyridin-7-yloxy}phenyl)amino]pyrrolo[2,1-f][1,2,4]triazin-5-yl}-2-azabicyclo[2.2.1]heptane-2-carboxylate

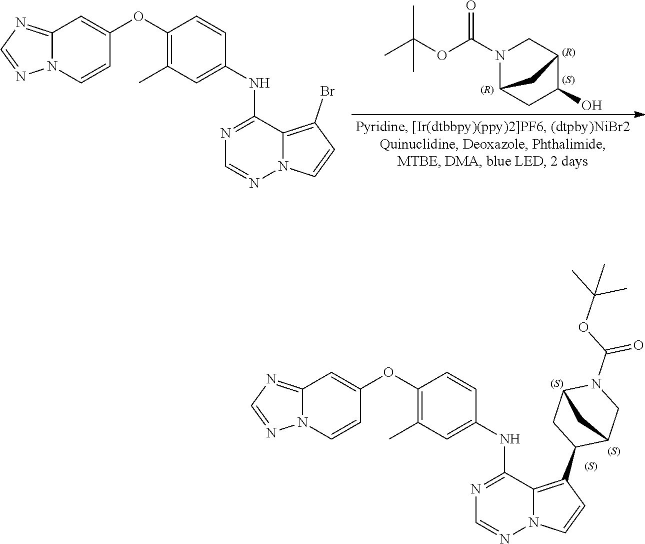

A sealed tube was charged with deoxazole (0.331 g, 0.836 mmol, 1.6 eq) and tert-butyl (1R,4R,5S) 5-hydroxy-2-azabicyclo[2.2.1]heptane-2-carboxylate (0.195 g, 0.915 mmol, 1.75 eq). The mixture was degasses under vacuum and charged with argon. This process was repeated two times. Then dry MTBE (5.23 ml, 0.1 M) was added via syringe to the tube and the mixture was stirred for 5 min at room temperature. A solution of dry pyridine (0.068 ml, 0.836 mmol, 1.6 eq) in dry MTBE (0.99 ml, 15.0 vol) was then added dropwise over 2 min and the resulting solution was stirred for 10 minutes. In a 7 mL vial, 5-bromo-N-(3-methyl-4-{[1,2,4]triazolo[1,5-a]pyridin-7-yloxy}phenyl)pyrrolo[2,1-f][1,2,4]triazin-4-amine (0.24 g, 0.523 mmol, 1.0 eq), [Ir(dtbbpy)(ppy)₂]PF₆ (0.007 g, 0.008 mmol, 0.015 eq), [4,4′-bis(tert-butyl)-2,2′-bipyridine]nickel dibromide (0.019 g, 0.039 mmol, 0.075 eq), phthalimide (0.017 g, 0.118 mmol, 0.225 eq) and quinuclidine (0.102 g, 0.915 mmol, 1.75 eq) were added and the mixture was suspended in dry dimethylacetamide (4.8 ml, 20.0 vol) under argon. MTBE solution was transferred to a syringe and filtered prior to addition. The reaction mixture was purged with argon for 15 minutes and sealed with parafilm. The vial was placed in a PennPhD Photoreactor stirring at 1500 rpm under 450 nm LED irradiation at 100% intensity with a fan speed of 2800 rpm. Stirring was carried out for 2 days. Reaction mixture was filtered through celite pad and washed with ethyl acetate.

Solvents were evaporated and purification was performed. Purification via flash chromatography (DCM/MeOH 100:0->95:5) was performed to give a crude product. Additional re-purification was performed via preparative TLC (plate was developed 3 times; DCM/MeOH 100:0->96:4). Tert-butyl (1S,4S,5S)-5-{4-[(3-methyl-4-{[1,2,4]triazolo[1,5-a]pyridin-7-yloxy}phenyl)amino]pyrrolo[2,1-f][1,2,4]triazin-5-yl}-2-azabicyclo[2.2.1]heptane-2-carboxylate (0.03 g, 8%) was isolated. ¹H NMR (300 MHz, DMSO-d6) δ 8.94 (dd, J=7.5, 3.1 Hz, 1H), 8.43 (s, 1H), 8.39 (s, 1H), 7.92 (s, 1H), 7.73-7.65 (m, 2H), 7.22 (d, J=8.4 Hz, 1H), 7.03 (dd, J=7.5, 2.6 Hz, 1H), 6.81 (d, J=2.0 Hz, 1H), 6.71 (d, J=2.7 Hz, 1H), 4.19 (d, J=15.1 Hz, 1H), 3.90 (dd, J=9.4, 4.1 Hz, 1H), 3.30 (s, 1H), 2.70 (dd, J=4.9, 2.1 Hz, 1H), 2.19 (s, 4H), 1.88-1.78 (m, 1H), 1.66 (d, J=10.8 Hz, 1H), 1.62-1.54 (m, 1H), 1.42 (s, 9H), 1.24 (s, 2H). UPLC (ESI) [M+H]⁺=553.10.

### Step 2: 5-[(1S,4S,5S)-2-azabicyclo[2.2.1]heptan-5-yl]-N-(3-methyl-4-{[1,2,4]triazolo[1,5-a]pyridin-7-yloxy}phenyl)pyrrolo[2,1-f][1,2,4]triazin-4-amine

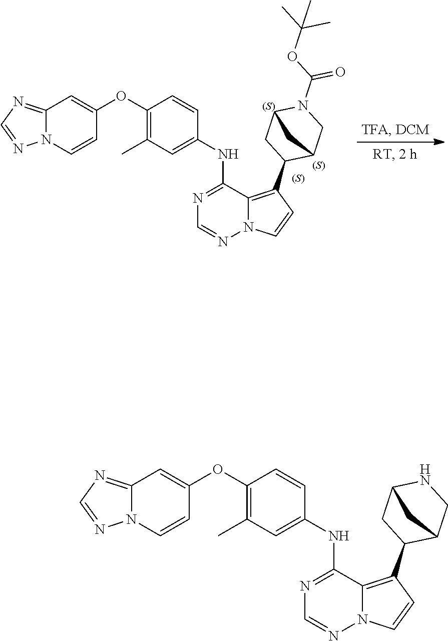

To a stirring solution of tert-butyl (1S,4S,5S)-5-{4-[(3-methyl-4-{[1,2,4]triazolo[1,5-a]pyridin-7-yloxy}phenyl)amino]pyrrolo[2,1-f][1,2,4]triazin-5-yl}-2-azabicyclo[2.2.1]heptane-2-carboxylate (0.03 g, 0.044 mmol, 1.0 eq) in DCM (0.6 ml, 20.0 vol) was added TFA (0.3 ml, 10.0 vol). The reaction was stirred at RT for 2 hours. Solvents were evaporated and the crude was used in the next step without further purification. 5-[(1S,4S,5S)-2-azabicyclo[2.2.1]heptan-5-yl]-N-(3-methyl-4-{[1,2,4]triazolo[1,5-a]pyridin-7-yloxy}phenyl)pyrrolo[2,1-f][1,2,4]triazin-4-amine (0.045 g, crude) was isolated in the form of trifluoroacetic acid salt. ¹H NMR (300 MHz, Methanol-d4) δ 8.82 (d, J=7.5 Hz, 1H), 8.42 (s, 1H), 7.77 (s, 1H), 7.67 (d, J=2.8 Hz, 1H), 7.56-7.52 (m, 1H), 7.47 (dd, J=8.8, 2.8 Hz, 1H), 7.27 (d, J=8.5 Hz, 1H), 7.17 (dd, J=7.6, 2.6 Hz, 1H), 6.89 (d, J=2.5 Hz, 1H), 6.79 (d, J=2.8 Hz, 1H), 4.25 (s, 1H), 3.91 (dd, J=8.4, 4.8 Hz, 1H), 3.37 (d, J=7.8 Hz, 1H), 3.03 (s, 1H), 2.47 (ddd, J=14.4, 8.9, 2.4 Hz, 1H), 2.30 (s, 1H), 2.27 (s, 3H), 2.18-2.11 (m, 2H), 1.87-1.78 (m, 1H). UPLC (ESI) [M+H]⁺=492.95.

### Step 3: 1-[(1S,4S,5S)-5-{4-[(3-methyl-4-{[1,2,4]triazolo[1,5-a]pyridin-7-yloxy}phenyl)amino]pyrrolo[2,1-f][1,2,4]triazin-5-yl}-2-azabicyclo[2.2.1]heptan-2-yl]prop-2-en-1-one

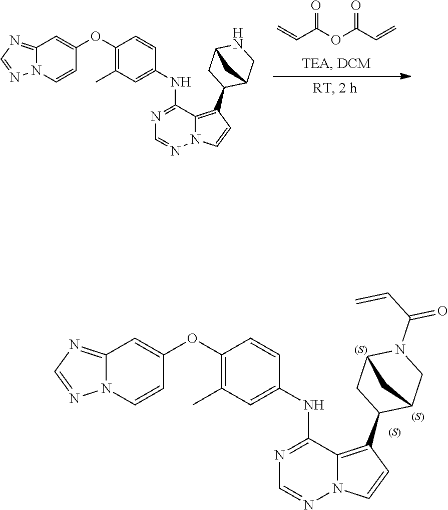

To a stirring solution of 5-[(1S,4S,5S)-2-azabicyclo[2.2.1]heptan-5-yl]-N-(3-methyl-4-{[1,2,4]triazolo[1,5-a]pyridin-7-yloxy}phenyl)pyrrolo[2,1-f][1,2,4]triazin-4-amine (0.045 g, 0.086 mmol, 1.0 eq) in dry DCM (0.9 ml, 20.0 vol) was added triethylamine (0.072 ml, 0.513 mmol, 6.0 eq) and acrylic anhydride (0.008 g, 0.064 mmol, 0.75 eq) at room temperature. The reaction was stirred at room temperature for 2 hour. The solvent was evaporated, followed by purification using preparative TLC (plate was developed 4 times at DCM/MeOH 100:0->98:2->96:4->95:5). 1-[(1S,4S,5S)-5-{4-[(3-methyl-4-{[1,2,4]triazolo[1,5-a]pyridin-7-yloxy}phenyl)amino]pyrrolo[2,1-f][1,2,4]triazin-5-yl}-2-azabicyclo[2.2.1]heptan-2-yl]prop-2-en-1-one, Compound 2 (0.011 g, 25%) was isolated. LCMS (ESI) [M+H]⁺=507.33. ¹H NMR (300 MHz, DMSO-d6) δ 8.95 (d, J=7.4 Hz, 1H), 8.41 (d, J=12.2 Hz, 2H), 7.91 (s, 1H), 7.75-7.65 (m, 2H), 7.58 (dt, J=8.5, 2.1 Hz, 1H), 7.22 (d, J=8.6 Hz, 1H), 7.03 (dd, J=7.4, 2.4 Hz, 1H), 6.81 (d, J=2.4 Hz, 1H), 6.75 (dd, J=5.7, 2.3 Hz, 1H), 6.16 (ddd, J=16.7, 5.7, 2.4 Hz, 1H), 5.67 (td, J=9.7, 2.4 Hz, 1H), 4.60 (d, J=9.0 Hz, 1H), 3.88 (dd, J=8.5, 5.2 Hz, 1H), 3.58 (s, 1H), 3.46 (d, J=11.0 Hz, 1H), 2.79 (d, J=8.1 Hz, 1H), 2.25-2.21 (m, 1H), 2.19 (s, 3H), 1.91-1.74 (m, 2H), 1.74-1.55 (m, 2H).

### Example 3. Compound 3

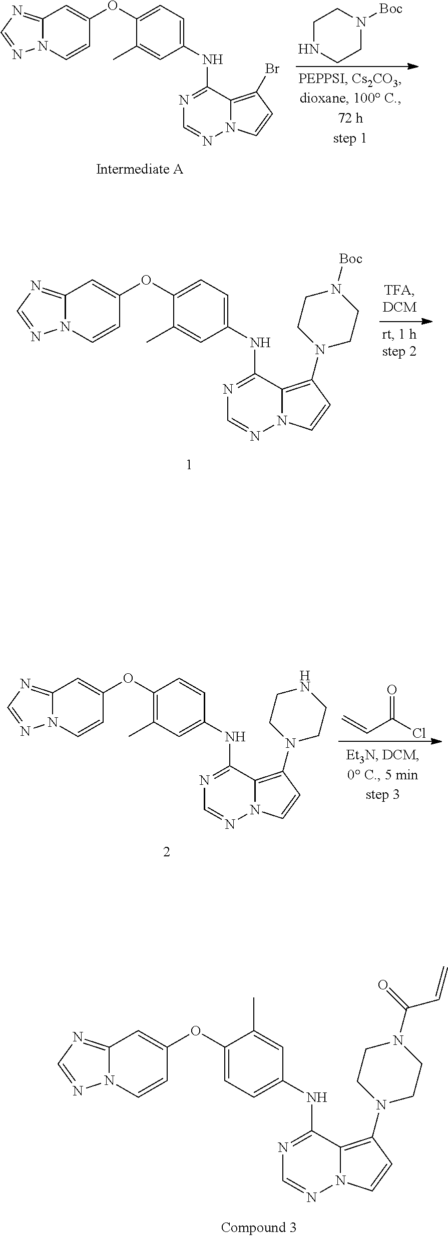

### Step 1. Tert-butyl-4-(4-((4-([1,2,4]triazolo[1,5-a]pyridin-7-yloxy)-3-methylphenyl)amino)pyrrolo[2,1-f][1,2,4]triazin-5-yl)piperazine-1-carboxylate

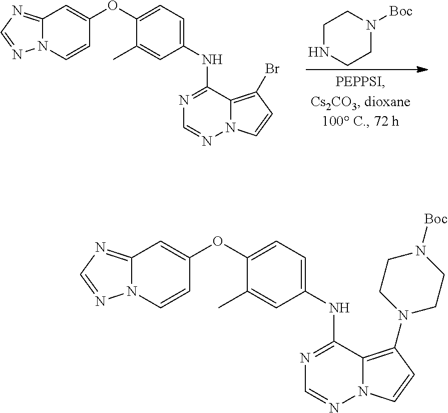 [SMILES: Cc1cc(Nc2ncnn3ccc(Br)c23)ccc1Oc1ccn2ncnc2c1 ; CC(C)(C)OC(=O)N1CCNCC1 ; Cc1cc(Nc2ncnn3ccc(N4CCN(C(=O)OC(C)(C)C)CC4)c23)ccc1Oc1ccn2ncnc2c1]

A mixture of N-(4-([1,2,4]triazolo[1,5-a]pyridin-7-yloxy)-3-methylphenyl)-5-bromopyrrolo[2,1-f][1,2,4]triazin-4-amine (200 mg, 0.45 mmol), tert-butyl piperazine-1-carboxylate (93.92 mg, 0.50 mmol), Pd₂(dba)₃ (41.98 mg, 0.05 mmol), BINAP (57.09 mg, 0.09 mmol) and t-BuONa (88.11 mg, 0.91 mmol) in dioxane (4 mL) was stirred for 72 hours at 100° C. under nitrogen atmosphere. The reaction mixture was filtered and the filtrate was concentrated to afford the crude product. The crude product was purified by column chromatography (silica gel, 25 g, eluted with ethyl acetate in petroleum ether from 0% to 80% with 20 mL/min flow rate), the desired fractions were combined and concentrated under vacuum to afford the desired product tert-butyl 4-(4-((4-([1,2,4]triazolo[1,5-a]pyridin-7-yloxy)-3-methylphenyl)amino)pyrrolo [2,1-f][1,2,4]triazin-5-yl)piperazine-1-carboxylate (200 mg, 69%). LCMS (ESI-MS) m/z=542.3 [M+H]⁺.

### Step 2. N-(4-([1,2,4]triazolo[1,5-a]pyridin-7-yloxy)-3-methylphenyl)-5-(piperazin-1-yl)pyrrolo[2,1-f][1,2,4]triazin-4-amine

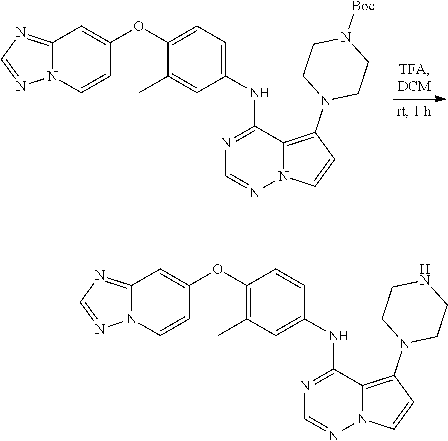 [SMILES: Cc1cc(Nc2ncnn3ccc(N4CCN(C(=O)OC(C)(C)C)CC4)c23)ccc1Oc1ccn2ncnc2c1 ; Cc1cc(Nc2ncnn3ccc(N4CCNCC4)c23)ccc1Oc1ccn2ncnc2c1]

A mixture of tert-butyl 4-(4-((4-([1,2,4]triazolo[1,5-a]pyridin-7-yloxy)-3-methylphenyl)amino)pyrrolo[2,1-f][1,2,4]triazin-5-yl)piperazine-1-carboxylate (200 mg, 0.37 mmol) and TFA (3 mL, 39.99 mmol) in DCM (1 mL) was stirred for 1 hour at room temperature. The resulting mixture was concentrated under vacuum to afford the crude product. The crude product was purified by column chromatography (silica-gel, 25 g, eluted with methanol in dichloromethane from 0% to 10% with 20 mL/min flowrate), the desired fractions were combined and concentrated under vacuum to afford N-(4-([1,2,4]triazolo[1,5-a]pyridin-7-yloxy)-3-methylphenyl)-5-(piperazin-1-yl)pyrrolo[2,1-f][1,2,4]triazin-4-amine (210 mg crude). LCMS (ESI-MS) m/z=442.2 [M+H]⁺.

### Step 3. 1-(4-(4-((4-([1,2,4]triazolo[1,5-a]pyridin-7-yloxy)-3-methylphenyl)amino)pyrrolo[2,1-f][1,2,4]triazin-5-yl)piperazin-1-yl)prop-2-en-1-one

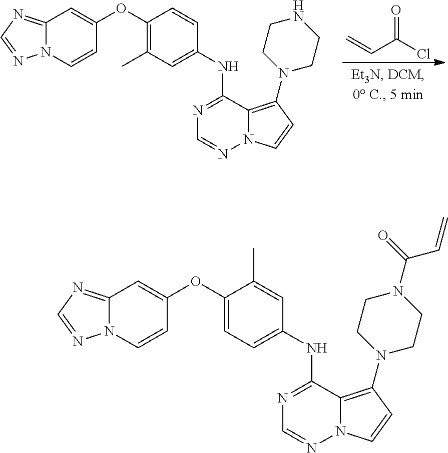

A mixture of N-(4-([1,2,4]triazolo[1,5-a]pyridin-7-yloxy)-3-methylphenyl)-5-(piperazin-1-yl)pyrrolo[2,1-f][1,2,4]triazin-4-amine (210 mg, 0.47 mmol), acryloyl chloride (43.05 mg, 0.47 mmol) and Et₃N (144.40 mg, 1.41 mmol) in DCM (2 mL) was stirred for 5 minutes at 0° C. The reaction mixture was purified by column chromatography (silica-gel, 25 g, eluted with ethyl acetate in petroleum ether from 0% to 80% with 20 mL/min). The fractions with desired mass signal were combined and concentrated under vacuum to afford the desired product 1-(4-(4-((4-([1,2,4]triazolo[1,5-a]pyridin-7-yloxy)-3-methylphenyl)amino)pyrrolo[2,1-f][1,2,4]triazin-5-yl)piperazin-1-yl)prop-2-en-1-one, Compound 3 (27.5 mg, 11.62%). LCMS (ESI-MS) m/z=496.2 [M+H]⁺.

### Example 4. Compound 4

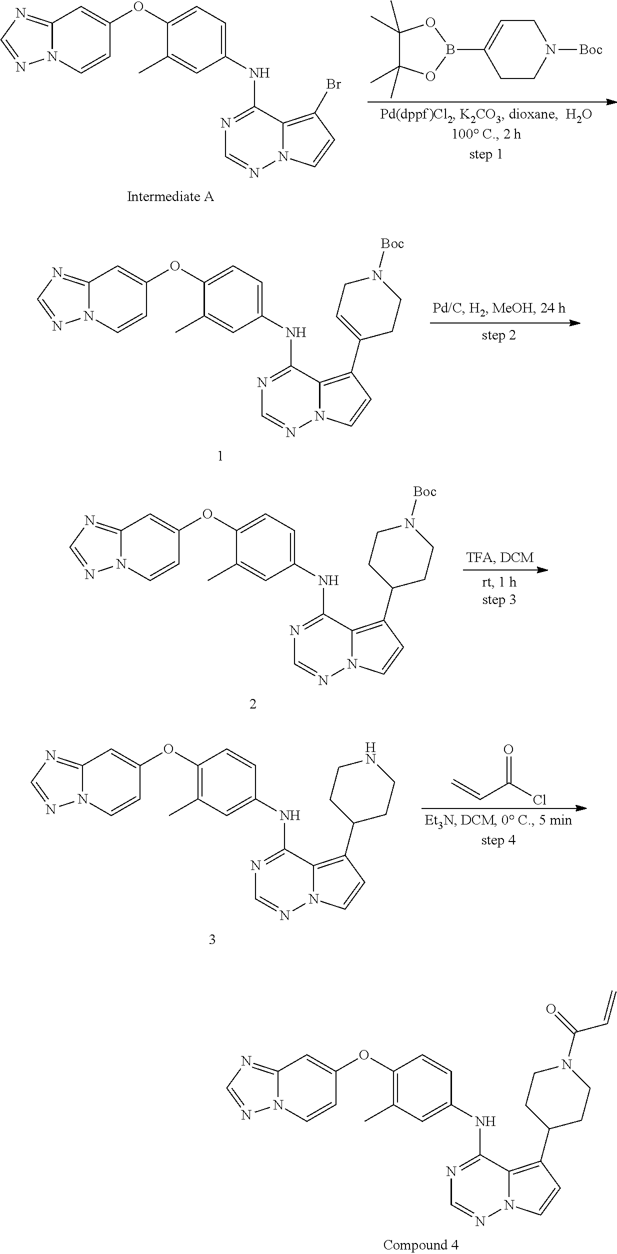

### Step 1. tert-butyl 4-(4-((4-([1,2,4]triazolo[1,5-a]pyridin-7-yloxy)-3-methylphenyl)amino)pyrrolo[2,1-f][1,2,4]triazin-5-yl)-3,6-dihydropyridine-1(2H)-carboxylate

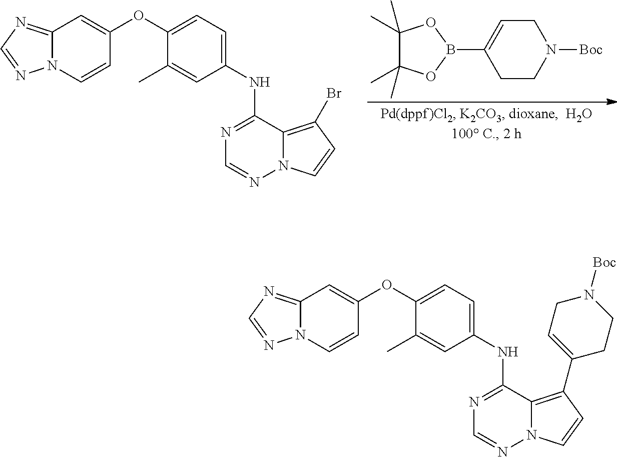

A solution of N-(4-([1,2,4]triazolo[1,5-a]pyridin-7-yloxy)-3-methylphenyl)-5-bromopyrrolo[2,1-f][1,2,4]triazin-4-amine (200 mg, 0.46 mmol), tert-butyl 4-(4,4,5,5-tetramethyl-1,3,2-dioxaborolan-2-yl)-3,6-dihydropyridine-1(2H)-carboxylate (141.75 mg, 0.46 mmol), Pd(dppf)Cl₂ (37.34 mg, 0.05 mmol) and K₂CO₃ (126.71 mg, 0.92 mmol) in dioxane (4 mL) and H₂O (1.2 mL) was stirred for 2 hours at 100° C. under nitrogen atmosphere. The resulting mixture was concentrated under vacuum to afford the crude product. The crude product was purified by Prep-TLC (petroleum ether/ethyl acetate 1:10) to afford tert-butyl 4-(4-((4-([1,2,4]triazolo[1,5-a]pyridin-7-yloxy)-3-methylphenyl)amino)pyrrolo[2,1-f][1,2,4]triazin-5-yl)-3,6-dihydropyridine-1(2H)-carboxylate (220 mg, purity=96.8%). LCMS (ESI-MS) m/z=539.2 [M+H]⁺.

### Step 2. Tert-butyl 4-(4-((4-([1,2,4]triazolo[1,5-a]pyridin-7-yloxy)-3-methylphenyl)amino)pyrrolo[2,1-f][1,2,4]triazin-5-yl)piperidine-1-carboxylate

A solution of 4-(4-((4-([1,2,4]triazolo[1,5-a]pyridin-7-yloxy)-3-methylphenyl)amino)pyrrolo[2,1-f][1,2,4]triazin-5-yl)-3,6-dihydropyridine-1(2H)-carboxylate (200 mg, 0.37 mmol) and Pd/C (395.16 mg, 3.71 mmol) in MeOH was stirred overnight at room temperature under hydrogen atmosphere. The mixture was filtered off and the filtrate was concentrated under vacuum to afford crude product. The crude product was used in the next step directly without further purification. LCMS (ESI-MS) m/z=541.3 [M+H]⁺.

### Step 3. N-(4-([1,2,4]triazolo[1,5-a]pyridin-7-yloxy)-3-methylphenyl)-5-(piperidin-4-yl)pyrrolo[2,1-f][1,2,4]triazin-4-amine

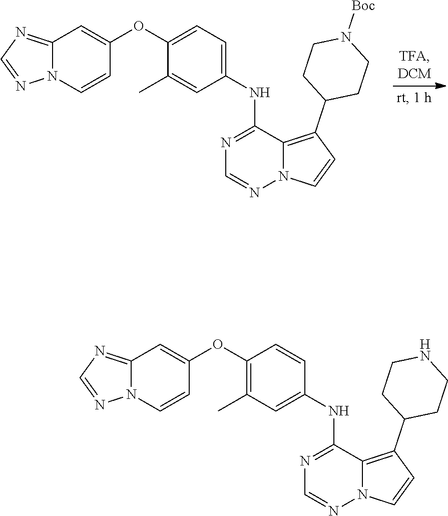 [SMILES: Cc1cc(Nc2ncnn3ccc(C4CCN(C(=O)OC(C)(C)C)CC4)c23)ccc1Oc1ccn2ncnc2c1 ; Cc1cc(Nc2ncnn3ccc(C4CCNCC4)c23)ccc1Oc1ccn2ncnc2c1]

A solution of tert-butyl 4-(4-((4-([1,2,4]triazolo[1,5-a]pyridin-7-yloxy)-3-methylphenyl)amino)pyrrolo[2,1-f][1,2,4]triazin-5-yl)piperidine-1-carboxylate (140 mg, 0.26 mmol) in TFA (2 mL) was stirred for 1 hour at room temperature. The resulting mixture was concentrated under vacuum to afford the crude product. The crude product was used in the next step directly without further purification. LCMS (ESI-MS) m/z=441.2 [M+H]⁺.

### Step 4. 1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)prop-2-en-1-one

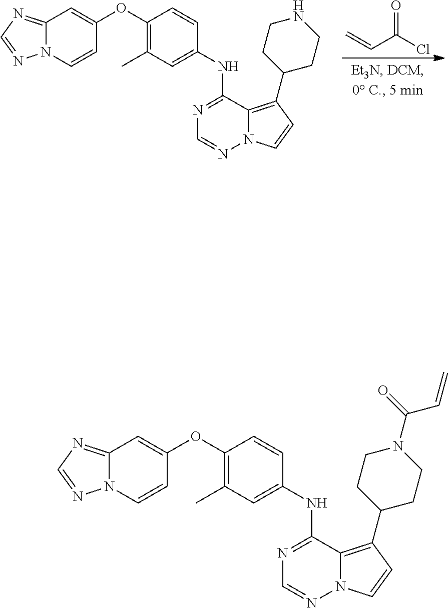

A solution of N-(4-([1,2,4]triazolo[1,5-a]pyridin-7-yloxy)-3-methylphenyl)-5-(piperidin-4-yl)pyrrolo[2,1-f][1,2,4]triazin-4-amine (70 mg, 0.16 mmol), acryloyl chloride (14.38 mg, 0.16 mmol) and Et₃N (32.16 mg, 0.32 mmol) in DCM (2 mL) was stirred for 5 minutes at 0° C. The resulting mixture was purified by Prep-TLC (ethyl acetate) to afford 1-(4-(4-((4-([1,2,4]triazolo[1,5-a]pyridin-7-yloxy)-3-methylphenyl) amino)pyrrolo[2,1-f][1,2,4]triazin-5-yl)piperidin-1-yl)prop-2-en-1-one, Compound 4 (24.6 mg, 30.55%). LCMS (ESI-MS) m/z=495.2 [M+H]⁺.

### Example 5. Compound 5

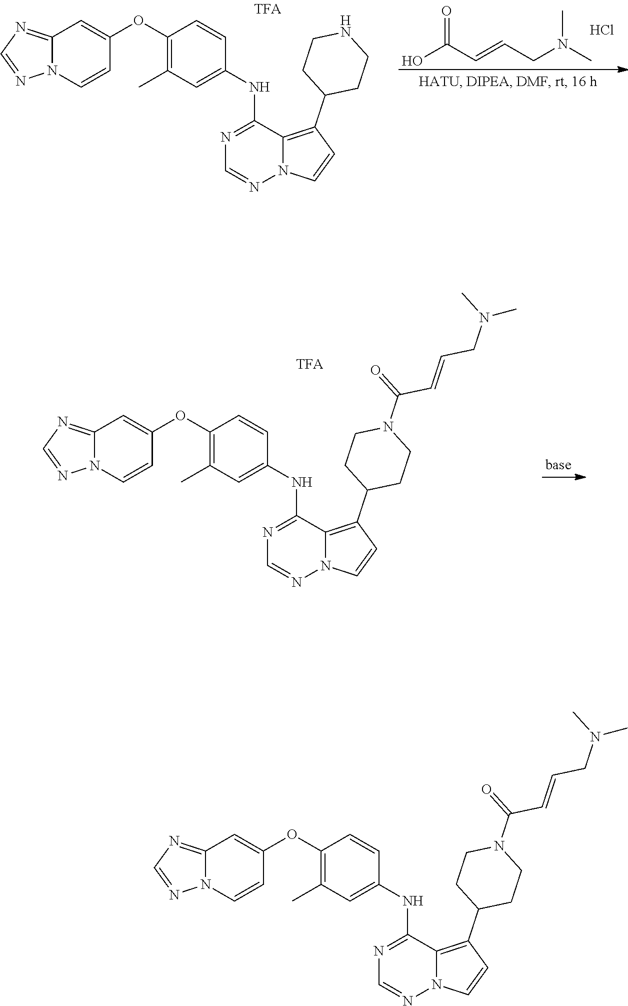

### (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-(dimethylamino)but-2-en-1-one

To a 20 mL scintillation vial with Teflon-coated stir bar was added N-(4-([1,2,4]triazolo[1,5-a]pyridin-7-yloxy)-3-methylphenyl)-5-(piperidin-4-yl)pyrrolo[2,1-f][1,2,4]triazin-4-amine (described in step 3 of Example 4) (TFA salt, 50.0 mg, 1 Eq, 90.2 μmol), (E)-4-(dimethylamino)but-2-enoic acid hydrochloride (22.4 mg, 1.5 Eq, 135 μmol), and HATU (51.4 mg, 1.5 Eq, 135 μmol). The vial was then capped with a rubber septum and evacuated and refilled with N₂ (3×). Then DMF (3.0 mL) and Diisopropylethylamine (46.6 mg, 62.3 μL, 4 Eq, 361 μmol) were added via syringe and the reaction was stirred overnight at rt. The resulting mixture was filtered then purified by preparative reverse phase HPLC (acetonitrile/water gradient with 0.1% TFA) to afford 2,2,2-trifluoroacetaldehyde-(E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-(dimethylamino)but-2-en-1-one (2.0 mg, 3.3% yield. LCMS (ESI) [M+H]⁺=552.3. TFA was subsequently removed by exposure to base to yield Compound 5.

### Example 6. Compound 6

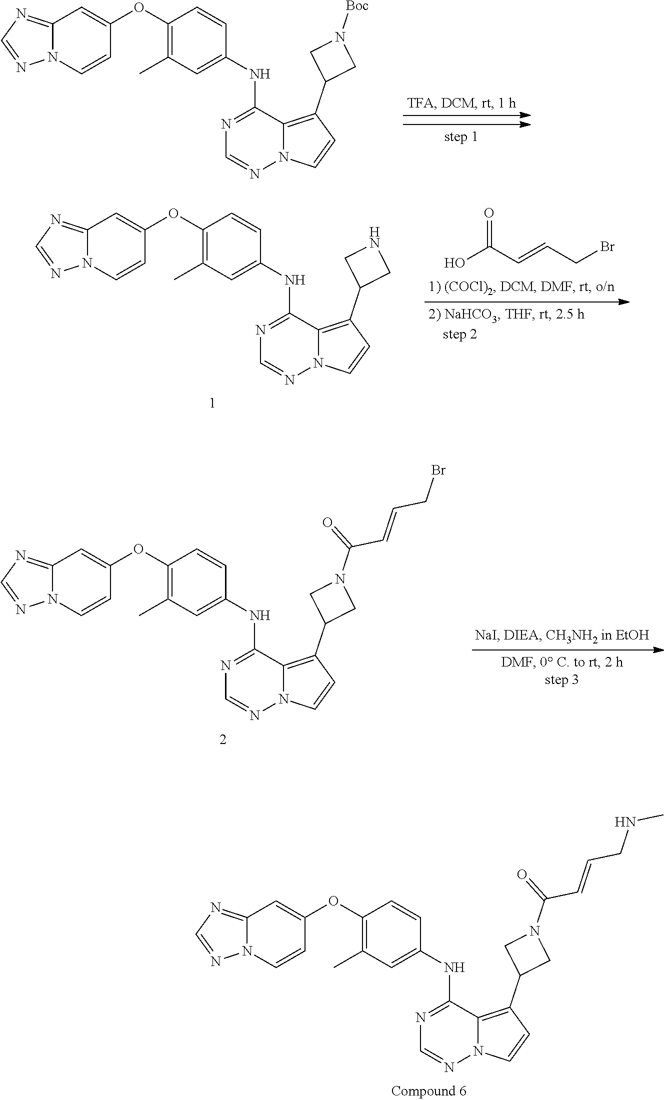

### Step 1. N-(4-([1,2,4]triazolo[1,5-a]pyridin-7-yloxy)-3-methylphenyl)-5-(azetidin-3-yl)pyrrolo[2,1-f][1,2,4]triazin-4-amine

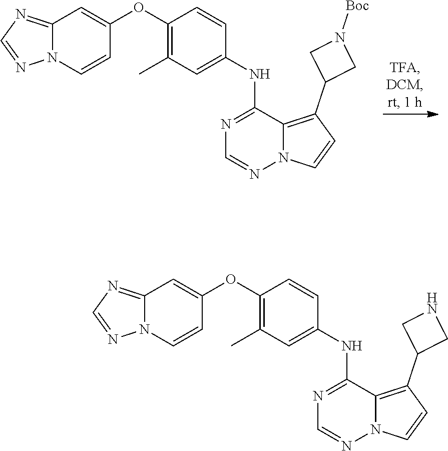 [SMILES: Cc1cc(Nc2ncnn3ccc(C4CN(C(=O)OC(C)(C)C)C4)c23)ccc1Oc1ccn2ncnc2c1 ; Cc1cc(Nc2ncnn3ccc(C4CNC4)c23)ccc1Oc1ccn2ncnc2c1]

TFA (2 mL) was added into a stirred mixture of tert-butyl 3-(4-((4-([1,2,4]triazolo[1,5-a]pyridin-7-yloxy)-3-methylphenyl)amino)pyrrolo[2,1-f][1,2,4]triazin-5-yl)azetidine-1-carboxylate (300 mg, 0.58 mmol) in DCM (5 mL). The resulting mixture was stirred for 1 hour at room temperature and then concentrated under high vacuum to afford the crude product N-(4-([1,2,4]triazolo[1,5-a]pyridin-7-yloxy)-3-methylphenyl)-5-(azetidin-3-yl)pyrrolo[2,1-f][1,2,4]triazin-4-amine (200 mg crude). The crude product was used in the next step immediately without further purification. LCMS (ESI-MS) m/z=413.2 [M+H]⁺.

### Step 2. (E)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-4-bromobut-2-en-1-one

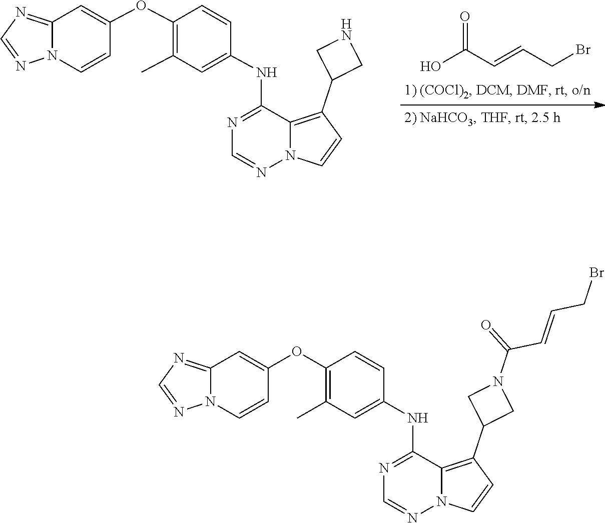 [SMILES: Cc1cc(Nc2ncnn3ccc(C4CNC4)c23)ccc1Oc1ccn2ncnc2c1 ; O=C(O)/C=C/CBr ; Cc1cc(Nc2ncnn3ccc(C4CN(C(=O)/C=C/CBr)C4)c23)ccc1Oc1ccn2ncnc2c1]

Oxalyl chloride (462 mg, 3.63 mmol) was added to a mixture of (E)-4-bromobut-2-enoic acid (240 mg, 1.45 mmol) in DCM (3 mL) at 0° C., followed by addition of catalytic amount of DMF (0.01 mL). The resulting mixture was stirred overnight at room temperature and concentrated under vacuum to afford the crude (E)-4-bromobut-2-enoyl chloride. A mixture of N-(4-([1,2,4]triazolo[1,5-a]pyridin-7-yloxy)-3-methylphenyl)-5-(azetidin-3-yl)pyrrolo[2,1-f][1,2,4]triazin-4-amine (200 mg, 0.48 mmol) and NaHCO₃ (407 mg, 4.85 mmol) in THF (3 mL) was stirred for 1.5 hours at room temperature. Then a solution of (E)-4-bromobut-2-enoyl chloride in 3 mL THF was added to the reaction mixture dropwise and stirred for another hour. The resulting mixture was quenched by addition of water (50 mL) and extracted with ethyl acetate (3×50 mL). The combined organic layers were washed with brine, dried over anhydrous Na₂SO₄ and concentrated under vacuum to afford the crude product. The residue was purified by Prep-TLC (DCM/MeOH10:1) to afford the desired product (E)-1-(3-(4-((4-([1,2,4]triazolo[1,5-a]pyridin-7-yloxy)-3-methylphenyl)amino)-pyrrolo[2,1-f][1,2,4]triazin-5-yl)azetidin-1-yl)-4-bromobut-2-en-1-one (150 mg, 46.5% yield for two steps). LCMS (ESI-MS) m/z=559.2 [M+H]⁺.

### Step 3. (E)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-4-(methylamino)but-2-en-1-one

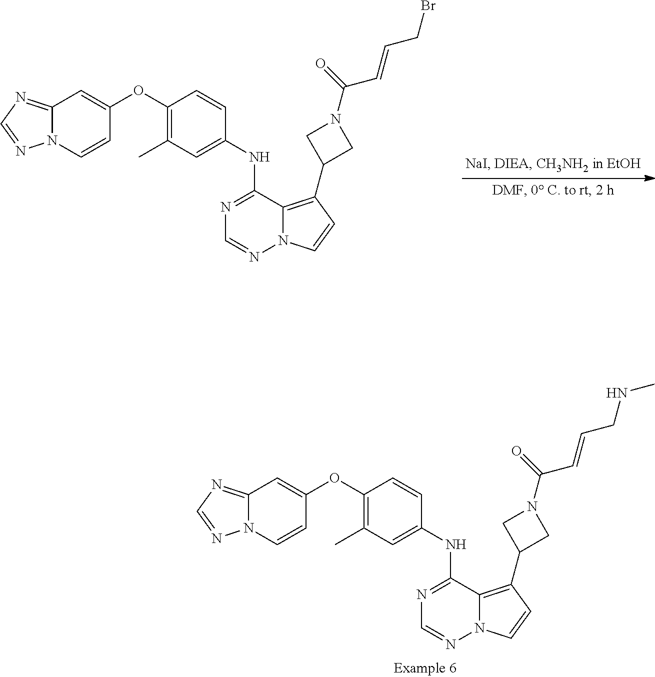 [SMILES: Cc1cc(Nc2ncnn3ccc(C4CN(C(=O)/C=C/CBr)C4)c23)ccc1Oc1ccn2ncnc2c1 ; CNC/C=C/C(=O)N1CC(c2ccn3ncnc(Nc4ccc(Oc5ccn6ncnc6c5)c(C)c4)c23)C1]

A solution of methylamine in EtOH (33% wt, 100 mg, 1.06 mmol) was added to a mixture of (E)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-4-bromobut-2-en-1-one (150 mg, 0.27 mmol) and diisopropylethylamine (104 mg, 0.8 mmol) in DMF (2 mL) at 0° C. The resulting mixture was stirred for 2 hours at room temperature and concentrated under vacuum to afford the crude product. The residue was purified by reverse phase flash chromatography; Mobile Phase A: Water (0.1% FA), Mobile Phase B: ACN; Gradient: 4% B to 31% B to afford the desired product (E)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-4-(methylamino)but-2-en-1-one, Compound 6 (9.5 mg, 6.9% yield). LCMS (ESI-MS) m/z=510.2 [M+H]⁺. ¹H NMR (400 MHz, DMSO-d₆) δ (ppm) 8.94 (d, J=7.4 Hz, 1H), 8.56 (s, 1H), 8.38 (s, 1H), 7.95 (s, 1H), 7.81 (s, 1H), 7.68 (s, 2H), 7.20 (d, J=8.5 Hz, 1H), 7.05-7.04 (m, 1H), 6.95 (s, 1H), 6.80 (s, 1H), 6.69-6.54 (m, 1H), 6.34 (d, J=15.3 Hz, 1H), 4.70 (s, 3H), 4.42 (s, 1H), 4.26 (s, 1H), 4.05 (s, 1H), 3.61 (d, J=5.9 Hz, 2H), 2.47 (s, 3H), 2.24-2.13 (s, 3H).

### Example 7. Compound 7

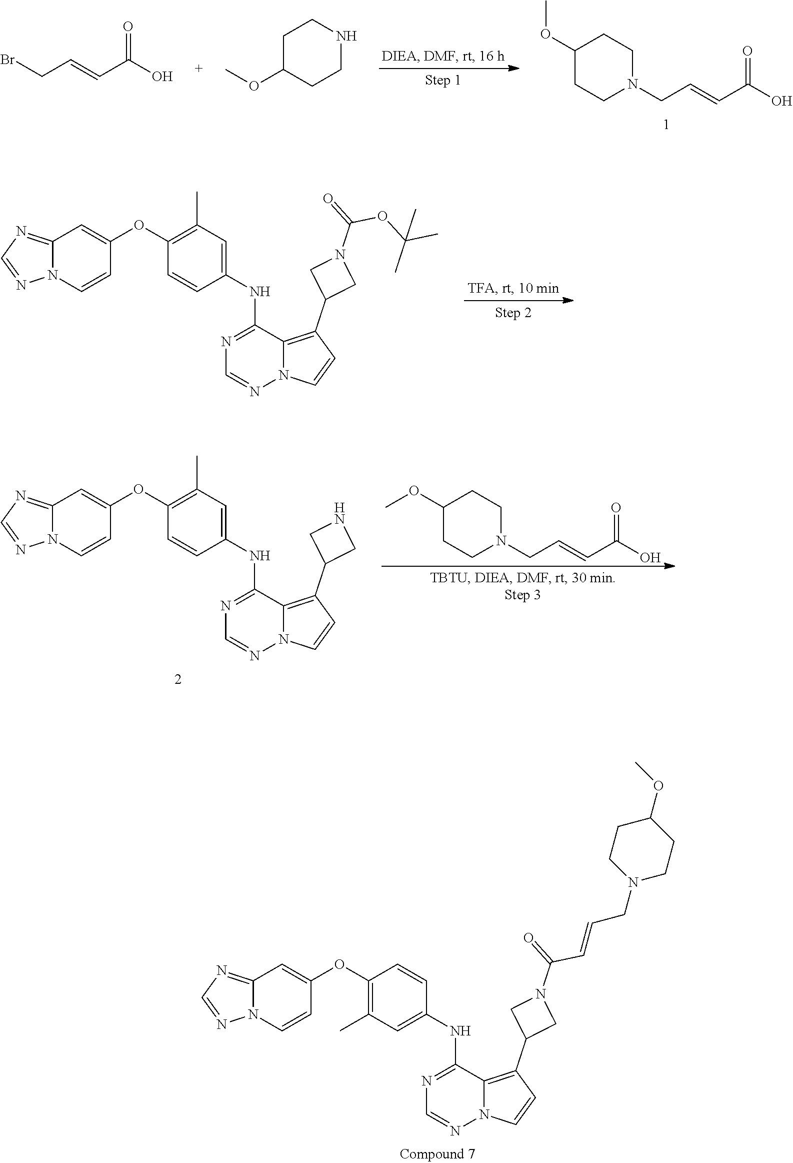

### Step 1. (E)-4-(4-methoxypiperidin-1-yl)but-2-enoic acid

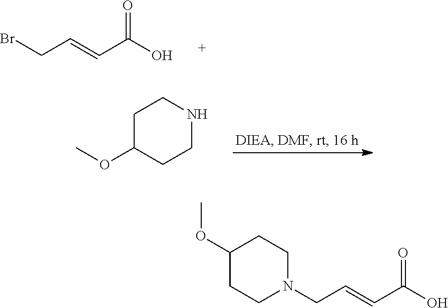

To a stirred mixture of (E)-4-bromobut-2-enoic acid (50 mg, 0.30 mmol) and 4-methoxy-piperidine HCl (51 mg, 0.33 mmol) in DMF (1 mL), diisopropylethylamine (0.12 g, 0.91 mmol) was added, and the reaction stirred at RT overnight. The reaction mixture was used as is in the following step.

### Step 2. N-(4-([1,2,4]triazolo[1,5-a]pyridin-7-yloxy)-3-methylphenyl)-5-(azetidin-3-yl)pyrrolo[2,1-f][1,2,4]triazin-4-amine

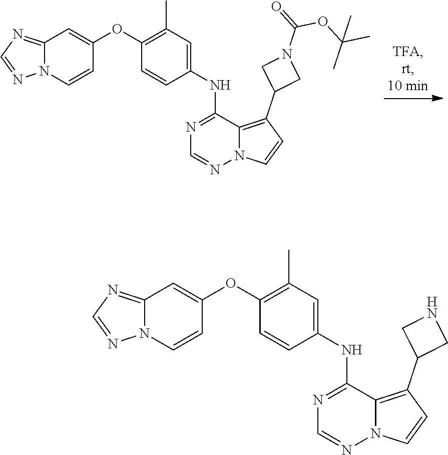 [SMILES: Cc1cc(Nc2ncnn3ccc(C4CN(C(=O)OC(C)(C)C)C4)c23)ccc1Oc1ccn2ncnc2c1 ; Cc1cc(Nc2ncnn3ccc(C4CNC4)c23)ccc1Oc1ccn2ncnc2c1]

TFA (3 mL) was added to tert-butyl 3-(4-((4-([1,2,4]triazolo[1,5-a]pyridin-7-yloxy)-3-methylphenyl)amino)pyrrolo[2,1-f][1,2,4]triazin-5-yl)azetidine-1-carboxylate (438 mg, 855 mol) at RT and the mixture was stirred for 10 min. The resulting mixture was concentrated under vacuum to afford the crude product. The crude was diluted with ethyl acetate (2×20 mL) and washed with NaHCO₃ (20 mL). The organic layers were dried over MgSO₄, and was concentrated to provide N-(4-([1,2,4]triazolo[1,5-a]pyridin-7-yloxy)-3-methylphenyl)-5-(azetidin-3-yl)pyrrolo[2,1-f][1,2,4]triazin-4-amine that was used crude in the next step. LCMS (ESI-MS) m/z=413.2 [M+H]⁺.

### Step 3. (E)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-4-(4-methoxypiperidin-1-yl)but-2-en-1-one

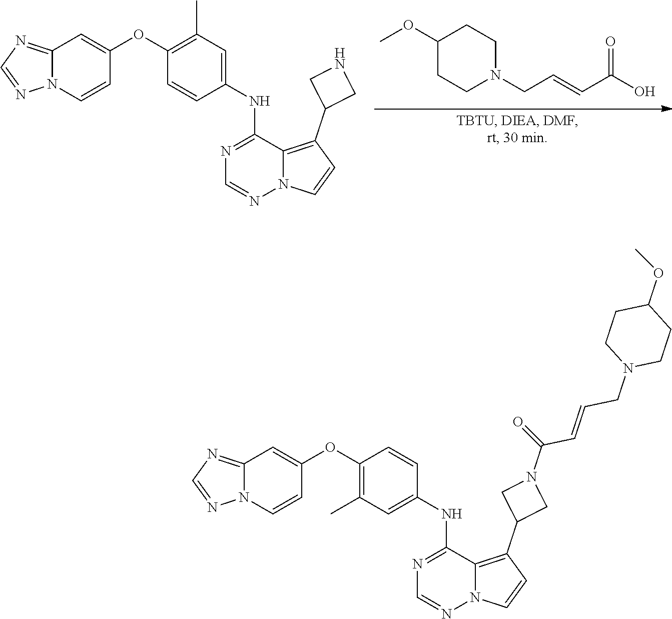

O-(Benzotriazol-1-yl)-N,N,N′,N′-tetramethyluronium Tetrafluoroborate (TBTU) (93 mg, 0.29 mmol) and N-(4-([1,2,4]triazolo[1,5-a]pyridin-7-yloxy)-3-methylphenyl)-5-(azetidin-3-yl)pyrrolo[2,1-f][1,2,4]triazin-4-amine (40 mg, 97 mol) were added to the crude mixture of (E)-4-(4-methoxypiperidin-1-yl)but-2-enoic acid (58 mg, 0.29 mmol) in DMF (1 mL) and diisopropylethylamine (from step 1). The reaction stirred at room temperature for 30 min. The reaction mixture was filtered and purified by prep HPLC, eluted with 10-40% ACN/water/0.1% TFA. Fractions were diluted with ethyl acetate (20 mL) and washed with saturated NaHCO₃ (20 mL), the organic layer was filtered over MgSO₄, and the solvent was evaporated to provide (E)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-4-(4-methoxypiperidin-1-yl)but-2-en-1-one, Compound 7 (17.3 mg, 29%). ¹H NMR (499 MHz, CHLOROFORM-d) 6=8.50 (d, J=7.4 Hz, 1H), 8.23 (s, 1H), 8.00 (s, 1H), 7.63 (d, J=2.7 Hz, 1H), 7.60 (dd, J=2.6, 8.6 Hz, 1H), 7.57 (d, J=2.5 Hz, 1H), 7.10 (d, J=8.5 Hz, 2H), 6.94 (td, J=6.2, 15.3 Hz, 1H), 6.89 (dd, J=2.7, 7.4 Hz, 1H), 6.85 (d, J=2.5 Hz, 1H), 6.73 (d, J=2.7 Hz, 1H), 6.11 (br d, J=15.3 Hz, 1H), 4.83-4.72 (m, 1H), 4.65 (br t, J=8.5 Hz, 1H), 4.46-4.37 (m, 1H), 4.37-4.25 (m, 2H), 3.33 (s, 3H), 3.23 (br d, J=3.3 Hz, 1H), 3.21-3.14 (m, 2H), 2.74 (br s, 2H), 2.34-2.19 (m, 5H), 1.90 (br d, J=12.0 Hz, 2H), 1.63 (br d, J=8.8 Hz, 2H). LCMS (ESI-MS) m/z=594.3 [M+H]⁺.

### Example 8. Compound 8

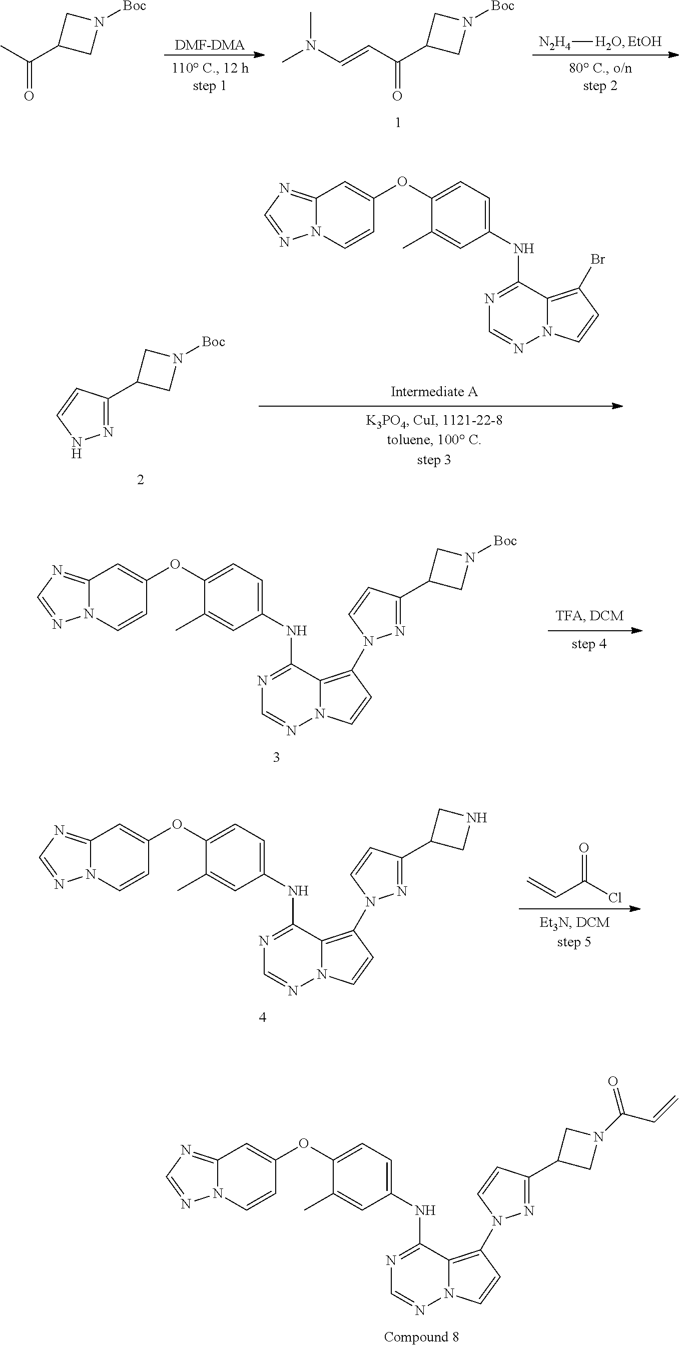

### Step 1. Tert-butyl (E)-3-(3-(dimethylamino)acryloyl)azetidine-1-carboxylate

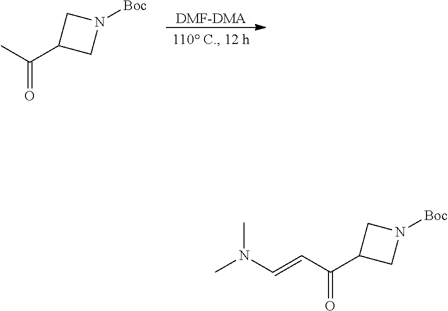

A solution of tert-butyl 3-acetylazetidine-1-carboxylate (2 g, 10.03 mmol) in DMF-DMA (15 mL) was stirred overnight at 110° C. The resulting mixture was concentrated under vacuum to afford tert-butyl 3-[(2E)-3-(dimethylamino)prop-2-enoyl]azetidine-1-carboxylate (2.3 g, crude). LCMS (ESI-MS) m/z=255.2 [M+H]⁺

### Step 2. Tert-butyl 3-(1H-pyrazol-3-yl)azetidine-1-carboxylate

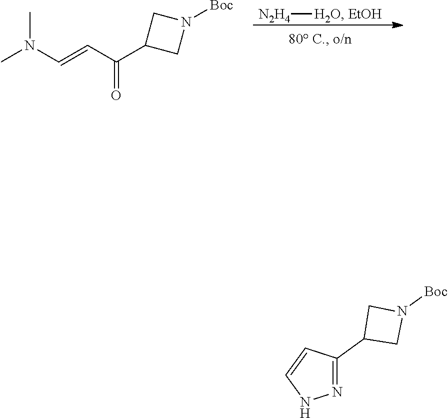

A solution of tert-butyl 3-[(2E)-3-(dimethylamino)prop-2-enoyl]azetidine-1-carboxylate (2.1 g, 8.25 mmol) in hydrazine hydrate (20 mL) was stirred overnight at 80° C. The resulting mixture was concentrated under vacuum. The residue was purified by silica column chromatography (0-40% ethyl acetate in hexanes) to provide the title compound tert-butyl 3-(1H-pyrazol-3-yl)azetidine-1-carboxylate (1.5 g, 66.92%). LCMS (ESI-MS) m/z=447.3 [2M+H]⁺

### Step 3. Tert-butyl 3-(1-(4-((4-([1,2,4]triazolo[1,5-a]pyridin-7-yloxy)-3-methylphenyl)amino)pyrrolo[2,1-f][1,2,4]triazin-5-yl)-1H-pyrazol-3-yl)azetidine-1-carboxylate

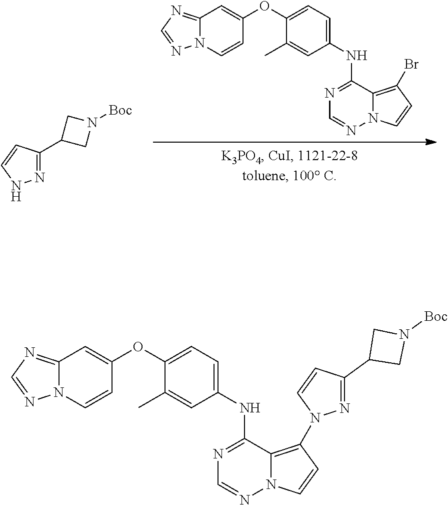 [SMILES: CC(C)(C)OC(=O)N1CC(c2cc[nH]n2)C1 ; Cc1cc(Nc2ncnn3ccc(Br)c23)ccc1Oc1ccn2ncnc2c1 ; Cc1cc(Nc2ncnn3ccc(-n4ccc(C5CN(C(=O)OC(C)(C)C)C5)n4)c23)ccc1Oc1ccn2ncnc2c1]

To a stirred mixture of tert-butyl 3-(1H-pyrazol-3-yl)azetidine-1-carboxylate (200 mg, 0.89 mmol) and N-(4-([1,2,4]triazolo[1,5-a]pyridin-7-yloxy)-3-methylphenyl)-5-bromopyrrolo[2,1-f][1,2,4]triazin-4-amine (781.59 mg, 0.89 mmol) in toluene (3 mL) was added (1R,2R)-cyclohexane-1,2-diamine (153.43 mg, 1.34 mmol) and potassium phosphate (380.27 mg, 1.79 mmol) and cuprous iodide (85.30 mg, 0.44 mmol) under nitrogen atmosphere. The resulting mixture was stirred at 100° C. and overnight. The reaction mixture was filtered off and the filtrate was concentrated. The residue was purified by silica column chromatography (0-40% ethyl acetate in hexanes) to provide the title compound tert-butyl 3-(1-(4-((4-([1,2,4]triazolo[1,5-a]pyridin-7-yloxy)-3-methylphenyl)amino)pyrrolo[2,1-f][1,2,4]triazin-5-yl)-1H-pyrazol-3-yl)azetidine-1-carboxylate (80 mg, 15.43%). LCMS (ESI-MS) m/z=579.3 [M+H]⁺.

### Step 4. N-(4-([1,2,4]triazolo[1,5-a]pyridin-7-yloxy)-3-methylphenyl)-5-(3-(azetidin-3-yl)-1H-pyrazol-1-yl)pyrrolo[2,1-f][1,2,4]triazin-4-amine

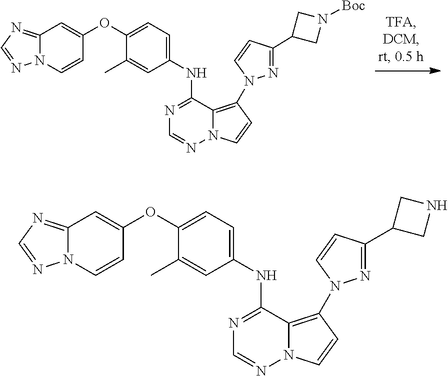 [SMILES: Cc1cc(Nc2ncnn3ccc(-n4ccc(C5CN(C(=O)OC(C)(C)C)C5)n4)c23)ccc1Oc1ccn2ncnc2c1 ; Cc1cc(Nc2ncnn3ccc(-n4ccc(C5CNC5)n4)c23)ccc1Oc1ccn2ncnc2c1]

To a stirred solution of tert-butyl 3-(1-(4-((4-([1,2,4]triazolo[1,5-a]pyridin-7-yloxy)-3-methylphenyl)amino)pyrrolo[2,1-f][1,2,4]triazin-5-yl)-1H-pyrazol-3-yl)azetidine-1-carboxylate (80 mg, 0.13 mmol) in dichloromethane (10 mL) was added trifluoroacetic acid (1 mL). The resulting mixture was stirred at room temperature for 0.5 hour and concentrated under vacuum to afford N-(4-([1,2,4]triazolo[1,5-a]pyridin-7-yloxy)-3-methylphenyl)-5-(3-(azetidin-3-yl)-1H-pyrazol-1-yl)pyrrolo[2,1-f][1,2,4]triazin-4-amine (80 mg crude). LCMS (ESI-MS) m/z=479.2 [M+H]⁺.

### Step 5. 1-(3-(1-(4-((4-([1,2,4]triazolo[1,5-a]pyridin-7-yloxy)-3-methylphenyl)amino)pyrrolo[2,1-f][1,2,4]triazin-5-yl)-1H-pyrazol-3-yl)azetidin-1-yl)prop-2-en-1-one

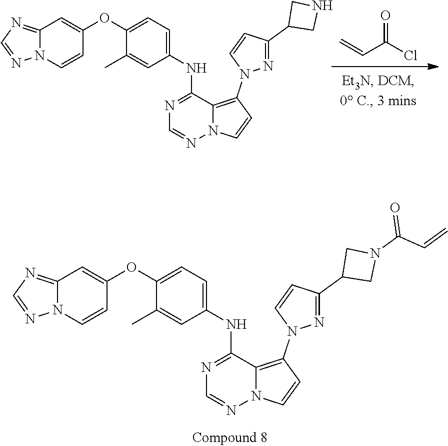

To a stirred mixture of N-(4-([1,2,4]triazolo[1,5-a]pyridin-7-yloxy)-3-methylphenyl)-5-(3-(azetidin-3-yl)-1H-pyrazol-1-yl)pyrrolo[2,1-f][1,2,4]triazin-4-amine (80 mg crude) and Et₃N (67.67 mg, 0.66 mmol) in dichloromethane (2 mL) was added acryloyl chloride (15.13 mg, 0.16 mmol) dropwise at 0° C. and stirred for 3 minutes. The reaction mixture was purified by Prep-TLC to afford the crude product. The crude product (60 mg) was purified by Prep-HPLC with the following conditions (Column: XBridge Shield RP18 OBD Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 28% B to 58% B in 7 min, 58% B) to afford 1-(3-(1-(4-((4-([1,2,4]triazolo[1,5-a]pyridin-7-yloxy)-3-methylphenyl)amino)pyrrolo[2,1-f][1,2,4]triazin-5-yl)-1H-pyrazol-3-yl)azetidin-1-yl)prop-2-en-1-one, Compound 8 (11.2 mg, 12.57%). LCMS (ESI-MS) m/z=533.1 [M+H]⁺.

### Example 9. Compound 9

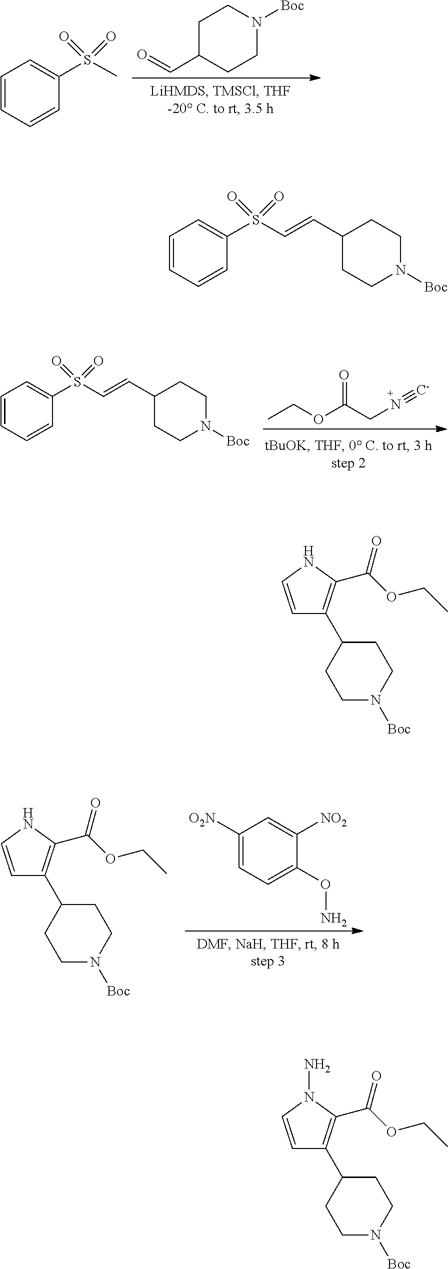

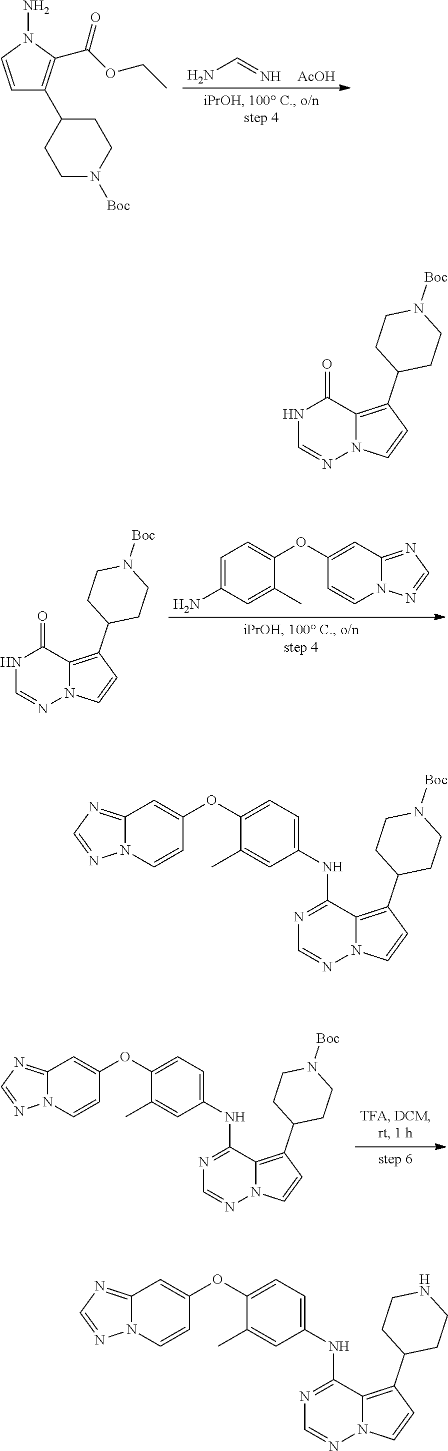

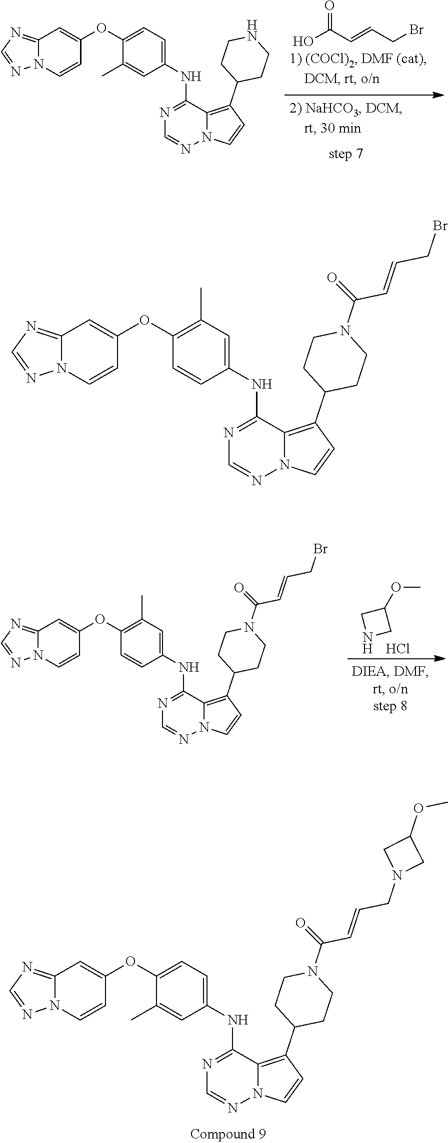

### Step 1. tert-butyl (E)-4-(2-(phenylsulfonyl)vinyl)piperidine-1-carboxylate

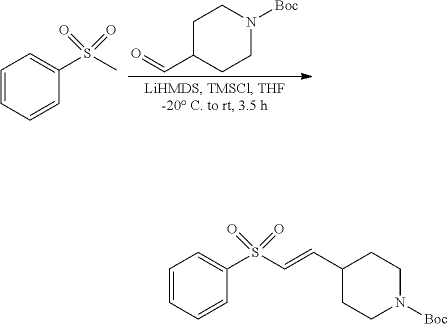

A solution of LiHMDS (1 M solution in THF, 384.6 mL, 384.6 mmol) was added dropwise to a solution of (methylsulfonyl)benzene (50 g, 320.5 mmol) in anhydrous THF (2.5 L) at −20° C. under nitrogen atmosphere. The reaction mixture was allowed to stir for 30 minutes at −20° C. followed by addition of TMSCl (48.8 ml, 99.2 mmol). The resulting mixture was slowly allowed to warm up to room temperature and stirred for another 3 hours. The reaction mixture was quenched by addition of saturated aqueous NH₄Cl (1 L) and extracted with EA (2×1 L). The combined organic layers were washed with brine (2 L), dried over anhydrous Na₂SO₄ and concentrated under vacuum to afford the product. The residue was purified by silica gel column chromatography, eluted with ethyl acetate in petroleum ether from 0% to 20% to afford the desired product tert-butyl (E)-4-(2-(phenylsulfonyl)vinyl)piperidine-1-carboxylate (32.0 g, 44.9% yield). LCMS (ESI-MS) m/z=352.1 [M+H]⁺.

### Step 2. tert-butyl 4-(2-(ethoxycarbonyl)-1H-pyrrol-3-yl)piperidine-1-carboxylate

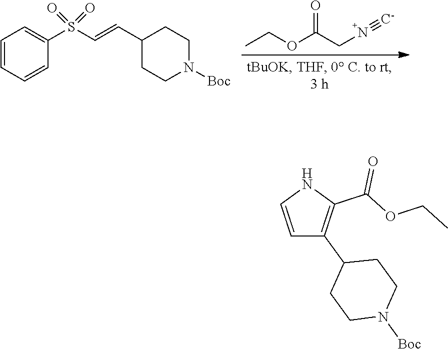

tBuOK (18.8 g, 168 mmol) was added to a solution of ethyl 2-isocyanoacetate (14.5 g, 128.18 mmol) in THF (320 mL) under nitrogen atmosphere. The resulting mixture was stirred at 0° C. for 30 minutes. Then a solution of tert-butyl 4-(2-(ethoxycarbonyl)-1H-pyrrol-3-yl)piperidine-1-carboxylate (32 g, 91.0 mmol) in THF (320 mL) was added to the mixture above. The resulting mixture was warmed to room temperature, stirred for 3 hours and quenched by the addition of saturated aqueous NH₄Cl (100 mL) at 0° C. The resulting mixture was extracted with ethyl acetate (3×250 mL). The combined organic layers were washed with brine (400 mL), dried over anhydrous Na₂SO₄ and concentrated under vacuum to afford the crude product. The residue was purified by silica gel column chromatography, eluted with ethyl acetate in petroleum ether from 0% to 20% to afford the desired product tert-butyl 4-(2-(ethoxycarbonyl)-1H-pyrrol-3-yl)piperidine-1-carboxylate (11.2 g, 38.1% yield). LCMS (ESI-MS) m/z=323.1 [M+H]⁺.

### Step 3. tert-butyl 4-(1-amino-2-(ethoxycarbonyl)-1H-pyrrol-3-yl)piperidine-1-carboxylate

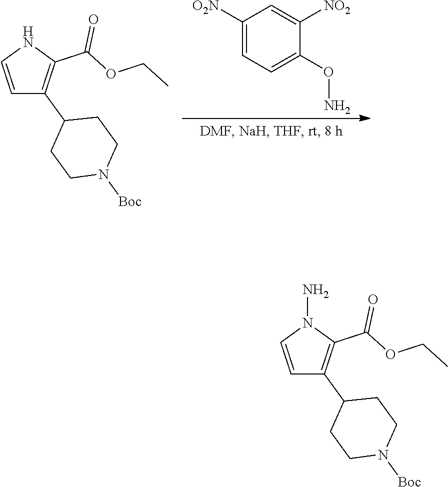 [SMILES: CCOC(=O)c1[nH]ccc1C1CCN(C(=O)OC(C)(C)C)CC1 ; NOc1ccc([N+](=O)[O-])cc1[N+](=O)[O-] ; CCOC(=O)c1c(C2CCN(C(=O)OC(C)(C)C)CC2)ccn1N]

NaH (60% in mineral oil, 5.09 g, 127.17 mmol) in DMF (180 mL) was added to a solution of tert-butyl 4-(2-(ethoxycarbonyl)-1H-pyrrol-3-yl)piperidine-1-carboxylate (20 g, 62.03 mmol) in THF (900 mL) at 0° C. The resulting mixture was stirred for 1 hour at room temperature, followed by addition of O-(2,4-dinitrophenyl)hydroxylamine (23.47 g, 117.86 mmol) portion wise. The resulting mixture was stirred at room temperature for 8 hours, quenched by the addition of saturated aqueous NH₄Cl (300 mL) at 0° C. and extracted with ethyl acetate (3×500 mL). The combined organic layers were washed with brine (100 mL), dried over anhydrous Na₂SO₄ and concentrated under vacuum. The residue was purified by silica gel column chromatography, eluted with ethyl acetate in petroleum ether from 0% to 20% to afford the desired product tert-butyl 4-(1-amino-2-(ethoxycarbonyl)-1H-pyrrol-3-yl)piperidine-1-carboxylate (14 g, 66.8% yield). LCMS (ESI-MS) m/z=338.2 [M+H]⁺.

### Step 4. tert-butyl 4-(4-oxo-3,4-dihydropyrrolo[2,1-f][1,2,4]triazin-5-yl)piperidine-1-carboxylate

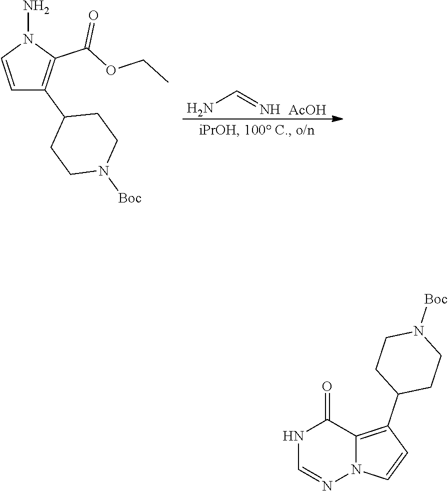

Formamidine acetate (14.62 g, 332 mmol) was added to a solution of tert-butyl 4-(1-amino-2-(ethoxycarbonyl)-1H-pyrrol-3-yl)piperidine-1-carboxylate (14 g, 41.49 mmol) in iPrOH (14 mL) at 25° C. The resulting mixture was stirred overnight at 100° C. After cooled to room temperature, the reaction mixture was quenched by the addition of water (100 mL) and extracted with ethyl acetate (3×100 mL). The combined organic layers were washed with brine (200 mL), dried over anhydrous Na₂SO₄ and concentrated under reduced pressure to afford the crude product. The residue was purified by silica gel column chromatography, eluted with ethyl acetate in petroleum ether from 0% to 50% to afford the desired product tert-butyl 4-(4-oxo-3,4-dihydropyrrolo[2,1-f][1,2,4]triazin-5-yl)piperidine-1-carboxylate (10.4 g, 92.0% yield). LCMS (ESI-MS) m/z=319.2 [M+H]⁺. ¹H NMR (400 MHz, DMSO-d₆) δ 11.47 (d, J=4.0 Hz, 1H), 7.72 (d, J=4.0 Hz, 1H), 7.46 (d, J=2.8 Hz, 1H), 6.45 (d, J=2.7 Hz, 1H), 4.05 (s, 2H), 3.33-3.34 (m, 1H), 2.79 (s, 2H), 1.83-1.74 (m, 2H), 1.51-1.25 (m, 2H), 1.41 (s, 9H).

### Step 5. tert-butyl 4-(4-((4-([1,2,4]triazolo[1,5-a]pyridin-7-yloxy)-3-methylphenyl)amino)pyrrolo[2,1-f][1,2,4]triazin-5-yl)piperidine-1-carboxylate

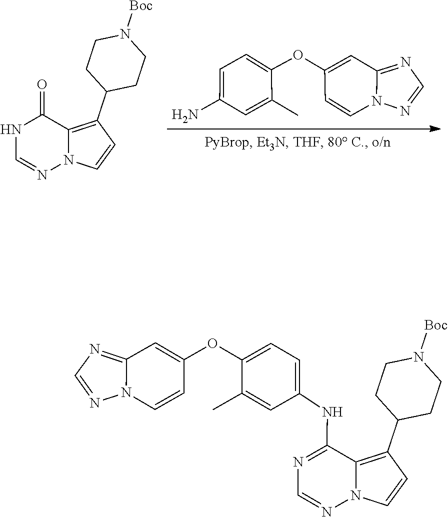 [SMILES: CC(C)(C)OC(=O)N1CCC(c2ccn3nc[nH]c(=O)c23)CC1 ; Cc1cc(N)ccc1Oc1ccn2ncnc2c1 ; Cc1cc(Nc2ncnn3ccc(C4CCN(C(=O)OC(C)(C)C)CC4)c23)ccc1Oc1ccn2ncnc2c1]

Et₃N (3.2 g, 31.5 mmol) was added to a mixture of tert-butyl 4-(4-oxo-3,4-dihydropyrrolo[2,1-f][1,2,4]triazin-5-yl)piperidine-1-carboxylate (3.2 g, 10.5 mmo), 4-([1,2,4]triazolo[1,5-a]pyridin-7-yloxy)-3-methylaniline (4.1 g, 10.5 mmol) and PyBrop (12 g, 25.8 mmol) in THE (50 mL). The resulting mixture was stirred at 80° C. overnight then cooled to room temperature. The reaction mixture was filtered, the filter cake was washed with DCM (300 mL). The filtrate was concentrated under vacuum to afford the crude product. The residue was purified by silica gel column chromatography, eluted with ethyl acetate in petroleum ether from 0% to 100% to afford the desired product tert-butyl 4-(4-((4-([1,2,4]triazolo[1,5-a]pyridin-7-yloxy)-3-methylphenyl)amino)pyrrolo[2,1-f][1,2,4]triazin-5-yl)piperidine-1-carboxylate (1.7 g, 31.1% yield). LCMS (ESI-MS) m/z=541.3 [M+H]⁺. ¹H NMR (400 MHz, DMSO-d₆) δ 8.94-8.92 (m, 1H), 8.47 (s, 1H), 8.38 (d, J=4.4 Hz, 1H), 7.90 (s, 1H), 7.72 (d, J=2.7 Hz, 1H), 7.66 (d, J=2.5 Hz, 1H), 7.61-7.60 (m, 1H), 7.22 (d, J=8.6 Hz, 1H), 7.16-6.99 (m, 1H), 6.79 (d, J=2.7 Hz, 1H), 4.12-4.03 (m, 4H), 3.56-3.55 (m, 1H), 3.17 (d, J=3.9 Hz, 1H), 2.19 (s, 3H), 2.12-2.01 (m, 2H), 1.95-1.87 (m, 2H), 1.41 (s, 9H).

### Step 6. N-(4-([1,2,4]triazolo[1,5-a]pyridin-7-yloxy)-3-methylphenyl)-5-(piperidin-4-yl)pyrrolo[2,1-f][1,2,4]triazin-4-amine

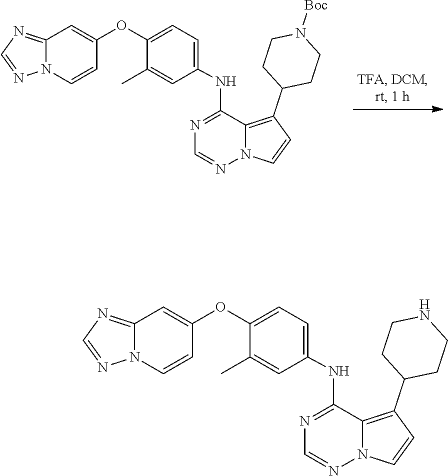 [SMILES: Cc1cc(Nc2ncnn3ccc(C4CCN(C(=O)OC(C)(C)C)CC4)c23)ccc1Oc1ccn2ncnc2c1 ; Cc1cc(Nc2ncnn3ccc(C4CCNCC4)c23)ccc1Oc1ccn2ncnc2c1]

A solution of tert-butyl 4-(4-((4-([1,2,4]triazolo[1,5-a]pyridin-7-yloxy)-3-methylphenyl)amino)pyrrolo[2,1-f][1,2,4]triazin-5-yl)piperidine-1-carboxylate (1 g, 1.85 mmol) and TFA (5 mL) in DCM (5 mL) was stirred for 1 hour at room temperature. The resulting mixture was concentrated under vacuum to afford the crude product N-(4-([1,2,4]triazolo[1,5-a]pyridin-7-yloxy)-3-methylphenyl)-5-(piperidin-4-yl)pyrrolo[2,1-f][1,2,4]triazin-4-amine (1 g crude). LCMS (ESI-MS) m/z=441.2 [M+H]⁺.

### Step 7. (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-bromobut-2-en-1-one

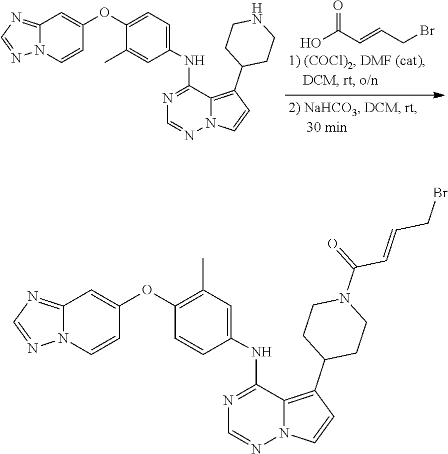 [SMILES: Cc1cc(Nc2ncnn3ccc(C4CCNCC4)c23)ccc1Oc1ccn2ncnc2c1 ; O=C(O)/C=C/CBr ; Cc1cc(Nc2ncnn3ccc(C4CCN(C(=O)/C=C/CBr)CC4)c23)ccc1Oc1ccn2ncnc2c1]

To a cold mixture (0° C.) of (E)-4-bromobut-2-enoic acid (1.46 g, 8.82 mmol) in DMF (0.1 mL) and DCM (15 mL) was added oxalyl chloride (2.42 g, 17.6 mmol) dropwise. The reaction mixture was stirred overnight at room temperature and concentrated under vacuum. The residue was dissolved in DCM (15 mL). The solution was added to a stirred mixture of N-(4-([1,2,4]triazolo[1,5-a]pyridin-7-yloxy)-3-methylphenyl)-5-(piperidin-4-yl)pyrrolo[2,1-f][1,2,4]triazin-4-amine (1 g, 2.27 mmol) and NaHCO₃ (1.14 g, 13.5 mmol) in THF (20 mL). The resulting mixture was stirred for 30 minutes at room temperature and concentrated under vacuum to afford the crude product. The crude product was purified by silica gel column chromatography, eluted with DCM/MeOH (10:1 volume ratio) to afford the desired product (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-bromobut-2-en-1-one (800 mg, 73.6% yield for two steps). LCMS (ESI-MS) m/z=587.1 [M+H]⁺.

### Step 8. (E)-1-(4-(4-((4-([1,2,4]triazolo[1,5-a]pyridin-7-yloxy)-3-methylphenyl)amino)pyrrolo[2,1-f][1,2,4]triazin-5-yl)piperidin-1-yl)-4-bromobut-2-en-1-one

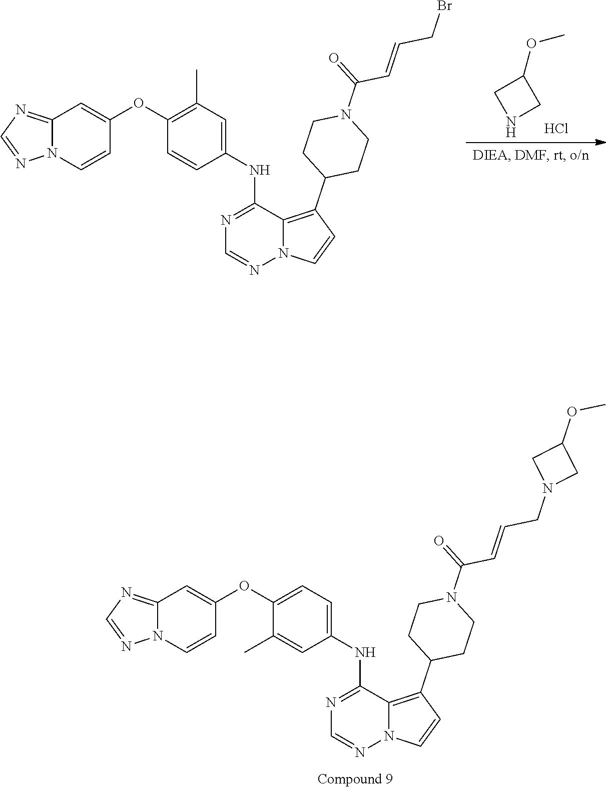

Diisopropylethylamine (66 mg, 0.51 mmol) was added to a mixture of (E)-1-(4-(4-((4-([1,2,4]triazolo[1,5-a]94midazol-7-yloxy)-3-methylphenyl)amino)pyrrolo[2,1-f][1,2,4]triazin-5-yl)piperidin-1-yl)-4-bromobut-2-en-1-one (100 mg, 0.17 mmol) and 3-methoxyazetidine hydrochloride (19 mg, 0.15 mmol) in DMF (1 mL). The resulting mixture was stirred overnight at room temperature. The reaction mixture was purified by Prep-HPLC; Mobile Phase A: Water (10 mmol/L NH₄HCO₃+0.05%/NH₃H₂O), Mobile Phase B: ACN; Gradient: 24% B to 54% to afford the desired product (E)-1-(4-(4-((4-([1,2,4]triazolo[1,5-a]95midazol-7-yloxy)-3-methylphenyl)amino)pyrrolo[2,1-f][1,2,4]triazin-5-yl)piperidin-1-yl)-4-(3-methoxyazetidin-1-yl)but-2-en-1-one, Compound 9 (14.4 mg, 13.6% yield). LCMS (ESI-MS) m/z=594.3 [M+H]⁺. ¹H NMR (400 MHz, Chloroform-d) δ (ppm) 8.50-8.45 (m, 1H), 8.23 (s, 1H), 7.97 (s, 1H), 7.59-7.50 (m, 3H), 7.12 (d, J=8.6 Hz, 1H), 7.04 (s, 1H), 6.93-6.83 (m, 2H), 6.82-6.74 (m, 1H), 6.57 (d, J=2.8 Hz, 1H), 4.89 (s, 1H), 4.33 (s, 1H), 4.15 (s, 1H), 3.90 (s, 1H), 3.45 (d, J=7.1 Hz, 1H), 3.29 (s, 3H), 3.15 (s, 3H), 2.85 (s, 1H), 2.26 (s, 3H), 2.17-2.10 (m, 3H), 1.84-1.80 (m, 3H), 1.25 (s, 2H).

### Example 10. Compound 10

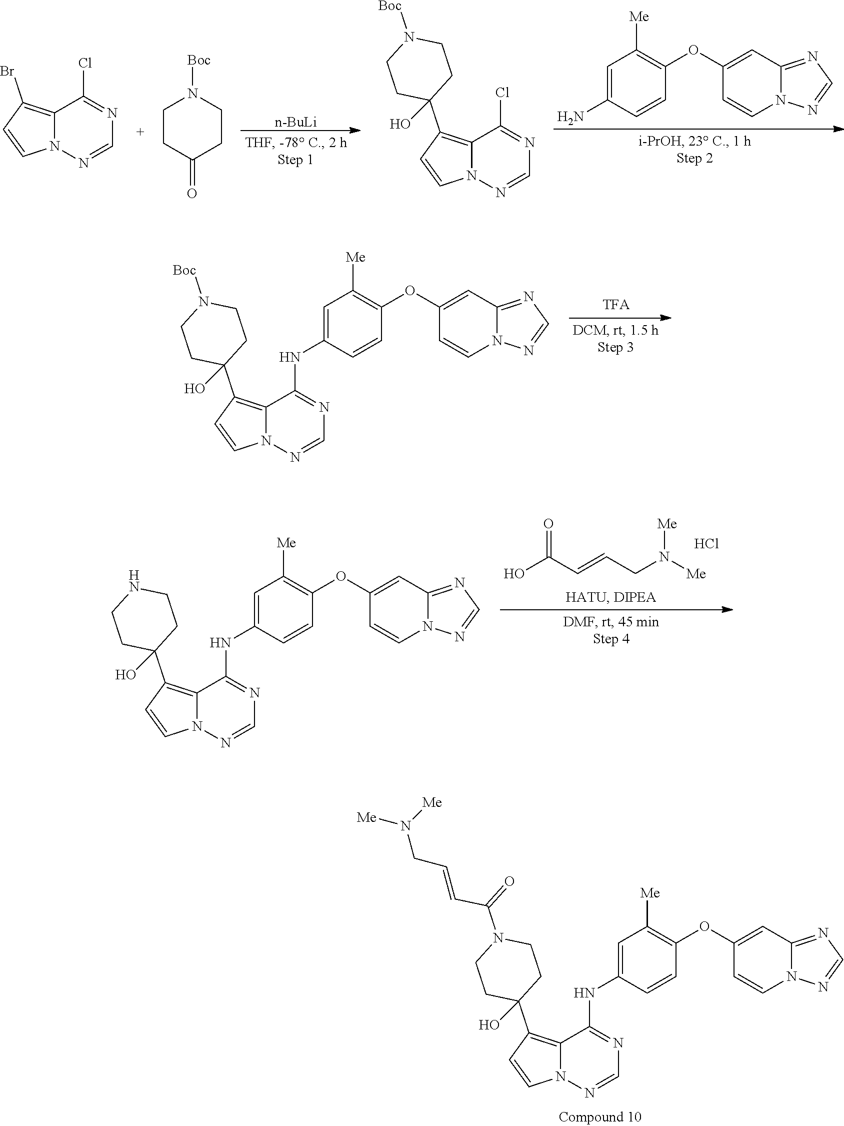

### Step 1. tert-butyl 4-(4-chloropyrrolo[2,1-f][1,2,4]triazin-5-yl)-4-hydroxypiperidine-1-carboxylate

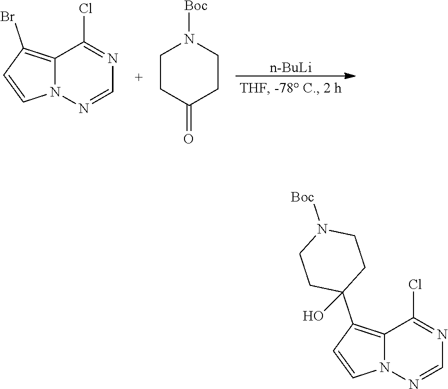

To a reaction tube with a stir bar was added 5-bromo-4-chloropyrrolo[2,1-f][1,2,4]triazine (100 mg, 430 mol). The tube was capped and evacuated and refilled with N₂ (3×). Dry THF (4.0 mL) was added via syringe and the reaction mixture was cooled to −78° C. for 15 min. Then nBuLi (33.1 mg, 215 μL, 2.4 molar, 516 mol) was added slowly via syringe and the reaction was stirred for 30 min. Then tert-butyl 4-oxopiperidine-1-carboxylate (103 mg, 516 mol) in THF (1.0 mL) was added via syringe and the reaction mixture was stirred at −78° C. for 2 h. Then the reaction was quenched with sat NaHCO₃ and warmed to rt. Water was then added (10 mL) and the reaction mixture was extracted with DCM (3×10 mL). The combined organic layers were washed with brine, dried with MgSO₄, and concentrated. Purification via column chromatography (10-100% EtOAc in hexanes) provided tert-butyl 4-(4-chloropyrrolo[2,1-f][1,2,4]triazin-5-yl)-4-hydroxypiperidine-1-carboxylate (77 mg, 51%), used for the next step without further purification. LCMS (ESI) [M+H]⁺=352.1.

### Step 2. tert-butyl 4-(4-((4-([1,2,4]triazolo[1,5-a]pyridin-7-yloxy)-3-methylphenyl)amino)pyrrolo[2,1-f][1,2,4]triazin-5-yl)-4-hydroxypiperidine-1-carboxylate

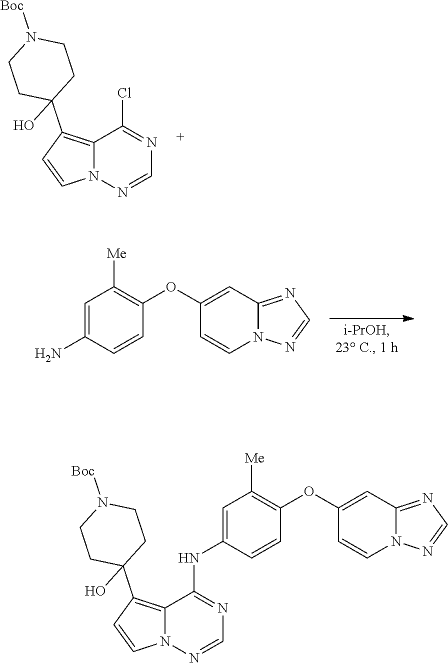 [SMILES: CC(C)(C)OC(=O)N1CCC(O)(c2ccn3ncnc(Cl)c23)CC1 ; Cc1cc(N)ccc1Oc1ccn2ncnc2c1 ; Cc1cc(Nc2ncnn3ccc(C4(O)CCN(C(=O)OC(C)(C)C)CC4)c23)ccc1Oc1ccn2ncnc2c1]

To a 40 mL scintillation vial with a stir bar was added tert-butyl 4-(4-chloropyrrolo[2,1-f][1,2,4]triazin-5-yl)-4-hydroxypiperidine-1-carboxylate (656 mg, 1.86 mmol) and 4-([1,2,4]triazolo[1,5-a]pyridin-7-yloxy)-3-methylaniline (447 mg, 1.86 mmol). The vial was capped and evacuated and refilled with N₂ (3×), then dry isopropanol (12.0 mL) was added via syringe and the reaction mixture stirred at rt. After 1 h the reaction mixture was washed with sat. NaHCO₃ (10 mL) and extracted with DCM (3×10 mL). Purification via column chromatography 10-100% EtOAc/EtOH (3:1 mixture) in hexanes provided tert-butyl 4-(4-((4-([1,2,4]triazolo[1,5-a]pyridin-7-yloxy)-3-methylphenyl)amino)pyrrolo[2,1-f][1,2,4]triazin-5-yl)-4-hydroxypiperidine-1-carboxylate (766 mg, 58%). LCMS (ESI) [M+H]⁺=557.3.

### Step 3. 4-(4-((4-([1,2,4]triazolo[1,5-a]pyridin-7-yloxy)-3-methylphenyl)amino)pyrrolo[2,1-f][1,2,4]triazin-5-yl)piperidin-4-ol

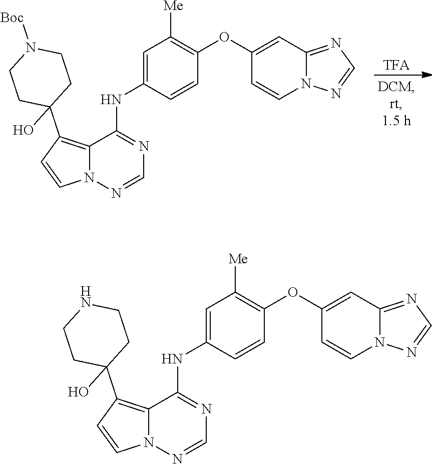 [SMILES: Cc1cc(Nc2ncnn3ccc(C4(O)CCN(C(=O)OC(C)(C)C)CC4)c23)ccc1Oc1ccn2ncnc2c1 ; Cc1cc(Nc2ncnn3ccc(C4(O)CCNCC4)c23)ccc1Oc1ccn2ncnc2c1 ; "TFA"]

To a 1-dram vial with a stir bar was added tert-butyl 4-(4-((4-([1,2,4]triazolo[1,5-a]pyridin-7-yloxy)-3-methylphenyl)amino)pyrrolo[2,1-f][1,2,4]triazin-5-yl)-4-hydroxypiperidine-1-carboxylate (100 mg, 180 mol) and TFA (246 mg, 166 μL, 2.16 mmol). The reaction mixture was stirred at rt for 1.5 h then concentrated in vacuo. The crude product was used in the next step without any further purification. LCMS (ESI) [M+H]=456.2.

### Step 4. (E)-1-(4-(4-((4-([1,2,4]triazolo[1,5-a]pyridin-7-yloxy)-3-methylphenyl)amino)pyrrolo[2,1-f][1,2,4]triazin-5-yl)-4-hydroxypiperidin-1-yl)-4-(dimethylamino)but-2-en-1-one

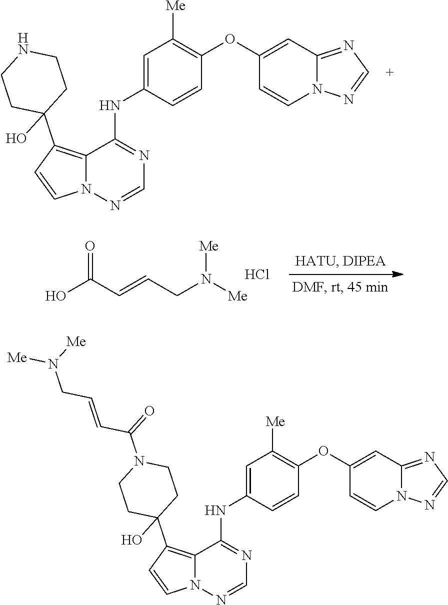 [SMILES: Cc1cc(Nc2ncnn3ccc(C4(O)CCNCC4)c23)ccc1Oc1ccn2ncnc2c1 ; CN(C)C/C=C/C(=O)O ; Cl ; Cc1cc(Nc2ncnn3ccc(C4(O)CCN(C(=O)/C=C/CN(C)C)CC4)c23)ccc1Oc1ccn2ncnc2c1]

To a 2-dram vial with a stir bar added the crude product from the previous step, DMF (2.5 mL), diisopropylethylamine (139 mg, 188 μL, 1.08 mmol), (E)-4-(Dimethylamino)but-2-enoic acid hydrochloride (32.7 mg, 198 mol), and HATU (102 mg, 269 mol), then the reaction mixture was stirred at rt. After 45 min, the reaction mixture was purified directly via prepHPLC 10-50% ACN in 0.1% TFA water. The fractions containing the desired product mass were combined, neutralized with sat. aq. NaHCO₃ (10 mL) and extracted with DCM (4×15 mL). The combined organic layers were washed with brine, dried over MgSO₄, and concentrated to provide (E)-1-(4-(4-((4-([1,2,4]triazolo[1,5-a]pyridin-7-yloxy)-3-methylphenyl)amino)pyrrolo[2,1-f][1,2,4]triazin-5-yl)-4-hydroxypiperidin-1-yl)-4-(dimethylamino)but-2-en-1-one. Compound 10 (59.13 mg, 57%). ¹H NMR (499 MHz, CHLOROFORM-d) δ ppm 11.22 (s, 1H) 8.47 (d, J=7.4 Hz, 1H) 8.06 (s, 1H) 7.99 (s, 1H) 7.79 (d, J=2.5 Hz, 1H) 7.72 (dd, J=8.6, 2.6 Hz, 1H) 7.48 (d, J=2.7 Hz, 1H) 7.07 (d, J=8.8 Hz, 1H) 6.91 (dd, J=7.5, 2.6 Hz, 1H) 6.67-6.81 (m, 2H) 6.43-6.53 (m, 2H) 5.36 (br s, 1H) 4.57 (br d, J=11.8 Hz, 1H) 3.92 (br d, J=12.0 Hz, 1H) 3.67 (br t, J=12.5 Hz, 1H) 3.23 (br t, J=12.2 Hz, 1H) 3.10 (br d, J=6.0 Hz, 2H) 2.27 (s, 6H) 2.22 (s, 3H) 1.89-2.19 (m, 4H).

### Example 11. Compound 11

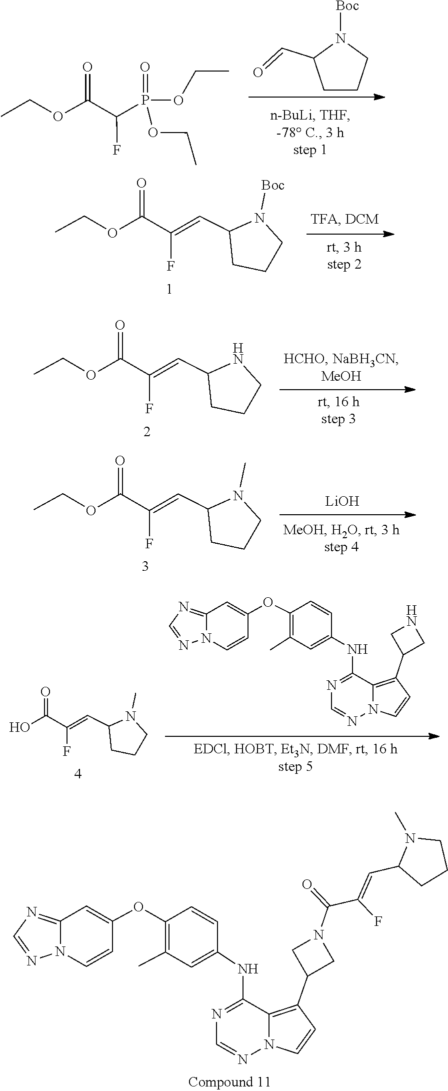

### Step 1. tert-butyl (Z)-2-(3-ethoxy-2-fluoro-3-oxoprop-1-en-1-yl)pyrrolidine-1-carboxylate

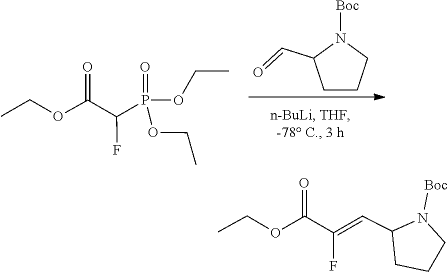

To a cold solution (−78° C.) of ethyl 2-(diethoxyphosphoryl)-2-fluoroacetate (5 g, 20.6 mmol) in THF (50 mL) was added n-BuLi (2.5 M in hexane, 9.91 mL, 24.7 mmol) dropwise. The resulting mixture was stirred for 30 min at −78° C., followed by addition of tert-butyl 2-formylpyrrolidine-1-carboxylate (4.11 g, 20.6 mmol). The resulting solution was stirred at −78° C. for another 3 h. The reaction mixture was quenched with sat. aqueous NH₄Cl at −78° C. and extracted with ethyl acetate (50 mL×2). The combined organic layers were washed with brine (100 mL), dried over anhydrous Na₂SO₄ and concentrated to afford crude product tert-butyl (Z)-2-(3-ethoxy-2-fluoro-3-oxoprop-1-en-1-yl)pyrrolidine-1-carboxylate (5 g, crude). The crude product was used for next step without further purification. LCMS (ESI-MS) m/z=288.1 [M+H]⁺

### Step 2. ethyl (Z)-2-fluoro-3-(pyrrolidin-2-yl)acrylate

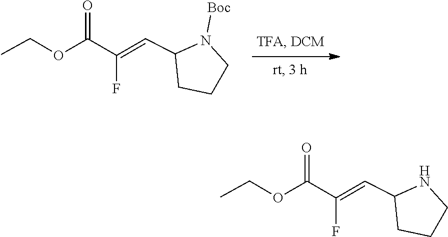 [SMILES: CCOC(=O)/C(F)=C/C1CCCN1C(=O)OC(C)(C)C ; CCOC(=O)/C(F)=C/C1CCCN1]

To a solution of tert-butyl (Z)-2-(3-ethoxy-2-fluoro-3-oxoprop-1-en-1-yl)pyrrolidine-1-carboxylate (5 g, 17.4 mmol) in DCM (50 mL) was added TFA (10 mL). The resulting solution was stirred at room temperature for 3 h and concentrated under vacuum to afford the crude product ethyl (Z)-2-fluoro-3-(pyrrolidin-2-yl)acrylate (4 g). The crude product was used for next step without further purification. LCMS (ESI-MS) m/z=188.2 [M+H]⁺

### Step 3. ethyl (Z)-2-fluoro-3-(1-methylpyrrolidin-2-yl)acrylate

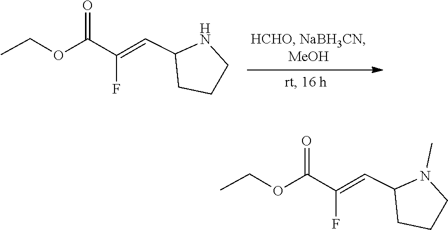

To a solution of ethyl (Z)-2-fluoro-3-(pyrrolidin-2-yl)acrylate (2 g, 10.7 mmol) in methanol (40 mL) was added formaldehyde (1.28 g, 42.7 mmol) and NaBH₃CN (1.01 g, 16.0 mmol). The resulting mixture was stirred at room temperature for 16 h, quenched by addition of sat. aqueous NH₄Cl (50 mL) and extracted with ethyl acetate (50 mL×3). The combined organic phase were washed with brine (100 mL), dried with anhydrous Na₂SO₄, filtered and concentrated to afford ethyl (Z)-2-fluoro-3-(1-methylpyrrolidin-2-yl)acrylate (1 g, 27.7% yield). LCMS (ESI-MS) m/z=201.9 [M+H]⁺.

### Step 4. (Z)-2-fluoro-3-(1-methylpyrrolidin-2-yl)acrylic acid

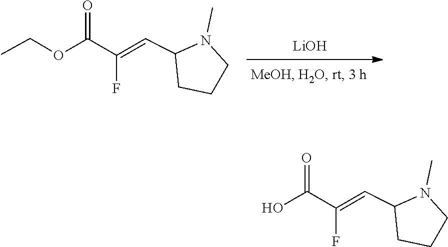

Lithium hydroxide (335.6 mg, 14.0 mmol) was added to a stirred mixture of ethyl (2Z)-2-fluoro-3-(1-methylpyrrolidin-2-yl)prop-2-enoate (940 mg, 4.67 mmol) in methanol (10 mL) and water (3 mL). The resulting mixture was stirred at room temperature for 3 h and concentrated to afford (2Z)-2-fluoro-3-(1-methylpyrrolidin-2-yl)prop-2-enoic acid (crude) without neutralization. LCMS (ESI-MS) m/z=174.1 [M+H]⁺.

### Step 5. (Z)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-2-fluoro-3-(1-methylpyrrolidin-2-yl)prop-2-en-1-one

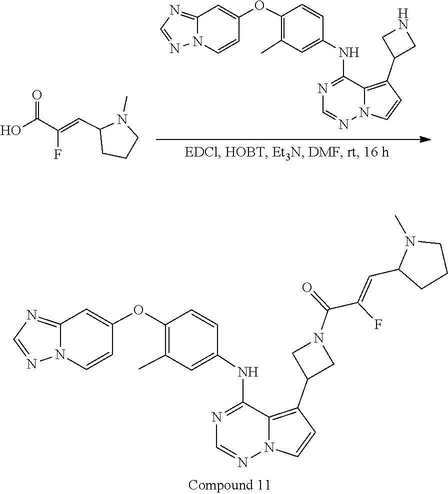 [SMILES: CN1CCCC1/C=C(\F)C(=O)O ; Cc1cc(Nc2ncnn3ccc(C4CNC4)c23)ccc1Oc1ccn2ncnc2c1 ; Cc1cc(Nc2ncnn3ccc(C4CN(C(=O)/C(F)=C/C5CCCN5C)C4)c23)ccc1Oc1ccn2ncnc2c1]

A mixture of (2Z)-2-fluoro-3-(1-methylpyrrolidin-2-yl)prop-2-enoic acid (200 mg, crude, 1.16 mmol), N-(4-([1,2,4]triazolo[1,5-a]pyridin-7-yloxy)-3-methylphenyl)-5-(azetidin-3-yl)pyrrolo[2,1-f][1,2,4]triazin-4-amine (476.3 mg, 1.16 mmol), EDCI (198.5 mg, 1.39 mmol), HOBT (187.3 mg, 1.39 mmol) and Et₃N (233.7 mg, 2.31 mmol) in DMF (5 mL) was stirred at room temperature for 16 h. The reaction mixture was purified by Prep-HPLC, Mobile Phase A: Water (0.1% FA), Mobile Phase B: ACN; Gradient: 11% B to 31% B to afford (Z)-1-(3-(4-((4-([1,2,4]triazolo[1,5-a]pyridin-7-yloxy)-3-methylphenyl)amino)pyrrolo[2,1-f][1,2,4]triazin-5-yl)azetidin-1-yl)-2-fluoro-3-(1-methylpyrrolidin-2-yl)prop-2-en-1-one, Compound 11 (11.5 mg, 2% yield). LCMS (ESI-MS) m/z=568.4 [M+H]⁺. ¹H NMR (400 MHz, DMSO-d₆) δ (ppm) 8.95 (s, 1H), 8.54 (s, 1H), 8.38 (s, 1H), 8.18 (s, 1H), 7.95 (s, 1H), 7.82 (s, 1H), 7.67-7.33 (m, 2H), 7.22-7.12 (m, 1H), 7.10-6.79 (m, 2H), 6.78-6.56 (m, 1H), 5.70-5.58 (m, 1H), 4.80-4.63 (m, 2H), 4.51-4.37 (m, 2H), 4.08-4.04 (m, 1H), 3.70-3.58 (m, 1H), 3.03-2.95 (m, 1H), 2.19-2.13 (m, 6H), 2.04-1.81 (m, 2H), 1.71 (s, 1H), 1.47 (s, 1H).

### Example 12. Compound 12

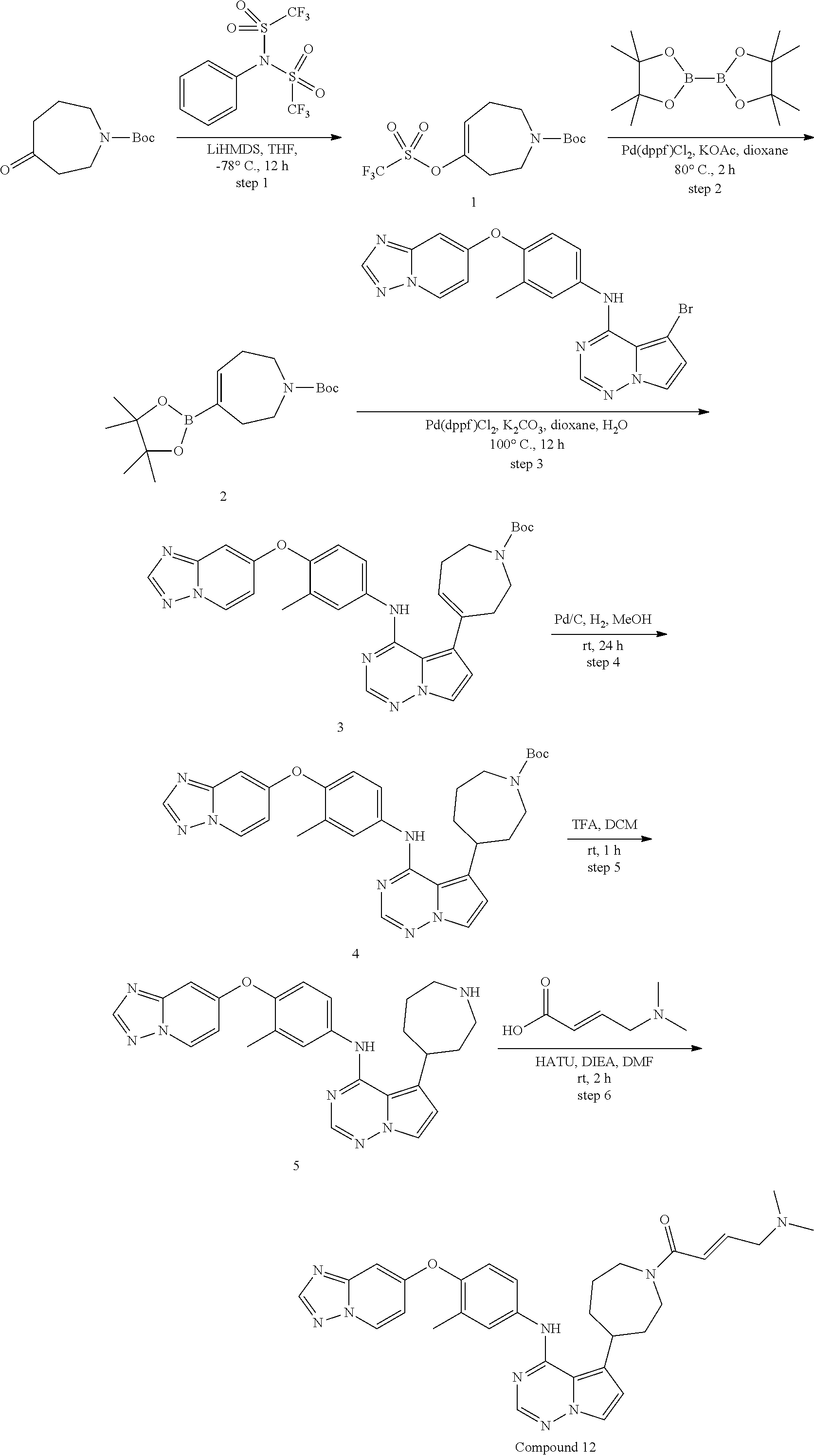

### Step 1. tert-butyl 4-(((trifluoromethyl)sulfonyl)oxy)-2,3,6,7-tetrahydro-1H-azepine-1-carboxylate

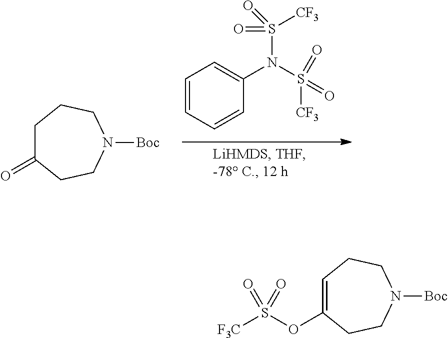 [SMILES: CC(C)(C)OC(=O)N1CCCC(=O)CC1 ; O=S(=O)(N(c1ccccc1)S(=O)(=O)C(F)(F)F)C(F)(F)F ; CC(C)(C)OC(=O)N1CCC=C(OS(=O)(=O)C(F)(F)F)CC1]

A solution of tert-butyl 4-oxoazepane-1-carboxylate (5 g, 23.47 mmol) in THF (100 mL) was treated with a solution of LiHMDS (1 M in THF, 25.8 mL, 25.78 mmol) for 1 hour at −78° C. under nitrogen atmosphere. A solution of 1,1,1-trifluoro-N-phenyl-N-((trifluoromethyl)sulfonyl)methanesulfonamide (9.21 g, 25.82 mmol) in THF (150 mL) was then added dropwise at −78° C. over a period of 0.5 hour. The resulting mixture was stirred for 3 hours at −78° C. and overnight at room temperature under nitrogen atmosphere. The reaction mixture was quenched with water (1000 mL) at 0° C. and extracted with ethyl acetate (3×1000 mL). The combined organic layers were dried over anhydrous Na₂SO₄, filtered and concentrated under vacuum to afford the crude product. The crude product was purified by silica gel column chromatography, eluted with petroleum ether/ethyl acetate=9:1 to afford the crude product tert-butyl 4-(((trifluoromethyl)sulfonyl)oxy)-2,3,6,7-tetrahydro-1H-azepine-1-carboxylate (6.8 g crude). LCMS (ESI-MS) m/z=346.0 [M+H]⁺.

### Step 2. tert-butyl 4-(4,4,5,5-tetramethyl-1,3,2-dioxaborolan-2-yl)-2,3,6,7-tetrahydro-1H-azepine-1-carboxylate

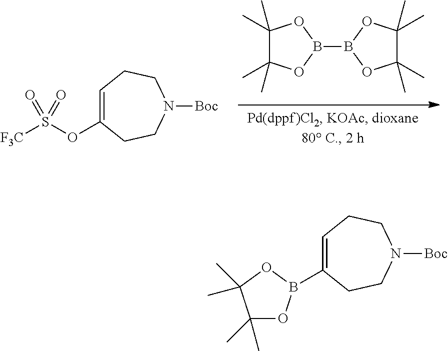

A mixture of tert-butyl 4-(((trifluoromethyl)sulfonyl)oxy)-2,3,6,7-tetrahydro-1H-azepine-1-carboxylate (6.7 g, 19.40 mmol), 4,4,4′,4′,5,5,5′,5′-octamethyl-2,2′-bi(1,3,2-dioxaborolane) (9.85 g, 38.80 mmol), KOAc (3.81 g, 38.80 mmol) and Pd(dppf)Cl₂ (1.42 g, 1.94 mmol) in dioxane (200 mL) was stirred for 2 hours at 80° C. under nitrogen atmosphere. The reaction mixture was allowed to cool down to room temperature, filtered and the filtrate was concentrated under vacuum to afford the crude product. The crude product was purified by silica gel column chromatography, eluted with petroleum ether/ethyl acetate=2:1 to afford tert-butyl 4-(4,4,5,5-tetramethyl-1,3,2-dioxaborolan-2-yl)-2,3,6,7-tetrahydro-1H-azepine-1-carboxylate (1.8 g, 23.7% yield). LCMS (ESI-MS) m/z=324.1 [M+H]⁺

### Step 3. tert-butyl 4-(4-((4-([1,2,4]triazolo[1,5-a]pyridin-7-yloxy)-3-methylphenyl)amino)pyrrolo[2,1-f][1,2,4]triazin-5-yl)-2,3,6,7-tetrahydro-1H-azepine-1-carboxylate

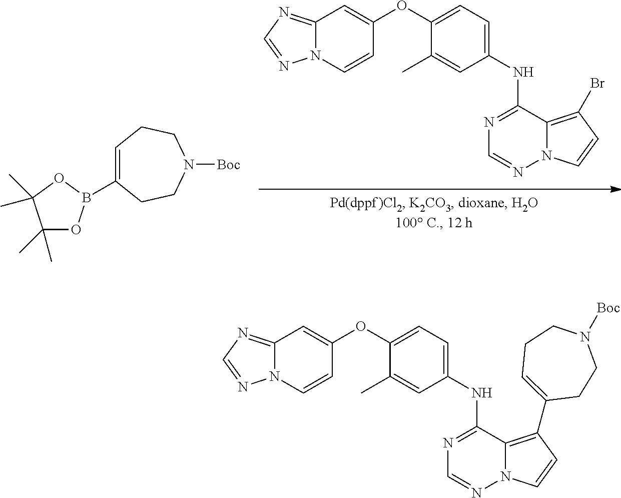 [SMILES: CC(C)(C)OC(=O)N1CCC=C(B2OC(C)(C)C(C)(C)O2)CC1 ; Cc1cc(Nc2ncnn3ccc(Br)c23)ccc1Oc1ccn2ncnc2c1 ; Cc1cc(Nc2ncnn3ccc(C4=CCCN(C(=O)OC(C)(C)C)CC4)c23)ccc1Oc1ccn2ncnc2c1]

A mixture of tert-butyl 4-(4,4,5,5-tetramethyl-1,3,2-dioxaborolan-2-yl)-2,3,6,7-tetrahydro-1H-azepine-1-carboxylate (1.7 g, 5.25 mmol), 5-bromo-N-(4-([1,2,4]triazolo[1,5-a]pyridin-7-yloxy)-3-methylphenyl)-5-bromopyrrolo[2,1-f][1,2,4]triazin-4-amine (2.29 g, 5.25 mmol), K₂CO₃ (1.45 g, 10.51 mmol) and Pd(dppf)Cl₂ (0.38 g, 0.52 mmol) in 1,4-dioxane (24 mL) and water (6 mL) was stirred for 12 hours at 100° C. under nitrogen atmosphere. The resulting mixture was filtered and the filtrate was concentrated under vacuum to afford the crude product. The crude product was purified by silica gel column chromatography, eluted with petroleum ether/ethyl acetate=2:1 to afford tert-butyl 4-(4-((4-([1,2,4]triazolo[1,5-a]pyridin-7-yloxy)-3-methylphenyl)amino)pyrrolo[2,1-f][1,2,4]triazin-5-yl)-2,3,6,7-tetrahydro-1H-azepine-1-carboxylate (1.3 g, 42.2% yield). LCMS (ESI-MS) m/z=553.2 [M+H]⁺

### Step 4. tert-butyl 4-(4-((4-([1,2,4]triazolo[1,5-a]pyridin-7-yloxy)-3-methylphenyl)amino)pyrrolo[2,1-f][1,2,4]triazin-5-yl)azepane-1-carboxylate

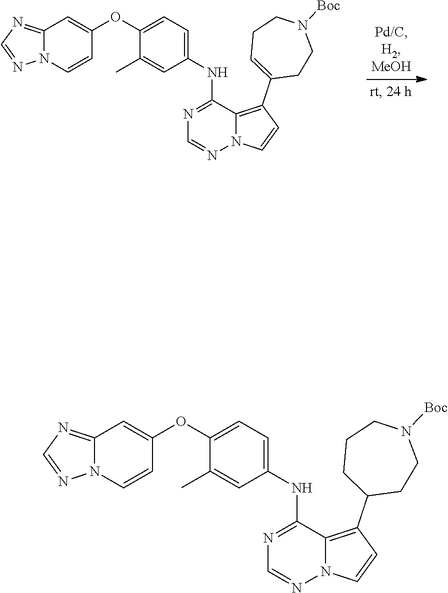 [SMILES: Cc1cc(Nc2ncnn3ccc(C4=CCCN(C(=O)OC(C)(C)C)CC4)c23)ccc1Oc1ccn2ncnc2c1 ; Cc1cc(Nc2ncnn3ccc(C4CCCN(C(=O)OC(C)(C)C)CC4)c23)ccc1Oc1ccn2ncnc2c1]

A mixture of tert-butyl 4-(4(4-([1,2,4]triazolo[1,5-a]pyridin-7-yloxy)-3-methylphenyl)amino)pyrrolo[2,1-f][1,2,4]triazin-5-yl)-2,3,6,7-tetrahydro-1H-azepine-1-carboxylate (1.3 g, 2.35 mmol) and Pd/C (2.5 g, 23.52 mmol) in MeOH (20 mL) was degassed under vacuum and charged with an atmospheric pressure of hydrogen. The resulting mixture was stirred for 24 hours at room temperature. The solids were filtered off and the filtrate was concentrated under vacuum to afford the crude product tert-butyl 4-(4-((4-([1,2,4]triazolo[1,5-a]pyridin-7-yloxy)-3-methylphenyl)amino)pyrrolo[2,1-f][1,2,4]triazin-5-yl)azepane-1-carboxylate (700 mg crude). The crude product was used in the next step directly without further purification. LCMS (ESI-MS) m/z=555.4 [M+H]⁺.

### Step 5. N-(4-([1,2,4]triazolo[1,5-a]pyridin-7-yloxy)-3-methylphenyl)-5-(azepan-4-yl)pyrrolo[2,1-f][1,2,4]triazin-4-amine

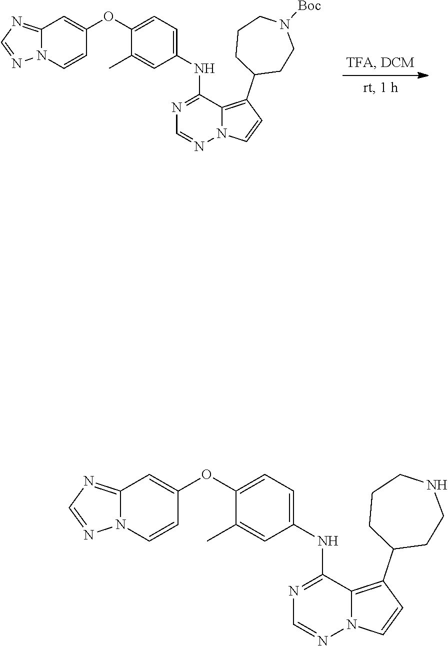 [SMILES: Cc1cc(Nc2ncnn3ccc(C4CCCN(C(=O)OC(C)(C)C)CC4)c23)ccc1Oc1ccn2ncnc2c1 ; Cc1cc(Nc2ncnn3ccc(C4CCCNCC4)c23)ccc1Oc1ccn2ncnc2c1]

A mixture of tert-butyl 4-(4-((4-([1,2,4]triazolo[1,5-a]pyridin-7-yloxy)-3-methylphenyl)amino)pyrrolo[2,1-f][1,2,4]triazin-5-yl)azepane-1-carboxylate (300 mg, 0.54 mmol) and TFA (1 mL, 13.46 mmol) in DCM (10 mL) was stirred for 1 hour at room temperature and then concentrated under vacuum to afford the crude product N-(4-([1,2,4]triazolo[1,5-a]pyridin-7-yloxy)-3-methylphenyl)-5-(azepan-4-yl)pyrrolo[2,1-f][1,2,4]triazin-4-amine (360 mg crude). The crude product was used in the next step without further purification. LCMS (ESI-MS) m/z=455.3 [M+H]⁺

### Step 6. (E)-1-(4-(4-((4-([1,2,4]triazolo[1,5-a]pyridin-7-yloxy)-3-methylphenyl)amino)pyrrolo[2,1-f][1,2,4]triazin-5-yl)azepan-1-yl)-4-(dimethylamino)but-2-en-1-one

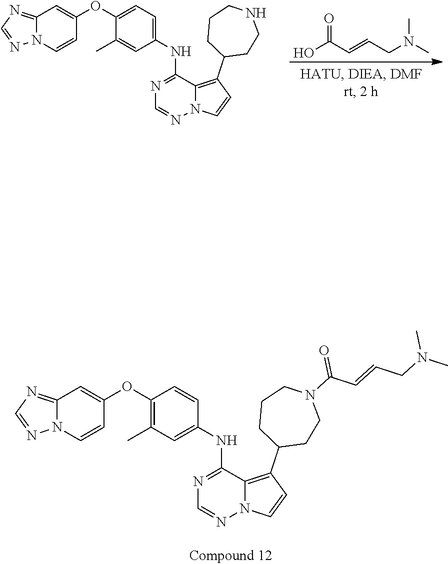

A mixture of N-(4-([1,2,4]triazolo[1,5-a]pyridin-7-yloxy)-3-methylphenyl)-5-(azepan-4-yl)pyrrolo[2,1-f][1,2,4]triazin-4-amine (360 mg, 0.79 mmol), (E)-4-(dimethylamino)but-2-enoic acid (133 mg, 1.03 mmol), diisopropylethylamine (204.73 mg, 1.58 mmol) and HATU (451.73 mg, 1.18 mmol) in DMF (4 mL) was stirred at room temperature for 2 hours. The reaction mixture was filtered and the filtrate was purified by Prep-HPLC, Mobile Phase A: Water (0.1% FA), Mobile Phase B: ACN; Gradient: 15% B to 30% B to afford the desired product (E)-1-(4-(4-((4-([1,2,4]triazolo[1,5-a]pyridin-7-yloxy)-3-methylphenyl)amino)pyrrolo[2,1-f][1,2,4]triazin-5-yl)azepan-1-yl)-4-(dimethylamino)but-2-en-1-one, Compound 12 (6.7 mg, 2% yield). LCMS (ESI-MS) m/z=566.4 [M+H]⁺. ¹H NMR (400 MHz, methanol-d₄) δ (ppm) 8.74 (s, 1H), 8.32 (s, 2H), 7.72 (s, 1H), 7.62 (s, 1H), 7.56-7.41 (m, 2H), 7.19-7.17 (m, 1H), 7.10-7.06 (m, 1H), 6.95-6.88 (m, 1H), 6.84 (s, 1H), 6.77-6.70 (m, 1H), 6.64-6.62 (m, 1H), 3.96-3.61 (m, 6H), 3.55-3.39 (m, 1H), 2.82-2.73 (m, 6H), 2.35-2.30 (m, 1H), 2.24 (s, 5H), 1.98-1.73 (m, 3H).

### Example 13. Compound 13

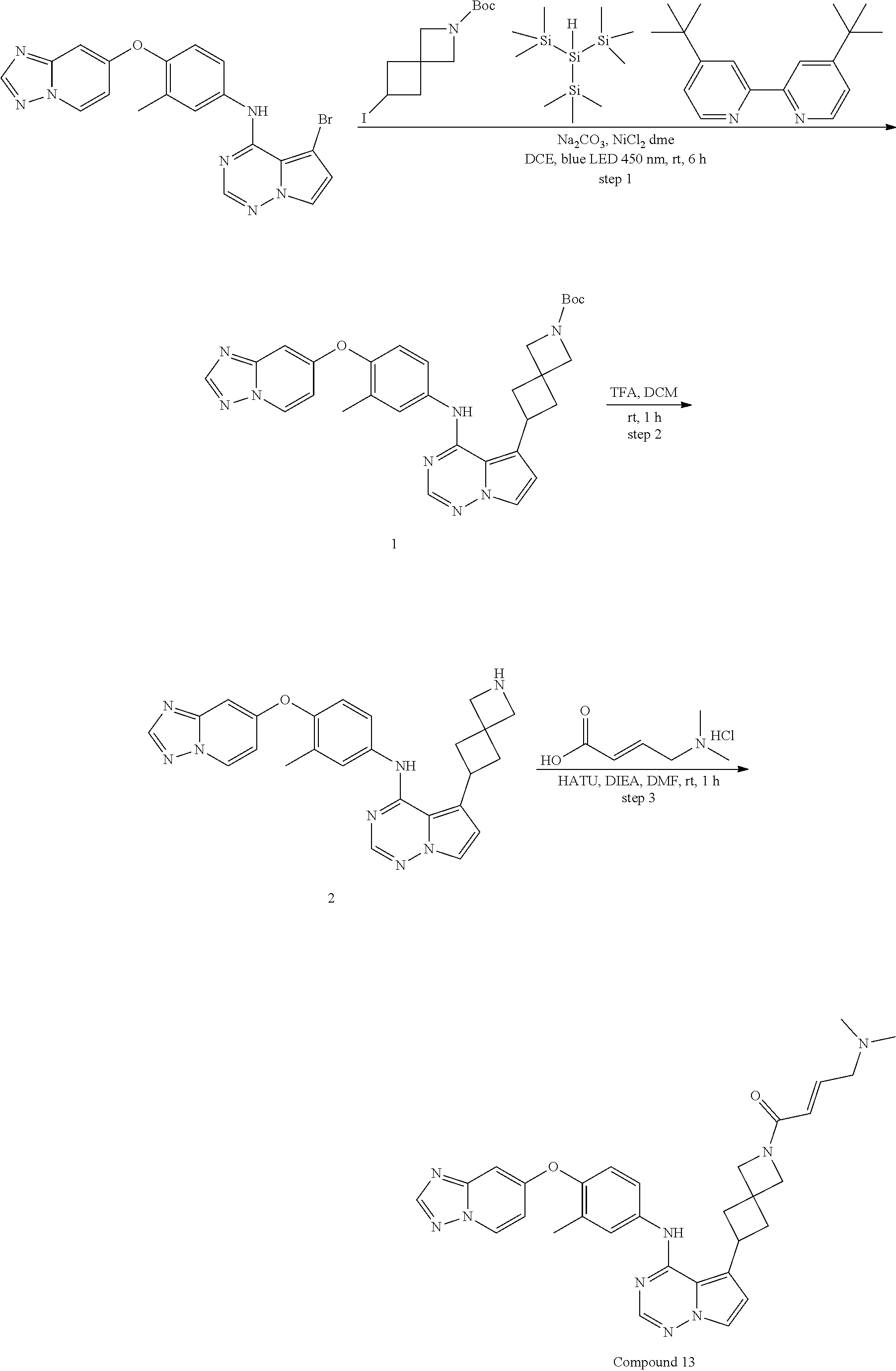

### Step 1. tert-butyl 6-(4-((4-([1,2,4]triazolo[1,5-a]pyridin-7-yloxy)-3-methylphenyl)amino)pyrrolo[2,1-f][1,2,4]triazin-5-yl)-2-azaspiro[3.3]heptane-2-carboxylate

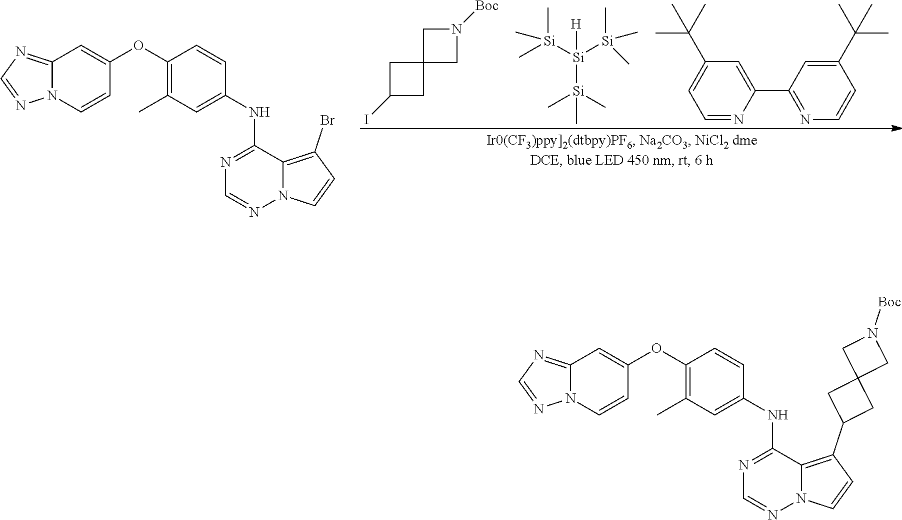

A mixture of 4,4′-di-tert-butyl-2,2′-bipyridine (30.76 mg, 0.11 mmol) and NiCl₂·dme (25.18 mg, 0.11 mmol) in DCE (1 mL) was heated to 60° C. for 10 minutes under nitrogen atmosphere. The solution was allowed to cool to room temperature to produce solution 1. Ir[dF(CF₃)ppy]₂(dtbpy)PF₆ (128.58 mg, 0.11 mmol) was added to a mixture of N-(4-([1,2,4]triazolo[1,5-a]pyridin-7-yloxy)-3-methylphenyl)-5-bromopyrrolo[2,1-f][1,2,4]triazin-4-amine (500 mg, 1.14 mmol), tert-butyl 6-iodo-2-azaspiro[3.3]heptane-2-carboxylate (740.76 mg, 2.29 mmol), 1,1,1,3,3,3-hexamethyl-2-(trimethylsilyl)trisilane (313.48 mg, 1.26 mmol) and Na₂CO₃ (364.41 mg, 3.43 mmol) in DCE (10 mL) under nitrogen atmosphere, followed by addition of the solution 1 via syringe. The resulting mixture was maintained under nitrogen, stirred at room temperature and irradiated by blue LED (450 nm) in Penn Photoreactor m2 for 6 hours. The reaction mixture was diluted with water (20 mL) and extracted with ethyl acetate (3×100 mL). The combined organic layers were washed with brine, dried over anhydrous Na₂SO₄, filtered and concentrated under vacuum to afford the crude product. The crude product was purified by column chromatography, eluted with ethyl acetate in petroleum ether from 0% to 30% to afford the desired product tert-butyl 6-(4-((4-([1,2,4]triazolo[1,5-a]pyridin-7-yloxy)-3-methylphenyl)amino)-pyrrolo[2,1-f][1,2,4]triazin-5-yl)-2-azaspiro[3.3]heptane-2-carboxylate (200 mg crude), the crude product was used for next step directly without further purification. LCMS (ESI-MS) m/z=553.2 [M+H]⁺.

### Step 2. N-(4-([1,2,4]triazolo[1,5-a]pyridin-7-yloxy)-3-methylphenyl)-5-(2-azaspiro[3.3]heptan-6-yl)pyrrolo[2,1-f][1,2,4]triazin-4-amine

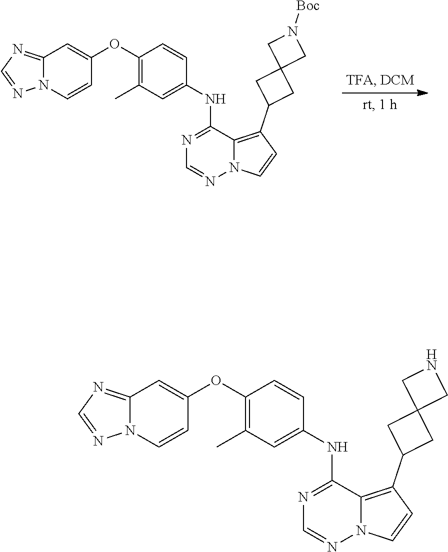 [SMILES: Cc1cc(Nc2ncnn3ccc(C4CC5(C4)CN(C(=O)OC(C)(C)C)C5)c23)ccc1Oc1ccn2ncnc2c1 ; Cc1cc(Nc2ncnn3ccc(C4CC5(CNC5)C4)c23)ccc1Oc1ccn2ncnc2c1]

TFA (6 mL) was added to a stirred mixture of tert-butyl 6-(4-((4-([1,2,4]triazolo[1,5-a]pyridin-7-yloxy)-3-methylphenyl)amino)pyrrolo[2,1-f][1,2,4]triazin-5-yl)-2-azaspiro[3.3]heptane-2-carboxylate (200 mg, 0.36 mmol) in DCM (2 mL). The resulting mixture was stirred for 1 hour at room temperature and concentrated under vacuum to afford the crude product. The crude product was purified by column chromatography, eluted with MeOH in DCM from 0% to 10% to afford the desired product N-(4-([1,2,4]triazolo[1,5-a]pyridin-7-yloxy)-3-methylphenyl)-5-(2-azaspiro[3.3]heptan-6-yl)pyrrolo[2,1-f][1,2,4]triazin-4-amine (60 mg crude), the crude product was used for next step directly without further purification. LCMS (ESI-MS) m/z=453.2 [M+H]⁺.

### Step 3. (E)-1-(6-(4-((4-([1,2,4]triazolo[1,5-a]pyridin-7-yloxy)-3-methylphenyl)amino)pyrrolo[2,1-f][1,2,4]triazin-5-yl)-2-azaspiro[3.3]heptan-2-yl)-4-(dimethylamino)but-2-en-1-one

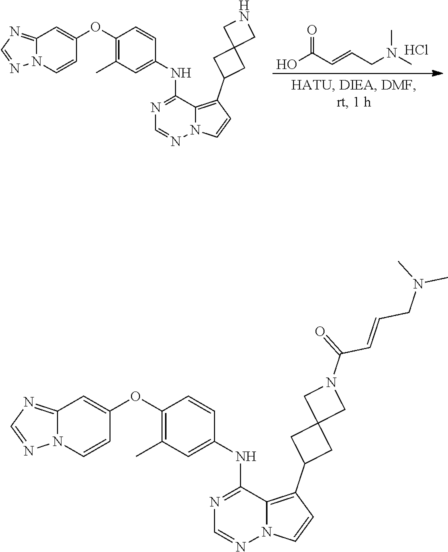

Diisopropylethylamine (51.4 mg, 0.39 mmol) was added to a stirred mixture of N-(4-([1,2,4]triazolo[1,5-a]pyridin-7-yloxy)-3-methylphenyl)-5-(2-azaspiro[3.3]heptan-6-yl)pyrrolo[2,1-f][1,2,4]triazin-4-amine (60 mg, 0.13 mmol), (E)-4-(dimethylamino)but-2-enoic acid hydrochloride (33 mg, 0.20 mmol) and HATU (75.6 mg, 0.20 mmol) in DMF (1 mL). The resulting mixture was stirred for 1 hour at room temperature and purified by Prep-HPLC, Mobile Phase A: Water (10 mmol/L NH₄HCO₃), Mobile Phase B: ACN; Gradient: 17% B to 47% B to afford the desired product (E)-1-(6-(4-((4-([1,2,4]triazolo[1,5-a]pyridin-7-yloxy)-3-methylphenyl)amino)pyrrolo[2,1-f][1,2,4]triazin-5-yl)-2-azaspiro-[3.3]heptan-2-yl)-4-(dimethylamino)but-2-en-1-one, Compound 13 (16.7 mg, 22.1% yield). LCMS (ESI-MS) m/z=564.4 [M+H]⁺. ¹H NMR (400 MHz, DMSO-d₆) δ 8.57-8.37 (m, 1H), 8.32-8.13 (m, 1H), 8.05-7.88 (m, 1H), 7.71-7.47 (m, 3H), 7.16-7.03 (m, 1H), 7.01-6.78 (m, 4H), 6.68-6.53 (m, 1H), 6.17-5.96 (m, 1H), 4.51-4.36 (m, 1H), 4.31-4.13 (m, 2H), 4.11-4.00 (m, 1H), 3.94-3.79 (m, 1H), 3.18-3.01 (m, 2H), 2.91-2.73 (m, 2H), 2.70-2.52 (m, 2H), 2.41-2.15 (m, 9H).

### Example 14. Compound 14

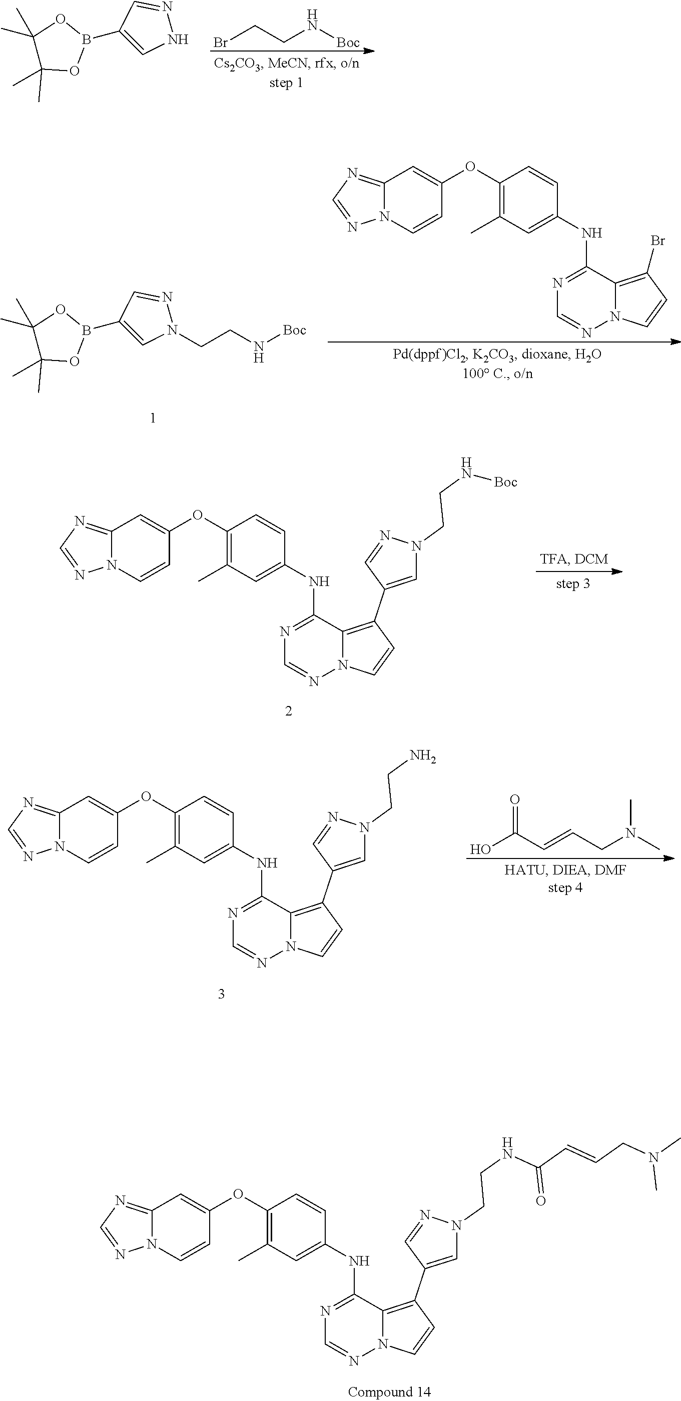

### Step 1. tert-butyl (2-(4-(4,4,5,5-tetramethyl-1,3,2-dioxaborolan-2-yl)-1H-pyrazol-1-yl)ethyl)carbamate

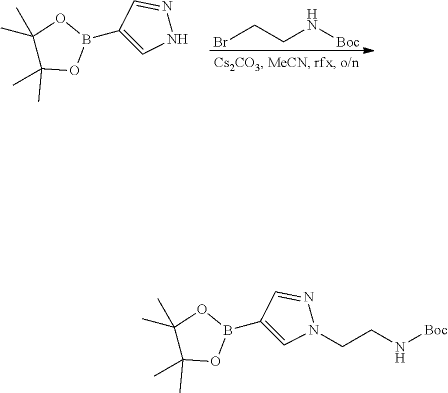 [SMILES: CC1(C)OB(c2cn[nH]c2)OC1(C)C ; CC(C)(C)OC(=O)NCCBr ; CC(C)(C)OC(=O)NCCn1cc(B2OC(C)(C)C(C)(C)O2)cn1]

A mixture of 4-(4,4,5,5-tetramethyl-1,3,2-dioxaborolan-2-yl)-1H-pyrazole (1.00 g, 5.15 mmol), tert-butyl (2-bromoethyl)carbamate (1.15 g, 5.15 mmol) and Cs₂CO₃ (3.36 g, 10.30 mmol) in MeCN (10 mL) was stirred with reflux overnight. The reaction mixture was diluted with water (20 mL), the resulting solution was then extracted with ethyl acetate (3×10 mL). The combined organic layers were washed with brine, dried over anhydrous Na₂SO₄, filtered and concentrated under vacuum to afford the crude product. The crude product was purified by column chromatography (eluted with ethyl acetate in petroleum ether from 0% to 30%), the desired fractions were combined and concentrated under vacuum to afford the desired product tert-butyl (2-(4-(4,4,5,5-tetramethyl-1,3,2-dioxaborolan-2-yl)-1H-pyrazol-1-yl)ethyl)carbamate (1.25 g, 72% yield). LCMS (ESI-MS) m/z=338.2 [M+H]⁺.

### Step 2. tert-butyl (2-(4-(4-((4-([1,2,4]triazolo[1,5-a]pyridin-7-yloxy)-3-methylphenyl)amino)pyrrolo[2,1-f][1,2,4]triazin-5-yl)-1H-pyrazol-1-yl)ethyl)carbamate

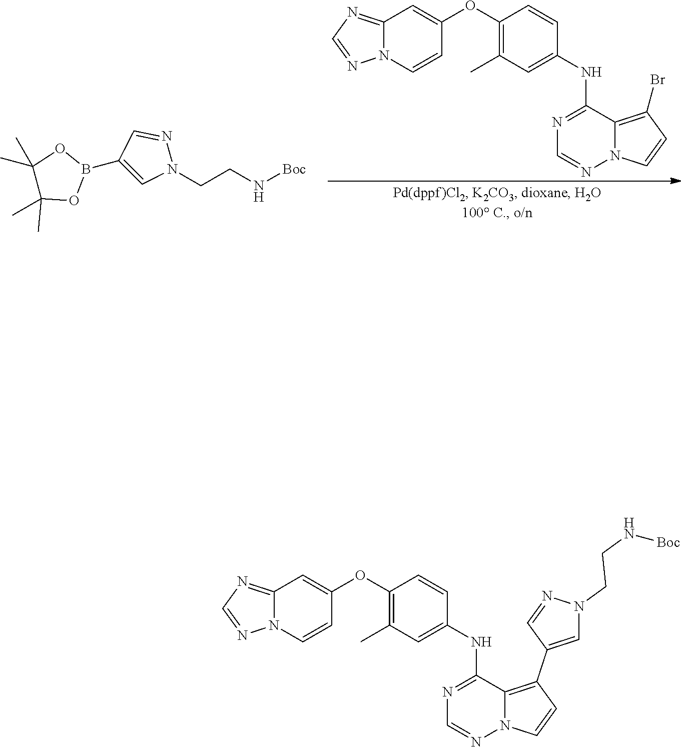 [SMILES: CC(C)(C)OC(=O)NCCn1cc(B2OC(C)(C)C(C)(C)O2)cn1 ; Cc1cc(Nc2ncnn3ccc(Br)c23)ccc1Oc1ccn2ncnc2c1 ; Cc1cc(Nc2ncnn3ccc(-c4cnn(CCNC(=O)OC(C)(C)C)c4)c23)ccc1Oc1ccn2ncnc2c1]

A mixture of tert-butyl (2-(4-(4,4,5,5-tetramethyl-1,3,2-dioxaborolan-2-yl)-1H-pyrazol-1-yl)ethyl)carbamate (400 mg, 1.18 mmol), N-(4-([1,2,4]triazolo[1,5-a]pyridin-7-yloxy)-3-methylphenyl)-5-bromopyrrolo[2,1-f][1,2,4]triazin-4-amine (517.48 mg, 1.18 mmol), Pd(dppf)Cl₂ (86.79 mg, 0.11 mmol), K₂CO₃ (327.86 mg, 2.37 mmol) in dioxane (4 mL) was stirred overnight at 100° C. under nitrogen atmosphere. The reaction mixture was diluted with water (15 mL), the resulting solution was extracted with ethyl acetate (3×5 mL). The combined organic layers were washed with brine, dried over anhydrous Na₂SO₄, filtered and concentrated under vacuum to afford the crude product. The crude product was purified by column chromatography (silica gel, 25 g, eluted with ethyl acetate in petroleum ether from 0% to 30%), the desired fractions were combined and concentrated under vacuum to afford the desired product tert-butyl (2-(4-(4-((4-([1,2,4]triazolo[1,5-a]pyridin-7-yloxy)-3-methylphenyl)amino)pyrrolo[2,1-f][1,2,4]triazin-5-yl)-1H-pyrazol-1-yl)ethyl)carbamate (480 mg, 71% yield). LCMS (ESI-MS) m/z=567.2 [M+H]⁺.

### Step 3. N-(4-([1,2,4]triazolo[1,5-a]pyridin-7-yloxy)-3-methylphenyl)-5-(1-(2-aminoethyl)-1H-pyrazol-4-yl)pyrrolo[2,1-f][1,2,4]triazin-4-amine

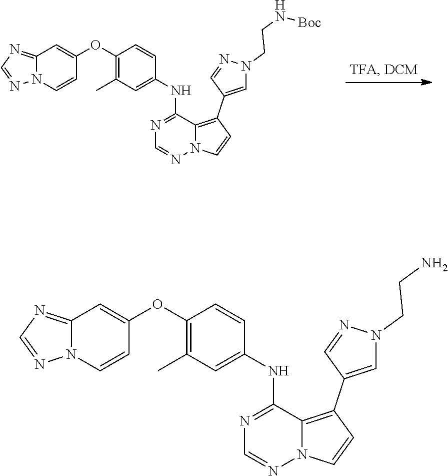 [SMILES: Cc1cc(Nc2ncnn3ccc(-c4cnn(CCNC(=O)OC(C)(C)C)c4)c23)ccc1Oc1ccn2ncnc2c1 ; Cc1cc(Nc2ncnn3ccc(-c4cnn(CCN)c4)c23)ccc1Oc1ccn2ncnc2c1]

A mixture of tert-butyl (2-(4-(4-((4-([1,2,4]triazolo[1,5-a]pyridin-7-yloxy)-3-methylphenyl)amino)pyrrolo[2,1-f][1,2,4]triazin-5-yl)-1H-pyrazol-1-yl)ethyl)carbamate (480 mg, 0.84 mmol) and TFA (1 mL, 13.46 mmol) in DCM (3 mL) was stirred for 1 hour at room temperature. The resulting mixture was concentrated under vacuum to afford the crude product N-(4-([1,2,4]triazolo[1,5-a]pyridin-7-yloxy)-3-methylphenyl)-5-(1-(2-aminoethyl)-1H-pyrazol-4-yl)pyrrolo[2,1-f][1,2,4]triazin-4-amine (480 mg), the crude product was used in next step directly without further purification. LCMS (ESI-MS) m/z=467.2 [M+H]⁺.

### Step 4. (E)-N-(2-(4-(4-((4-([1,2,4]triazolo[1,5-a]pyridin-7-yloxy)-3-methylphenyl)amino)pyrrolo[2,1-f][1,2,4]triazin-5-yl)-1H-pyrazol-1-yl)ethyl)-4-(dimethylamino)but-2-enamide

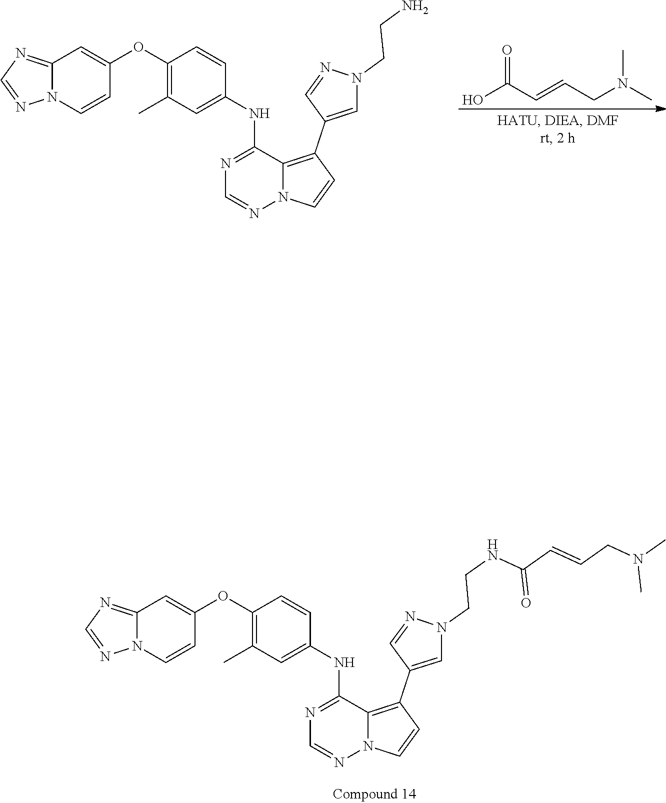

A solution of N-(4-([1,2,4]triazolo[1,5-a]pyridin-7-yloxy)-3-methylphenyl)-5-(1-(2-aminoethyl)-1H-pyrazol-4-yl)pyrrolo[2,1-f][1,2,4]triazin-4-amine (50 mg, 0.10 mmol), (E)-4-(dimethylamino)but-2-enoic acid (16.61 mg, 0.12 mmol), HATU (81.51 mg, 0.21 mmol) and diisopropylethylamine (41.56 mg, 0.32 mmol) in DMF (2 mL) was stirred for 2 hours at room temperature. The reaction mixture was diluted with water (20 mL), the resulting solution was extracted with ethyl acetate (3×20 mL), and the organic layers were concentrated under vacuum. The crude product was purified by Prep-HPLC, Mobile Phase A: Water (0.05% TFA), Mobile Phase B: ACN; Gradient: 14% B to 44% B to afford (E)-N-(2-(4-(4-((4-([1,2,4]triazolo[1,5-a]pyridin-7-yloxy)-3-methylphenyl)amino)pyrrolo[2,1-f][1,2,4]triazin-5-yl)-1H-pyrazol-1-yl)ethyl)-4-(dimethylamino)but-2-enamide, Compound 14 (47.2 mg, 76% yield). LCMS (ESI-MS) m/z=578.2 [M+H]⁺. ¹H NMR (400 MHz, DMSO-d₆) δ 9.63 (s, 1H), 8.97 (d, J=7.3 Hz, 1H), 8.45 (d, J=10.9 Hz, 2H), 8.10 (s, 1H), 8.05 (s, 1H), 7.85 (s, 1H), 7.79 (s, 1H), 7.65 (s, 1H), 7.60 (s, 1H), 7.21 (d, J=8.7 Hz, 1H), 7.06 (dd, J=7.5, 2.6 Hz, 1H), 6.81 (d, J=2.6 Hz, 1H), 6.76 (d, J=2.7 Hz, 1H), 6.53 (m, J=14.7, 7.2 Hz, 1H), 6.21 (m, J=15.4, 1.3 Hz, 1H), 4.30 (t, J=6.1 Hz, 2H), 3.83 (t, J=5.8 Hz, 2H), 3.64 (q, J=6.0 Hz, 2H), 2.72 (d, J=4.3 Hz, 6H), 2.17 (s, 3H).

### Example 15. Compound 15

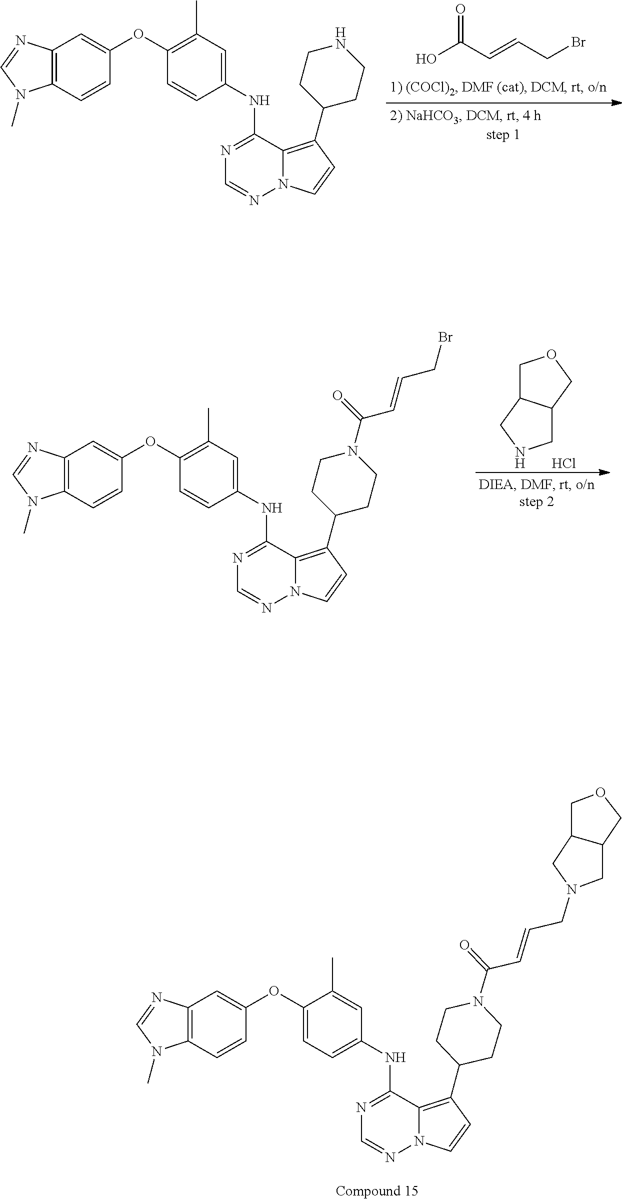 [SMILES: Cc1cc(Nc2ncnn3ccc(C4CCNCC4)c23)ccc1Oc1ccc2c(c1)ncn2C ; O=C(O)/C=C/CBr ; Cc1cc(Nc2ncnn3ccc(C4CCN(C(=O)/C=C/CBr)CC4)c23)ccc1Oc1ccc2c(c1)ncn2C ; C1NCC2COCC12 ; Cl ; Cc1cc(Nc2ncnn3ccc(C4CCN(C(=O)/C=C/CN5CC6COCC6C5)CC4)c23)ccc1Oc1ccc2c(c1)ncn2C]

### Step 1. (E)-4-bromo-1-(4-(4-((3-methyl-4-((1-methyl-1H-benzo[d]imidazol-5-yl)oxy)phenyl)amino)-pyrrolo[2,1-f][1,2,4]triazin-5-yl)piperidin-1-yl)but-2-en-1-one

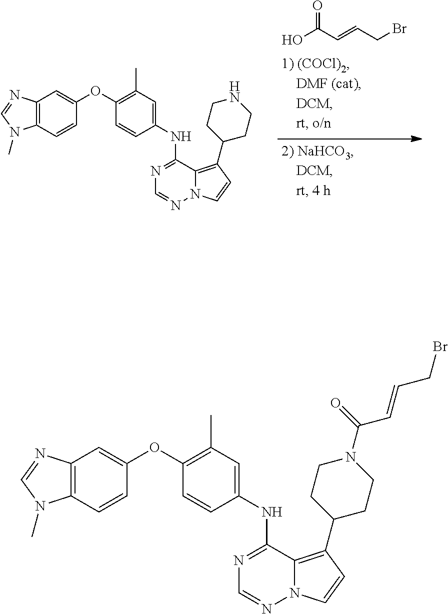 [SMILES: O=C(O)/C=C/CBr ; Cc1cc(Nc2ncnn3ccc(C4CCNCC4)c23)ccc1Oc1ccc2c(c1)ncn2C ; Cc1cc(Nc2ncnn3ccc(C4CCN(C(=O)/C=C/CBr)CC4)c23)ccc1Oc1ccc2c(c1)ncn2C]

To a cold (0° C.) solution of (E)-4-bromobut-2-enoic acid (0.73 g, 4.41 mmol) in DMF (0.05 mL) and DCM (10 mL) was added oxalyl chloride (1.12 g, 8.82 mmol) dropwise. The reaction mixture was stirred overnight at room temperature. The resulting crude mixture was concentrated under vacuum. The residue was dissolved in DCM (15 mL). The solution was added to a stirred mixture of N-(3-methyl-4-((1-methyl-1H-benzo[d]imidazol-5-yl)oxy)phenyl)-5-(piperidin-4-yl)pyrrolo[2,1-f][1,2,4]triazin-4-amine (2 g, 4.41 mmol) and NaHCO₃ (0.75 g, 8.92 mmol) in DCM (20 mL). The resulting mixture was stirred for 4 hours at room temperature and concentrated under vacuum to afford the crude product. The crude product was purified by silica gel column chromatography, eluted with petroleum ether/ethyl acetate=1:4 to afford the desired product (E)-4-bromo-1-(4-(4-((3-methyl-4-((1-methyl-1H-benzo[d]imidazol-5-yl)oxy)phenyl)amino)pyrrolo[2,1-f][1,2,4]triazin-5-yl)piperidin-1-yl)but-2-en-1-one (800 mg, 27.2% yield). LCMS (ESI-MS) m/z=600.2 [M+H]⁺

### Step 2. (E)-1-(4-(4-((3-methyl-4-((1-methyl-1H-benzo[d]imidazol-5-yl)oxy)phenyl)amino)pyrrolo[2,1-f][1,2,4]triazin-5-yl)piperidin-1-yl)-4-(tetrahydro-1H-furo[3,4-c]pyrrol-5(3H)-yl)but-2-en-1-one

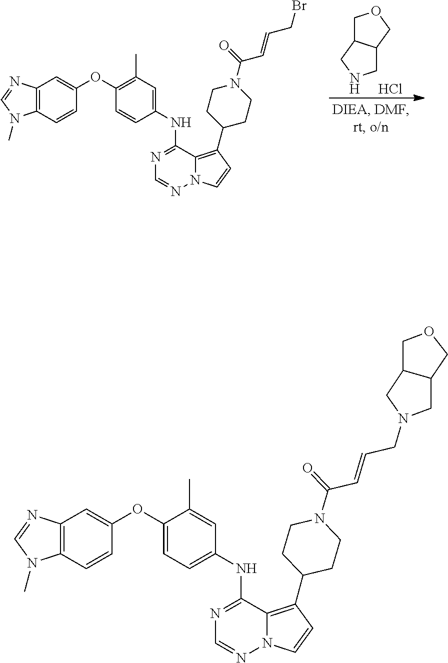 [SMILES: Cc1cc(Nc2ncnn3ccc(C4CCN(C(=O)/C=C/CBr)CC4)c23)ccc1Oc1ccc2c(c1)ncn2C ; C1NCC2COCC12 ; Cl ; Cc1cc(Nc2ncnn3ccc(C4CCN(C(=O)/C=C/CN5CC6COCC6C5)CC4)c23)ccc1Oc1ccc2c(c1)ncn2C]

Diisopropylethylamine (161 mg, 1.25 mmol) was added to a mixture of (E)-4-bromo-1-(4-(4-((3-methyl-4-((1-methyl-1H-benzo[d]imidazol-5-yl)oxy)phenyl)amino)pyrrolo[2,1-f][1,2,4]triazin-5-yl)piperidin-1-yl)but-2-en-1-one (150 mg, 0.25 mmol), hexahydro-1H-furo[3,4-c]pyrrole hydrochloride (25 mg, 0.22 mmol) in DMF (2 mL). The resulting mixture was stirred overnight at room temperature. The reaction mixture was purified by Prep-HPLC, Mobile Phase A: Water (0.1% FA), Mobile Phase B: ACN; Gradient: 2% B to 25% B to afford the desired product (E)-1-(4-(4-((3-methyl-4-((1-methyl-1H-benzo[d]imidazol-5-yl)oxy)phenyl)amino)pyrrolo[2,1-f][1,2,4]triazin-5-yl)piperidin-1-yl)-4-(tetrahydro-1H-furo[3,4-c]pyrrol-5(3H)-yl)but-2-en-1-one, Compound 15 (15.5 mg, 10% yield). LCMS (ESI-MS) m/z=633.5 [M+H]⁺. ¹H NMR (400 MHz, DMSO-d₆) δ(ppm) 8.43 (s, 1H), 8.17 (s, 2H), 7.85 (s, 1H), 7.68 (d, J=2.7 Hz, 1H), 7.57 (d, J=8.7 Hz, 1H), 7.52 (d, J=2.6 Hz, 1H), 7.40-7.35 (m, 1H), 7.10 (d, J=2.3 Hz, 1H), 7.00-6.98 (m, 1H), 6.87 (d, J=8.6 Hz, 1H), 6.70-6.60 (m, 3H), 4.57 (d, J=12.6 Hz, 1H), 4.12 (s, 1H), 3.71 (s, 1H), 3.39-3.35 (m, 2H), 3.31 (d, J=14.0 Hz, 1H), 2.84 (s, 1H), 2.70 (s, 2H), 2.53 (s, 2H), 2.47 (s, 4H), 2.33-2.35 (m, 2H), 2.30 (s, 1H), 2.25 (s, 3H), 1.96 (s, 2H), 1.55 (s, 3H).

### Example 16. HER2 Biochemistry Assay

The purpose of the wtERBB2, ERBB2-A775_G776insYVMA (ERBB2YVMA), wtEGFR biochemical assay was to evaluate the inhibition (% inhibition and IC₅₀ values) of the small molecule inhibitors by using the “HotSpot” radiometric kinase activity assay. HotSpot assays monitor the production of kinase substrate by the radioactive gamma phosphate of adenosine triphosphate (33P-ATP) during biochemical reactions. HotSpot assays was performed in steps upon completion of the kinase reaction: deposition of a reaction mixture aliquot onto P81 ion exchange paper, extensive washing of the aforementioned P81 paper using phosphoric acid to remove unbound radiolabeled 33P-ATP from the P81 paper, and finally visualization and quantification of the dried P81 paper utilizing a phosphoimager. The radioactive signal generated from the aliquot of buffer solution was proportional to the amount of radiolabeled substrate produced, and which is generally reflective of kinase activity. wtERBB2 was purchased from Reaction Biology (Cat: Kin-21-497), ERBB2-A775_G776insYVMA was purchased from SignalChem (Cat: E27-13BG), and wt EGFR was purchased from Invitrogen (Cat: PR7295B). Typical reaction solutions (10 μL final reaction volume) contained the following buffer conditions: 20 mM Hepes (pH 7.5), 10 mM MgCl₂, 1 mM EGTA, 0.01% Brij35, 0.02 mg/mL BSA, 0.1 mM Na3VO4, 2 mM DTT, 1% DMSO. The assay was primed by preparing a fresh 0.2 mg/mL solution of pEY (Sigma Cat: P7244) substrate in the reaction buffer and supplementing that solution with 2 mN of MnCl₂ as substrate cofactor (Sigma Cat: M9522). The kinase of interest was then added to the solution at the appropriate concentration (30 nM wtERBB2 from, or 20 nM ERBB2YVMA, or 4 nM wtEGFR) and gently mixed prior to delivery of compound in 100% DMSO by acoustic dispensing (Beckman Echo550). Compound, Kinase, and substrate were allowed to incubate for 20 minutes at room temperature prior to the initiation of the reaction by addition of 33P-ATP (PerkinElmer Cat: NEG602, final conc 10 μM). The reaction was allowed to run for 2 hours at room temperature and was then spotted onto P81 ion exchange paper, washed with a 0.75% Phosphoric acid solution, and imaged to quantify the amount of radioactivity. IC₅₀ determinations were made from a plot of kinase activity as a function of inhibitor concentration fit to the 4-parameter IC₅₀ equation with the enzyme concentration held constant using GraphPad Prism (San Diego). Table 1 provides biochemical activity of various compounds in this disclosure against HER2 WT, HER2 YVMA and EGFR WT kinases in HotSpot assay.

TABLE 1
Biochemical activity of selected examples against HER2 WT,
HER2 YVMA, and EGFR WT kinases in HotSpot assay in nM

| | HER2 - YVMA | HER2 WT | EGFR WT |
|---|---|---|---|
| Compound # | IC₅₀ (nM) | IC₅₀ (nM) | IC₅₀ (nM) |
| 1 | ++++ | ++++ | ++++ |
| 3 | ++++ | ++++ | ++++ |
| 4 | ++++ | ++++ | ++++ |
| 5 | ++++ | ++++ | +++ |
| 8 | ++++ | ++++ | ++++ |
| 12 | ++++ | ++++ | +++ |
| 13 | ++++ | ++++ | +++ |
| 14 | +++ | ++++ | + |
| 15 | ++++ | ++++ | ++++ |

++++ = IC50 < 200 nM;
+++ = 200 nM < IC50 < 500 nM;
++ = 500 nM < IC50 < 1500 nM;
+ = 1500 nM < IC50 < 5000 nM;
X = IC50 > 5000 nM

### Example 17. Cell Growth Inhibition Assay

BaF3_RTK (BaF3_HER2WT, BaF3_HER2YVMA and BaF3_EGFRWT) cells were used to evaluate the potency and selectivity of the HER2 inhibitors. BaF3_HER2WT and YVMA cells were maintained in RPMI media supplemented with 10% fetal bovine serum. BaF3_EGFRWT cells were maintained in RPMI media+10% FBS+80 ng/mL recombinant human EGF. For cell growth inhibition assay, HER2 inhibitors in DMSO solution were dispensed with HP D300e (Tecan) into 384-well plates (Corning #3765) and the 384-well plates were UV-sterilized prior to the assay. The inhibitors were tested in the 10-10,000 nM or 0.316-316 nM concentration range with half-log serial dilutions. BaF3_RTK cells (750 cells/30 uL/well) were added to each well using Multidrop Combi (ThermoFisher) and the treatment duration was 3 days. To counter screen against non-specific activities, 10 ng/mL IL-3 was supplemented to BaF3_HER2WT cells during the compound treatment. BaF3_HER2 WT cells were no longer susceptible to HER2 inhibition in the presence of IL-3. Any remaining cell growth inhibition is likely due to non-target related activities. Equal volume of CellTiterGlo 2.0 (Promega) was added to each well at the end of the 3-day treatment and ClarioStar (BMG) was used to read the luminescent signal. The % cell growth inhibition (% CGI) was calculated using the following formula % CGI=100−100*luminescenceₛₐₘₚₗₑ/luminescence_{control}. The half maximal inhibitory concentration (IC₅₀) was determined by nonlinear curve fitting (four parameters, variable slope). Table 2 provides cell growth inhibition activity of selected examples against BaF3_HER2 WT, BaF3_HER2 YVMA and BaF3 EGFR WT cells of various compounds in this disclosure.

TABLE 2
Cell growth inhibition activity of selected
examples against BaF3_HER2WT, BaF3_HER2YVMA
and BaF3_EGFRWT cells in nM

| Compound # | HER2 - YVMA | HER2 WT | EGFR WT |
|---|---|---|---|
| 1 | ++++ | ++++ | + |
| 2 | ++ | +++ | + |
| 3 | ++++ | ++++ | ++ |
| 4 | ++++ | ++++ | +++ |
| 5 | ++++ | ++++ | + |
| 6 | ++++ | ++++ | + |
| 7 | ++++ | ++++ | + |
| 8 | ++ | +++ | + |
| 9 | ++++ | ++++ | + |
| 10 | ++ | +++ | + |
| 11 | ++++ | ++++ | + |
| 12 | ++++ | ++++ | + |
| 13 | ++ | +++ | + |
| 14 | + | + | + |
| 15 | ++++ | ++++ | + |

++++ = IC50 < 200 nM;
+++ = 200 nM < IC50 < 500 nM;
++ = 500 nM < IC50 < 1500 nM;
+ = 1500 nM < IC50 < 5000 nM

### Example 18A. Treatment Protocols for Subjects with Advanced Cancers Harboring HER2 Alterations

A compound of this disclosure can be administered as a monotherapy to subjects that have HER2 bladder cancer, colorectal cancer (CRC), or non-small cell lung cancer (NSCLC); all with or without brain metastases. The compound for monotherapy can be a compound of Formula II or Compound 5. The compound can also be administered in combination with an HER2 monoclonal antibody or a HER monoclonal antibody drug conjugate to subjects that have breast cancer with brain metastases. The compound for combination therapy is a compound of Formula II or Compound 5. The total duration of intervention for each subject can include prescreening and screening for up to 28 days prior to the first dose of study drug; and treatment period for a daily treatment in 21-day treatment cycles until disease progression, unacceptable toxicity, or withdrawal from the administration.

Dosing Regimens and Dose Escalation for Subjects with Advanced Cancers Harboring HER2 Alterations

For monotherapy dosing, subjects with confirmed, relapsed/refractory malignancy with documented diagnosis of HER2 alterations can be enrolled sequentially into the dose cohorts starting with Cohort 1 (120 mg). Once a Subject completes a 21-day treatment cycle, in the absence of a grade 2 or greater adverse event or dose limiting toxicity, escalation may proceed to the next dosing (Cohort 2) which is 240 milligrams. This dose escalation may continue to proceed in this manner to Cohort 3 (360 mg), to Cohort 4 (480 mg), to Cohort 5 (960 mg), and then to Cohort 6 (1560 mg). An initial maximum tolerated dose (MTD) can be determined, and then to optimize dose selection, additional cohorts may be opened to further evaluate the safety and preliminary efficacy of the compounds of this disclosure. For the monotherapy dosing, the compound can be a compound of Formula II or Compound 5.

### Optimizing Dosing for Different Subject Populations

Once an initial MTD is determined, dosing may be further optimized by opening additional cohorts based on emerging data to further evaluate the safety and preliminary efficacy of the compounds of this disclosure in the following monotherapy dose optimization or combination therapy dose optimization. Monotherapy dose optimization includes: subject populations of colorectal cancer subjects with or without brain metastases or non-small lung cancer subjects with brain metastases; dose levels of a compound of this disclosure (e.g., a compound of Formula II or Compound 5) administered to subjects in at least 2 potential dose levels with 1:1 randomization of a certain number of subjects at each dose level. Combination therapy dose optimization includes: a compound of this disclosure (e.g., a compound of Formula II or Compound 5) in combination with a HER2 monoclonal antibody or a HER2 monoclonal antibody drug conjugate; subject population of breast cancer subjects with brain metastases or colorectal subjects with or without brain metastases; dose levels of a compound of this disclosure administered to subjects at 1 dose level below the monotherapy MTD in combination with a HER2 monoclonal antibody or a HER2 monoclonal antibody drug conjugate.

### Simon 2-Stage Evaluation

Compounds of this disclosure can be evaluated as monotherapy, and in combination with a HER2 monoclonal antibody (such as trastuzumab) or a HER2 monoclonal antibody drug conjugate by utilizing a Simon 2-stage minimax design in each of the following HER2 positive subject groups as by way of example: subject with bladder cancer, colorectal cancer, or non-small cell lung cancer (all with or without brain metastases, a compound of this disclosure (e.g., a compound of Formula II or Compound 5) can be administered monotherapy at the confirmed MTD; or for subject with breast cancer with brain metastases, Compound 5 can be administered at the confirmed MTD in combination with trastuzumab. Subjects may continue to receive a compound of this disclosure (such as a compound of Formula II or Compound 5) if trastuzumab is discontinued, but a subject can be discontinued from all study treatment if the compound of this disclosure is discontinued. The treatment period can be daily dosing in 21-day treatment cycles until disease progression, unacceptable toxicity, or withdrawal from the treatment.

The subject population can includes adult subjects with relapsed/refractory malignancy with documented HER2 alterations. The selection of the subject population for the compounds of this disclosure can be guided by a tangible and pressing medical need, e.g., effectively targeting HER2 oncogenic drivers in a range of solid tumors. This focus is driven by the role played by HER2 overexpression and mutation in various cancer types, including breast cancer, CRC, NSCLC, and bladder cancer. Subjects eligible to participate in the treatment protocols of this disclosure meet the following criteria: age≥18 years; have relapsed/refractory to standard therapy, or no standard therapy exists that is likely to provide clinical benefit; have HER2-altered malignancy (e.g., HER2 gene mutations or fusions, HER2 gene amplification, or HER2 protein overexpression); have progression of disease after the last systemic therapy, or be intolerant of last systemic therapy; or have radiographically measurable disease assessable by Response Evaluation Criteria in Solid Tumors version 1.1 (RECIST v1.1), with at least one site of disease that is measurable and that has not been previously irradiated or, if the subject has had previous radiation to the target lesion(s), there must be evidence of progression since the radiation. HER2 alteration can be established based on archival tissue sample, fresh biopsy, or blood sample (ctDNA). Biopsies can be obtained consistent with American Society of Clinical Oncology (ASCO) guidance.

Subjects eligible to participate in the treatment protocols of this disclosure have colorectal cancer and meet the following criteria: histologically and/or cytologically documented adenocarcinoma of the colon or rectum; or received and failed standard therapeutic regimens containing at least one of the following agents: fluoropyrimidine (e.g., 5-fluorouracil or capecitabine), oxaliplatin, irinotecan, an anti-VEGF monoclonal antibody (bevacizumab, ramucirumab, or zivaflibercept), an anti-PD-(L)1 therapy (nivolumab or pembrolizumab) if tumor has deficient mismatch repair proteins or is MSI-High, or an anti-EGFR inhibitor if tumor has known RAS wildtype. Alternatively, subjects may be eligible if standard therapeutic options with proven survival benefit have been refused by the subject or are contraindicated as per treating physician. Subject can also be eligible due to prior treatment with HER2-directed therapy; HER2 alterations based on test from an archival tumor tissue sample analyzed by Clinical Laboratory Improvement Act (CLIA)-certified or International Organization for Standardization (ISO)-accredited laboratory; HER2 amplification (by next generation sequencing [NGS], by fluorescence in situ hybridization [FISH], or chromogenic in situ hybridization [CISH], if immunohistochemistry [IHC] score 2+) based on HER2/CEP17 ratio≥2.0 or IHC 3+16. Additional HER2 alteration can be based on test from an archival tumor tissue sample analyzed by CLIA-certified or ISO-accredited laboratory for HER2 amplification (by NGS or by FISH or CISH, if IHC score 2+) with HER2/CEP17 ratio≥2.0 and HER2 signal≥4.0 per nucleus or HER2/CEP17 ratio<2.0 and HER2 signal≥6.0 per nucleus.

Subjects can also be eligible to participate in the treatment protocols of this disclosure have breast cancer and meet the following criteria: histologically confirmed breast carcinoma; or brain metastases measurable per RECIST v1.1 at enrollment classified as one of the following: treated and stable (e.g., prior local treatment and no evidence of progression at baseline brain MRI, including subjects treated during the screening period); treated and progressing (e.g., prior local treatment and evidence of progression of existing lesions, new lesions, or untreated lesions remaining after prior treatment at baseline brain MRI); or untreated (no prior local treatment and if >2 cm, per Investigator did not require immediate local therapy). Prior treatment can include: standard therapeutic regimens in the neoadjuvant or metastatic setting. Standard regimens in the neoadjuvant or metastatic setting may include: trastuzumab, pertuzumab T-DM1, T-DXd (ENHERTU®), or tucatinib. HER2-directed treatments given in the metastatic setting may include: T-DXd (ENHERTU®) or tucatinib. In some cases, the HER2-directed treatments given in the metastatic setting may not include lapatinib or neratinib.

Subjects can be eligible to participate in the treatment protocols of this disclosure have bladder cancer and meet the following criteria: histologically or cytologically confirmed diagnosis of inoperable, locally advanced or metastatic urothelial cancer of the bladder, renal pelvis, or ureter; progression or intolerance following≥1 prior therapy, which included platinum-based chemotherapy, PD-(L)1 inhibitor, and/or enfortumab vedotin for advanced or metastatic disease.; or HER2 alterations based on test from an archival tumor tissue sample analyzed by CLIA-certified or ISO-accredited laboratory such as (i) HER2 TKD mutation or gene fusion (by NGS), (ii) HER2 amplification (by FISH or CISH), or (iii) IHC 3+/2+.

Subjects can be eligible to participate in the treatment protocols of this disclosure have non-small cell lung cancer (NSCLC) and meet the following criteria: pathologically documented unresectable and/or metastatic non-squamous NSCLC; subjects must have received and failed≥1 prior therapy, which included a platinum-based chemotherapy; brain metastases that are measurable per RECIST v1.1; previously treated with T-DXd (ENHERTU®); HER2 alterations based on test from an archival tumor tissue or blood sample analyzed by CLIA-certified or ISO-accredited laboratory; or HER2 tyrosine kinase domain (TKD) mutation (by NGS, polymerase chain reaction [PCR], or Sanger sequencing).

The treatment protocols can be administered to subjects with water under fasting conditions. The subjects can fast for at least 2 hours before and 1 hour after taking the compound of this disclosure. The compound can be administered to subjects as an oral, immediate release, two-piece hard gelatin capsule containing about 30 mg or about 120 mg of the compound. The compound can be administered to subjects in need thereof in combination with a recombinant humanized monoclonal antibody directed against HER2 such as, for example, trastuzumab. The treatment protocols can include administering Compound 5 to subjects in need thereof with a recombinant humanized monoclonal antibody directed against HER2 such as, for example, trastuzumab. Trastuzumab is available as 150 mg lyophilized powder in a single-dose vial for reconstitution to be given by intravenous (IV) injection. Additional information about trastuzumab is available in the trastuzumab prescribing information. Subjects with breast cancer can be administered, in combination with the compound of this disclosure (such as Formula II or compound 5), trastuzumab as standard of care and as described in the prescribing information (4 mg/kg 90 minute IV followed by subsequent weekly doses of 2 mg/kg IV 30 minutes infusion weekly).

### Disease Response Assessments

Disease response assessments can be performed by computed tomography [CT] or magnetic resonance imaging [MRI] scans. In another embodiment, CT or MRI scans of the brain, chest, abdomen, and pelvis are performed, and a bone scan is done if clinically indicated. CT and MRI scans can be performed using institutional standard operating procedures.

### Fresh Tissue Biopsy and Peripheral Blood

To understand the trafficking of the compound of this disclosure into the tumor tissue and the impact of tumor environment on the function of the compound of this disclosure, tumor biopsies and peripheral blood may be obtained from subjects upon their consent. Standard institutional guidelines should be followed for tumor biopsy.

### Method of Analysis for Treatment Protocols

One variable that can be measured in the treatment protocols of this disclosure is overall response rate (ORR), defined as achieving a best response of complete response (CR) or partial response (PR). The analysis may be performed on the response evaluable set, including all subjects who have received at least one dose of the compound of this disclosure with or without trastuzumab, have at least one measurable lesion at baseline, and have at least one post-baseline response assessment. The summary statistic can be the ORR rate which can be supported by a 95% exact Clopper-Pearson confidence interval.

Another variable that can be measured in the treatment protocols of this disclosure is progression free survival (PFS) which is defined as the number of months from the date of first study treatment administration to the earliest of documented progressive disease or death without prior progression. A death will be considered a PFS event.

Another variable that can be measured in the treatment protocols of this disclosure is overall survival (OS) which is defined as the number of months from the date of first study treatment administration to the date of death, irrespective of cause.

Another variable that can be measured in the treatment protocols of this disclosure is best overall response (BoR) which is generally the best response across all assessments for a subject that can be measured from the first day of treatment up until disease progression.

Another variable that can be measured in the treatment protocols of this disclosure is duration of response defined as the time between first disease response and date of disease progression or death due to any cause.

Another variable that can be measured in the treatment protocols of this disclosure is disease control rate (DCR) which is defined as the percentage of subjects who achieved disease response or stable disease (SD) consecutively for 3 months.

Another variable that can be measured in the treatment protocols of this disclosure is clinical benefit rate (CBR) which is defined as the percentage of subjects who achieve complete response, partial response, or stable disease.

Another variable that can be measured in the treatment protocols for breast cancer and NSCLC subjects are efficacy endpoints that can be determined intracranially.

### Human Cancer Cell Line Evaluation

HER2 activation through amplification, overexpression or mutation drives many types of human cancers, and has been the target of several therapeutic approaches. The demonstration of clinical benefit of available pan-ERBB and HER2 inhibitors has been hampered by the dose-limiting side effects from their collateral or residual EGFR inhibition. Oral administration of a compound encompassed by Formula II has been found to lead to tumor regression in a diverse set of HER2-driven mouse tumor models well below maximum tolerated doses, including models for breast cancer, non-small cell lung cancer, gastric cancer, and esophageal squamous cancer.

The cell growth inhibition activity of the compounds of this disclosure were evaluated in human cancer cell lines harboring endogenous HER2 or EGFR aberrations including: BT-474 (HER2 amplification, breast cancer); NCI-N87 (also known as N87, HER2 amplification, gastric cancer); NCI-H1781 (also known as H1781, HER2 exon 20 G776delinsVC mutation, lung cancer); or A-431 (EGFR overexpression, epidermoid carcinoma).

To directly measure the effect of compounds of this disclosure on oncogenic HER2 signaling, the effect of Compound 5 on the autophosphorylation of HER2 was examined in the HER2-amplified breast cancer cell lines BT-474 and gastric cancer cell line NCI-N87. In addition, the effect of Compound 5 on the phosphorylation of EGFR was examined in an EGFR-overexpressing skin cancer cell line, A-431. After a 4-hour administration, Compound 5 exhibited IC₅₀s of 31 nM in both BT-474 and NCI-N87 q3w assays and 4,058 nM in A431 phosphorylated EGFR (pEGFR) assay, with a selectivity for pHER2 of 131-fold over pEGFR. The IC₅₀ of Compound 5 for pHER2 inhibition in engineered Ba/F3 cells expressing the HER2 exon 20 YVMA insertion mutation after 4 hours was also measured, and the result of 29 nM closely matched the IC₅₀ values observed in HER2-amplified BT-474 cells and NCI-N87 cells.

### Example 18B. Treatment Protocols for Subjects with Advanced Cancers Harboring HER2 Alterations

A compound of this disclosure can be administered as a monotherapy to subjects that have HER2 bladder cancer, colorectal cancer (CRC), or non-small cell lung cancer (NSCLC); all with or without brain metastases. The compound for monotherapy can be a compound of Formula II or Compound 5. The total duration of intervention for each subject can include prescreening and screening for up to 28 days prior to the first dose of study drug; and treatment period for a daily treatment in 21-day treatment cycles until disease progression, unacceptable toxicity, or withdrawal from the administration.

Dosing Regimens and Dose Escalation for Subjects with Advanced Cancers Harboring HER2 Alterations

For monotherapy dosing, subjects with confirmed, relapsed/refractory malignancy with documented diagnosis of HER2 alterations can be enrolled sequentially into the dose cohorts starting with Cohort 1 (120 mg). Once a Subject completes a 21-day treatment cycle, in the absence of a grade 2 or greater adverse event or dose limiting toxicity, escalation may proceed to the next dosing (Cohort 2) which is 240 milligrams. This dose escalation may continue to proceed in this manner to Cohort 3 (480 mg), to Cohort 4 (960 mg), to Cohort 5 (1560 mg), and then to Cohort 6 (1080 mg dosed BID). An initial maximum tolerated dose (MTD) can be determined, and then to optimize dose selection, additional cohorts may be opened to further evaluate the safety and preliminary efficacy of the compounds of this disclosure.

### Primary Objectives

Part 1. To determine the MTD/MAD of Compound 5 as a monotherapy. Endpoints include: incidence and severity of DLTs, incidence of TEAEs/and SAEs graded according to CTCAE v5.0, and PK and changes from baseline in safety parameters: Cₘₐₓ, Tₘₐₓ, AUC₀₋ₗₐₛₜ, AUCₜₐᵤ and accumulation ratio.

Part 2. To determine the recommended dose of Compound 5 as monotherapy. Endpoints include: Incidence of TEAE/SAEs graded according to CTCAE v5.0 and PK parameters: Cmax, Tmax, AUC0-last, AUCtau and accumulation ratio.

Part 3. To characterize the efficacy of Compound 5 as monotherapy. Endpoints include: confirming objective response rate (cORR) (CR+PR) per RECIST v1.1 by investigator assessment, CNS confirmed objective response rate (CNS—ORR [CR+PR)] per RECIST v1.1, and percentage of patients who achieve a confirmed IC—ORR (IC) (CNSIC-CR+CNSIC-PR) per RANO-BM by investigator assessment.

### Part 1: Monotherapy Dose Escalation

Different dose levels, schedules, and/or frequencies may be evaluated. During “3+3” dose escalation, if none of the first 3 participants in a cohort experiences a DLT, then 3 additional participants can be enrolled at the same dose level (to extend the cohort to 6 participants) and/or 3 participants can be enrolled at the next highest dose level. Dose escalation may continue until the maximum tolerated dose (MTD) is determined.

Once a dose level has been established as tolerated (0 or 1 DLT in 6 DLT evaluable participants) then it can be backfilled with up to 6 additional participants. Backfill enrollment may occur only when no dose escalation slot is available. Participants enrolled to backfill a cohort will not be considered part of the 3+3 dose-escalation design and AEs that occur within the DLT window period will not be counted as DLTs for the purposes of dose finding. The number of backfill participants will be limited to 6 in any cohort.

If an MTD is not identified, a maximum administered dose (MAD) will be defined. The MTD/MAD will be determined based on a minimum of 6 participants at a particular dose level.

To accommodate the potential for patients being DLT unevaluable, over-enrollment of a 3 participant cohort by an additional participant may be allowed provided no DLT has been observed in that cohort. All participants will be considered for DLT assessments.

### Part 2. Monotherapy Dose Optimization

Once Part 1 is complete, to optimize dose selection, cohorts will be opened in Part 2. Recommended doses (different dose levels or dose regimens) selected for Part 2 will be at or below the MTD/MAD from Part 1, and will be determined based on evaluation of emerging safety, PK and efficacy data from Part 1, as well as preclinical data. A 1:1 randomization of 2 doses (different dose levels or dose regimens) with a sufficient number of participants (N=20 evaluable participants; unevaluable participants will be replaced) in each arm will be tested to further evaluate the tolerability, PK, and preliminary efficacy of Compound 5.

Part 2a (Monotherapy Dose Optimization): The population in Part 2 will include: participants with advanced HER TKD mutant cancers with or without brain metastases and HER2-positive cancers with brain metastases.

### Part 3. Simon 2-Stage Evaluation

Evaluation of Compound 5 as monotherapy will utilize a Simon 2-Stage Minimax Design in each of the following cohorts:
Cohort A: Advanced HER2 tyrosine kinase domain (TKD) mutant cancers with no prior HER2 tyrosine kinase inhibitor (TKI) treatment
Cohort B: Advanced HER2-positive cancers with no prior HER2 TKI treatment
Cohort C: Advanced HER2 TKD mutant and HER2-positive cancers with prior HER2 TKI treatment

### Inclusion Criteria

Have radiographically measurable disease by either or both of the following: 1. CNS disease: Response Assessment in Neuro-Oncology Brain Metastases (RANO-BM) or 2. Non-CNS disease: Response Evaluation Criteria in Solid Tumors version 1.1 (RECIST v1.1), with sites of disease that have not been previously irradiated or, if the participant has had previous radiation to the target lesion(s), there must be evidence of progression since the radiation.

Brain metastases at time of enrollment is allowed in all parts/cohorts; required for all participants with HER2-positive cancers in Part 2, with the exception of participants with HER2-positive NSCLC where presence of brain metastases is not required; must not require immediate local therapy per Investigator's judgment; and should be classified as measurable or non-measurable per RANO-BM criteria

Participants in Part 1 must have a HER2-altered solid tumors (e.g., HER2 gene mutations or fusions, HER2 gene amplification, HER2 protein overexpression); HER2 alteration should be established based on a recent archival tissue sample, fresh biopsy, or blood sample (ctDNA).

For participants with HER2-positive breast cancer enrolled in Part 2 and 3 only histologically confirmed breast carcinoma. HER2 positivity based on testing from an archival tumor tissue sample analyzed by CLIA-certified or ISO-accredited laboratory, defined as IHC3+ or IHC2+ and HER2 gene amplified by in situ hybridization (ISH) using ASCO-CAP criteria for HER2 testing in breast cancer (Part 2 and Part 3 Cohort C). Standard therapeutic regimens in the neo/adjuvant and/or metastatic setting.

Part 2: 1. Must have no standard treatment options with proven clinical benefit available or are otherwise not a candidate for standard treatment options based on the judgment of the treating physician. 2. Specifically, participants should have received the following HER2-directed agents in any setting: trastuzumab, pertuzumab, T-DXd and either T-DM1 or tucatinib; neratinib is allowed only if given in the neo/adjuvant setting. 3. Participants cannot have received lapatinib, neratinib, pyrotinib, or other experimental TKI in the metastatic setting.

Part 3: Cohort C prior treatment with trastuzumab, pertuzumab, T-DXd and at least 1 prior HER2 TKI in the metastatic setting is required; no limit to number of prior TKIs.

For participants with HER2-positive gastroesophageal cancer (including gastric and GEJ) enrolled in Part 2 and 3 only: Histologically confirmed gastric, GEJ, gastroesophageal adenocarcinoma (GEA). HER2 positivity fluoropyrimidine defined as IHC3+, or IHC2+ and HER2 gene amplified by in situ hybridization (ISH) using ASCO-CAP criteria for HER2 testing in gastroesophageal adenocarcinoma.

Prior treatment as follows: must have no standard treatment options with proven clinical benefit available or are otherwise not a candidate for standard treatment options based on the judgment of the treating physician, Standard therapeutic regimens in the metastatic setting. Specifically, participants should have received the following HER2-directed agents in the metastatic setting: trastuzumab and T-DXd, For participants in Part 2 and Part 3 Cohort B, no prior treatment with HER2 TKI is allowed, For part 3 Cohort C prior treatment with at least 1 HER2 TKI is required.

For participants with HER2-positive cancers, other than breast cancer or GEA, enrolled in Part 2 and 3 HER2 positivity based on testing from an archival tumor tissue sample analyzed by CLIA-certified or ISO-accredited laboratory, defined as any of IHC 3+ or IHC2+ and HER2 amplification by ISH (HER2/CEP17 ratio≥2.0) HER2 amplification (by next generation sequencing [NGS].

Prior treatment as follows: Standard therapeutic regimens in the metastatic setting. Specifically, standard regimens may include: trastuzumab and T-DXd (ENHERTU®). Participants must have no standard treatment options with proven clinical benefit available, or will otherwise be prevented/contraindicated from receiving any standard treatment options based on the judgment of the treating physician.

For participants in Part 2 and Cohort B, no prior treatment with HER2 TKI is allowed. For part 3 Cohort C prior treatment with at least 1 HER2 TKI is required. Participants in any part of the study who did not receive prior treatment with required agents because of lack of access (e.g., due to reimbursement/insurance coverage or because they were treated prior to regulatory/HTA agency approval) are eligible. For participants with HER2 TKD mutant NSCLC enrolled in Part 2 and 3 only: Pathologically documented unresectable and/or metastatic non-squamous NSCLC and HER2 TKD mutation (by NGS, polymerase chain reaction [PCR], or Sanger sequencing) from an archival tumor tissue sample analyzed by CLIA-certified or ISO-accredited laboratory. Mutations include: HER2/ERBB2 TKD domain covering amino acid sequence from codons 720-993 and HER2 gene fusions.

Participants in Part 2 and Part 3 Cohorts A and C must have received and failed≥1 prior therapy in the metastatic setting, which included a platinum-based chemotherapy. Participants in Part 3 Cohort A must not have received prior treatment with a HER2 TKI. Participants in Part 3 Cohort C are required to have received prior treatment with at least 1 HER2 TKI; no limit to number of prior TKIs.

For participants with HER2 TKD mutant cancers, other than NSCLC, enrolled in Part 2 and 3 only: HER2 TKD mutation (by NGS, polymerase chain reaction [PCR], or Sanger sequencing) from an archival tumor tissue sample analyzed by CLIA-certified or ISO-accredited laboratory. Mutations include: HER2/ERBB2 TKD domain covering amino acid sequence from codons 720-993 and HER2 gene fusions. Participants in Part 2 and Part 3 Cohorts A and C must have received and progressed on at least 1 prior standard therapy in the metastatic setting; previous treatment with T-DXd is allowed. Participants in Part 3 Cohort A must not have received prior treatment with a HER2 TKI. Participants in Part 3 Cohort C are required to have received prior treatment with at least 1 HER2 TKI.

### Rationale for Starting Dose

Toxicology studies of Compound 5 were conducted in rat and dog. Using surface area-based scaling, the human equivalent dose (HED) for the rat no observed adverse effect level (NOAEL; 160 mg/kg) was calculated to be 1550 mg, resulting in a maximum recommended starting dose (MRSD) of 155 mg. For the dog NOAEL (25 mg/kg), the HED was calculated to be 833 mg, and applying a ⅙ factor yields an MRSD of 140 mg. The MRSD values derived from the two toxicology species show a close proximity (155 mg vs 140 mg). Opting for a slightly more conservative approach, a starting dose of 120 mg was chosen for Compound 5. At this dosage, the safety margins stand at 12.9× and 6.9×, based on rat and dog NOAEL levels, respectively.

### Example 19. Biological Activity Assay of the Compounds Described Herein

The following experimental procedures and results for the compounds of this disclosure using various mouse models demonstrates potent in vivo anti-tumor activity across multiple HER2-driven mouse tumor models. These experimental observations exemplify just some of the advantages of the compounds in this disclosure and are not meant to limit the scope of this disclosure.

Breast Cancer Study with the BT-474 Cell Line

The BT-474 cell line was maintained in vitro as monolayer culture in DMEM supplemented with 10% fetal bovine serum and 0.01 mg/mL BI at 37° C. in an atmosphere of 5% CO2 in air. The tumor cells were routinely sub-cultured after confluence by trypsin-EDTA treatment, not to exceed 4-5 passages. The cells growing in an exponential growth phase were harvested and counted for tumor inoculation. One day before inoculating the BT-474 tumor cells, estradiol benzoate injection was administered by subcutaneous (s.c.) injection at the dose of 40 g/20 L/mouse, twice weekly till the end of the study. Each mouse was inoculated subcutaneously on the right flank (efficacy study and part II of PK study) or orthotopic on 4th right mammary fat pad (part I of PK study) with tumor cells with the single cell suspension (1×107) in 200 μL DMEM and Matrigel mixture (1:1 ratio) without serum for the tumor development.

The mouse grouping and test material treatment for tumor growth inhibition is listed in Table 3. Tumor size and body weight measurement was conducted twice weekly.

TABLE 3
Mouse grouping and test material for tumor growth inhibition

| | | Number | |
|---|---|---|---|
| Group | Test Material | of Mice | Treatment and sample collection |
| 1 | Vehicle | 6 | Vehicle |
| 2 | Compound 5 | 6 | 250 mg/kg, QD, PO, 21 days |
| 3 | Compound 5 | 6 | 100 mg/kg, QD, PO, 21 days |
| 4 | SPH5030 | 6 | 40 mg/kg, QD, PO, 21 days |
| 5 | T-DXd | 6 | 5 mg/kg, Q3W. IV |
| 6 | Tucatinib | 6 | 100 mg/kg, QD, PO, 21 days |

The mouse grouping and test material treatment for plasma and orthotopic tumor pharmacokinetic (PK) analysis is listed in Table 4. The plasma and tumor samples were analyzed for test material exposure level.

TABLE 4
Mouse grouping and test material treatment for plasma
and orthotopic tumor pharmacokinetic (PK) analysis

| | Test | Number | |
|---|---|---|---|
| Group | Material | of Mice | Treatment and sample collection |
| 7 | Compound 5 | 3 | 250 mg/kg, PO, 1x, 4 hours post dose |
| | | | collect tumor and plasma |
| 8 | Compound 5 | 3 | 250 mg/kg, PO, 1x, 12 hours post dose |
| | | | collect tumor and plasma |

The mouse grouping and test material treatment for plasma and subcutaneous (SC) tumor PK analysis is described in Table 5. The plasma and tumor samples were analyzed for test material exposure level.

TABLE 5
Mouse grouping and test material treatment for
plasma and subcutaneous (SC) tumor PK analysis

| | Test | Number | |
|---|---|---|---|
| Group | Material | of Mice | Treatment and sample collection |
| 7 | Compound 5 | 3 | 250 mg/kg, PO, 1x, 4 hours post dose |
| | | | collect tumor and plasma |
| 8 | Compound 5 | 3 | 250 mg/kg, PO, 1x, 12 hours post dose |
| | | | collect tumor and plasma |
| 9 | Compound 5 | 3 | 250 mg/kg, PO, 1x, 4 hours post dose |
| | | | collect tumor and plasma |
| 10 | Compound 5 | 3 | 250 mg/kg, PO, 1x, 24 hours post dose |
| | | | collect tumor and plasma |
| 11 | SPH5030 | 3 | 40 mg/kg, PO, 1x, 4 hours post dose |
| | | | collect tumor and plasma |
| 12 | SPH5030 | 3 | 40 mg/kg, PO, 1x, 24 hours post dose |
| | | | collect tumor and plasma |
| 13 | Tucatinib | 3 | 100 mg/kg, PO, 1x, 4 hours post dose |
| | | | collect tumor and plasma |
| 14 | Tucatinib | 3 | 100 mg/kg, PO, 1x, 24 hours post dose |
| | | | collect tumor and plasma |

Table 6 illustrates administration of the mice at 250 mg/kg once/day (QD) for 21 days. Compound 5 exhibited tumor regression (TR) starting from Day 5 (TR=56%) to the end of study (TR=92%). Administering Compound 5 at 100 mg/kg/dose also resulted in 87% TR. These data indicate that Compound 5 was efficacious in treating BT474 xenograft tumor with HER2 amplification. SPH5030, T-DXd, or tucatinib were tested as clinical reference compounds and demonstrated significantly less TR than Compound 5. No body weight loss was observed in mice treated with Compound 5. No other clinical abnormality was observed during the treatment period.

TABLE 6
Tumor regression (TR) measurement in mice

| Model | HER2 Status | Compound 5 | Comparator(s) |
|---|---|---|---|
| CDX: BT-474 breast | HER2 | Dose-dependent antitumor | T-DXd: 64% TR at 5 |
| cancer cell line; | amplification/ | effect (end of study): | mg/kg-Q3W (clinically |
| female NOD SCID | overexpression | 87% TR at 100 mg/kg | relevant dose) |
| mice | | 92% TR at 250 mg/kg. | Tucatinib: 67% TR at |
| | | | 100 mg/kg (clinically |
| | | | relevant dose) |

Breast Cancer Study with the HCC1954 Cell Line

HCC1954 cells were thawed and cultured in vitro, and the HCC1954 cells were harvested for in vivo inoculation. After one week of acclimation, 16 female 6-8 weeks-old mice were used. After inoculation, the tumor volume and body weight of the mice were measured three times per week. When the average tumor volume reaches about 200 mm³, mice were randomly grouped into 2 groups according to the tumor volume and body weight. Test drugs were administrated immediately afterward. Tumor volume and body weight measurements were taken twice a week. At the end of the study, tumor and plasma samples were collected. The mouse grouping and test material treatment for tumor growth inhibition is listed Table 7. Tumor size and body weight measurement were conducted twice weekly. At the end of study, Group 1 and Group 2 were split into 2 sub-groups, dosed and samples collected as described Table 8. The spare mice with tumor growth to large size were grouped and dosed as listed in Table 9. At the end of treatment, tumor samples were collected for pharmacodynamic (PD) analysis.

TABLE 7
mouse grouping and test material treatment
for tumor growth inhibition

| | | Number | |
|---|---|---|---|
| Group | Test Material | of Mice | Treatment and sample collection |
| 1 | Vehicle | 4 | Vehicle |
| 2 | Compound 5 | 4 | 250 mg/kg, QD, PO, 21 days |

TABLE 8
Mouse grouping split and analysis

| | | Number | |
|---|---|---|---|
| Group | Test Material | of Mice | Treatment and sample collection |
| 1a | Compound 5 | 2 | 250 mg/kg, PO, 1x, 4 hours post dose |
| | | | collect tumor and plasma and PK |
| | | | analysis |
| 1b | Compound 5 | 2 | 250 mg/kg, PO, 1x, 12 hours post |
| | | | dose collect tumor and plasma and |
| | | | PK analysis |
| 2a | Compound 5 | 2 | 250 mg/kg, PO, 1x, 4 hours post dose |
| | | | collect tumor and plasma and PK |
| | | | analysis |
| 2b | Compound 5 | 2 | 250 mg/kg, PO, 1x, 12 hours post |
| | | | dose collect tumor and plasma and |
| | | | PK analysis |

TABLE 9
Pharmacodynamic (PD) analysis

| | | Number | |
|---|---|---|---|
| Group | Test Material | of Mice | Treatment and sample collection |
| 3 | Vehicle | 2 | Vehicle |
| 4 | Compound 5 | 3 | 250 mg/kg, QD, PO, 21 days |
| 5 | Tucatinib | 3 | 100 mg/kg, QD, PO, 21 days |

Compound 5 exhibited superior anti-tumor efficacy compared to reference compound tucatinib in the HCC1954 tumor model, harboring a complex oncogenic profile characterized by HER2+, EGFR+, MET+ and PTK3CA mutation (Table 10). The enhanced effectiveness of Compound 5 can be attributed to its enrichment within the HCC1954 tumor, a property not observed in tucatinib.

TABLE 10
Anti-tumor efficacy of Compound 5

| Model | HER2 Status | Compound 5 | Comparator(s) |
|---|---|---|---|
| (FOR BREAST | HER2+, EGFR+, | Strong anti- | Tucatinib: |
| CANCER) CDX: | MET+ and | tumor effect: | tumor stasis |
| HCC1954; female | PIK3CA | 65% TR at 250 | |
| NOD SCID mice | H1047R | mg/kg | |
| Breast Cancer | mutation | | |

Lung Cancer Study with the LU11717 PDX Tumor Model

Tumor fragments from stock mice were harvested and used for inoculation into mice. Each mouse was inoculated subcutaneously in the right upper flank with each PDX model tumor fragment (2-3 mm in diameter) for tumor development. The randomization started when the mean tumor size reached approximately 500-900 mm³. 9 mice were enrolled in the study. All animals were randomly allocated to 3 study groups, 3 mice in each group. Randomization was performed based on “Matched distribution” method/“Stratified” method (StudyDirector™ software, version 3.1.399.19)/randomized block design. The treatment can be initiated on the same day of randomization per study design.

After tumor cells inoculation, the animals were checked daily for morbidity and mortality. During routine monitoring, the animals were checked for any effects of tumor growth and treatments on behavior such as mobility, food and water consumption, body weight gain/loss (Body weights were measured twice per week after randomization), eye/hair matting and any other abnormalities. Mortality and observed clinical signs were recorded for individual animals in detail. Tumor volumes were measured twice per week after randomization in two dimensions using a caliper, and the volume could be expressed in mm³ using the formula: “V=(L×W×W)/2, where V was tumor volume, L was tumor length (the longest tumor dimension) and W was tumor width (the longest tumor dimension perpendicular to L). Dosing as well as tumor and body weight measurements were conducted in a Laminar Flow Cabinet.

For the efficacy section of the study, 40 mice were randomized into 5 study groups, 8 mice in each group as shown Table 11, at the start of treatment. Tumor size and body weight measurements were conducted twice weekly.

TABLE 11
Mouse grouping for lung cancer study

| | | Number | |
|---|---|---|---|
| Group | Test Material | of Mice | Treatment and sample collection |
| 1 | Vehicle | 8 | Vehicle |
| 2 | M-135 | 8 | 250 mg/kg, QD, PO, 21 days |
| 3 | Compound 5 | 8 | 100 mg/kg, QD, PO, 21 days |
| 4 | Compound 5 | 8 | 250 mg/kg, QD, PO, 21 days |
| 5 | BI-4142 | 8 | 100 mg/kg, QD, PO, 21 days |

For tumor pharmacokinetics (PK) portion of the study, 9 mice were randomized into 3 study groups, 3 mice in each group (Table 12). The plasma and tumor samples were analyzed for Compound 5 concentrations.

TABLE 12
Pharmacokinetics (PK) study

| | Test | Number | |
|---|---|---|---|
| Group | Material | of Mice | Treatment and sample collection |
| 6 | Vehicle | 3 | PO (per os or orally) 1x, 4 hours post |
| | | | dose collect tumor and plasma |
| 7 | Compound 5 | 3 | 100 mg/kg, PO, 1x, 4 hours post dose |
| | | | collect tumor and plasma |
| 8 | Compound 5 | 3 | 100 mg/kg, PO, 1x, 12 hours post dose |
| | | | collect tumor and plasma |

For the efficacy part of the study, dosing started when the mean tumor volume reached ˜150 mm³. Compound 5 at both dosages of 100 and 250 mg/kg once daily (QD) demonstrated strong antitumor activity. As shown in Table 13, treatment with Compound 5 at 100 mg/kg/dose resulted in >30% TR on Day 7, which further increased to 61% on Day 20. Treatment with Compound 5 at 250 mg/kg/dose led to greater level of tumor regression, starting with 20% TR on Day 4 and culminating at 85% on Day 20. These data indicate that Compound 5 effectively inhibited the tumor growth of PDX with HER2 (A775_G776insYVMA) mutation. The reference compound, BI-4142, resulted in tumor growth inhibition (TGI) of ˜80%, but no tumor regression, in the first 7 days of treatment. On Day 11, the BI-4142 group started to show a TR of 8%, and gradually increased to TR of 4300 on Day 20. These data suggest that Compound 5 significantly outperformed BI-4142.

TABLE 13
Tumor growth inhibition (TGI) and tumor regression (TR)

| Param- | | Day | | | | | |
|---|---|---|---|---|---|---|---|
| eter | Test Article | 4 | 7 | 11 | 14 | 18 | 20 |
| TGI % | Compound 5 | 112% | 132% | 146% | 153% | 154% | 161% |
| | 100 mg/kg QD | | | | | | |
| | Compound 5 | 120% | 151% | 167% | 177% | 182% | 185% |
| | 250 mg/kg QD | | | | | | |
| | BI-4142 | 78% | 82% | 108% | 124% | 130% | 143% |
| | 100 mg/kg QD | | | | | | |
| TR % | Compound 5 | 12% | 32% | 46% | 53% | 54% | 61% |
| | 100 mg/kg QD | | | | | | |
| | Compound 5 | 20% | 51% | 67% | 77% | 82% | 85% |
| | 250 mg/kg QD | | | | | | |
| | BI-4142 | NA | NA | 8% | 24% | 30% | 43% |
| | 100 mg/kg QD | | | | | | |

In a PDX lung cancer model harboring the HER2-YVMA insertion mutation, the administration of Compound 5 at 100 or 250 mg/kg led to significant tumor regression, 61 and 85D, respectively (Table 14). In comparison, B-4142, a covalent HER2-selective compound, demonstrated 4300 tumor regression at its maximum tolerated dose. In addition, analysis of Compound 5 concentrations in the PDX model revealed a notably high tumor Kp (tumor/plasma concentration ratio). No body weight loss was observed in mice treated with Compound 5. No other clinical abnormality was observed during the treatment period.

TABLE 14
Tumor growth inhibition and tumor regression by Compound
5 in NSCLC PDX Model with HER2 (A775_G776insYVMA) Mutation

| Model | HER2 Status | Compound 5 | Comparator(s) |
|---|---|---|---|
| PDX: HuPrime ® | HER2- | Dose-dependent | BI-4142: 43% |
| lung cancer | A775_{—} | anti-tumor effect | TR at 100 |
| model (Crown | G776insYVMA | (end of study): | mg/kg (MTD) |
| Bioscience); | | 61% TR at 100 | |
| female NOD | | mg/kg | |
| SCID mice | | 85% TR at 250 | |
| | | mg/kg | |

Gastric Cancer Study with the NCI-N87 Cell Line

The mouse grouping and test article treatment for tumor growth inhibition is listed in the Table 15. Tumor size and body weight measurement was conducted twice weekly.

TABLE 15
Gastric cancer study with the NCI-N87 Cell Line

| | | Number | |
|---|---|---|---|
| Group | Test Material | of Mice | Treatment and sample collection |
| 1 | Vehicle | 10 | Vehicle |
| 2 | Compound 5 | 10 | 250 mg/kg, QD, PO, 36 days |
| 3 | Compound 5 | 10 | 100 mg/kg, QD, PO, 36 days |
| 4 | Tucatinib | 10 | 100 mg/kg, QD, PO, 36 days |
| 5 | T-DXd | 10 | 5 mg/kg, Q3W, IV, 36 days |

The mouse grouping and test article treatment for plasma, normal brain, and brain tumor PK analysis is described in Table 16. The plasma, tumor and contralateral normal brain samples were analyzed for dosed test article exposure level.

TABLE 16
Mouse grouping and test article treatment for plasma,
normal brain, and brain tumor PK analysis

| | Test | Number | |
|---|---|---|---|
| Group | Material | of Mice | Treatment and sample collection |
| 6 | Compound 5 | 3 | 250 mg/kg, PO, 1x, 4 hours post dose collect tumor, |
| | | | contralateral normal brain and plasma |
| 7 | Compound 5 | 3 | 250 mg/kg, PO, 1x, 12 hours post dose collect tumor, |
| | | | contralateral normal brain and plasma |
| 8 | Compound 5 | 3 | 100 mg/kg, PO, 1x, 4 hours post dose collect tumor, |
| | | | contralateral normal brain and plasma |
| 9 | Compound 5 | 3 | 100 mg/kg, PO, 1x, 12 hours post dose collect tumor, |
| | | | contralateral normal brain and plasma |
| 10 | Tucatinib | 3 | 100 mg/kg, PO, 1x, 4 hours post dose collect tumor, |
| | | | contralateral normal brain and plasma |
| 11 | Tucatinib | 3 | 100 mg/kg, PO, 1x, 12 hours post dose collect tumor, |
| | | | contralateral normal brain and plasma |
| 12 | ENB0003 | 2 | 5 mg/kg, PO, 1x, 24 hours post dose collect tumor, |
| | | | contralateral normal brain and plasma |
| 13 | ENB0003 | 2 | 5 mg/kg, PO, 1x, 168 hours post dose collect tumor, |
| | | | contralateral normal brain and plasma |

The dosing duration was 37 days, started 12 days after cell inoculation. The dosing Day 1 was Day 12 post cell inoculation. After dosing Day 26 (as “D26”), the control group mice started to drop out. The last day of TGI calculation was set at Day 26, whereas TR was calculated until the last day of dosing (Day 37; Table 4). At 250 mg/kg (QD, for 37 days), Compound 5 resulted in TR from Day 5 (TR=370) to the end of study (TR=700). Compound 5 at 100 mg/kg also resulted in TR with a range from 110%-46% in the treatment duration of Day 1-30. These data indicate that Compound 5 reduced the size of intracranial human NCI-N87 tumor with HER2 amplification, in a dose-dependent manner. No body weight loss was observed in mice treated with Compound 5. No other clinical abnormality was observed during the treatment period.

ENB0003 (T-Dxd) was dosed intravenously (IV) every 3 weeks (Q3W) at 5 mg/kg causing significant tumor growth inhibition (TGI close to 1000%), but no tumor regression. Tucatinib dosed at 100 mg/kg caused a modest tumor growth inhibition (TGI≤30%; Table 17). Table 18 illustrates Compound 5 tumor regression in intracranial human NCI-N87 tumor model.

TABLE 17
Compound 5 and ENB0003 tumor growth inhibition
in intracranial human NCI-N87 tumor model

| | Tumor Growth Inhibition | | | | | | |
|---|---|---|---|---|---|---|---|
| Test Article | Day | Day | Day | Day | Day | Day | Day |
| and Dose | 5 | 9 | 13 | 16 | 19 | 23 | 26 |
| Compound 5 | 137% | 137% | 149% | 163% | 160% | 172% | 169% |
| 250 mg/kg QD | | | | | | | |
| Compound 5 | 146% | 146% | 116% | 141% | 136% | 133% | 120% |
| 100 mg/kg QD | | | | | | | |
| Tucatinib | 9% | −11% | −14% | 18% | 45% | 25% | 30% |
| 100 mg/kg QD | | | | | | | |
| ENB0003 | 47% | 97% | 97% | 99% | 98% | 97% | 96% |
| 5 mg/kg Q3W | | | | | | | |

TABLE 18
Compound 5 tumor regression in intracranial human NCI-N87 tumor model

| | Tumor Regression | | | | | | | | | |
|---|---|---|---|---|---|---|---|---|---|---|
| Test Article | | | | Day | Day | Day | Day | Day | Day | Day |
| and Dose | Day 5 | Day 9 | Day 13 | 16 | 19 | 23 | 26 | 30 | 33 | 37 |
| Compound 5 | 37% | 37% | 49% | 63% | 60% | 72% | 69% | 72% | 67% | 70% |
| 250 mg/kg QD | | | | | | | | | | |
| Compound 5 | 46% | 46% | 16% | 41% | 36% | 33% | 20% | 11% | NA | NA |
| 100 mg/kg QD | | | | | | | | | | |
| Intracranial | HER2 | Dose- | T-DXd: 99% | | | | | | | |
| CDX: NCI- | amplification/ | dependent | TGI (e.g. | | | | | | | |
| N87-luciferase | overexpression | antitumor | tumor stasis) at | | | | | | | |
| gastric cancer | | effect (best | 5 mg/kg-Q3W | | | | | | | |
| cell line; | | response): | (clinically | | | | | | | |
| female | | 46% TR at | relevant dose) | | | | | | | |
| BALB/c mice | | 100 mg/kg | Tucatinib: | | | | | | | |
| | | 72% TR at | modest (30%) | | | | | | | |
| | | 250 mg/kg | TGI at | | | | | | | |
| | | | 100 mg/kg | | | | | | | |
| | | | (clinically | | | | | | | |
| | | | relevant dose) | | | | | | | |

HER2-driven tumors have a high propensity to metastasize to the brain. The efficacy of Compound 5 was evaluated in an intracranial xenograft model generated using luciferase expressing NCI-N87 human cancer cells (NCI-N87-luc). The total bioluminescent signals (photons/s) emitted from the brains after luciferin injection were used as an indicator of tumor growth and antitumor activity. The administration of Compound 5 at doses of 100 and 250 mg/kg exhibited dose-dependent tumor regression effects. In comparison, T-DXd achieved tumor stasis, while tucatinib, despite demonstrating clinical efficacy when used alongside trastuzumab and capecitabine in the treatment of subjects with breast cancer who have brain metastasis, displayed only modest single agent effect in this model.

Unique among known HER2 inhibitors, the compounds of this disclosure as described by Formulae I, II IIa and in the examples showed preferential tumor enrichment, a phenomenon that has been observed across tumor types and anatomic sites. This property is linked to HER2 expression, resulting in target engagement with high precision enhancing its tumor-specific action while minimizing systemic side effects. Compound 5 has shown high brain penetrance in brain PK evaluations, and superior efficacy in an IC tumor model compared with tucatinib and T-DXd. Compound 5 also inhibited the growth of the HER2 exon 20 mutant lung cancer cell line while sparing the EGFR overexpressing/HER2-low epidermoid carcinoma A-431 cells that demonstrated that the compounds of this disclosure are potent toward HER2 amplified and mutant cell lines and selective against cancer cells with EGFR overexpression in vitro.

### Example 20. Compound 5 Usage in Colorectal Cancer (CRC) and Other Indications with KRAS Wild Type or Mutant

KRAS mutations are present in approximately 25% of tumors, making them one of the most common gene mutations linked to cancer. They are frequent drivers in lung, colorectal, and pancreatic cancers. KRAS drives 32% of lung cancers, 40% of colorectal cancers, and 85% to 90% of pancreatic cancer cases. KRAS mutation has been reported as a clinical mechanism of resistance to HER2 inhibitors such as trastuzumab. A small molecule HER2 inhibitor tucatinib was approved in HER2+CRC only with RAS wild type, not KRAS mutants. In a preclinical model KYSE410 with HER2+ and KRAS G12C mutant, tucatinib was found to have weak/no activity (FIG. 1). KYSE410 also showed resistance to approved KRAS inhibitor AMG510 (FIG. 2). Together, there is no selective HER2 inhibitor validated to work on HER2+ tumors bearing KRAS mutation. Thus, there is an unmet medical need on HER2 wild type KRAS-mutant cancers.

As shown in this example, Compound 5 inhibited HER2+ KRAS mutant KYSE410 tumor. Compound 5 is a covalent HER2 inhibitor with preferential tumor enrichment feature. Biological activity of Compound was tested in KYSE410 model (HER2+ and KRAS G12C) in comparison to AMG510. As shown in FIG. 3, Compound 5 at 250 mg/kg dosage caused near complete tumor regression. AMG510 showed marginal activity as reported. These data suggest a clinical potential of Compound 5 (and its derivatives with similar activity profile) in treatment for HER2+ KRAS mutant tumors and metastasis in human subjects.

Combination of Compound 5 and KRAS G12C inhibitor MRTX849 synergistically inhibited HER2+ KRAS mutant KYSE410 cell growth. The combinatorial effect of Compound 5 and KRAS G12C inhibitor MRTX849 was tested on inhibiting HER2+ KRAS mutant KYSE410 cell growth. As shown in FIG. 4, the achieved combinatorial index (CI) was in the range of 0.024-0.248, indicating that Compound 5 and MRTX849 synergistically inhibited HER2+ KRAS mutant KYSE410 cell growth.

As shown in this example, the potential clinical application of Compound 5 includes treating: CRC and metastasis harboring HER2+ KRAS wild type and HER2+ KRAS mutant; other indications (lung, pancreatic, etc) and metastasis harboring HER2+ KRAS wild type and HER2+ KRAS mutant; combining with KRAS inhibitors, treating CRC and metastasis harboring HER2+ KRAS wild type and HER2+ KRAS mutant; and combining with KRAS inhibitors, treating other indications (lung, pancreatic, etc) and metastasis harboring HER2+ KRAS wild type and HER2+ KRAS mutant.

### Example 21. Compound 5 in Ba/F3 HER2 Xenograft Model

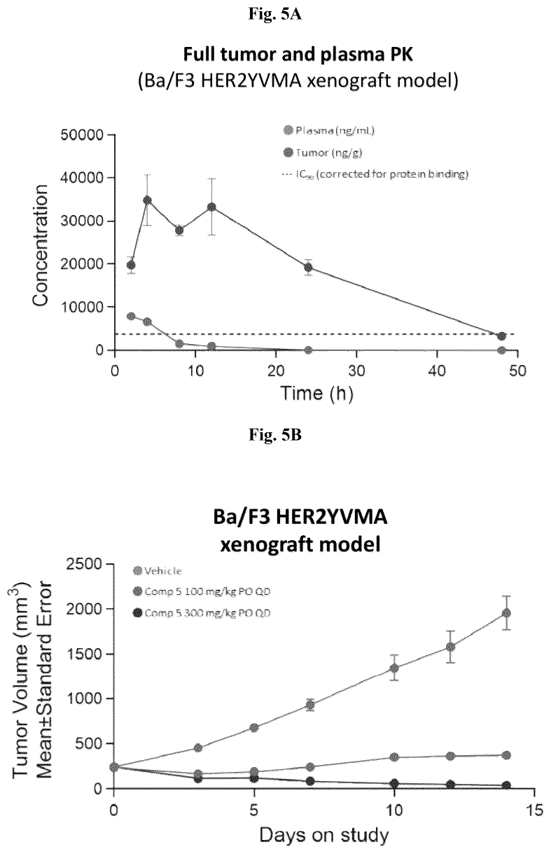

FIG. 5A and FIG. 5B demonstrate that Compound 5 has a preferential tumor tissue distribution profile. FIG. 5A indicates that Compound 5 exhibited sustained enriched tumor exposure after a single PO dose at 300 mg/kg in Ba/F3 HER2YVMA xenograft model. FIG. 5B demonstrates that Compound 5 treatment led to significant tumor inhibition and regression in Ba/F3-HER2YVMA tumor model.

Additionally, Table 19 showed improved tumor exposure resulted in high tumor-Kp multiple HER2-driven models.

TABLE 19
High tumor-Kp across HER2-driven tumor models.

| | BaF3 (HER2-YVMA) | | HCC1954 | |
|---|---|---|---|---|
| TGI model | Compound 5 | Poziotinib | Compound 5 | Zongertinib |
| 4 h | 3.1 | 0.36 | 16.4 | 0.3 |
| 12 h | 68 | NA | 119 | 0.7 |

Compound 5 demonstrated improved tumor exposure resulting in high tumor-Kp in multiple HER2-driven models. The high tumor-Kp is a differentiated property of Compound 5 and is not observed with other covalent HER2 inhibitors such as zongertinib or poziotinib. Enriched tumor targeting yielded>1000× HER2/EGFR selectivity window.

### Example 22. Compound 5 in Xenograft Resistance to HER2 ADC in p95HER2

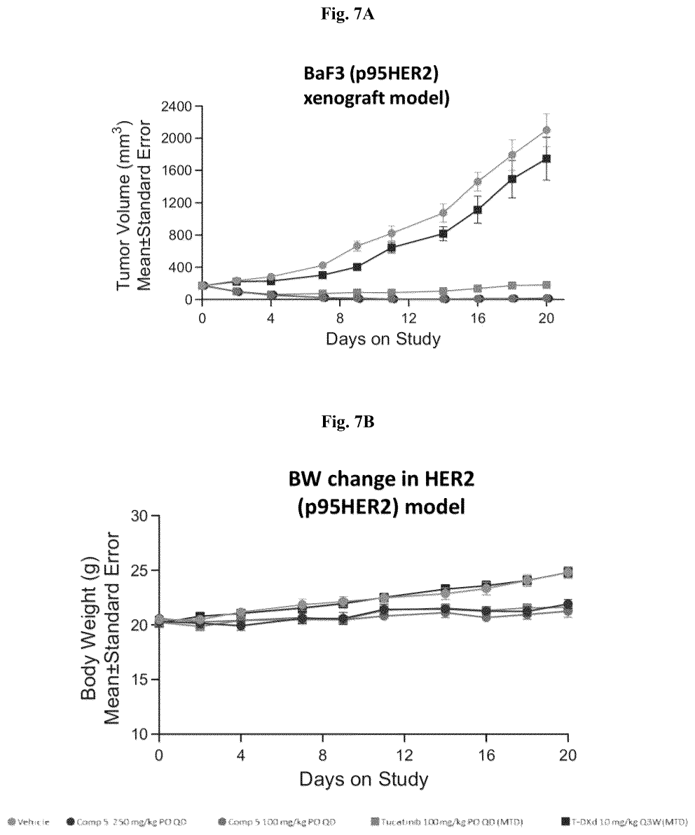

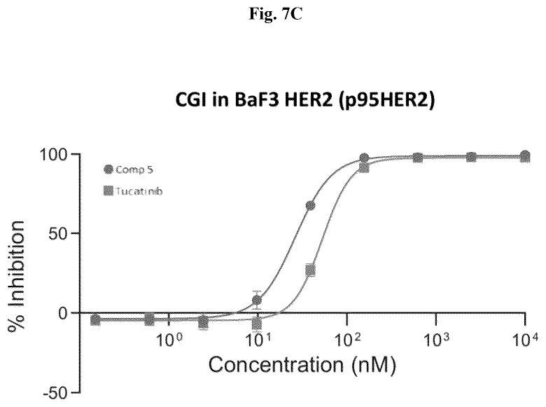

Treatment with Compound 5 led to significant tumor regression at 100 and 250 mg/kg dose levels in N-terminally truncated HER2 (p95HER2) tumor model that was resistant to HER2 ADC T-DXd (seen in FIG. 7A). FIG. 7B shows that both dose levels of Compound 5 were well tolerated throughout the treatment duration and that no body weight loss was observed. FIG. 7C illustrates that Compound 5 showed cellular proliferation IC₅₀s in the low nanomolar range. Comparisons against tucatinib 100 mg/kg and T-DXd 10 mg/kg demonstrated the superiority or parity of Compound 5 at both treatment regimens.

### Example 23. Compound 5 in Xenografts Harboring PIK3CA and KRAS Mutations

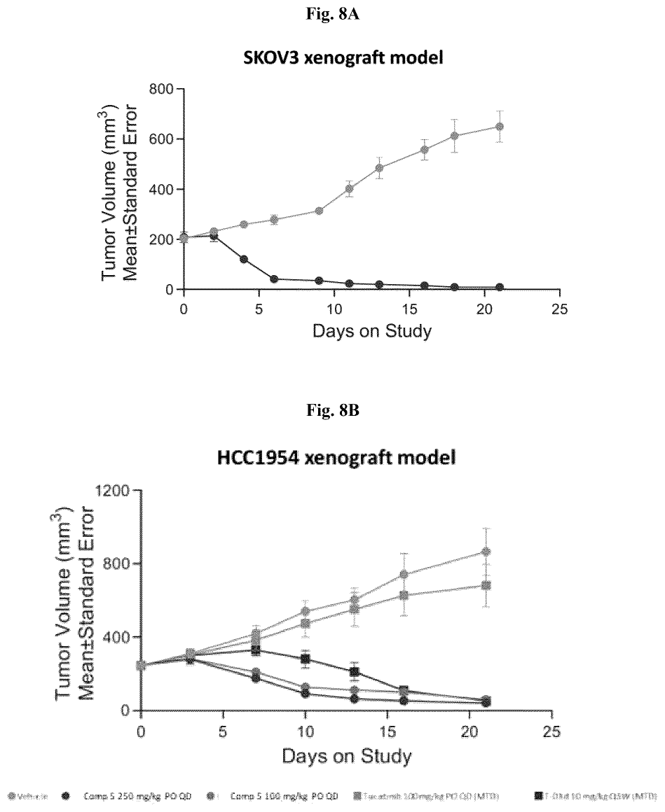

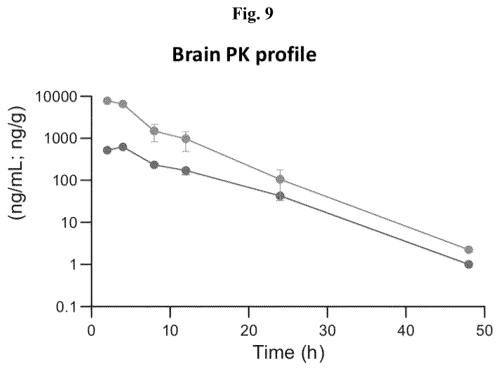

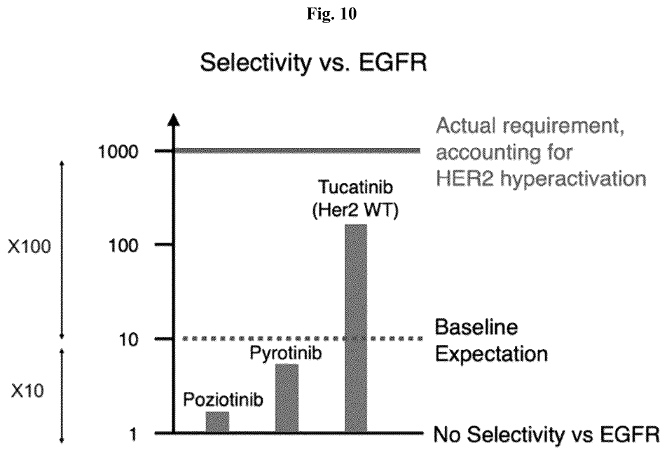

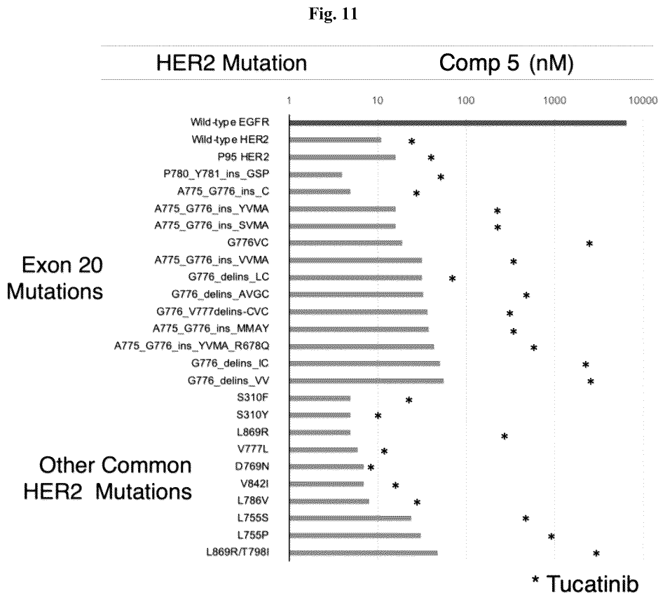

Compound 5 was shown to be efficacious across a broad range of HER2-driven tumor models. FIG. 8A showed deep regression was achieved in PIK3CA H1047R mutant SKOV3 TGI model while FIG. 8B showed that Compound 5 had strong efficacy when compared to reference compounds in PIK3CA H1047R mutant HCC1954 TGI model. Table 20 showed that Compound 5 dosed at 100 mg/kg PO QD or 250 mg/kg PO QD was efficacious in models with variable levels of HER2 expression and co-occurring mutations.

TABLE 20
Compound 5 efficacy in TGI models.

| | | | PIK3CA | | |
|---|---|---|---|---|---|
| | | | signaling | KRAS | |
| | | | mutation | mutation | |
| Model | Indication | ERBB2 | status | status | TGI |
| BT474 | Breast | 3.64 | K111N | N | 192% |
| N87 | Gastric | 5.27 | N | N | 120% |
| HCC1954 | Breast | 5.88 | H1047R | N | 176% |
| KYSE410 | Esophageal | 5.26 | N | G12C | 192% |
| SKOV3 | Ovarian | 2.26 | H1047R | N | 195% |
| Calu3 | Lung | 5.1 | N | N | 180% |
| HCC827 | Lung | 1.41 | N | N | 191% |
| MFE-280 | Endometrial | 1.89 | H1047Y | N | 88% |
| H2122 | Lung | 1.15 | N | G12C | 63% |
| A549 | Lung | 1.28 | N | G12S | 71% |
| A431 | Epidermoid | 1.10 | N | N | 78% |

### Example 24. Compound 5 in N87 IC Xenograft Model

Compound 5 was brain permeable and showed efficacy in IC models. FIG. 9A shows sustained brain exposure in Ba/F3 HER2 YVMA tumor bearing mice when dosed with Compound 5 at 300 mg/kg. Samples were measured 2 to 48 hours after dosing. FIG. 9B shows that treatment with Compound 5 dosed at 250 mg/kg (QD, PO) resulted in regression of N87 intracranial tumors. A comparison was made to tumor static effect of T-DXd (5 mg/kg, Q3W).

Table 21 shows that Compound 5 was brain penetrant in mouse (CD-1) and monkey (cyno).

TABLE 21
Brain Kp:uu.

| | mouse | monkey |
|---|---|---|
| | Compound 5 | Compound 5 |
| 2 h | 0.29 | 0.31 |
| 6-8 h | 0.36 | 0.40 |

### Example 25. Compound 5 C₀-Crystal Binding Model

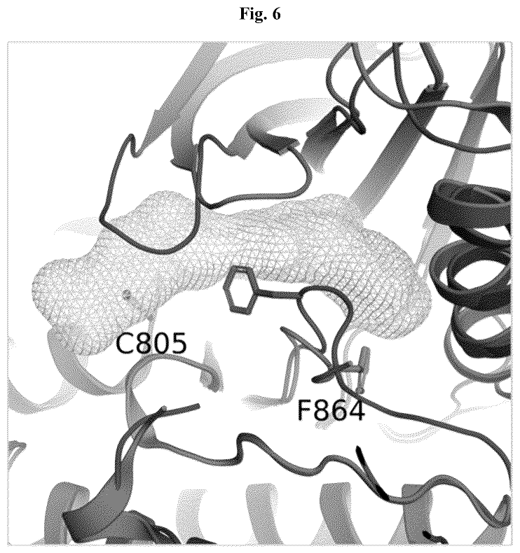

Compound 5 is a highly selective type II kinase inhibitor. FIG. 6 shows covalent HER2 inhibitor binding to the inactive DFG-out conformation of HER2 WT. A co-crystal structure (3.4 Å) of Compound 5 is overlaid with PDB structure 7PCD.

Table 22 indicates that Compound 5 demonstrates enhanced HER2/EGFR kinetic selectivity vs. reference molecules.

TABLE 22
Enzyme inactivation kinetic parameters.

| | HER2 | | | EGFR | | | Kinetic |
|---|---|---|---|---|---|---|---|
| | Kinact | Ki | kinact/Ki | Kinact | Ki | kinact/Ki | EGFR/HER2 |
| | (s⁻¹) | (nM) | (M⁻¹ s⁻¹ ) | (s⁻¹) | (nM) | (M⁻¹ s⁻¹ ) | selectivity |
| Compound 5 | 3.4E−02 | 16 | 2.2E+06 | 1.2E−03 | 293 | 4.2E+03 | 515 |
| Zongertinib | 1.7E−02 | 94 | 1.8E+05 | 8.6E−04 | 455 | 1.9E+03 | 93 |

### Example 26. Kinetic Analysis of Compound 5

Compound 5 is a highly selective in comparison to competing HER2 inhibitors Zongertinib and ELVN-002 even before accounting for preferential tumor distribution. Table 23 shows the comparative selectively of Compound 5, which exhibits 5 to 10 fold selectively of HER2 over EGRF.

TABLE 23
Kinetic Analysis

| | HER2 | | | EGFR | | | HER2 vs. EGFR |
|---|---|---|---|---|---|---|---|
| | kinact | KI | kinact/KI | kinact | KI | kinact/KI | Kinetic |
| Compound | (s − 1) | (nM) | (s − 1 nM − 1) | (s − 1) | (nM) | (s − 1 nM − 1) | Selectivity |
| Compound 5 | 3.4 × 10⁻² | 16 | 2.2 × 10⁶ | 1.2 × 10⁻³ | 290 | 4.2 × 10³ | 520 |
| Zongertinib | 1.7 × 10⁻² | 94 | 1.8 × 10⁵ | 8.6 × 10⁻⁴ | 460 | 1.9 × 10³ | 93 |
| ELVN-002* | 1.3 × 10⁻² | 18 | 7.3 × 10⁵ | 1.1 × 10⁻² | 680 | 1.6 × 10⁴ | 47 |

### Example 27. Preferential Tumor Distribution of Compound 5

Compound 5 exhibited a high tumor/plasma ratio in mouse xenograft models. Such preferential tumor distribution maximizes target engagement while reducing systemic side effects. Table 24 shows high tumor-Kp across HER2 driven tumor models, in addition to a model with low HER2 as a negative control.

TABLE 24
Compound 5 across models

| | BaF3 | PDX | | A431 (low |
|---|---|---|---|---|
| | (HER2- | (HER2- | Orthotopic | HER2) negative |
| Time | YVMA) | YVMA) | (HER2-Amp) | control |
| 4 hr | 3.1/3.2 | 4.7 | 8 | 0.6 |
| 12 hr | 68/55 | 203 | 43 | 2.9 |

Table 25 compares active ingredients and shows that a high tumor-Kp is unique to Compound 5.

TABLE 25
Kp comparison

| | BaF3-HER2-YVMA xenograft model | | | HCC1954 xenograft model | |
|---|---|---|---|---|---|
| Time | Compound 5 | BI-4142 | Poziotinib | Compound 5 | Zongertinib |
| 4 hr | 3.1 | 0.44 | 0.36 | 16 | 0.26 |
| 12 hr | 68 | 0.90 | NA | 119 | 0.67 |

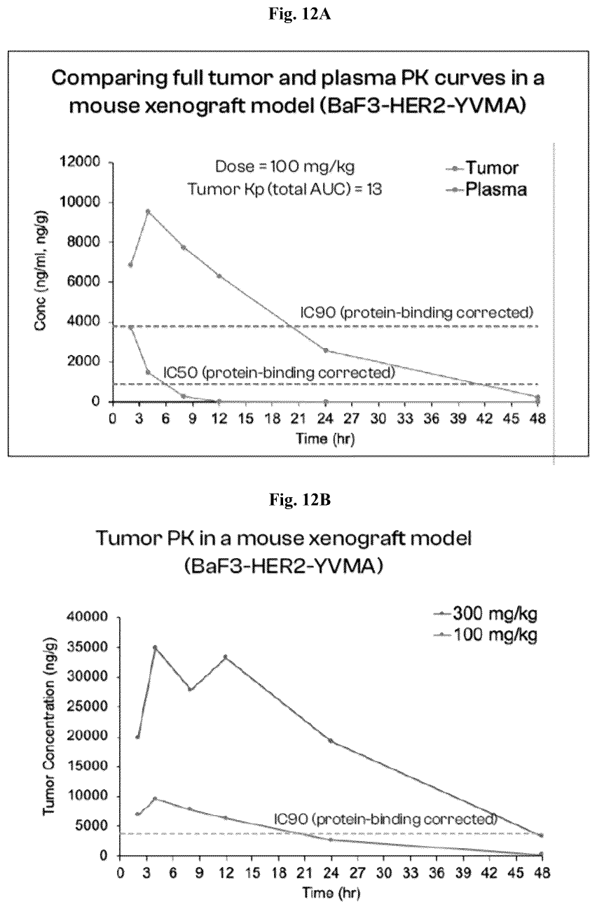

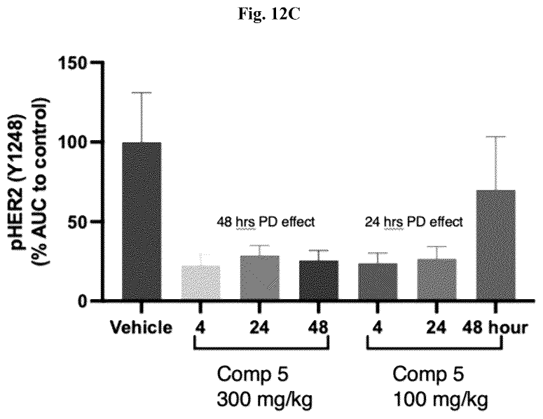

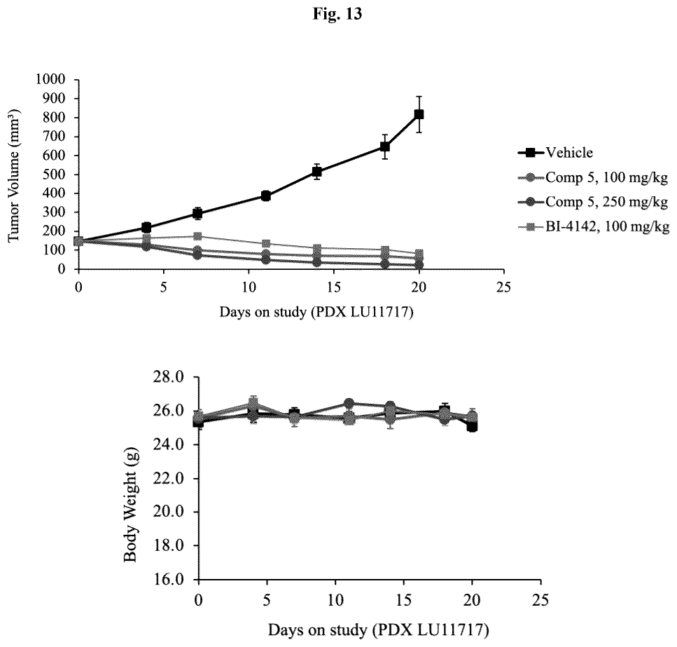

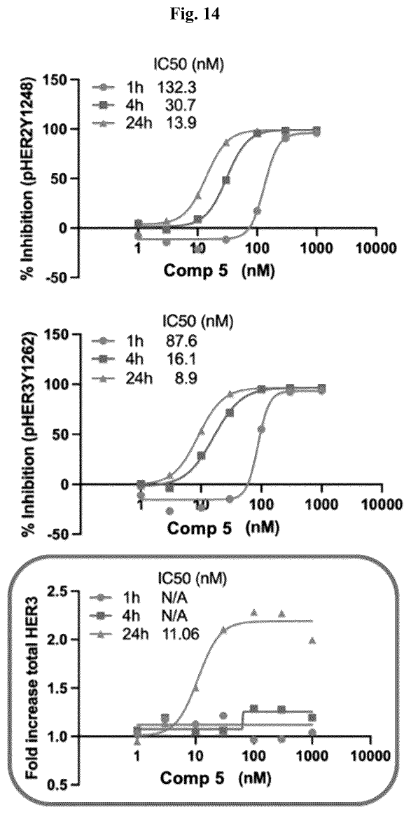

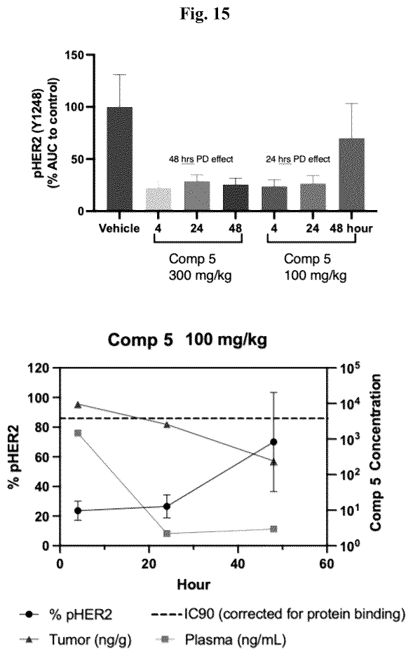

FIG. 12A shows the comparison of full tumor and plasma PK curves in a mouse xenograft model (BaF3-HER2-YVMA). Dose of Compound is 100 mg/kg. FIG. 12B shows the comparison of tumor PK in a mouse xenograft model (BaF3-HER2-YVMA) between 100 and 300 mg/kg doses of Compound 5. FIG. 12C shows the peak comparison of pHER2 (Y1248) between 100 and 300 mg/kg doses of Compound 5 at various time points. These data show that Compound 5 achieves over 1000× in vivo selectivity against EGFR by combining 10× preferential tumor distribution and 100× enzymatic selectively. The increased selectivity leads to superior efficacy and a greater therapeutic window.

### Example 28. Compound 5 in MDAMB453 Breast Cancer Model

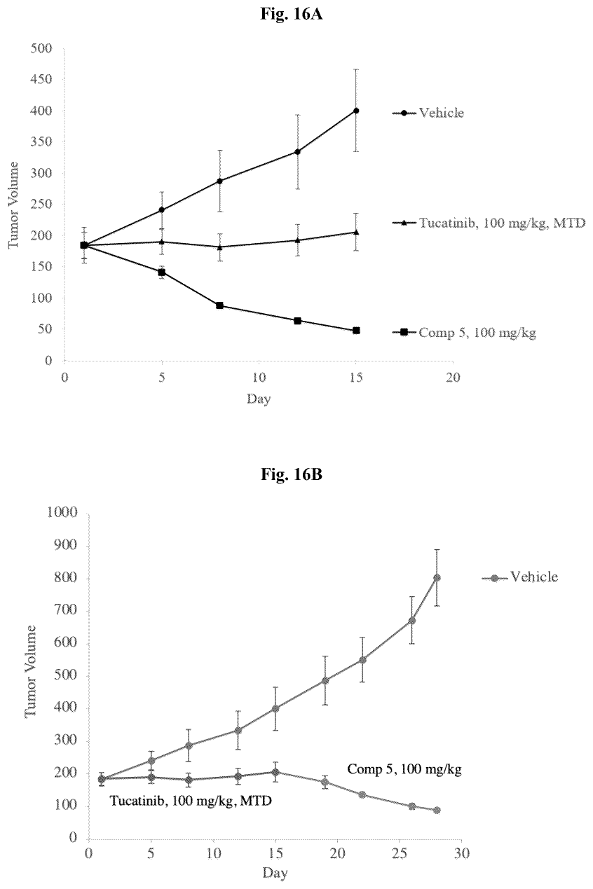

FIG. 16A illustrates that Compound 5 (100 mg/kg) has superior efficacy when compared to tucatinib (100 mg/kg). In the MDAMB453 breast cancer model (HER2+, PIK3CA-H1047R), dosing of Compound 5 at 100 mg/kg PO daily for 14 days and caused TGI (tumor growth inhibition) of 174% in the animals (N=4 per group), whereas dosing of tucatinib was dosed in a group of mice at 100 mg/kg PO daily for 14 days and caused TGI of 90%.

FIG. 16B illustrates that Compound 5 dosed at 100 mg/kg provides for regression following tucatinib resistance. In MDAMB453 breast tumor model (HER2-amplified), the animals (N=4 per group) were dosed with tucatinib at 100 mg/kg PO daily for 14 days and caused TGI of 90%. In post tucatinib dosing, the same mice were dosed with Compound 5 at 100 mg/kg PO daily for an extended 14 days and achieved TGI of 157% in the MDAMB453 breast cancer model (HER2+, PIK3CA-H1047R).

### Example 29. Compound 5 in NCI-N87 Luciferase Cell Model

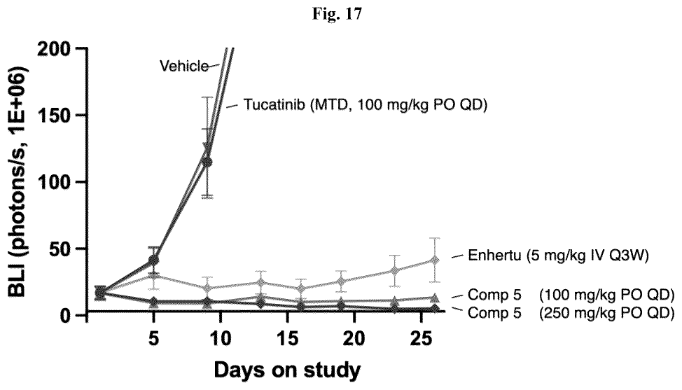

FIG. 17 illustrates that Compound 5 is efficacious in a brain metastasis tumor model. Nude mice were intracranially implanted with NCI-N87 luciferase cells. In N87 gastric intracranial tumor model (HER2-amplified), the animals (N=10 per group) were dosed with compound 5 at 250 and 100 mg/kg PO daily for 26 days and caused TGI of 169% and 120%, respectively. Enhertu was dosed in a group of mice at 5 mg/kg IV Q3W for 26 days and caused TGI of 96%. Tucatinib was dosed in a group of mice at 100 mg/kg PO daily for 26 days and caused TGI of 30%.

### Example 30. Compound 5 in NCI-H1693 NSCLC Model

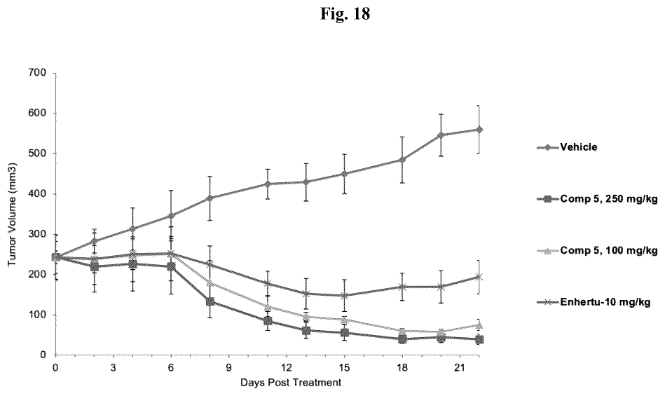

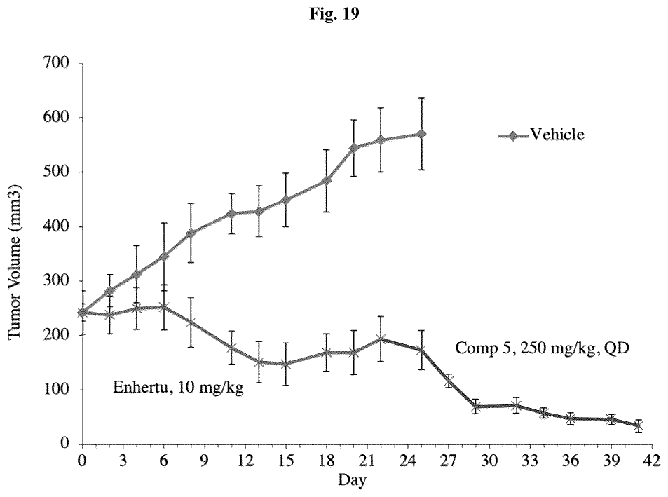

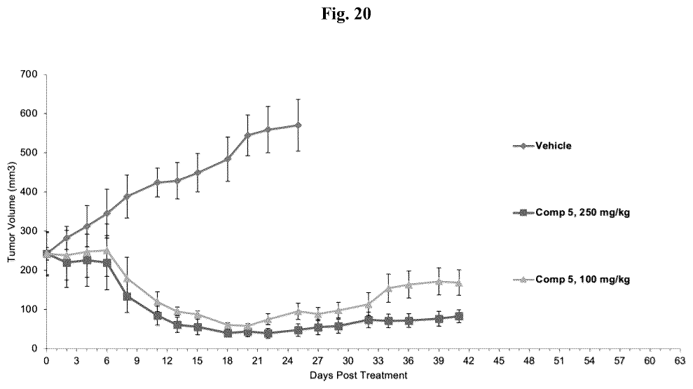

FIG. 18 illustrates that Compound 5 dosed at 250 and 100 mg/kg caused TGI of 182% and 169%, respectively in a NCI-H1693 NSCLC model (HER2-amplified). Dosing of enhertu at 10 mg/kg caused TGI of 119%. FIG. 19 illustrates post enhertu dosing. In NCI-H1693 NSCLC tumor model (HER2-amplified), the animals (N=4 per group) were dosed with enhertu at 10 mg/kg IV once for 21 days and caused TGI of 119%. Post enhertu dosing, the same group of mice were dosed of compound 5 at 250 mg/kg PO daily for an extended 21 days and achieved TGI of 182%. FIG. 20 illustrates mouse tumor growth post administration of Compound 5. In NCI-H1693 NSCLC tumor model (HER2-amplified), the animals (N=4 per group) were dosed with compound 5 at 250 and 100 mg/kg PO daily for 21 days and caused TGI of 182% and 169%, respectively. After dosing stopped, the mouse tumor growth was followed up for 21 days (recovery period). In the recovery period, the tumors in the mice dosed with compound 5 at 250 mg/kg retained a TGI of 166%, where the tumors in the mice dosed with compound 5 at 100 mg/kg retained a TGI of 130%.

### Example 31. Compound 5 in NCI-H2030 Tumor Model

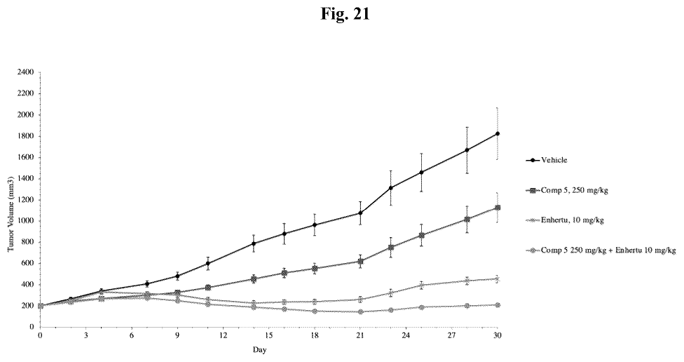

FIG. 21 illustrates that Compound 5 and enhertu have synergistic effect on inhibiting HER2-low NSCLC cancer. In NCI-H2030 tumor model (NSCLC cancer with Her2-low and Kras mutation), the animals (N=8 per group) were dosed with compound 5 at 250 mg/kg PO daily for 30 days and caused TGI of 43%. In another group of mice, dosing of enhertu at 10 mg/kg IV Q3W for 30 days caused TGI of 84%. Combined dosing of compound 5 at 250 mg/kg PO daily and enhertu at 10 mg/kg IV Q3W for 30 days caused TGI of 99.5%. A synergy of was observed for the combination of Compound 5 and enhertu.

### Example 32. Compound 5 in NCI-H2122 Tumor Model

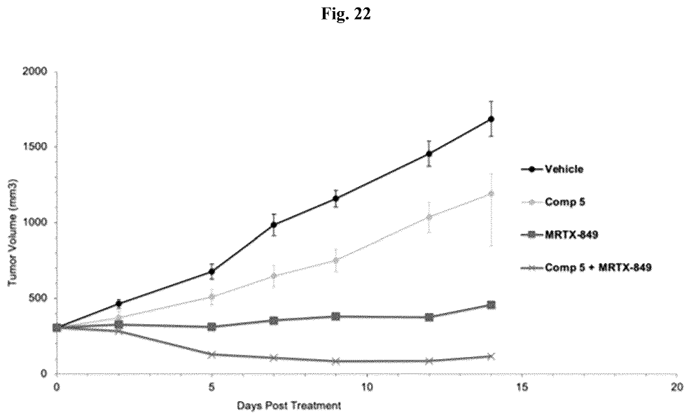

FIG. 22 illustrates that Compound 5 in combination with and a Kras inhibitor has synergistic effect on inhibiting NSCLC cancer. In NCI-H2122 tumor model (NSCLC cancer with Her2-low and Kras mutation), the animals (N=8 per group) were dosed with compound 5 at 100 mg/kg PO daily for 14 days and caused TGI of 36%. In another group of mice, dosing of MRTX-849 at 100/75 mg/kg PO daily for 6 (at 100 mg/kg) then 8 days (at 7.5 mg/kg) caused TGI of 95%. Combined dosing of compound 5 at 100 mg/kg PO daily and MRTX-849 at 100/75 mg/kg PO daily for 6 (at 100 mg/kg) then 8 days (at 75 mg/kg) caused TGI of 99.5%. A strong synergy with the combination of Compound 5 and MRTX-849 was observed.

### Example 33. Compound 5 in ASPC-1 Tumor Model

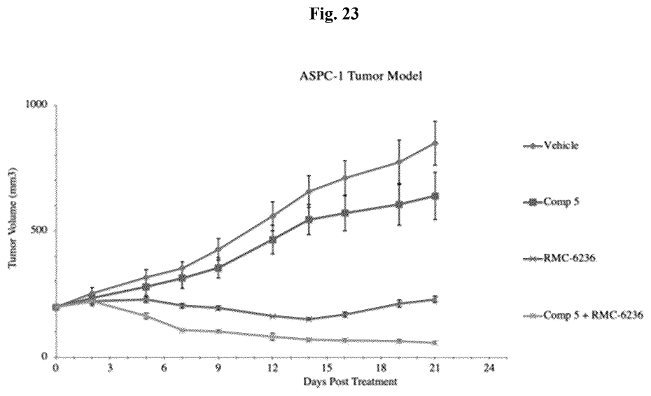

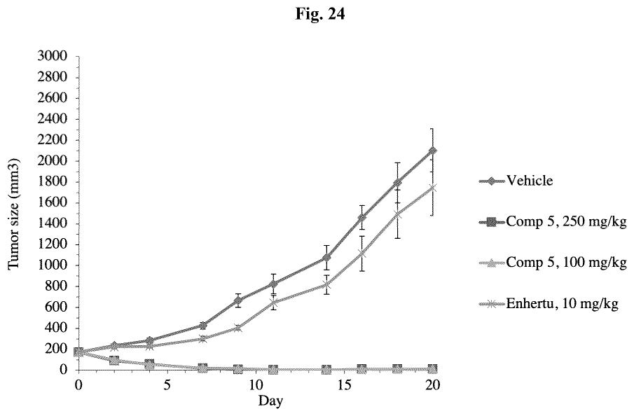

FIG. 23 illustrates that Compound 5 and a Kras inhibitor have synergistic effect on inhibiting pancreatic cancer. In ASPC-1 tumor model (Pancreatic cancer with Her2-low and Kras mutation), the animals (N=4 per group) were dosed with compound 5 at mostly 100 mg/kg PO daily for 21 days and caused TGI of 32%. In another group of mice, dosing of RMC-6236 at 10/7.5 mg/kg PO daily for 15 (at 10 mg/kg) then 6 days (at 7.5 mg/kg) caused TGI of 89%. Combined dosing of compound 5 at mostly 100 mg/kg PO daily and RMC-6236 at 10/7.5 mg/kg PO daily for 21 days caused TGI of 171%. A strong synergy with the combination of Compound 5 and RMC-6236 was observed.

### Example 34. Compound 5 in BaF3 P95Her2 Tumor Model

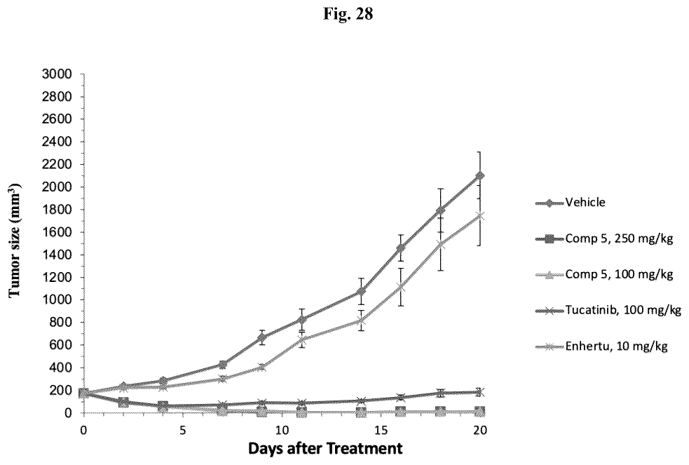

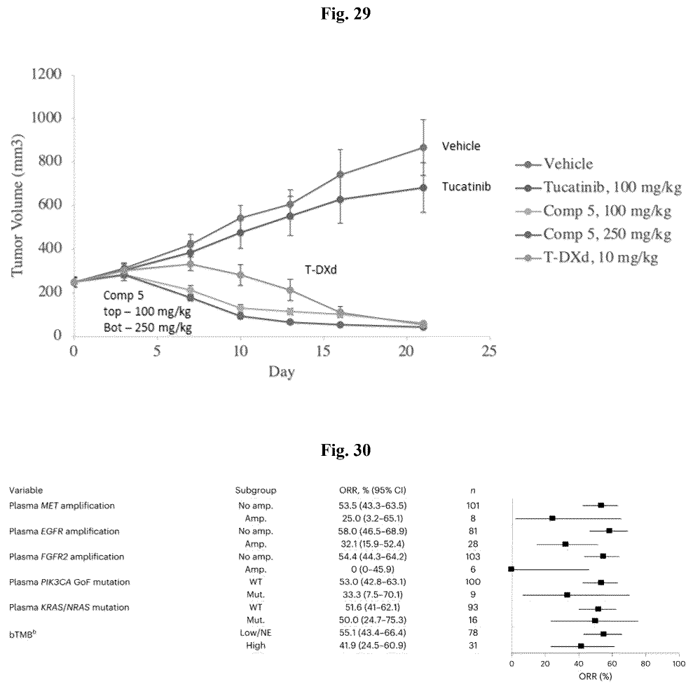

FIG. 24 illustrates that Compound 5 shows significant in vivo activity in BaF3 p95Her2 cancer model resistant to enhertu. P95Her2 is a truncated form of Her2 without extracellular domain (ECD), which is needed for enhertu to bind to. It was considered as a mechanism of resistance to Her2 antibody therapies, including enhertu. In BaF3 P95Her2 tumor model (engineered to overexpress P95Her2), the animals (N=6 per group) were dosed with compound 5 at 250 and 100 mg/kg PO daily for 20 days and caused TGI of 193% and 190%, respectively. Enhertu was dosed in a group of mice at 10 mg/kg IV once for 20 days and caused TGI of 19%, showing that this model is resistant to enhertu treatment. FIG. 28 illustrates a comparison of Compound 5 (100 and 250 mg/kg), tucatinib (100 mg/kg), and enhertu (10 mg/mg) in HER2 extracellular truncation mouse xenograft models (p95 HER2 mutation Ba/F3 cell line). In BaF3 P95Her2 tumor model, the animals (N=6 per group) were dosed with compound 5 at 250 and 100 mg/kg PO daily for 20 days and caused TGI of 193% and 190%, respectively. Enhertu was dosed in a group of mice at 10 mg/kg IV once for 20 days and caused TGI of 19%. Tucatinib was dosed in a group of mice at 100 mg/kg PO daily for 20 days and caused TGI of 99%. Application of Compound 5 achieved complete responsiveness (tumor volume=0) in half of treated mice.

### Example 35. Compound 5 in HCC1954 Breast Tumor Model

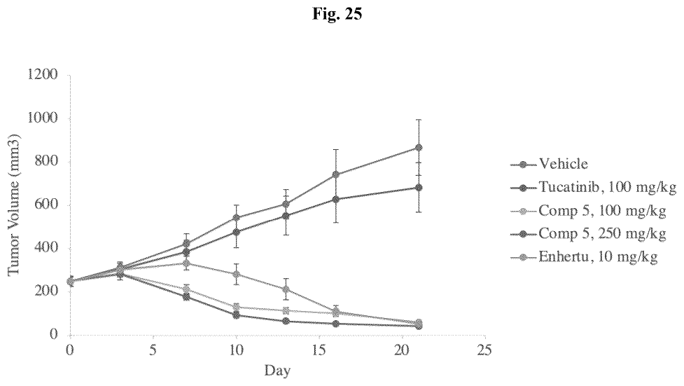

FIG. 25 illustrates that Compound 5 shows superior in vivo activity in HER2+ breast cancer resistant to tucatinib. In HCC1954 breast tumor model (Her2-amplified), the animals (N=8 per group) were dosed with compound 5 at 250 and 100 mg/kg PO daily for 21 days and caused TGI of 184% and 176%, respectively. Enhertu was dosed in a group of mice at 10 mg/kg IV once for 21 days and caused TGI of 179%. Tucatinib was dosed in a group of mice at 100 mg/kg PO daily for 21 days and caused TGI of 30%. These results show that the model is sensitive to Compound 5 treatment.

### Example 36. Compound 5 in JIMT-1 Breast Tumor Model

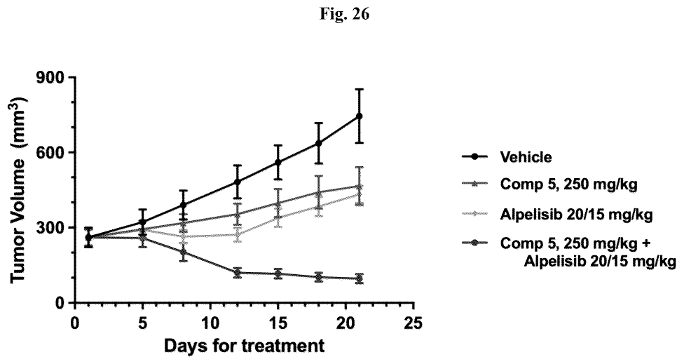

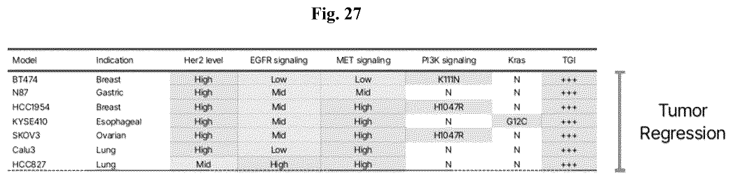

FIG. 26 illustrates that Compound 5 and a PIK3CA inhibitor have a synergistic effect on inhibiting breast cancer. In JIMT-1 breast tumor model (Her2-amplified), the animals (N=6 per group) were dosed with compound 5 at 250 mg/kg PO daily for 21 days and caused TGI of 57%. In another group of mice, dosing of alpilisib at 20/15 mg/kg PO daily for 9 (at 20 mg/kg) then 12 days (at 15 mg/kg) caused TGI of 64%. Combined dosing of compound 5 at mostly 250 mg/kg PO daily and alpilisib at 20/1.5 mg/kg PO daily for 21 days caused TGI of 164%. Overall, a strong synergy of combo with Compound 5+alpelisib was observed.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A pharmaceutical composition comprising a compound and optionally one or more pharmaceutical excipients, wherein:
the composition is in a unit dosage form; and
the compound has a structure of Formula (I): 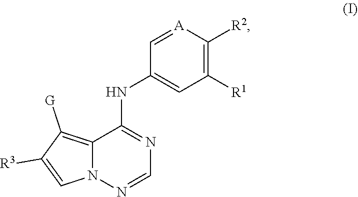
or a pharmaceutically acceptable salt thereof, wherein:
A is N or CH;
R¹ is C₁-C₄ alkyl, C₁-C₄ haloalkyl or halogen;
R² is —O-(5-10 membered) aryl, —O-(5-10 membered) heteroaryl, —O-(4-7 membered) cycloalkyl, —O-(4-7 membered) heterocycloalkyl, —O-(5-10 membered) heteroaryl-C1-C4alkylene-phenyl, —NH-(5-10 membered) aryl, or —NH—-(5-10 membered) heteroaryl, wherein each of the aryl, heteroaryl, cycloalkyl and heterocycloalkyl moieties are optionally substituted with 1-3 J¹ groups;
R³ is H or F;
G is -L¹-R³, L^{1a}-R^{3a}, or —W—X—Y;
L¹ is a bond, —C(O)—, —S(O)₂—, —N(R^{c})—, alkylene, aryl, heteroaryl, heterocycloalkyl, or cycloalkyl, wherein the alkylene, aryl, heteroaryl, heterocycloalkyl, or cycloalkyl are each optionally substituted with 1-4 J² groups, provided that when L¹ is CH₂, L¹ is not attached to carbon or nitrogen of a saturated ring;
L^{1a} is —C₀-C₆alkylene-C(O)N(H)—, —C₀-C₆alkylene-S(O)₂N(H)—;
R³ is a 4-9 membered heterocyclic ring containing at least one nitrogen ring atom, wherein R³ is optionally substituted with 1-4 J³ groups, and wherein one nitrogen atom of R³ is substituted with -L²-R; or R³ is a 7-11 membered spirocyclic group containing at least one nitrogen ring atom, wherein the 7-11 membered spirocyclic group containing at least one nitrogen ring atom is optionally substituted with 1-4 J³ groups, and wherein one nitrogen atom of the 7-11 membered spirocyclic group is substituted with -L²-R;
R^{3a} is C₁-C₆alkylene-NR^{a}R^{b} optionally substituted with 1-4 J² groups;
W is a bond, —C(O)— or —S(O)₂—;
X is aryl, heteroaryl, heterocycloalkyl, or cycloalkyl, each of which is optionally substituted with 1-4 J² groups;
Y is —C₀-C₄alkylene-N(R^{d})-L²-R, —C(O)-4-7 membered heterocycloalkyl containing at least one nitrogen atom and substituted with 1-2 oxo groups, -4-7 membered heterocycloalkyl-L²R, —C₀-C₄alkylene-1-yl-1H-pyrrole-2,5-dione, —C₀-C₄alkylene-C(H)═C(O)—NH₂, —C₀-C₄alkylene-C(H)═C(H)—C(O)—O-alkyl, —C₀-C₄alkylene-ethynylene-C(O)—O-alkyl, —C₀-C₄alkylene-C(H)═C(H)—CN, —C₀-C₄alkylene-N═C═S, —C₀-C₄-etheyny, —C₀-C₄alkylene-ethynyl, —C₀-C₄alkylene-CN, —C₀-C₄alkylene-C(H)=N—N(H)Boc, —C₀-C₄alkylene-C(O)—CH₂—Br, —C₀-C₄alkylene-CH₂—Cl, —C₀-C₄alkylene-oxiranyl, —C₀-C₄alkylene-SH, —C₀-C₄alkylene-F, and —C₀-C₄alkylene-C(H)=O, wherein the C₀-C₄alkylene moiety is optionally substituted with 1-4 groups independently selected from halogen, cycloalkyl, alkoxy alkoxyalkyl, or hydroxy;
L² is —SO₂— or —C(O)—;
R is ethenyl optionally substituted with 1-3 Q groups, ethynyl optionally substituted with Q, C₁-C₄ alkylene-NR^{a}R^{b}, —CH₂—CN, or haloalkyl wherein one halogen of haloalkyl is on the carbon atom adjacent to L²;
each Q is independently selected from the group consisting of halogen, haloalkyl, alkyl, alkene, alkyne, —NR^{a}R^{b}, —C₁-C₆alkylene-NR^{a}R^{b}, —C₁-C₆alkylene-OR^{c}, cyano, hydroxyalkyl, —C₀-C₆alkylene-C(O)OH, —C₁-C₆alkylene-C(O)O-alkyl, alkoxyalkyl, —C₀-C₄alkylene-cycloalkyl optionally substituted with 1-3 J⁴ groups, —C₀-C₄alkylene-cycloalkenyl optionally substituted with 1-3 J⁴ groups, —C₀-C₄alkylene-7-11 membered spirocyclic cycloalkyl, optionally substituted with 1-3 J⁴ groups, —C₀-C₄alkylene-7-11 membered spirocyclic heterocycloalkyl optionally substituted with 1-3 J⁴ groups, —C₀-C₄alkylene-heterocycloalkyl optionally substituted with 1-3 J⁴ groups, and —C₀-C₄alkylene-heterocycloalkenyl optionally substituted with 1-3 J⁴ groups;
or -L²-R is —C═N—OH;
each J¹ is independently selected from the group consisting of halogen, C₁-C₆alkyl, C₁-C₆haloalkyl, hydroxy, C₁-C₆hydroxyalkyl, —C₀-C₄alkylene-N(H)R^{c}, C₁-C₆alkoxy, and —C₁-C₆alkyl-C₁-C₆alkoxy;
each J² is independently selected from the group consisting of halogen, alkyl, haloalkyl, hydroxy, hydroxyalkyl, alkoxy, and alkoxyalkyl;
each J³ is attached to a carbon atom and is independently selected from the group consisting of halogen, haloalkyl, CN, alkyl, hydroxy, hydroxyalkyl, alkoxy, and alkoxyalkyl, or two of the optional 1-4 J³ groups form an oxo group or a 3-6 membered spiro group, or two of the optional 1-4 J³ groups are on different ring carbon and join to form a 1-3 carbon bridge;
each J⁴ is independently selected from the group consisting of halogen, alkyl, haloalkyl, hydroxy, hydroxyalkyl, alkoxy, alkoxyalkyl, oxo, and —C₀-C₄alkylene-NR^{a}R^{b}, provided that J⁴ groups can only include up to two oxo groups and up to one —C₀-C₄alkylene-NR^{a}R^{b} group;
R^{a} and R^{b} each are independently selected from the group consisting of H, alkyl, alkenyl, alkynyl, haloalkyl, hydroxyalkyl, alkoxyalkyl, and —C₀-C₃alkylene-alkynyl optionally substituted with alkyl, alkenyl, alkynyl, haloalkyl, hydroxyalkyl, or alkoxyalkyl;
R^{c} is selected from the group consisting of H, alkyl, haloalkyl, hydroxyalkyl, alkoxyalkyl, cycloalkyl, heterocycloalkyl, aryl, and heteroaryl, wherein the cycloalkyl, heterocycloalkyl, aryl, and heteroaryl groups are each optionally substituted with 1-3 groups selected from the group consisting of halogen, alkyl, alkoxy and alkoxyalkyl; and
R^{d} is selected from the group consisting of H, alkyl, and haloalkyl,
wherein the compound of Formula (I) is in an amount that ranges from about 50 mg to 2160 mg within the unit dosage form.

2. The pharmaceutical composition of claim 1, wherein:
G is -L¹-R³;
L¹ is a bond;
R³ is a 4-9 membered heterocyclic ring containing at least one nitrogen ring atom, wherein R³ is optionally substituted with 1-4 J³ groups, and wherein one nitrogen atom of R³ is substituted with -L²-R;
L² is —C(O)—;
R is ethenyl substituted with 1 Q group;
Q is —C₁-C₆alkylene-NR^{a}R^{b};
and
R^{a} and R^{b} are alkyl.

3. The pharmaceutical composition of claim 1, wherein:
A is CH;
R¹ is C₁-C₃ alkyl;
R² is —O-(5-10 membered) heteroaryl;
G is -L¹-R³;
L¹ is a bond;
R³ is a 5-6 membered heterocyclic ring containing at least one nitrogen ring atom, wherein one nitrogen atom of R³ is substituted with -L²-R;
L² is —C(O)—;
R is ethenyl substituted with 1 Q group;
Q is —C₁-C₃alkylene-NR^{a}R^{b}; and
R^{a} and R^{b} are methyl.

4. The pharmaceutical composition of claim 1, wherein the compound is: 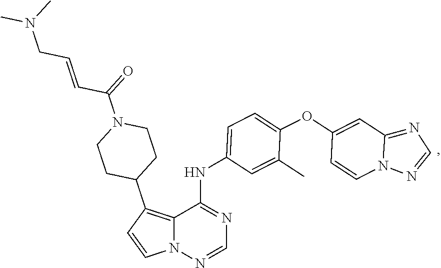 [SMILES: Cc1cc(Nc2ncnn3ccc(C4CCN(C(=O)/C=C/CN(C)C)CC4)c23)ccc1Oc1ccn2ncnc2c1]
or a pharmaceutically acceptable salt thereof.

5. The pharmaceutical composition of claim 1, wherein the compound ranges from about 120 mg to about 600 mg per unit dose, 120 mg to about 720 mg per unit dose, 120 mg to about 840 mg per unit dose, 120 mg to about 960 mg per unit dose, 120 mg to about 1080 mg per unit dose, 120 mg to about 1200 mg per unit dose, 120 mg to about 1320 mg per unit dose, 120 mg to about 1440 mg per unit dose, 120 mg to about 1560 mg per unit dose, 120 mg to about 1680 mg per unit dose, 120 mg to about 1800 mg per unit dose, 120 mg to about 1920 mg per unit dose, or 120 mg to about 2160 mg per unit dose.

6. The pharmaceutical composition of claim 1, wherein the compound ranges from about 480 mg to about 1560 mg per unit dose.

7. The pharmaceutical composition of claim 1, wherein the compound ranges from about 960 mg to about 1560 mg per unit dose.

8. The pharmaceutical composition of claim 1, wherein the unit dosage of the compound is about 60 mg, about 120 mg, about 180 mg, about 240 mg, about 360 mg, about 480 mg, about 600 mg, about 720 mg, about 780 mg, about 840 mg, about 960 mg, about 1080 mg, about 1200 mg, about 1320 mg, about 1440 mg, about 1560 mg, about 1680 mg, about 1800 mg, about 1920 mg, or about 2160 mg.

9. The pharmaceutical composition of claim 1, wherein the unit dosage of the compound is about 240 mg.

10. The pharmaceutical composition of claim 1, wherein the unit dosage of the compound is about 360 mg.

11. The pharmaceutical composition of claim 1, wherein the unit dosage of the compound is about 480 mg.

12. The pharmaceutical composition of claim 1, wherein the unit dosage of the compound is about 600 mg.

13. The pharmaceutical composition of claim 1, wherein the unit dosage of the compound is about 720 mg.

14. The pharmaceutical composition of claim 1, wherein the unit dosage of the compound is about 780 mg.

15. The pharmaceutical composition of claim 1, wherein the unit dosage of the compound is about 840 mg.

16. The pharmaceutical composition of claim 1, wherein the unit dosage of the compound is about 960 mg.

17. The pharmaceutical composition of claim 1, wherein the unit dosage of the compound is about 1080 mg.

18. The pharmaceutical composition of claim 1, wherein the unit dosage is in capsule or tablet form.

19. A method for treating cancer modulated by HER2 in a subject, the method comprising administering to the subject an effective amount of the pharmaceutical composition of claim 1.

20. A method of treating cancer modulated by HER2 in a subject, comprising administering to the subject:
(a) a compound that binds to HER2 in a type II DFG-out conformation having Formula (II): 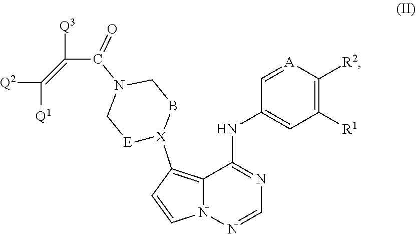
or a pharmaceutically acceptable salt thereof, wherein:
A is CH or N;
B is CH₂;
E is CH₂;
X is CH, CF, C(OH) or N, or X is CH and B and E are both absent;
Q¹ is selected from the group consisting of H, and —C₁-C₆alkyl;
Q² is selected from the group consisting of H, —C₁-C₆alkylene-NR^{a}R^{b};
Q³ is H or F;
R^{a} and R^{b} each are independently selected from the group consisting of H, C₁-C₆alkyl, C₁-C₆haloalkyl, C₁-C₆hydroxyalkyl, and —C₁-C₆alkyl-C₁-C₆alkoxy, provided that at least one of R^{a} or R^{b} is not H;
R¹ is alkyl, haloalkyl or halogen; and
R² is 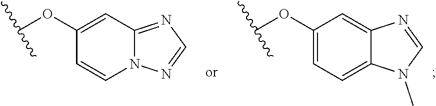 [SMILES: *Oc1ccn2ncnc2c1 ; *Oc1ccc2c(c1)ncn2C]
and (b) optionally one or more additional therapeutic agents.
